(19) United States
(12) Patent Application Publication
(10) Pub. No.: US 2024/0116919 A1
(43) Pub. Date: Apr. 11, 2024

(54) ORGANIC COMPOUND AND ORGANIC ELECTROLUMINESCENT DEVICE USING SAME

(71) Applicant: SOLUS ADVANCED MATERIALS CO., LTD., Jeollabuk-do (KR)

(72) Inventors: Hojun SON, Yongin-si, Gyeonggi-do (KR); Minsik EUM, Yongin-si, Gyeonggi-do (KR); Hoemoon KIM, Yongin-si, Gyeonggi-do (KR); Hwasoon JUNG, Yongin-si, Gyeonggi-do (KR); Hyungchan BAE, Yongin-si, Gyeonggi-do (KR); Jin-woong KIM, Yongin-si, Gyeonggi-do (KR); Gahyeon KIM, Yongin-si, Gyeonggi-do (KR)

(73) Assignee: SOLUS ADVANCED MATERIALS CO., LTD., Jeollabuk-do (KR)

(21) Appl. No.: 18/014,001

(22) PCT Filed: Jul. 2, 2021
(86) PCT No.: PCT/KR2021/008429

§ 371 Date: Jun. 12, 2023

(30) Foreign Application Priority Data
Jul. 2, 2020 (KR) 10-2020-0081744

(51) Int. Cl. C07D 471/04; H10K 50/11; H10K 50/16; H10K 85/60

(52) U.S. Cl. CPC C07D 471/04; H10K 50/11; H10K 50/16; H10K 85/6572; C09K 11/06

(57) ABSTRACT

The present invention relates to a novel organic compound and an organic electroluminescent device using same and, more specifically, to a compound having excellent electron injection and transport capabilities, and an organic electroluminescent device of which properties such as luminous efficiency, driving voltage, lifespan, and the like, and progressive driving voltage are improved due to the inclusion of the same in at least one organic layer.

### TECHNICAL FIELD

The present disclosure relates to a novel organic compound and an organic electroluminescent device using same and, more specifically, to a compound having excellent electron injection and transport potentials, and an organic electroluminescent device that includes same in at least one organic layer and thus exhibits an improvement in luminous efficiency, driving voltage, and lifespan as well as progressive driving voltage.

### BACKGROUND ART

In the structure of an organic electroluminescence device (hereinafter referred to as “EL device”), application of a voltage between the two electrodes injects holes from the anode and electrons from the cathode into the organic layer. When the injected holes and electrons are combined with each other, excitons are generated and then return to a ground state, emitting light. Materials used in the organic layer may be classified into a light emitting material, a hole injection material, a hole transport material, an electron transport material, an electron injection material, etc. according to their functions.

Also, the material used in the light-emitting layer of the organic EL device can be divided into blue, green, and red light-emitting materials. In addition, yellow and orange light-emitting materials may be used for implementing colors closer to natural colors. Furthermore, a host/dopant system may be used as a light-emitting material in order to increase the color purity and enhance the light-emitting efficiency through energy transfer. Dopant materials may be divided into phosphorescent dopants accounted for by organic materials and phosphorescent dopants accounted for by metal complex compounds bearing heavy atoms such as Ir and Pt. The development of such a phosphorescent material can theoretically improve luminous efficiency up to four times, compared to fluorescence. Thus, attention has been focused on phosphorescent host materials as well as phosphorescent dopants.

Until now, NPB, BCP, Alq₃, and the like represented by the following chemical formulas are widely known for use in hole injection, hole transport, hole block, and electron transport layers, and anthracene derivatives have been reported as fluorescent dopant/host materials in the light-emitting layer. With respect to phosphorescent materials, which are advantageous in terms of luminous efficiency over other luminescent materials, Ir-bearing metal complex compounds, such as Firpic, Ir(ppy)₃, (acac)Ir(btp)₂, etc., are used as blue, green, and red dopant materials. So far, CBP has shown excellent properties as a phosphorescent host material.

However, conventional materials are advantageous in terms of emission properties, but have low glass transition temperatures and very poor thermal stability, so they are not satisfactory in terms of lifespan for organic EL devices.

### DISCLOSURE OF INVENTION

### Technical Problem

The present disclosure aims to provide a novel organic compound that is superb in terms of all of electron injection and transport potential, electrochemical stability, and thermal stability and can be used as an organic layer material in an organic EL device, specifically as an electron transport layer material or an N-type charge generation layer material.

Also, the present disclosure aims to provide an organic EL device including the aforementioned novel organic composition, which exhibits a low driving voltage and high luminous efficiency and has an improved lifespan.

### Solution to Problem

In order to achieve the aims, the present disclosure provides an organic compound represented by the following Chemical Formula 1:

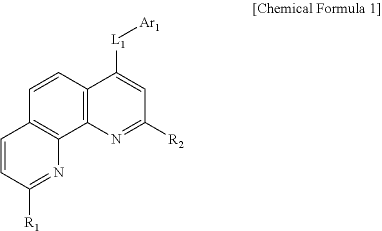

(wherein,

R₁ and R₂, which are same or different, are each independently selected from the group consisting of a hydrogen atom, a deuterium atom (D), an alkyl group of C₁-C₆₀, a cycloalkyl group of C₃-C₆₀, and a heteroaryl group having 5 to 60 nuclear atoms, wherein a case where both of R₁ and R₂ are a hydrogen atom is excluded,

L₁ is selected from the group consisting of a single bond, an arylene group of C₆-C₆₀, and a heteroarylene group having 5 to 60 nuclear atoms, and

Ar₁ is selected from the group consisting of an aryl group of C₆-C₆₀, —P(═O)(R₃)(R₄), and —Si(R₅)(R₆)(R₇),

wherein R₃ to R₇, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀,

the alkyl group, the cycloalkyl group, and the heteroalkyl group in R₁ and R₂, the arylene group and the heteroarylene group in L₁, the arylene group in Ar₁, and the hydrazino group, the hydrazono group, the alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and the arylamine group in R₃ to R₇ are each independently substituted or unsubstituted with one or more substituents of: a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other).

Also, the present disclosure provides an organic electroluminescent device including: an anode; a cathode, and at least one organic layer disposed between the anode and the cathode, wherein the at least one organic layer includes the organic compound described above. In this regard, the organic layer including the compound may be an electron transport layer.

Furthermore, the present disclosure provides an organic electroluminescent device including an anode and a cathode spaced apart from each other; a plurality of light-emitting units interposed between the anode and the cathode; and an N- and a P-type charge generation layer disposed between adjacent light-emitting units, wherein each light-emitting unit includes a hole transport layer, a light-emitting layer, and an electron transport layer and the N-type charge generation layer includes the compound described above.

### Advantageous Effects of Invention

With excellency in electron transport and emission potentials, electrochemical stability, and thermal stability, the compound of the present disclosure can be used as a material for an organic layer in an organic EL device. Particularly, when used as a material for any one of an electron transport layer, an electron transport auxiliary layer, and an N-type charge generation layer, the compound of the present disclosure allows for the fabrication of an organic EL device that superb emission performance, low driving voltage, high luminous efficiency, and prolonged lifespan characteristics, compared to conventional materials.

### BRIEF DESCRIPTION OF DRAWINGS

FIG. 1 is a schematic cross-sectional view of an organic EL device according to a first embodiment of the present disclosure.

FIG. 2 is a schematic cross-sectional view of an organic EL device according to a second embodiment of the present disclosure.

FIG. 3 is a schematic cross-sectional view of an organic EL device according to a third embodiment of the present disclosure.

FIG. 4 is a schematic cross-sectional view of an organic EL device according to a fourth embodiment of the present disclosure.

### DESCRIPTION OF REFERENCE NUMERALS

100: anode, 200: cathode,
300: organic layer, 310: hole injection layer,
320: hole transport layer, 330: light-emitting layer,
340: electron transport layer, 350: electron injection layer,
360: electron transport auxiliary layer, 400: first light-emitting unit, 410: first hole transport layer, 420: first light-emitting layer,
430: first electron transport layer, 440: hole injection layer,
500: second light-emitting unit, 510: second hole transport layer,
520: second light-emitting layer, 530: second electron transport layer,
600: charge generation layer, 610: N-type charge generation layer,
620: P-type charge generation layer

### BEST MODE FOR CARRYING OUT THE INVENTION

Below, a detailed description will be given of the present disclosure.

<Novel Compound>

The present disclosure provides a novel compound that is superb in terms of electron injection and transport potential, electrochemical stability, and thermal stability and can be used as an electron transport auxiliary layer material or an N-type charge generation layer material that can improve luminous efficiency, lifespan, driving voltage, and progressive driving voltage characteristics in an organic EL device.

Specifically, the compound represented by Chemical Formula 1 has the phenanthroline moiety-based structure in which an alkyl, a cycloalkyl, etc. are introduced into the carbon atoms at positions 2 and 9 and an aryl group or a phosphine oxide is introduced directly or via a linker (e.g., phenylene, biphenylene, or terphenylene) into the carbon atom at position 4. Here, numbering for the carbon/nitrogen atoms in the phenanthroline moiety is as follows:

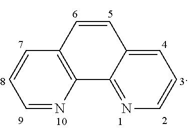 [SMILES: c1cnc2c(c1)ccc1cccnc12]

In the compound of the present disclosure, the phenanthroline moiety bears nitrogen atoms (N) of the sp2 hybrid orbital relatively rich in electron. Specifically, structured to bear two adjacent nitrogen atoms, the phenanthroline moiety can form a covalent bond with a neighboring hydrogen atom (H) or a coordination bond with an alkali metal or alkaline earth metal, such as Li or Yb. When the phenanthroline moiety-based compound of Chemical Formula 1 is applied to an electron transport layer or an N-type charge generation layer, the phenanthroline moiety traps an alkali metal or alkaline earth metal dope therein to increase the intramolecular electron density, thereby enhancing electron injection and transport potentials. For example, when the compound of the present disclosure is applied to an N-type charge generation layer in an OLED, the nitrogen atoms of the phenanthroline moiety may bind to the dopant alkali metal or alkaline earth metal in the N-type charge generation layer to form a gap state. Specifically, even though used as a host material alone without being mixed with a different host material, the compound of the present disclosure can smoothly transport electrons from the N-type charge generation layer to the electron transport layer due to the gap state. In addition, even when applied to the electron transport layer in an OLED, the compound of the present disclosure can smoothly transport electron toward the light-emitting layer. Therefore, the use of the compound of the present disclosure as a material for an N-type charge generation layer or an electron transport layer allows the organic EL device to decrease in driving voltage, increase in luminous efficiency, and enjoy a prolonged lifespan.

Moreover, having high electron absorptivity, the phenanthroline moiety of the compound can serve as an electron withdrawing group (EWG). In the phenanthroline moiety, an aryl group, phosphine oxide, or silyl is introduced into the carbon atom at position 4 directly or via a linker. Particularly, the compound of the present disclosure having an aryl group introduced into the carbon atom at position 4 exhibits increased luminous efficiency and a decreased driving voltage while retaining the intrinsic LUMO (lowest unoccupied molecular orbital) energy level of the phenanthroline derivative. Therefore, the application of the compound of the present disclosure to an EL device guarantees low deriving voltage, high current efficiency, and prolonged lifespan characteristics and enhances a progressive deriving voltage characteristic, thus preventing the device from increasing in consumption power and decreasing in lifespan.

With the introduction of the substituents such as an alkyl, a cycloalkyl, etc., into the carbon atoms at the active sites positions 2 and 9, the phenanthroline moiety can be increased in thermal stability due to the blockage of the active sites. However, the compound having an aryl group introduced into position 2 and/or 9 of the phenanthroline moiety increases in sublimation temperature because its large molecular weight. In this regard, an excess of high heat for sublimating the compound upon the fabrication of an organic EL device may damage the device. Thus, it is preferred that an alkyl or cycloalkyl, particularly a short alkyl or cycloalkyl, rather than an aryl group, is introduced into the carbon atom at position 2 and/or 9 in the phenanthroline moiety. Such compounds of the present disclosure have the active sites blocked therein through a minimal increase of molecular weight and thus can increase in thermal stability without deteriorating the device. In addition, the compound of the present disclosure is lower in sublimation temperature than compounds having aryl group introduced into the carbon atoms at positions 2 and 9 in the phenanthroline moiety. Hence, the compounds of the present disclosure can prevent the deterioration of the devices upon the fabrication thereof while increasing in thermal stability.

As described above, the compound represented by Chemical Formula 1 according to the present disclosure is superb in terms of electron injection and transport potential. Thus, the compounds of the present disclosure can be used as a material for an organic layer, preferably for an electron transport layer in an organic EL device. In addition, the compound of the present disclosure may be used as a material for an N-type charge generation layer in tandem organic EL devices. As such, the compound of the present disclosure, represented by Chemical Formula 1, when applied as a material for an electron transport layer or N-type charge generation layer in an organic EL device, can improve deriving voltage, luminous efficiency, and lifespan characteristics in the organic EL device and prevent the increase of a progressive driving voltage, and furthermore the performance of a full-color organic light-emitting panel to which the organic EL device is applied can be maximized.

In the compound represented by Chemical Formula 1, R₁ and R₂ are same or different and are each independently selected from the group consisting of a hydrogen atom, a deuterium atom (D), an alkyl group of C₁-C₆₀, a cycloalkyl group of C₃-C₆₀, and a heteroaryl group having 5 to 60 nuclear atoms, with a proviso that both of R₁ and R₂ are a hydrogen atom is excluded. In contrast to the compound wherein both of R₁ and R₂ are a hydrogen atom, the compound of Chemical Formula 1 exhibits improved thermal stability as either or both of the active sites of the phenanthroline moiety are blocked. In addition, the compound of Chemical Formula 1, unlike the compound wherein R₁ and R₂ are both aryl groups, can improve in thermal stability without deteriorating the device because the molecular weight is minimally increased, along with the blockage of the active sites.

In an embodiment, at least one of R₁ and R₂ may be an alkyl group of C₁-C₆₀ or a cycloalkyl group of C₃-C₆₀.

In another embodiment, R₁ and R₂, which are same or different, may each be independently selected from the group consisting of a hydrogen atom, an alkyl group of C₁-C₂₀, and a cycloalkyl group of C₃-C₂₀, with a proviso that at least one of R₁ and R₂ may be an alkyl group of C₁-C₂₀ or a cycloalkyl group of C₃-C₂₀.

In a further embodiment, R₁ and R₂, which are same or different, may each be selected from the group consisting of a hydrogen atom, an alkyl group of C₁-C₆, and a cycloalkyl group of C₃-C₆, with a proviso that at least one of R₁ and R₂ may be an alkyl group of C₁-C₆ or a cycloalkyl group of C₃-C₆. For example, R₁ and R₂ may each be independently selected from the group consisting of methyl group, ethyl group, propyl group, isopropyl group, n-butyl group, s-butyl group, isobutyl group, t-butyl group, and cyclohexyl group.

The alkyl group, the cycloalkyl group, the and the heteroaryl group of R₁ and R₂ may remain unsubstituted or may each independently be substituted with at least one substituent selected from the group consisting of a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other. Here, the heterocycloalkyl and the heteroaryl each bear at least one heteroatom selected from the group consisting of N, S, O, and Se.

According to R₁ and R₂, the compound represented by Chemical Formula 1 may be a compound represented by any one of the following Chemical Formulas 2 to 6, but with no limitations thereto:

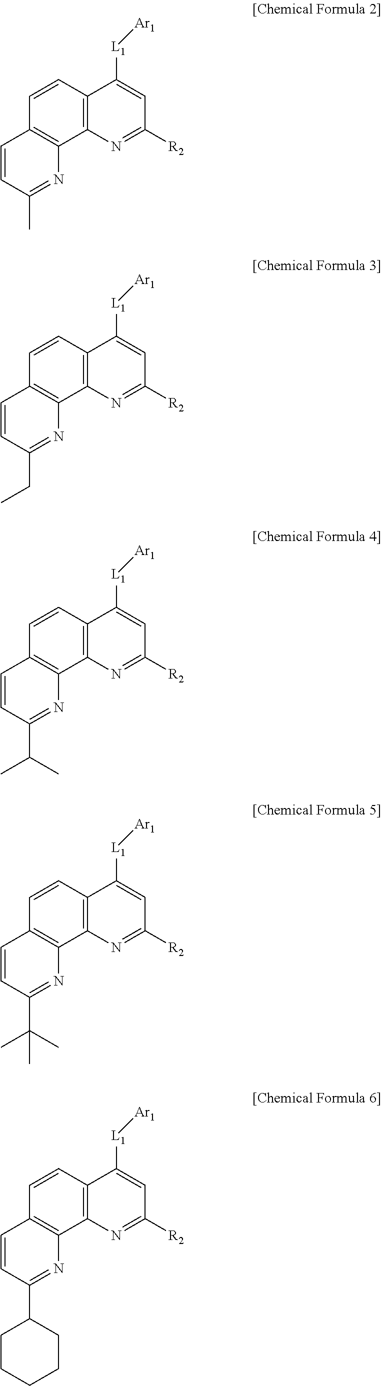

wherein,

L₁ and Ar₁ are each as defined in Chemical Formula 1, and

R₂ is an alkyl group of C₁-C₆ or a cycloalkyl group of C₃-C₆, and specifically may be selected from the group consisting of methyl group, ethyl group, propyl group, isopropyl group, n-butyl group, s-butyl group, isobutyl group, t-butyl group, and cyclohexyl group. In this regard, R₂ may be identical to or different from the substituents of Chemical Formula 2-6, which correspond to R₁ in Chemical Formula 1.

According to an embodiment, R₂ in the compounds of Chemical Formulas 2 to 6 may be an alkyl group of C₁-C₆ or a cycloalkyl group of C₃-C₆ and specifically, may be selected from the group consisting of methyl group, ethyl group, propyl group, isopropyl group, n-butyl group, s-butyl group, isobutyl group, t-butyl group, and cyclohexyl group. In this regard, R₂ may be identical to the substituents of Chemical Formula 2-6, which correspond to R₁ in Chemical Formula 1.

In Chemical Formula 1, L₁ is selected from the group consisting of a single bond, an arylene group of C₆-C₆₀, and a heteroarylene group having 5 to 60 nuclear atoms and specifically may be a single bond or may be selected from the group consisting of an arylene group of C₆-C₃₀ and a heteroarylene group having 5 to 30 nuclear atoms.

The arylene group and the heteroarylene group of L₁ may remain unsubstituted or may each independently be substituted with at least one substituent selected from the group consisting of a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other.

In an embodiment, L₁ may be an arylene group of C₆-C₆₀ or an N-bearing heteroarylene group having 5 to 60 nuclear atoms.

In another embodiment, L₁ may be a linker represented by the following Chemical Formula L:

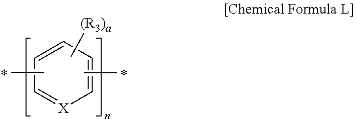

wherein,

n is an integer of 0 to 3,

a is an integer of 0 to 4,

X is C or N,

the plurality of R₃ are the same as or different from each other, and

R₃ or two or more R₃'s, if present, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀.

In another embodiment, L₁ may be selected from the group consisting of the following linkers L1 to L8. In this regard, the phenanthroline moiety and the substituent Ar₁ in the compound of the present disclosure are bonded at para or meta positions or at para-para or meta-meta positions to the linker. With such a framework, the compound of the present discloses forms a plate-like structure and induces stacking between molecules, thereby increasing in electron mobility and thus having better electron transport properties. In addition, the compound of the present disclosure can significantly increase in physical, electrochemical, and thermal stability because the compound has minimal interaction between the phenanthroline moiety and the substituent Ar₁, increased molecular structural stability, and minimal intramolecular steric hindrance. Moreover, compared to compounds in which the phenanthroline moiety and the substituent Ar₁ are bonded at the ortho positions or at the ortho-ortho position, the compound of the present disclosure is effective for suppressing the crystallization of the organic layer and thus can greatly enhance the durability and lifespan characteristics of the organic EL device.

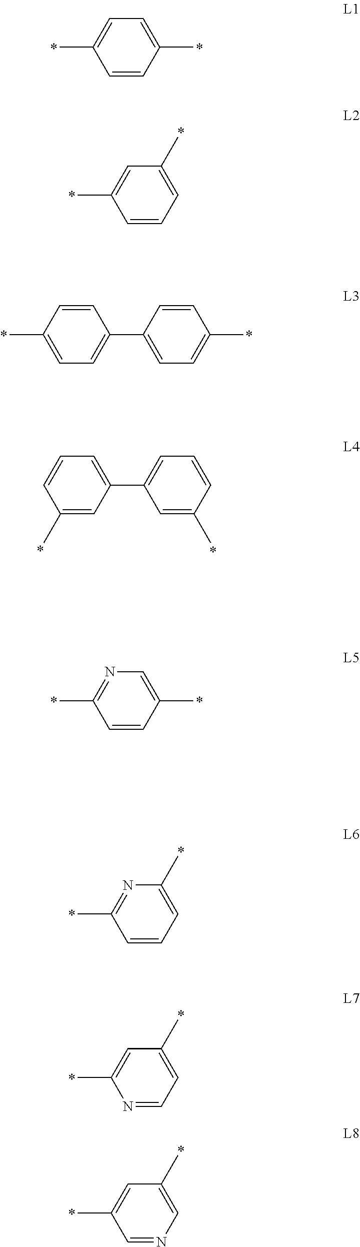

Hydrogen atoms on the linkers L1 to L8 may be replaced by at least one substituent such as a deuterium atom (D), a halogen group, a cyano group, a nitro group, an alkyl group of C₁-C₁₂, an aryl group of C₆-C₁₀, a heteroaryl group having 5 to 9 nuclear atoms, etc.

In Chemical Formula 1, Ar₁ is selected from the group consisting of an aryl group of C₆-C₆₀, —P(═O)(R₃)(R₄), and —Si(R₅)(R₆) R₇) and specifically, may be selected from the group consisting of an aryl group of C₆-C₃₀, —P(═O)(R₃)(R₄), and —Si(R₅)(R₆)(R₇).

In an embodiment, Ar₁ may be an aryl group of C₆-C₆₀ and particularly an aryl group of C₆-C₃₀. In this case, the compound can improve in luminous efficiency and decrease in driving voltage while retaining the intrinsic LUMO (lowest unoccupied molecular orbital) energy level of the phenanthroline derivative itself.

R₃ to R₇, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀. Specifically, R₃ to R₇, which are same or different, may each be independently selected from the group consisting of a deuterium atom, a halogen group, a hydroxy group, a cyano group, a nitro group, an amino group, an amidino group, a hydrazino group, a hydrazono group, an alkyl group of C₁-C₂₀, an alkynyl group of C₂-C₂₀, a cycloalkyl group of C₃-C₂₀, a heterocycloalkyl group having 3 to 30 nuclear atoms, a cycloalkenyl group of C₃-C₂₀, a heterocycloalkenyl group having 3 to 30 nuclear atoms, an aryl group of C₆-C₃₀, a heteroaryl group having 5 to 30 nuclear atoms, an alkyloxy group of C₁-C₃₀, and an aryloxy group of C₆-C₃₀.

In an embodiment, R₃ to R₇, which are same or different, may each be independently an aryl group of C₆-C₃₀, and specifically an aryl group of C₆-C₃₀. Examples of the aryl include phenyl group, biphenyl group, terphenyl group, naphthyl group, phenanthryl group, anthryl group, naphthacenyl group, pyrenyl group, and chrysenyl group, but are not limited thereto.

The aryl group of Ar₁ and the hydrazino group, the hydrazone group, the alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and the arylamine group of R₃ to R₇ may each be independently unsubstituted, or substituted with a substituent selected from the group consisting of a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other.

In detail, Ar₁ may be a substituent represented by any one selected from the group consisting of the following Chemical Formulas S1 to S8, but with no limitations thereto:

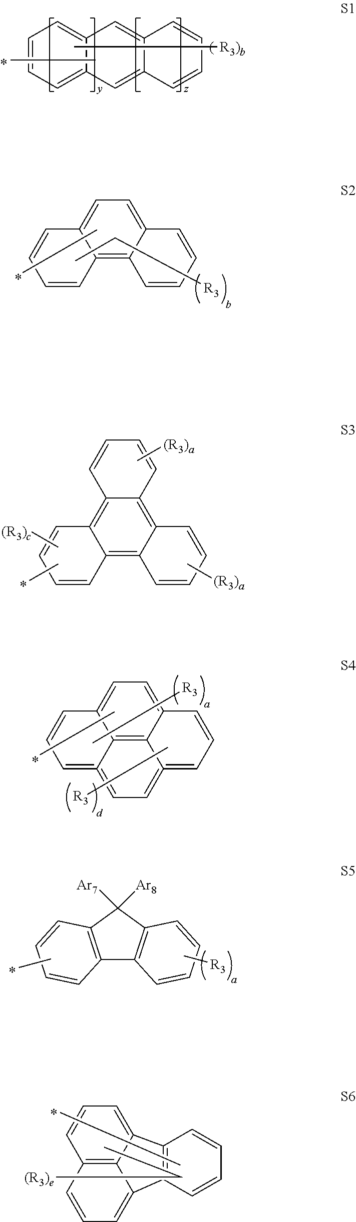

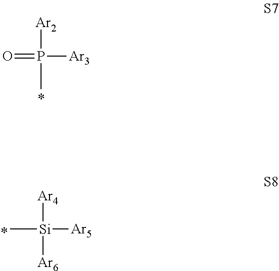

wherein,
a is an integer of 0 to 4,
b is an integer of 0 to 9,
c is an integer of 0 to 3,
d is an integer of 0 to 5,
e is an integer of 0 to 9,
the plurality of R₃ are the same as or different from each other,
R₃ or two or more R₃'s, if present, are each independently selected from the group consisting of a hydrogen atom, a deuterium atom, a halogen group, a hydroxy group, a cyano group, a nitro group, an amino group, an amidino group, an hydrazine, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀,
y and z are each 0 or 1,
Ar₂ to Ar₈, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆n, and an arylamine group of C₆-C₆n, or may be fused to an adjacent group (e.g., Ar₇ and Ar₈) to form a fuse ring (e.g., a fused aliphatic ring of C₃-C₄₀, a fused aromatic ring of C₆-C₆₀, a fused heteroaliphatic ring of 3 to 60 nuclear atoms, a fused heteroaromatic ring of 5 to 60 nuclear atoms, or a combination thereof),
the hydrazino group, the hydrazono group, the alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and the arylamine group of Ar₂ to Ar₈ are each independently substituted or unsubstituted with one or more substituents of: a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other.

More specifically, Ar₁ may be a substituent represented by any one selected from the group consisting of the following Chemical Formulas S-a1 to S-a22, but with no limitations thereto:

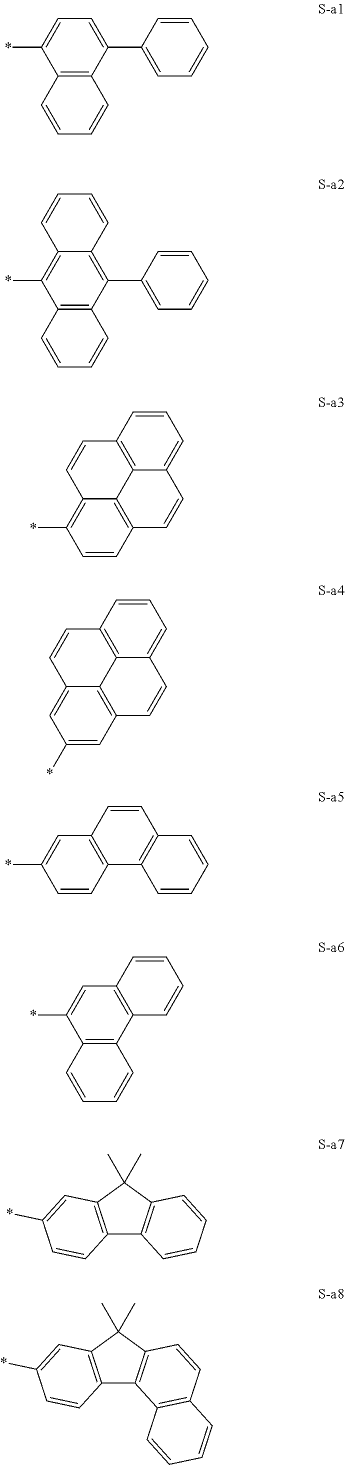

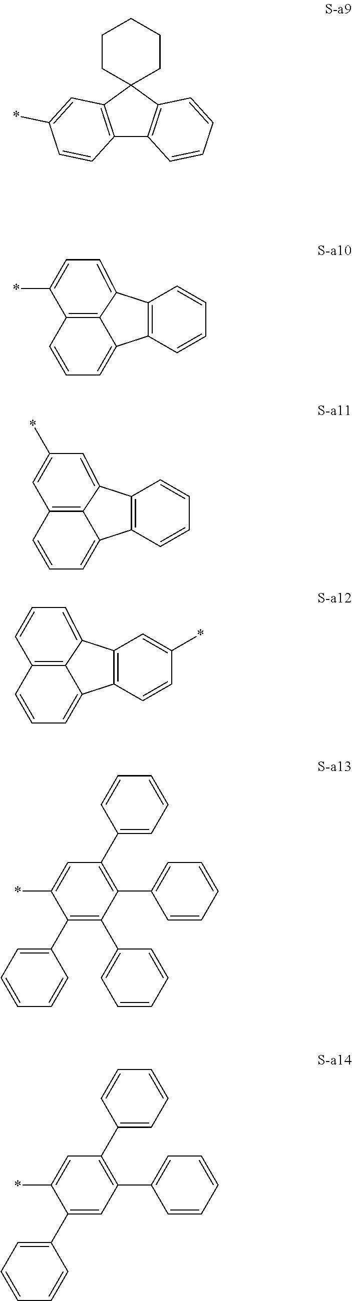

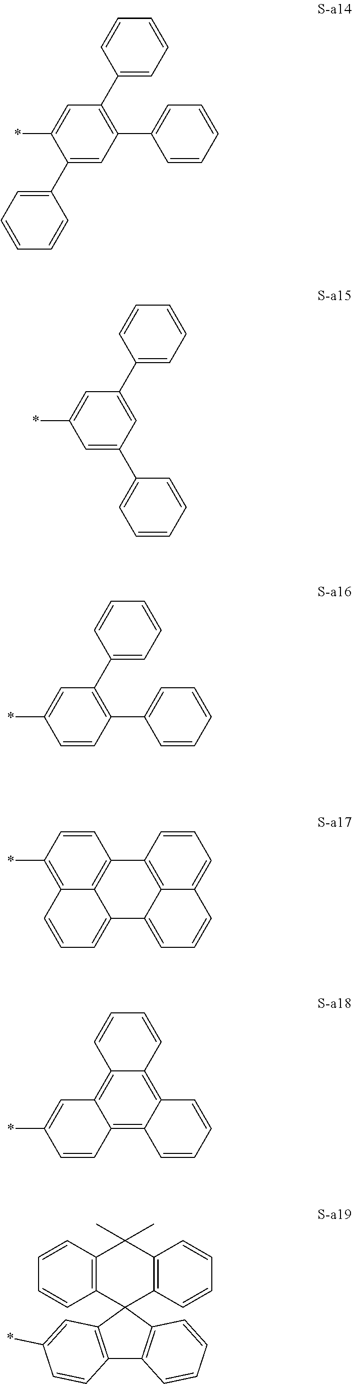 [SMILES: *c1cc(-c2ccccc2)c(-c2ccccc2)cc1-c1ccccc1 ; *c1cc(-c2ccccc2)cc(-c2ccccc2)c1 ; *c1ccc(-c2ccccc2)c(-c2ccccc2)c1 ; *c1ccc2c3cccc4cccc(c5cccc1c52)c43 ; *c1ccc2c3ccccc3c3ccccc3c2c1 ; *c1ccc2c(c1)C1(c3ccccc3-2)c2ccccc2C(C)(C)c2ccccc21]

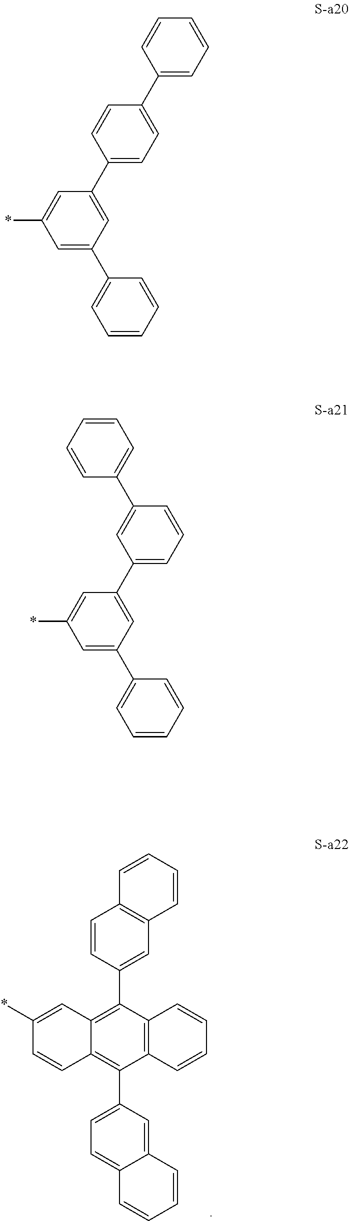 [SMILES: *c1cc(-c2ccccc2)cc(-c2ccc(-c3ccccc3)cc2)c1 ; *c1cc(-c2ccccc2)cc(-c2cccc(-c3ccccc3)c2)c1 ; *c1ccc2c(-c3ccc4ccccc4c3)c3ccccc3c(-c3ccc4ccccc4c3)c2c1]

The compound according to the present disclosure, represented by Chemical Formula 1, may be embodied into a compound represented by any one of the following Chemical Formulas 7 to 12, but with no limitations thereto:

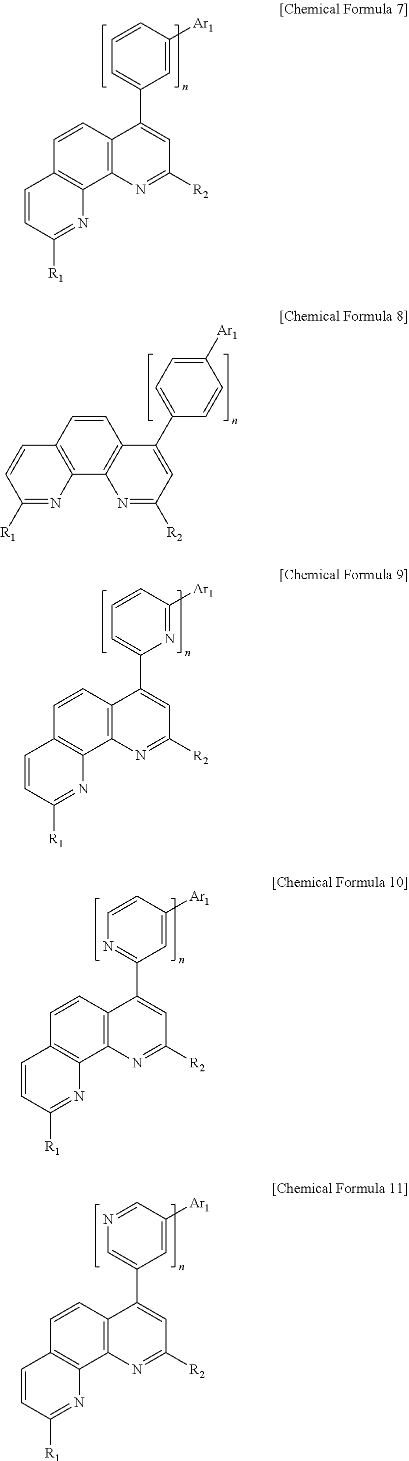

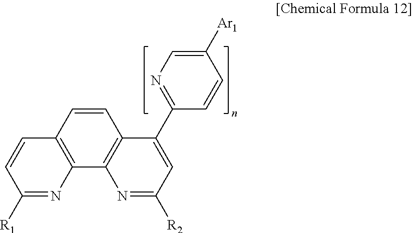

wherein,

R₁ and R₂, which are same or different, are each independently an alkyl group of C₁-C₆ or a cycloalkyl group of C₃-C₆,

n is 0 or 1, and

Ar₁ is selected from the group consisting of the following substituents S1 to S8,

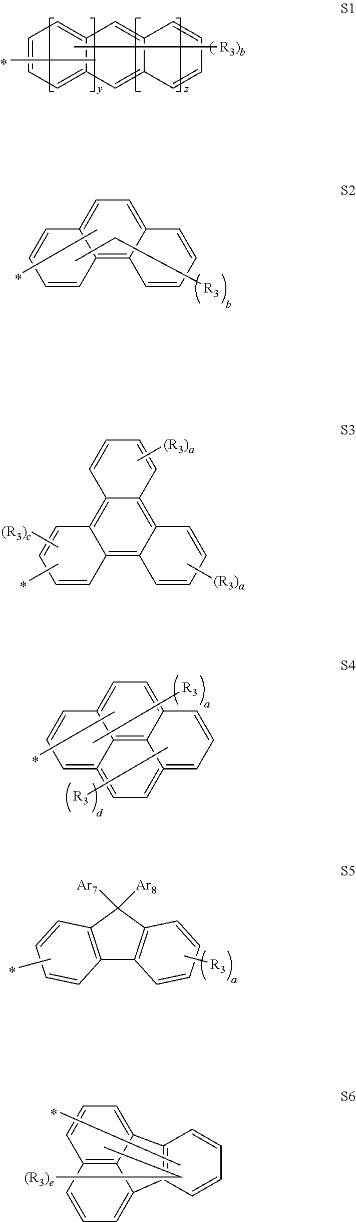

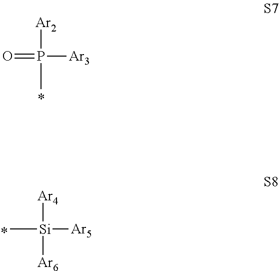

wherein,

a is an integer of 0 to 4,

b is an integer of 0 to 9,

c is an integer of 0 to 3,

d is an integer of 0 to 5,

e is an integer of 0 to 9,

the plurality of R₃ are the same as or different from each other,

R₃ or two or more R₃'s, if present, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀,

y and z are each 0 or 1,

Ar₂ to Ar₈, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, or may be fused to an adjacent group to form a fuse ring, and

the hydrazino group, the hydrazono group, the alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and arylamine group of Ar₂ to Ar₈ are each independently substituted or unsubstituted with one or more substituents of: a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, wherein two or more substituents, if present, are same or different.

The compound according to the present disclosure, represented by Chemical Formula 1, may be embodied into any one of the following Compounds 1 to 186. However, the compound according to the present disclosure, represented by Chemical Formula 1, is not limited thereto.

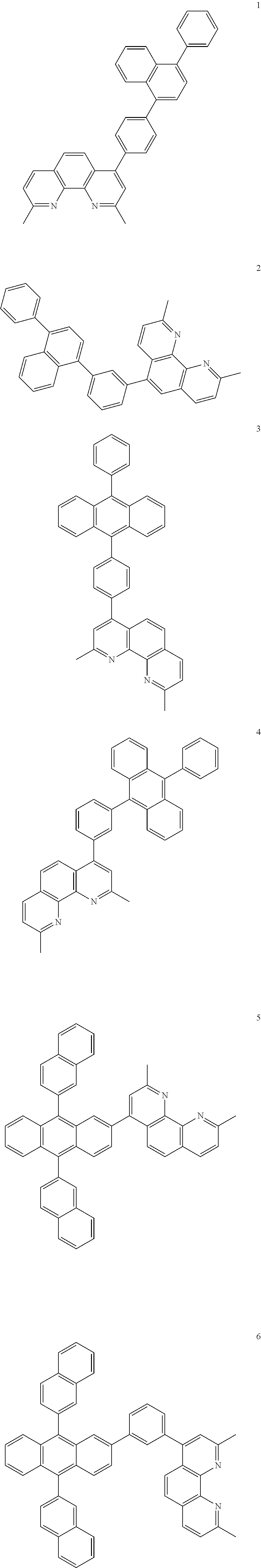

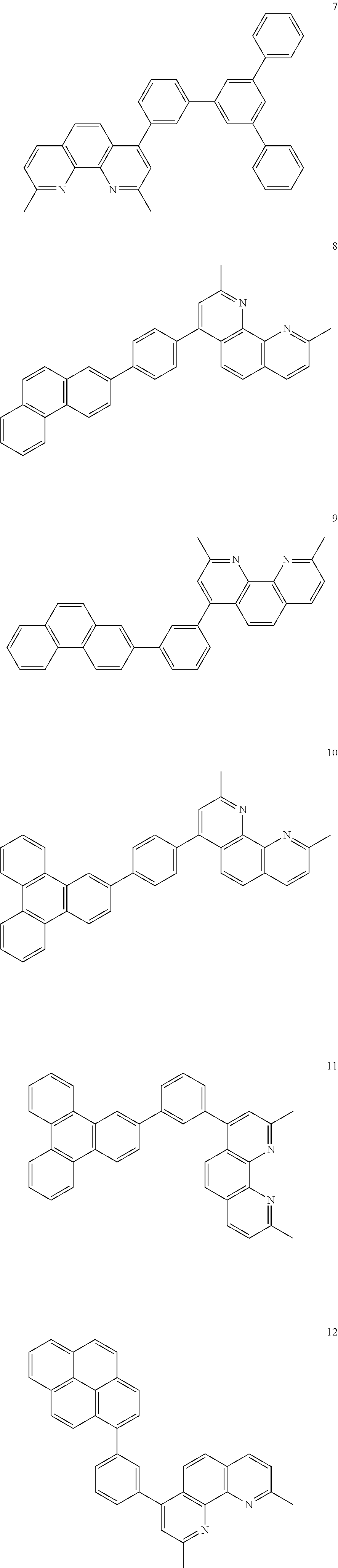 [SMILES: Cc1ccc2ccc3c(-c4cccc(-c5cc(-c6ccccc6)cc(-c6ccccc6)c5)c4)cc(C)nc3c2n1 ; Cc1ccc2ccc3c(-c4ccc(-c5ccc6c(ccc7ccccc76)c5)cc4)cc(C)nc3c2n1 ; Cc1ccc2ccc3c(-c4cccc(-c5ccc6c(ccc7ccccc76)c5)c4)cc(C)nc3c2n1 ; Cc1ccc2ccc3c(-c4ccc(-c5ccc6c7ccccc7c7ccccc7c6c5)cc4)cc(C)nc3c2n1 ; Cc1ccc2ccc3c(-c4cccc(-c5ccc6c7ccccc7c7ccccc7c6c5)c4)cc(C)nc3c2n1 ; Cc1ccc2ccc3c(-c4cccc(-c5ccc6ccc7cccc8ccc5c6c78)c4)cc(C)nc3c2n1]

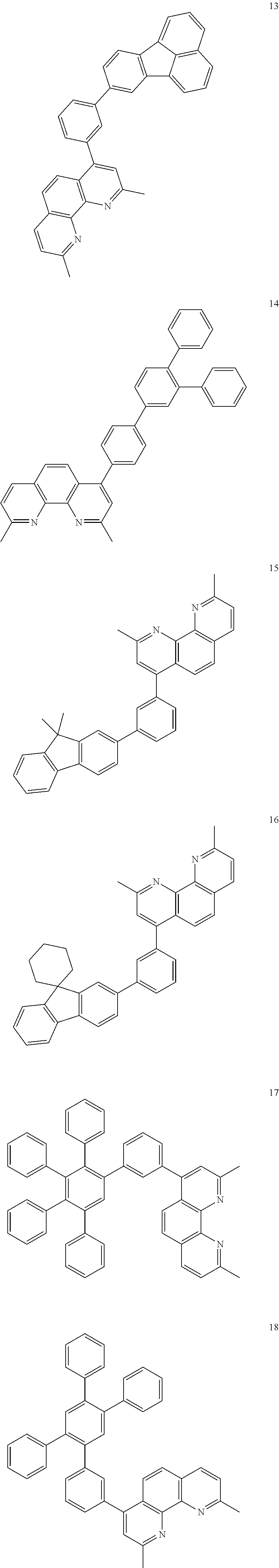 [SMILES: C/C1=C\C=C/c2cccc(c2)-c2cc(-c3cccc(-c4cc(C)nc5c4ccc4ccc(C)nc45)c3)ccc21 ; Cc1ccc2ccc3c(-c4ccc(-c5ccc(-c6ccccc6)c(-c6ccccc6)c5)cc4)cc(C)nc3c2n1 ; Cc1ccc2ccc3c(-c4cccc(-c5ccc6c(c5)C(C)(C)c5ccccc5-6)c4)cc(C)nc3c2n1 ; Cc1ccc2ccc3c(-c4cccc(-c5ccc6c(c5)C5(CCCCC5)c5ccccc5-6)c4)cc(C)nc3c2n1 ; Cc1ccc2ccc3c(-c4cccc(-c5cc(-c6ccccc6)c(-c6ccccc6)c(-c6ccccc6)c5-c5ccccc5)c4)cc(C)nc3c2n1 ; Cc1ccc2ccc3c(-c4cccc(-c5cc(-c6ccccc6)c(-c6ccccc6)cc5-c5ccccc5)c4)cc(C)nc3c2n1]

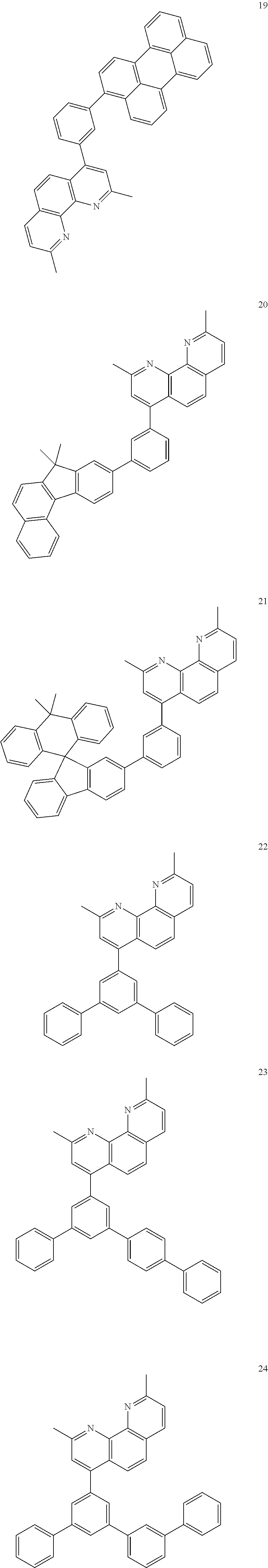 [SMILES: Cc1ccc2ccc3c(-c4cccc(-c5ccc6c7cccc8cccc(c9cccc5c96)c87)c4)cc(C)nc3c2n1 ; Cc1ccc2ccc3c(-c4cccc(-c5ccc6c(c5)C(C)(C)c5ccc7ccccc7c5-6)c4)cc(C)nc3c2n1 ; Cc1ccc2ccc3c(-c4cccc(-c5ccc6c(c5)C5(c7ccccc7-6)c6ccccc6C(C)(C)c6ccccc65)c4)cc(C)nc3c2n1 ; Cc1ccc2ccc3c(-c4cc(-c5ccccc5)cc(-c5ccccc5)c4)cc(C)nc3c2n1 ; Cc1ccc2ccc3c(-c4cc(-c5ccccc5)cc(-c5ccc(-c6ccccc6)cc5)c4)cc(C)nc3c2n1 ; Cc1ccc2ccc3c(-c4cc(-c5ccccc5)cc(-c5cccc(-c6ccccc6)c5)c4)cc(C)nc3c2n1]

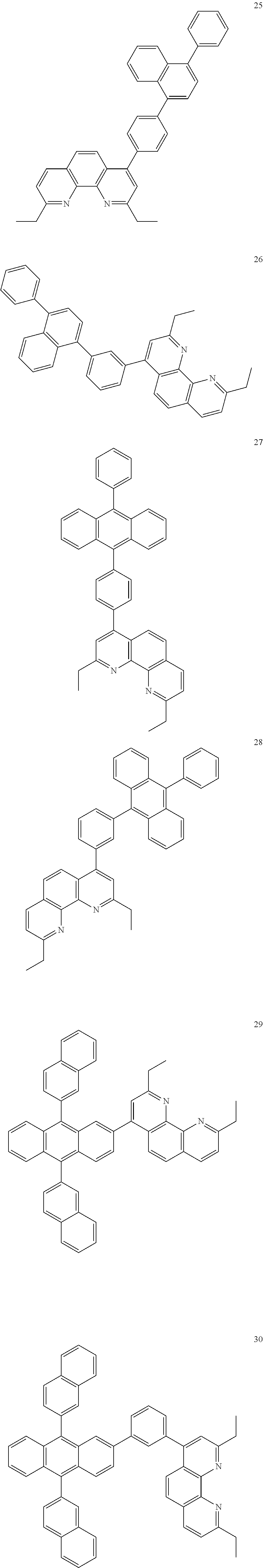

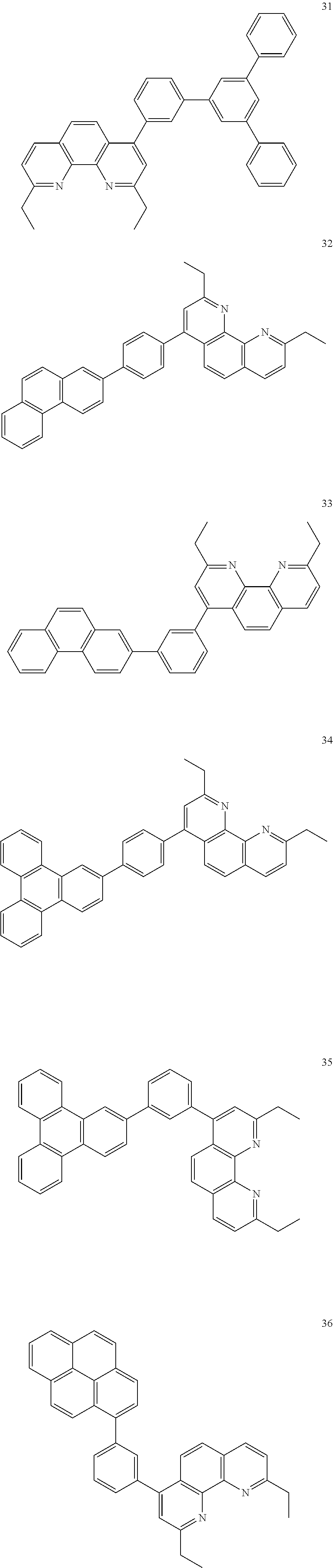 [SMILES: CCc1ccc2ccc3c(-c4cccc(-c5cc(-c6ccccc6)cc(-c6ccccc6)c5)c4)cc(CC)nc3c2n1 ; CCc1ccc2ccc3c(-c4ccc(-c5ccc6c(ccc7ccccc76)c5)cc4)cc(CC)nc3c2n1 ; CCc1ccc2ccc3c(-c4cccc(-c5ccc6c(ccc7ccccc76)c5)c4)cc(CC)nc3c2n1 ; CCc1ccc2ccc3c(-c4ccc(-c5ccc6c7ccccc7c7ccccc7c6c5)cc4)cc(CC)nc3c2n1 ; CCc1ccc2ccc3c(-c4cccc(-c5ccc6c7ccccc7c7ccccc7c6c5)c4)cc(CC)nc3c2n1 ; CCc1ccc2ccc3c(-c4cccc(-c5ccc6ccc7cccc8ccc5c6c78)c4)cc(CC)nc3c2n1]

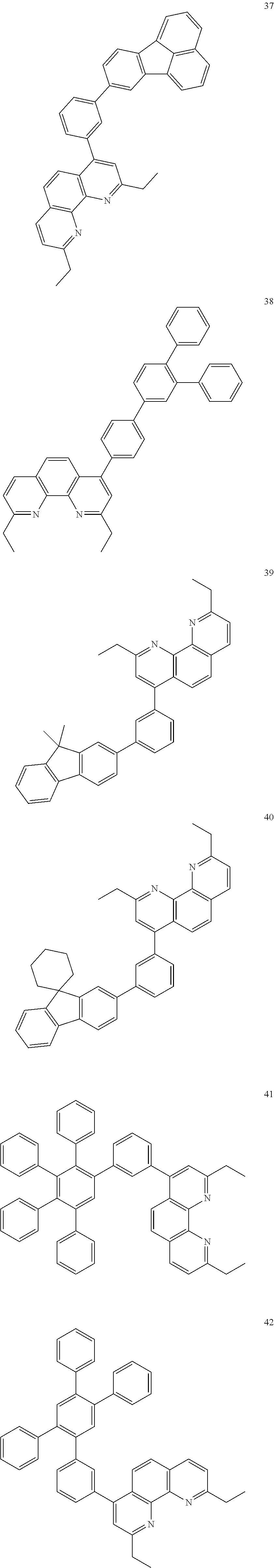

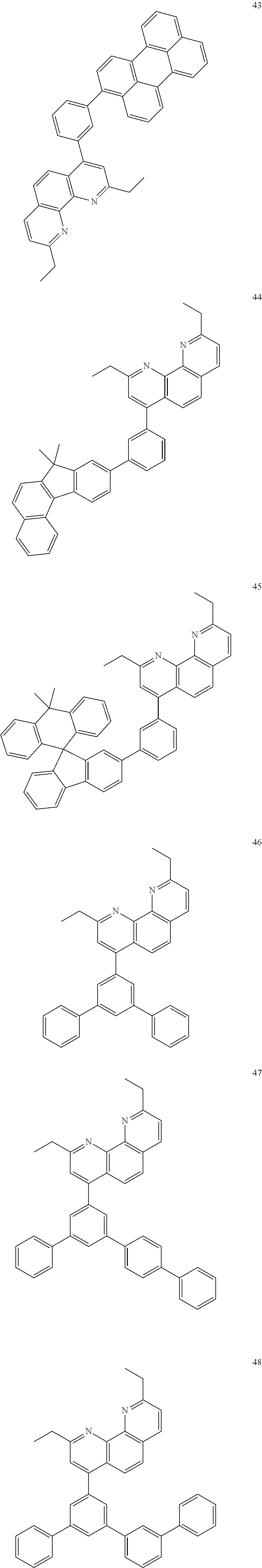 [SMILES: CCc1ccc2ccc3c(-c4cccc(-c5ccc6c7cccc8cccc(c9cccc5c96)c87)c4)cc(CC)nc3c2n1 ; CCc1ccc2ccc3c(-c4cccc(-c5ccc6c(c5)C(C)(C)c5ccc7ccccc7c5-6)c4)cc(CC)nc3c2n1 ; CCc1ccc2ccc3c(-c4cccc(-c5ccc6c(c5)C5(c7ccccc7-6)c6ccccc6C(C)(C)c6ccccc65)c4)cc(CC)nc3c2n1 ; CCc1ccc2ccc3c(-c4cc(-c5ccccc5)cc(-c5ccccc5)c4)cc(CC)nc3c2n1 ; CCc1ccc2ccc3c(-c4cc(-c5ccccc5)cc(-c5ccc(-c6ccccc6)cc5)c4)cc(CC)nc3c2n1 ; CCc1ccc2ccc3c(-c4cc(-c5ccccc5)cc(-c5cccc(-c6ccccc6)c5)c4)cc(CC)nc3c2n1]

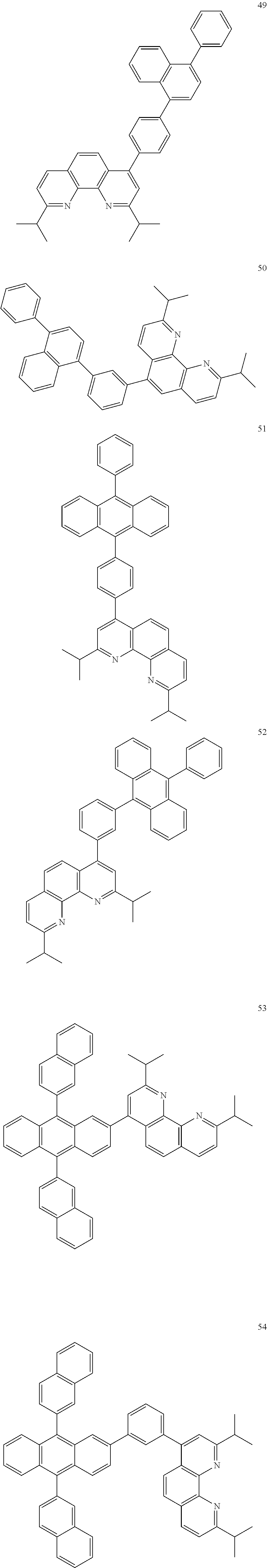

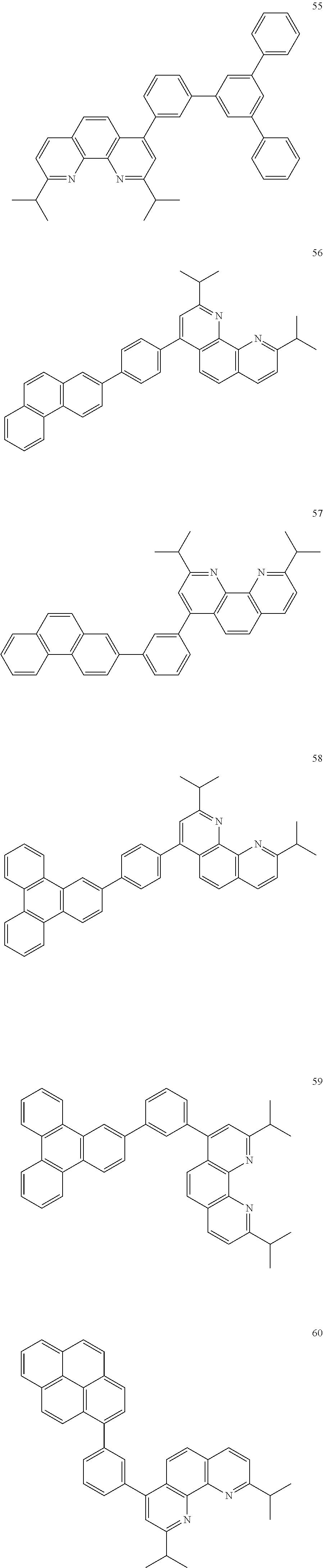 [SMILES: CC(C)c1ccc2ccc3c(-c4cccc(-c5cc(-c6ccccc6)cc(-c6ccccc6)c5)c4)cc(C(C)C)nc3c2n1 ; CC(C)c1ccc2ccc3c(-c4ccc(-c5ccc6c(ccc7ccccc76)c5)cc4)cc(C(C)C)nc3c2n1 ; CC(C)c1ccc2ccc3c(-c4cccc(-c5ccc6c(ccc7ccccc76)c5)c4)cc(C(C)C)nc3c2n1 ; CC(C)c1ccc2ccc3c(-c4ccc(-c5ccc6c7ccccc7c7ccccc7c6c5)cc4)cc(C(C)C)nc3c2n1 ; CC(C)c1ccc2ccc3c(-c4cccc(-c5ccc6c7ccccc7c7ccccc7c6c5)c4)cc(C(C)C)nc3c2n1 ; CC(C)c1ccc2ccc3c(-c4cccc(-c5ccc6ccc7cccc8ccc5c6c78)c4)cc(C(C)C)nc3c2n1]

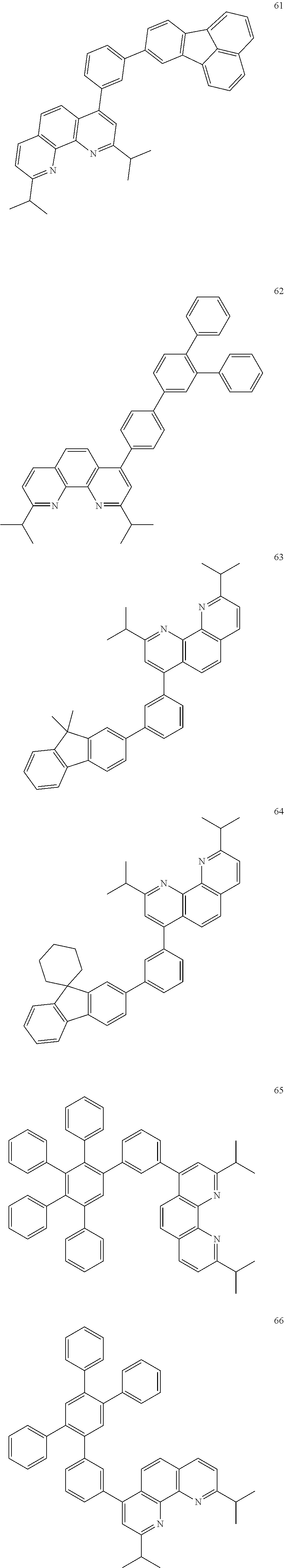

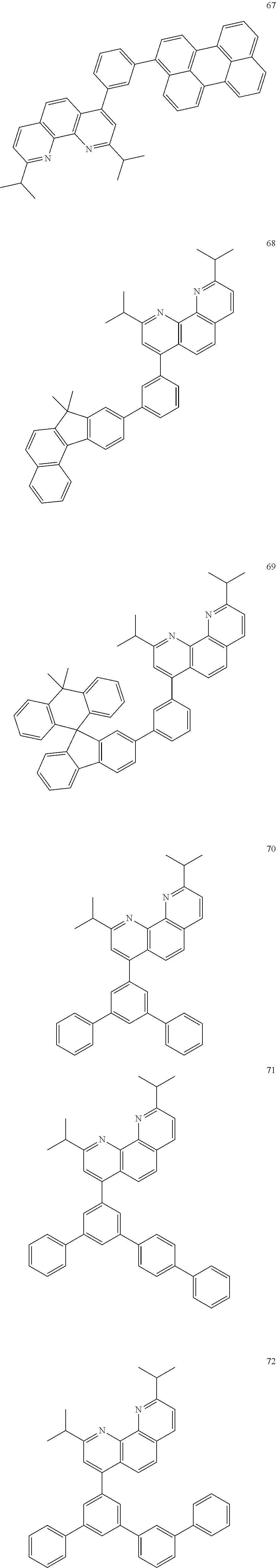

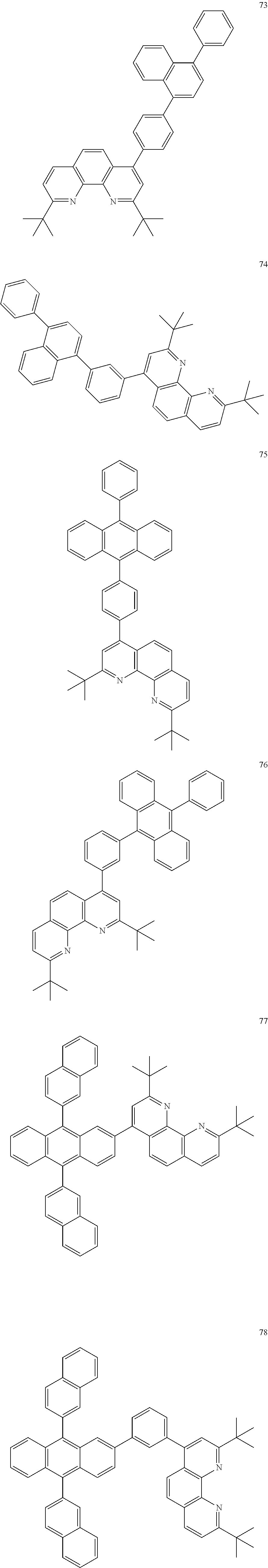

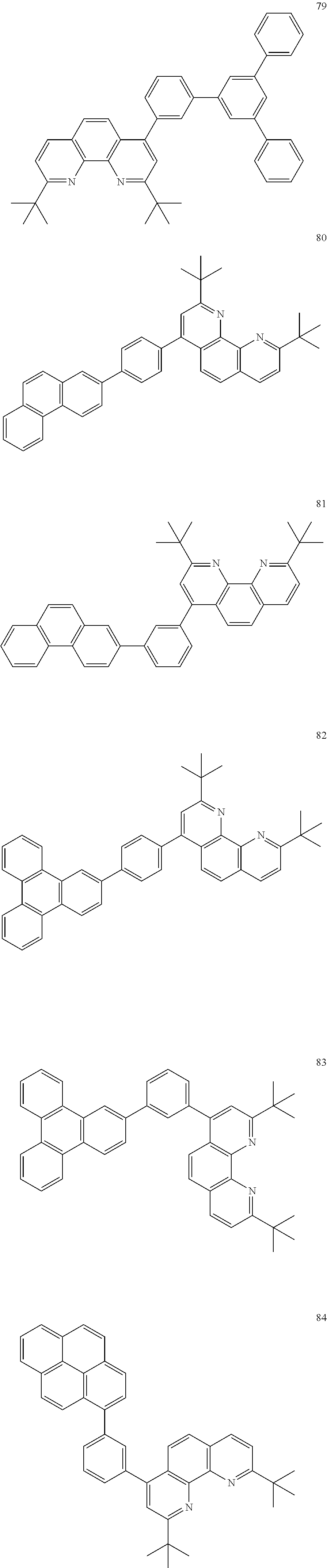 [SMILES: CC(C)(C)c1ccc2ccc3c(-c4cccc(-c5cc(-c6ccccc6)cc(-c6ccccc6)c5)c4)cc(C(C)(C)C)nc3c2n1 ; CC(C)(C)c1ccc2ccc3c(-c4ccc(-c5ccc6c(ccc7ccccc76)c5)cc4)cc(C(C)(C)C)nc3c2n1 ; CC(C)(C)c1ccc2ccc3c(-c4cccc(-c5ccc6c(ccc7ccccc76)c5)c4)cc(C(C)(C)C)nc3c2n1 ; CC(C)(C)c1ccc2ccc3c(-c4ccc(-c5ccc6c7ccccc7c7ccccc7c6c5)cc4)cc(C(C)(C)C)nc3c2n1 ; CC(C)(C)c1ccc2ccc3c(-c4cccc(-c5ccc6c7ccccc7c7ccccc7c6c5)c4)cc(C(C)(C)C)nc3c2n1 ; CC(C)(C)c1ccc2ccc3c(-c4cccc(-c5ccc6ccc7cccc8ccc5c6c78)c4)cc(C(C)(C)C)nc3c2n1]

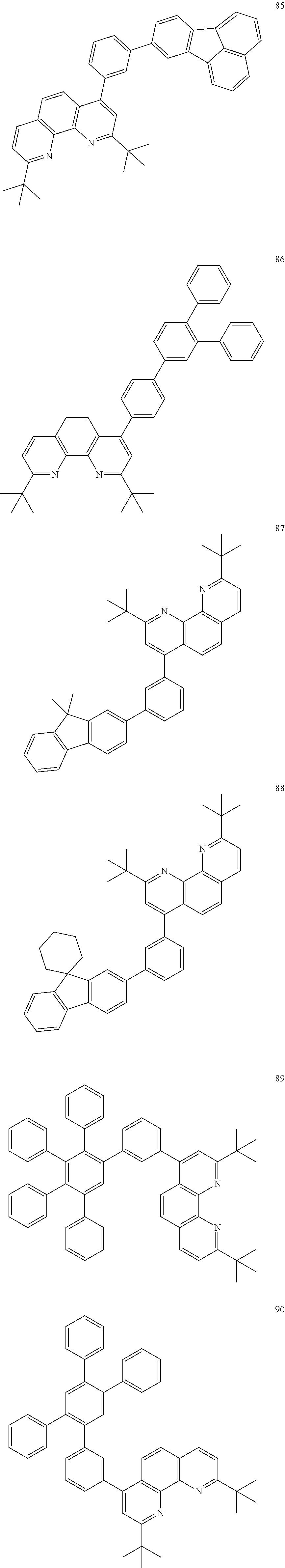

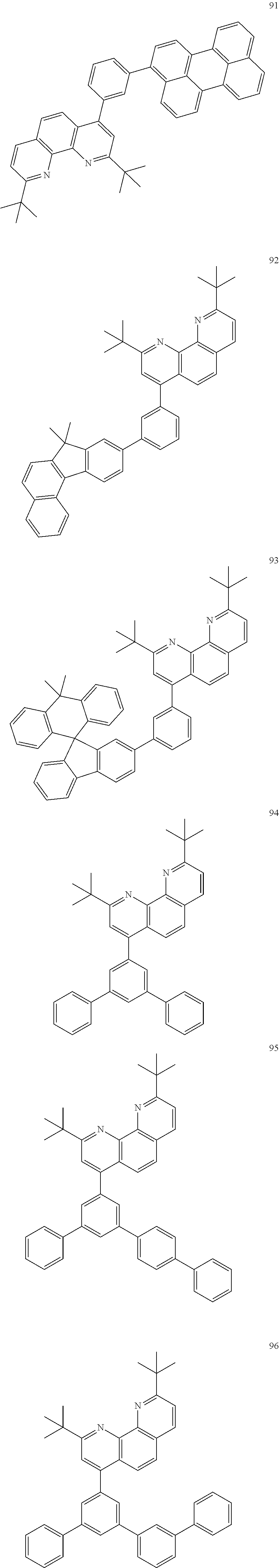

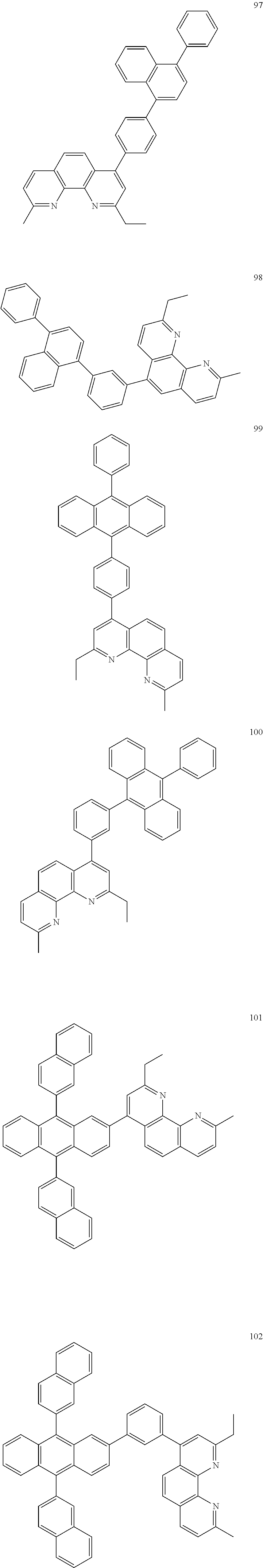

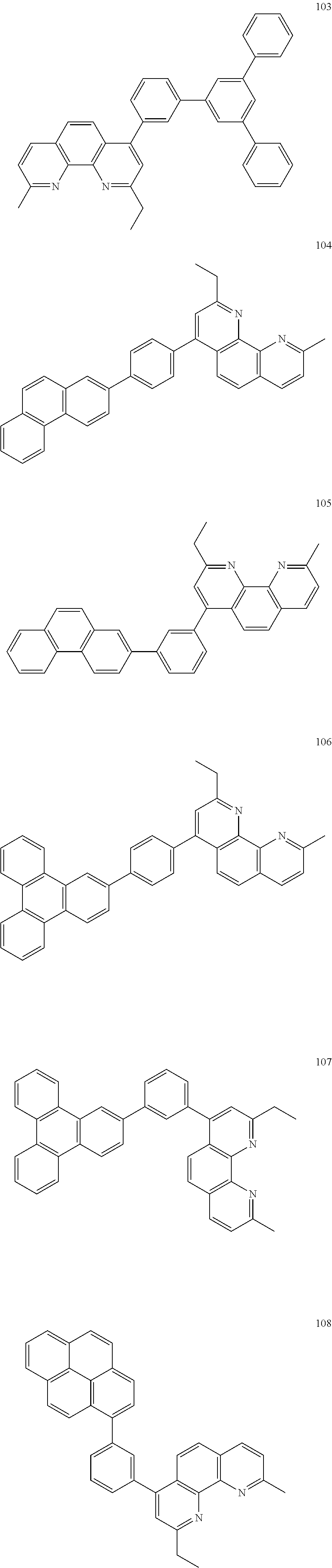 [SMILES: CCc1cc(-c2cccc(-c3cc(-c4ccccc4)cc(-c4ccccc4)c3)c2)c2ccc3ccc(C)nc3c2n1 ; CCc1cc(-c2ccc(-c3ccc4c(ccc5ccccc54)c3)cc2)c2ccc3ccc(C)nc3c2n1 ; CCc1cc(-c2cccc(-c3ccc4c(ccc5ccccc54)c3)c2)c2ccc3ccc(C)nc3c2n1 ; CCc1cc(-c2ccc(-c3ccc4c5ccccc5c5ccccc5c4c3)cc2)c2ccc3ccc(C)nc3c2n1 ; CCc1cc(-c2cccc(-c3ccc4c5ccccc5c5ccccc5c4c3)c2)c2ccc3ccc(C)nc3c2n1 ; CCc1cc(-c2cccc(-c3ccc4ccc5cccc6ccc3c4c56)c2)c2ccc3ccc(C)nc3c2n1]

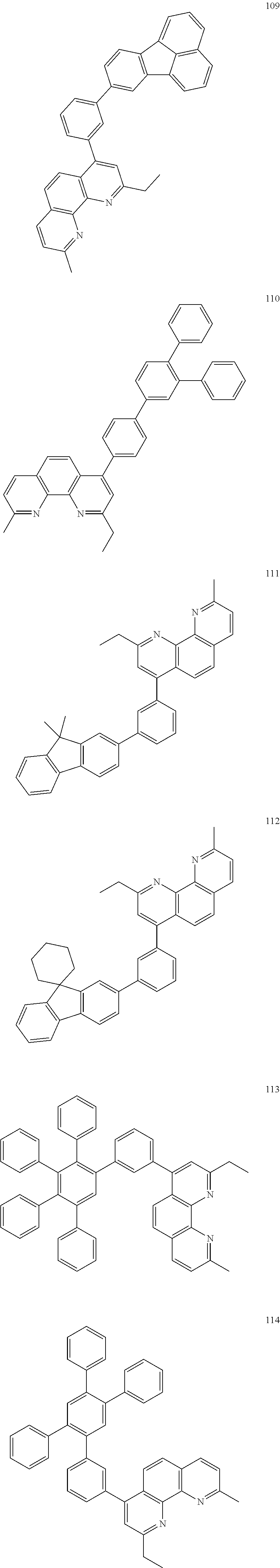 [SMILES: CCc1cc(-c2cccc(-c3ccc4c(c3)-c3cccc(c3)/C=C\C=C\4C)c2)c2ccc3ccc(C)nc3c2n1 ; CCc1cc(-c2ccc(-c3ccc(-c4ccccc4)c(-c4ccccc4)c3)cc2)c2ccc3ccc(C)nc3c2n1 ; CCc1cc(-c2cccc(-c3ccc4c(c3)C(C)(C)c3ccccc3-4)c2)c2ccc3ccc(C)nc3c2n1 ; CCc1cc(-c2cccc(-c3ccc4c(c3)C3(CCCCC3)c3ccccc3-4)c2)c2ccc3ccc(C)nc3c2n1 ; CCc1cc(-c2cccc(-c3cc(-c4ccccc4)c(-c4ccccc4)c(-c4ccccc4)c3-c3ccccc3)c2)c2ccc3ccc(C)nc3c2n1 ; CCc1cc(-c2cccc(-c3cc(-c4ccccc4)c(-c4ccccc4)cc3-c3ccccc3)c2)c2ccc3ccc(C)nc3c2n1]

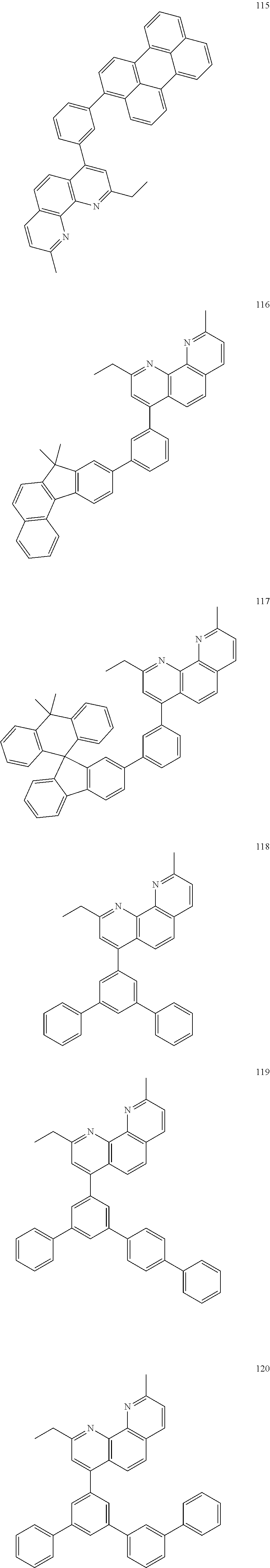 [SMILES: CCc1cc(-c2cccc(-c3ccc4c5cccc6cccc(c7cccc3c74)c65)c2)c2ccc3ccc(C)nc3c2n1 ; CCc1cc(-c2cccc(-c3ccc4c(c3)C(C)(C)c3ccc5ccccc5c3-4)c2)c2ccc3ccc(C)nc3c2n1 ; CCc1cc(-c2cccc(-c3ccc4c(c3)C3(c5ccccc5-4)c4ccccc4C(C)(C)c4ccccc43)c2)c2ccc3ccc(C)nc3c2n1 ; CCc1cc(-c2cc(-c3ccccc3)cc(-c3ccccc3)c2)c2ccc3ccc(C)nc3c2n1 ; CCc1cc(-c2cc(-c3ccccc3)cc(-c3ccc(-c4ccccc4)cc3)c2)c2ccc3ccc(C)nc3c2n1 ; CCc1cc(-c2cc(-c3ccccc3)cc(-c3cccc(-c4ccccc4)c3)c2)c2ccc3ccc(C)nc3c2n1]

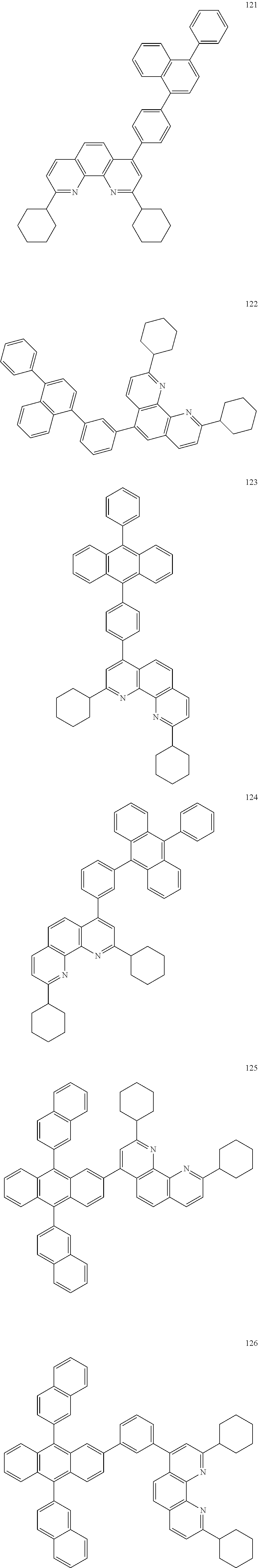

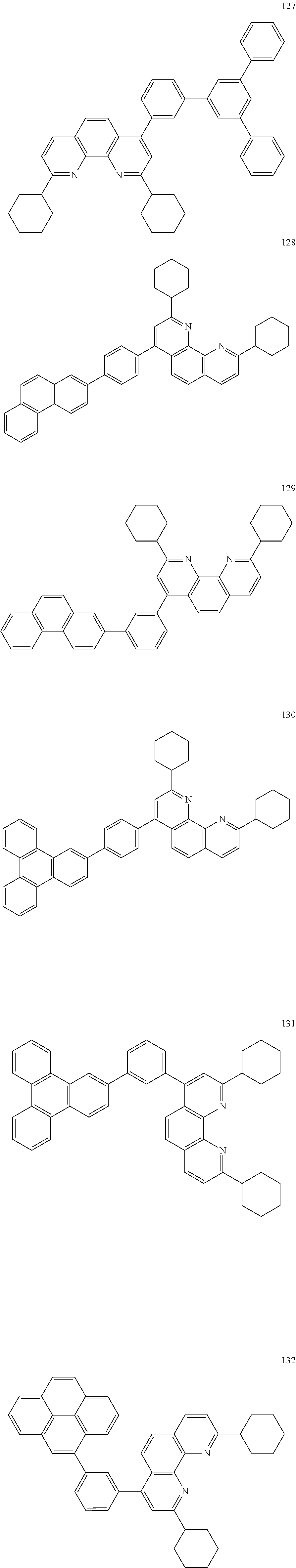

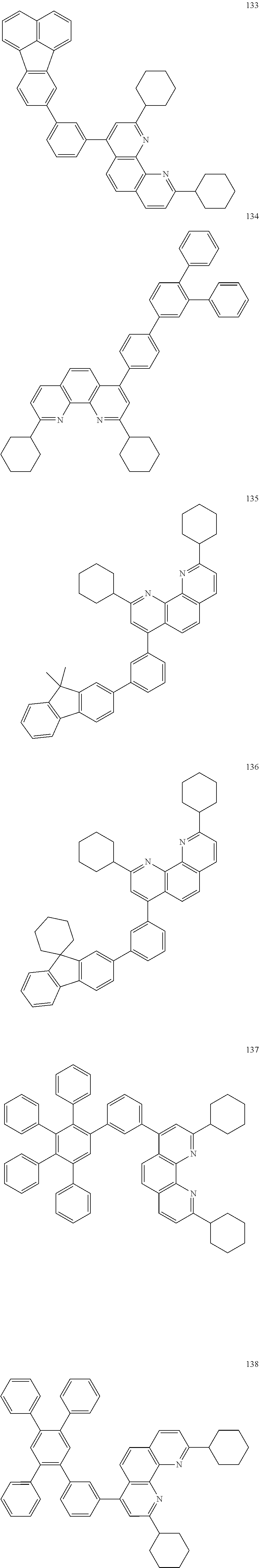

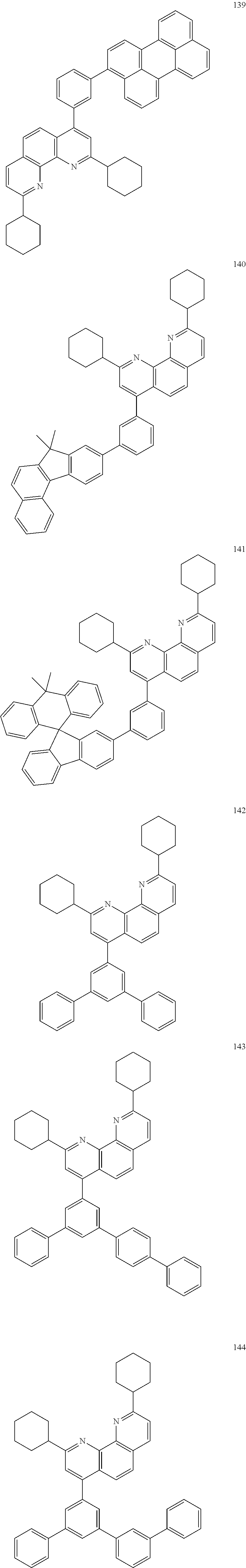

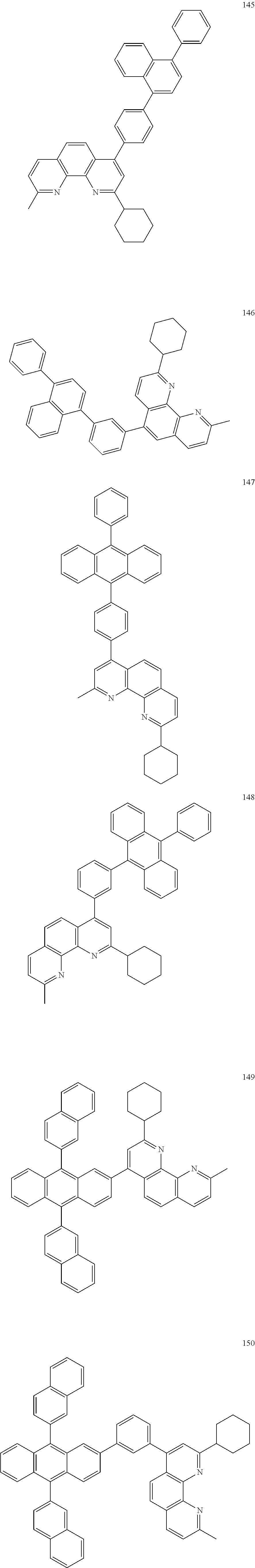 [SMILES: Cc1ccc2ccc3c(-c4ccc(-c5ccc(-c6ccccc6)c6ccccc56)cc4)cc(C4CCCCC4)nc3c2n1 ; Cc1ccc2cc(-c3cccc(-c4ccc(-c5ccccc5)c5ccccc45)c3)c3ccc(C4CCCCC4)nc3c2n1 ; Cc1cc(-c2ccc(-c3c4ccccc4c(-c4ccccc4)c4ccccc34)cc2)c2ccc3ccc(C4CCCCC4)nc3c2n1 ; Cc1ccc2ccc3c(-c4cccc(-c5c6ccccc6c(-c6ccccc6)c6ccccc56)c4)cc(C4CCCCC4)nc3c2n1 ; Cc1ccc2ccc3c(-c4ccc5c(-c6ccc7ccccc7c6)c6ccccc6c(-c6ccc7ccccc7c6)c5c4)cc(C4CCCCC4)nc3c2n1 ; Cc1ccc2ccc3c(-c4cccc(-c5ccc6c(-c7ccc8ccccc8c7)c7ccccc7c(-c7ccc8ccccc8c7)c6c5)c4)cc(C4CCCCC4)nc3c2n1]

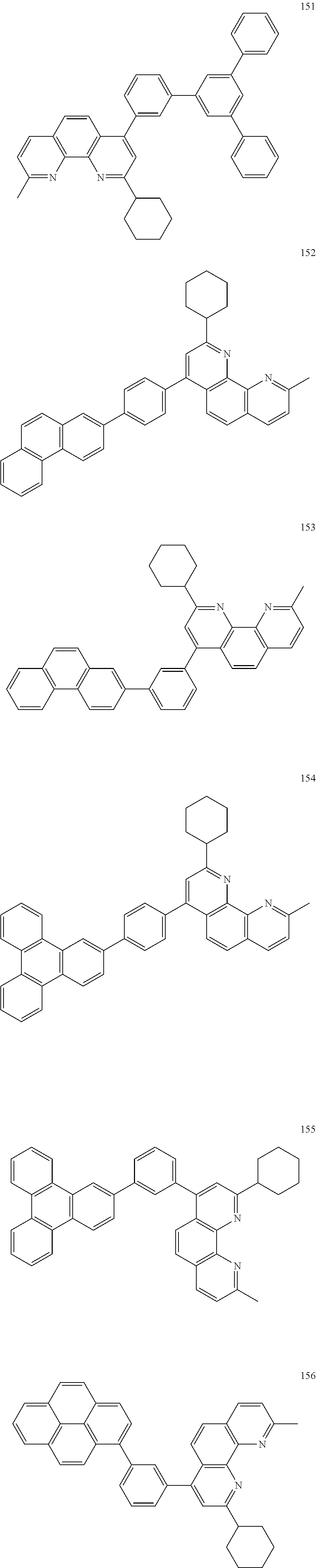 [SMILES: Cc1ccc2ccc3c(-c4cccc(-c5cc(-c6ccccc6)cc(-c6ccccc6)c5)c4)cc(C4CCCCC4)nc3c2n1 ; Cc1ccc2ccc3c(-c4ccc(-c5ccc6c(ccc7ccccc76)c5)cc4)cc(C4CCCCC4)nc3c2n1 ; Cc1ccc2ccc3c(-c4cccc(-c5ccc6c(ccc7ccccc76)c5)c4)cc(C4CCCCC4)nc3c2n1 ; Cc1ccc2ccc3c(-c4ccc(-c5ccc6c7ccccc7c7ccccc7c6c5)cc4)cc(C4CCCCC4)nc3c2n1 ; Cc1ccc2ccc3c(-c4cccc(-c5ccc6c7ccccc7c7ccccc7c6c5)c4)cc(C4CCCCC4)nc3c2n1 ; Cc1ccc2ccc3c(-c4cccc(-c5ccc6ccc7cccc8ccc5c6c78)c4)cc(C4CCCCC4)nc3c2n1]

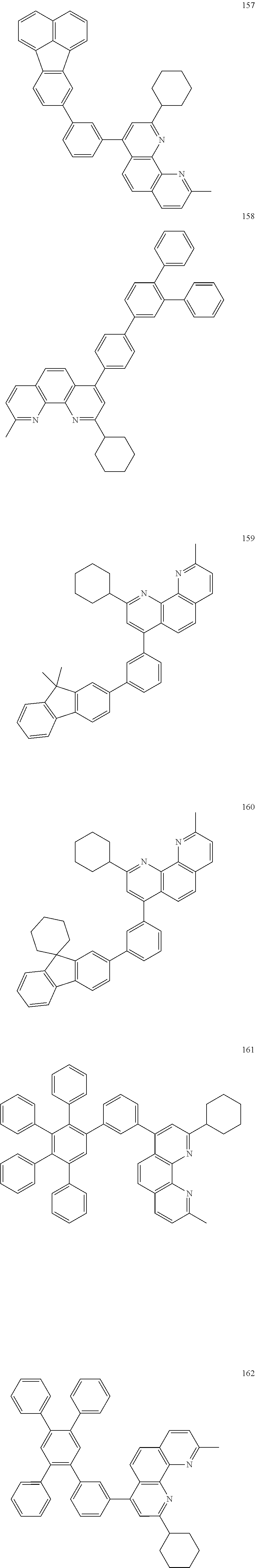

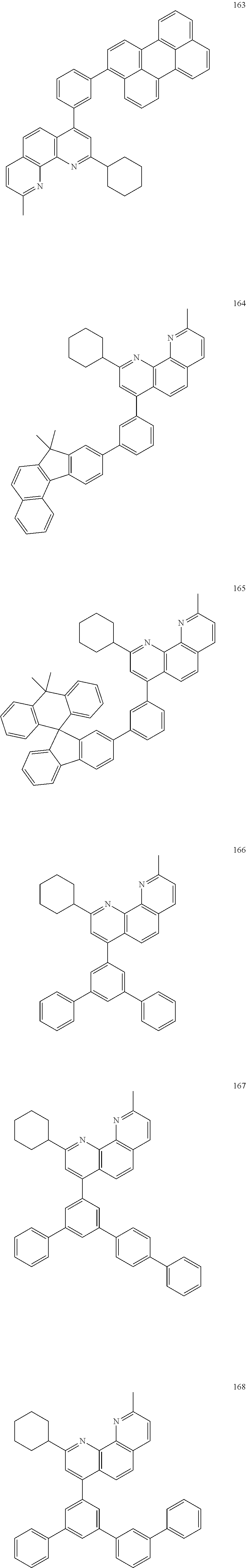 [SMILES: Cc1ccc2ccc3c(-c4cccc(-c5ccc6c7cccc8cccc(c9cccc5c96)c87)c4)cc(C4CCCCC4)nc3c2n1 ; Cc1ccc2ccc3c(-c4cccc(-c5ccc6c(c5)C(C)(C)c5ccc7ccccc7c5-6)c4)cc(C4CCCCC4)nc3c2n1 ; Cc1ccc2ccc3c(-c4cccc(-c5ccc6c(c5)C5(c7ccccc7-6)c6ccccc6C(C)(C)c6ccccc65)c4)cc(C4CCCCC4)nc3c2n1 ; Cc1ccc2ccc3c(-c4cc(-c5ccccc5)cc(-c5ccccc5)c4)cc(C4CCCCC4)nc3c2n1 ; Cc1ccc2ccc3c(-c4cc(-c5ccccc5)cc(-c5ccc(-c6ccccc6)cc5)c4)cc(C4CCCCC4)nc3c2n1 ; Cc1ccc2ccc3c(-c4cc(-c5ccccc5)cc(-c5cccc(-c6ccccc6)c5)c4)cc(C4CCCCC4)nc3c2n1]

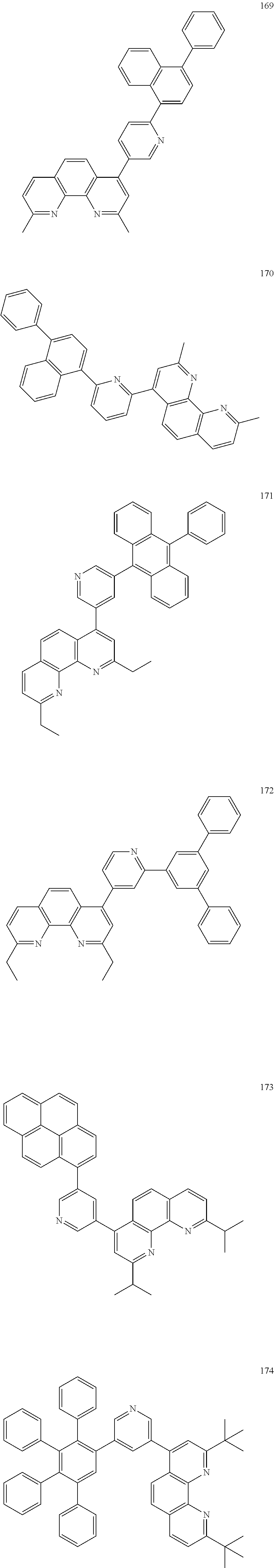 [SMILES: Cc1ccc2ccc3c(-c4ccc(-c5ccc(-c6ccccc6)c6ccccc56)nc4)cc(C)nc3c2n1 ; Cc1ccc2ccc3c(-c4cccc(-c5ccc(-c6ccccc6)c6ccccc56)n4)cc(C)nc3c2n1 ; CCc1ccc2ccc3c(-c4cncc(-c5c6ccccc6c(-c6ccccc6)c6ccccc56)c4)cc(CC)nc3c2n1 ; CCc1ccc2ccc3c(-c4ccnc(-c5cc(-c6ccccc6)cc(-c6ccccc6)c5)c4)cc(CC)nc3c2n1 ; CC(C)c1ccc2ccc3c(-c4cncc(-c5ccc6ccc7cccc8ccc5c6c78)c4)cc(C(C)C)nc3c2n1 ; CC(C)(C)c1ccc2ccc3c(-c4cncc(-c5cc(-c6ccccc6)c(-c6ccccc6)c(-c6ccccc6)c5-c5ccccc5)c4)cc(C(C)(C)C)nc3c2n1]

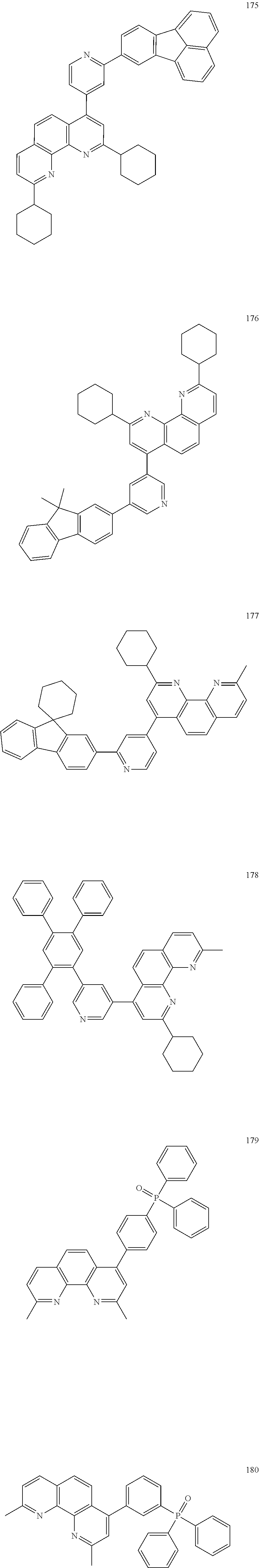

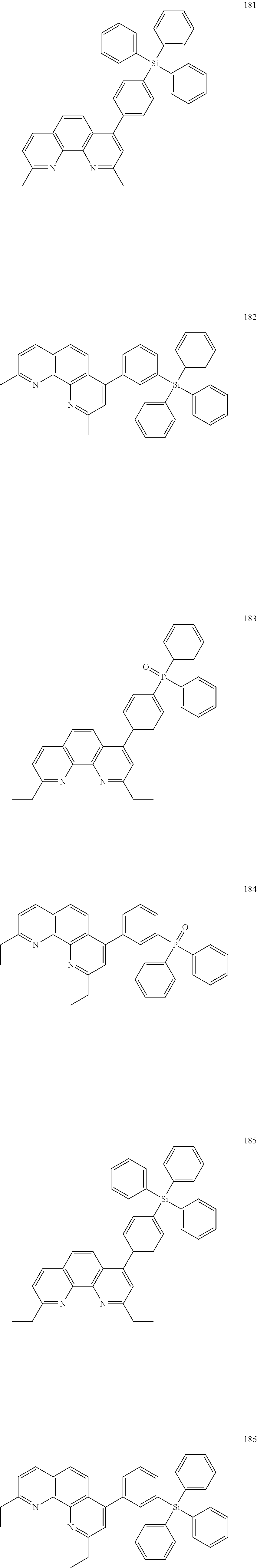 [SMILES: Cc1ccc2ccc3c(-c4ccc([Si](c5ccccc5)(c5ccccc5)c5ccccc5)cc4)cc(C)nc3c2n1 ; Cc1ccc2ccc3c(-c4cccc([Si](c5ccccc5)(c5ccccc5)c5ccccc5)c4)cc(C)nc3c2n1 ; CCc1ccc2ccc3c(-c4ccc(P(=O)(c5ccccc5)c5ccccc5)cc4)cc(CC)nc3c2n1 ; CCc1ccc2ccc3c(-c4cccc(P(=O)(c5ccccc5)c5ccccc5)c4)cc(CC)nc3c2n1 ; CCc1ccc2ccc3c(-c4ccc([Si](c5ccccc5)(c5ccccc5)c5ccccc5)cc4)cc(CC)nc3c2n1 ; CCc1ccc2ccc3c(-c4cccc([Si](c5ccccc5)(c5ccccc5)c5ccccc5)c4)cc(CC)nc3c2n1]

As used herein, the term “alkyl” refers to a monovalent substituent derived from a linear or branched saturated hydrocarbon of 1 to 40 carbon atoms. Its examples include methyl, ethyl, propyl, isobutyl, isopropyl, sec-butyl, pentyl, iso-amyl, and hexyl, but are not limited thereto.

As used herein, the term “alkenyl” refers to a monovalent substituent derived from a linear or branched unsaturated hydrocarbon of 2 to 40 carbon atoms bearing one or more carbon-carbon double bonds. Examples thereof include vinyl, allyl, isopropenyl, and 2-butenyl, but are not limited thereto.

The term “alkynyl”, as used herein, refers to a monovalent substituent derived from a linear or branched unsaturated hydrocarbon of 2 to 40 carbon atoms bearing one or more carbon-carbon triple bonds. Examples thereof include ethynyl and 2-propynyl, but are not limited thereto.

As used herein, the term “cycloalkyl” means a monovalent substituent derived from a monocyclic or polycyclic non-aromatic hydrocarbon of 3 to 40 carbon atoms. Examples of the cycloalkyl include cyclopropyl, cyclopentyl, cyclohexyl, norbornyl, and adamantine, but are not limited thereto.

As used herein, the term “heterocycloalkyl” means a monovalent substituent derived from a non-aromatic hydrocarbon of 3 to 40 nuclear atoms bearing, as ring members, one or more, preferably one to three heteroatoms such as N, O, S, or Se. Examples of the heterocycloalkyl include, but are not limited to, morpholine and piperazine.

The term, “aryl”, as used herein, means a monovalent substituent derived from an aromatic hydrocarbon of 6 to 60 carbon atoms composed of a single ring or a combination of two or more rings. Further, the aryl may also include a form in which two or more rings are simply pendant to or fused with each other. Examples of the aryl include phenyl, naphthyl, phenanthryl, and anthryl, but are not limited thereto.

The term “heteroaryl”, as used herein, refers to a monovalent substituent derived from a monoheterocyclic or polyheterocyclic aromatic hydrocarbon of 5 to 60 nuclear atoms, bearing, as ring members, one or more, preferably one to three heteroatoms, such as N, O, S, or Se. In addition, the heteroaryl may also include a form in which two or more rings are simply pendant to or fused with each other, and further a fused form with an aryl group. Examples of the heteroaryl include: a 6-membered monocyclic ring, such as pyridyl, pyrazinyl, pyrimidinyl, pyridazinyl, and triazinyl, a polycyclic ring, such as phenoxathienyl, indolizinyl, indolyl, purinyl, quinolyl, benzothiazole, and carbazolyl, 2-furanyl, N-imidazolyl, 2-isoxazolyl, 2-pyridinyl, and 2-pyrimidinyl, but are not limited thereto.

The term “alkyloxy”, as used herein, means a monovalent substituent represented by R′O—, in which R′ is an alkyl group of 1 to 40 carbon atoms and may include a linear, branched, or cyclic structure. Examples of the alkyloxy include methoxy, ethoxy, n-propoxy, i-propoxy, t-butoxy, n-butoxy, and pentoxy, but are not limited thereto.

The term “aryloxy”, as used herein, means a monovalent substituent represented by RO—, in which R is an aryl group of 6 to 40 carbon atoms. Examples of the aryloxy include phenyloxy, naphthyloxy, and diphenyloxy, but are not limited thereto.

As used herein, the term “alkylsilyl” refers to a silyl substituted with an alkyl group of 1 to 40 carbon atoms and is intended to encompass mono-, di-, and trialkylsilyl. The term “arylsilyl group” refers to a silyl substituted with an aryl group of 5 to 60 carbon atoms and is intended to encompass mono-, di, and triarylsilyl group.

As used herein, the term “alkylboron” refers to a boron group substituted with an alkyl group of 1 to 40 carbon atoms, and the term “arylboron: refers to a boron group substituted with an aryl group of 6 to 60 carbon atoms.

As used herein, the term “alkylphosphinyl” refers to a phosphine group substituted with an alkyl group of 1 to 40 carbon atoms and is indented to encompass mono- and dialkylphosphinyl. Also, the term “arylphosphinyl” refers to a phosphine group substituted with a mono- or diaryl of 6 to 60 carbon atoms and is intended to encompass mono- and diarylphosphinyl.

As used herein, the term “arylamine” refers to an amine substituted with an aryl group of 6 to 60 carbon atoms and is intended to encompass mono- and diarylamine.

The term “heteroarylamine”, as used herein, means an amine substituted with a heteroaryl group having 5 to 60 nuclear atoms and is intended to encompass mono- and diheteroarylamines.

The term “(aryl) (heteroaryl)amine”, as used herein, means an amine substituted with an aryl group of 6 to 60 carbon atoms and a heteroaryl group having 5 to 60 nuclear atoms.

The “fused ring”, as used herein, means a fused aliphatic ring of 3 to 40 carbon atoms, a fused aromatic ring of 6 to 60 carbon atoms, a fused heteroaliphatic ring of 3 to 60 nuclear atoms, a fused heteroaromatic ring of 5 to 60 nuclear atoms, or a combined form thereof.

<Organic Electroluminescent Device>

The present disclosure also provides an organic electroluminescent device (hereinafter referred to as “organic EL device”) including the compound represented by Chemical Formula 1.

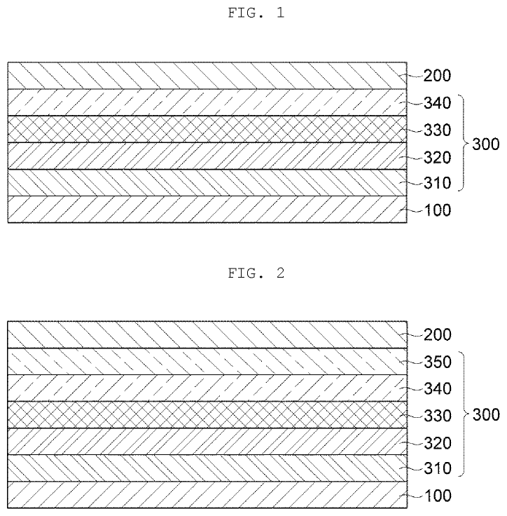

FIGS. 1 to 4 are schematic cross-sectional views of organic EL devices according to a first to a fourth embodiments of the present disclosure, respectively.

Below, the organic EL devices according to the first to the third embodiments of the present disclosure will be described in detail in conjunction with FIGS. 1 to 3.

As shown in FIGS. 1 to 3, the organic EL device according to the present disclosure includes an anode (100), a cathode (200), and at least one organic layer (300) interposed between the anode and the cathode, in which the at least one organic layer includes the compound represented by Chemical Formula 1. The compounds may be used singly or in combination.

The at least one organic layer (300) may include at least one of a hole injection layer (310), a hole transport layer (320), a light-emitting layer (330), an electron transport auxiliary layer (360), an electron transport layer (340), and an electron injection layer (350), wherein the organic layer (300) contains the compound represented by Chemical Formula 1. Specifically, the organic layer containing the compound of Chemical Formula 1 may be an electron transport layer (340). That is, the compound represented by Chemical Formula 1 is used as an electron transport layer material in an organic EL device. In such an organic EL device, electrons can be easily injected from the cathode or the electron injection layer to the electron transport layer with the aid of the compound of Chemical Formula 1 and then move toward the light-emitting layer, so that holes and electrons highly combine with each other. Thus, the organic EL device of the present disclosure is excellent in luminous efficiency, power efficiency, and luminance. Moreover, the compound of Chemical Formula 1 is superb in terms of thermal stability and electrochemical stability and as such, can enhance the performance of the organic EL device.

The compound of Chemical Formula 1 may be used alone or in combination with an electron transport layer material known in the art.

The electron transport layer material that may be used in combination with the compound of Chemical Formula 1 includes an electron transport material commonly known in the art. Non-limiting examples of available electron transport materials may include oxazole-based compounds, isoxazole-based compounds, triazole-based compounds, isothiazole-based compounds, oxadiazole-based compounds, thiadiazole-based compounds, perylene-based compounds, aluminum complexes (e.g., Alq₃, tris(8-quinolinolato)-aluminum), and gallium complexes (e.g., Gaq′2OPiv, Gag′2OAc, and 2(Gaq′2)). These may be used solely or two or more types thereof may be used in combination.

In the present disclosure, when the compound of Chemical Formula 1 and the material for the electron transport layer are used in combination, a mixing ratio thereof is not particularly limited, and may be appropriately adjusted within a range known in the art.

No particular limitations are imparted to the structure of the organic EL device of the present disclosure, but, for example, an anode (100), at least one organic layer (300), and a cathode (200) may be sequentially deposited on a substrate (see FIGS. 1 to 3). Although not shown, the structure may have an insulation layer or an adhesive layer inserted into the interface between the electrode and the organic layer.

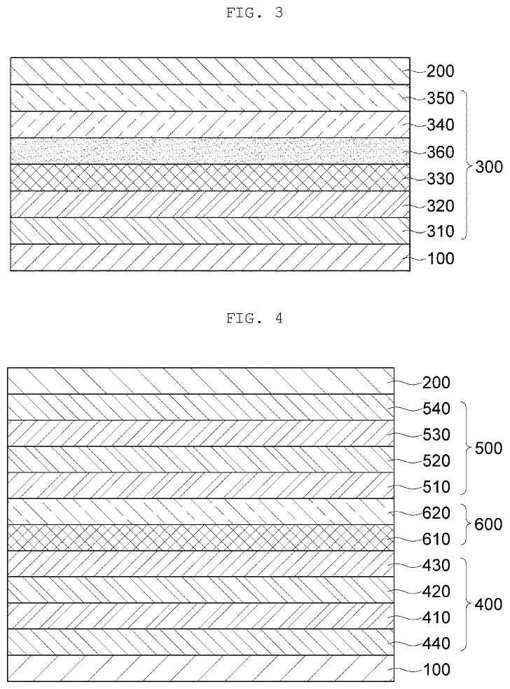

According to an embodiment, the organic EL device, as shown in FIG. 1, may have the structure in which an anode (100), a hole injection layer (310), a hole transport layer (320), a light-emitting layer (330), an electron transport layer (340), and a cathode (200) are sequentially deposited on a substrate. Optionally, as shown in FIG. 2, an electron injection layer (350) may be disposed between the electron transport layer (340) and the cathode (200). In addition, an electron transport auxiliary layer (360) may be positioned between the light-emitting layer (330) and the electron transport layer (340) (see FIG. 3).

The organic EL device of the present disclosure may be fabricated by forming organic layers and electrodes with materials and methods known in the art, except that at least one organic layer (300) [e.g., electron transport layer (340)] contains the compound represented by Chemical Formula 1.

The organic layer may be formed using a vacuum deposition method or a solution coating method. Examples of the solution coating method include, but are not limited to, spin coating, dip coating, doctor blading, inkjet printing, and thermal transfer.

No particular limitations are imparted to a substrate available in the present disclosure. Non-limiting examples of the substrate available in the present disclosure include silicon wafers, quartz, glass plates, metal plates, plastic films, and sheets.

In addition, examples of an anode material include, but are not limited to: metals such as vanadium, chromium, copper, zinc, and gold or an alloy thereof; metal oxides such as zinc oxide, indium oxide, indium tin oxide (ITO), and indium zinc oxide (IZO); combinations of metals and oxides such as ZnO:Al or SnO₂:Sb; conductive polymers such as polythiophene, poly(3-methylthiophene), poly [3,4-(ethylene-1,2-dioxy) thiophene] (PEDT), polypyrrole, or polyaniline; and carbon black.

Furthermore, examples of cathode materials available in the present disclosure include, but are not limited to, a metal such as magnesium, calcium, sodium, potassium, titanium, indium, yttrium, lithium, gadolinium, aluminum, silver, tin, or lead, or an alloy thereof; a multi-layered material such as LiF/Al or LiO₂/Al.

Moreover, so long as it is known in the art, any material for the hole injection layer, the hole transport layer, the light-emitting layer, and the electron injection layer may be used without particular limitations.

Referring to FIG. 4, an organic EL device according to a fourth embodiment of the present disclosure is described.

As shown in FIG. 4, the organic EL device according to the fourth embodiment of the present disclosure is a tandem device structured to include: an anode (100) and a cathode (200), which face each other; a plurality of light-emitting units (400, 500) interposed between the anode (100) and the cathode (200); and a charge generation layer (600), interposed between the adjacent light-emitting units (400, 500), including an N-type charge generation layer (610) and a P-type charge generation layer (620). In this regard, the N-type charge generation layer (610) contains the compound represented by Chemical Formula 1.

Such a tandem organic EL device includes at least two light-emitting units, with a charge generation layer interposed between adjacent light-emitting units and as such, can be configured to increase the number of light-emitting units.

According to an embodiment, the plurality of light-emitting units may include a 1st light-emitting unit (400), a 2nd light-emitting unit (500), . . . , and an m−1th light-emitting unit (m=an integer of 3 or greater, specifically 3-4). In this regard, a charge generation layer (600) including an N-type charge generation layer (610) and a P-type charge generation layer (620) is disposed between the adjacent light-emitting units, wherein the N-type charge generation layer (610) contains the compound represented by Chemical Formula 1.

Specifically, the organic EL device according to the present disclosure includes: an anode (100) and a cathode (200), which face each other; a first light-emitting unit (400) disposed on the anode (100); a second light-emitting unit (500) disposed on the first light-emitting unit (400); a charge generation layer (600), disposed between the first and the second light-emitting unit (400, 500), including an N-type charge generation layer (610) and a P-type charge generation layer (620). The N-type charge generation layer (610) contains the compound represented by Chemical Formula 1.

The light-emitting units (400, 500) each include a hole transport layer (410, 510), a light-emitting layer (420, 520), and an electron transport layer (430, 530). Specifically, the first light-emitting unit (400) may include a first hole transport layer (410), a first light-emitting layer (420), and a first electron transport layer (430) while a second light-emitting unit (500) may include a hole transport layer (510), a light-emitting layer (520), and an electron transport layer (530). Optionally, the first light-emitting unit (400) may further include a hole injection layer (440).

So long as it is known in the art, any material may be employed for the hole transport layer (410, 510), the light-emitting layer (420, 520), the electron transport layer (430, 530), and the hole injection layer (440).

Being disposed between adjacent light-emitting units (400, 500), the charge generation layer (CGL) (600) can control the charges between the light-emitting units (400, 500) to make a charge balance.

The charge generation layer (600) includes an N-type charge generation layer (610), positioned adjacent to the first light-emitting unit (400), for supplying electrons to the first light-emitting unit (400); and a P-type charge generation layer (620), positioned adjacent to a second light-emitting unit (500), for supplying holes to the second light-emitting unit (500).

The N-type charge generation layer (610) includes the compound represented by Chemical Formula 1. With excellent electron mobility, the compound of Chemical Formula 1 exhibits excellent electron injection and transport potentials. Hence, when applied as an N-type charge generation layer material to an organic EL device, the compound of Chemical Formula 1 can prevent the device from increasing in progressive driving voltage and decreasing in lifespan.

According to an embodiment, the N-type charge generation layer (610) contains one host having an electron transport property, and the host is the compound represented by Chemical Formula 1. In contrast to an N-type charge generation layer containing two hosts, the N-type charge generation layer (610) of the present disclosure can be prepared through co-deposition, which may lead to an improvement in process efficiency.

The N-type charge generation layer (610) may further include an N-type dopant.

So long as it is commonly used for an N-type charge generation layer in the art, any material may be available in the present disclosure without particular limitations. Examples of the material include: alkali metals, such as Li, Na, K, Rb, Cs, Fr, and so on; alkaline earth metals, such as Be, Mg, Ca, Sr, Ba, Ra, and so on; metals in Group 15, such as Bi (bismuth), Sb (antimony), and so on; lanthanide metals, such as La (lanthanum), Ce (cerium), Pr (preseodyminum), Nd (neodymium), Pm (promethium), Sm (samarium), Eu (europium), Gd (gadolinium), Tb (terbium), Dy (dysprosium), Ho (holmium), Er (erbium), Tm (thulium), Yb (ytterbium), and Lu (lutetium); and compounds of at least one of the metals. In addition, the N-type charge generation layer may be an organic N-type dopant that has an electron donor property and can donor at least a part of electric charges to an organic host (e.g., the compound of Chemical Formula 1) to form a charge-transfer complex with the organic host, and may be exemplified by bis(ethylenedithio)tetrathiafulvalene (BEDT-TTF) and tetrathiafulvalene (TTF).

The thickness of the N-type charge generation layer (610) is not particularly limited and may range, for example, from about 5 to 30 nm.

The P-type charge generation layer (620) may be composed of a metal or a P-type doped organic material. Here, the metal may be exemplified by Al, Cu, Fe, Pb, Zn, Au, Pt, W, In, Mo, Ni, and Ti and may be used alone or in the form of an alloy of two or more metals. In addition, no particular limitations are imparted to any P-type dopant and host that are commonly used for the P-type doped organic material. Examples of the P-type dopant include F4-TCNQ (2,3,5,6-tetrafluoro-7,7,8,8-tetracyano-quinodimethane), iodide, FeCl3, FeF3, and SbCl5. These dopants may be used solely or in combination. Non-limiting examples of the host include NPB (N,N′-bis(naphthaen-1-yl)-N,N′-bis(phenyl)-benzidine), TPD (N,N′-bis(3-methylphenyl)N,N′-bis(phenyl)-benzidine), and TNB (N,N,N′,N′-tetra-naphthalenyl-benzidine). These hosts may be used solely or in combination.

Because the anode (100) and the cathode (200) are the same as in the first to the third embodiment, a description thereof is omitted.

A better understanding of the present disclosure may be obtained through the following examples which are set forth to illustrate, but are not to be construed as limiting the present disclosure.

### [Synthesis Example 1] Compound 1

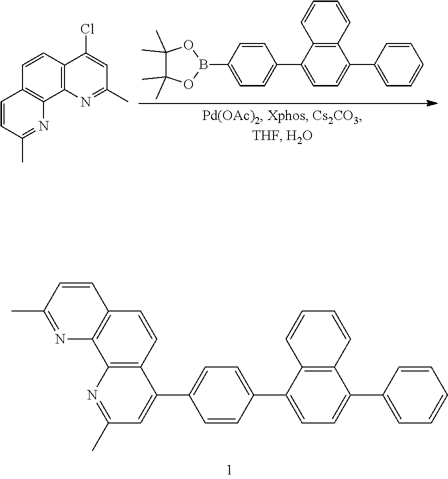

4-Chloro-2,9-dimethyl-1,10-phenanthroline (5 g, 20.6 mmol), 4,4,5,5-tetramethyl-2-(4-(4-phenylnaphthalen-1-yl)phenyl)-1,3,2-dioxaborolane (8.4 g, 20.6 mmol), Pd(OAc)₂ (0.2 g, 1.0 mmol), Xphos (1.0 g, 2.1 mmol), and Cs₂CO₃ (13.5 g, 41.3 mmol) were added to a mixture of toluene (50 ml), EtOH (10 ml), and H₂O (10 ml) and heated for 12 hours under reflux. After completion of the reaction, extraction was conducted with methylene chloride. The extract was added with MgSO₄ and then filtered. The solvent was removed from the filtered organic layer which was then purified by column chromatography to afford the target compound (7.0 g, yield: 70%).

[LCMS]: 487

### [Synthesis Example 2] Synthesis of Compound 2

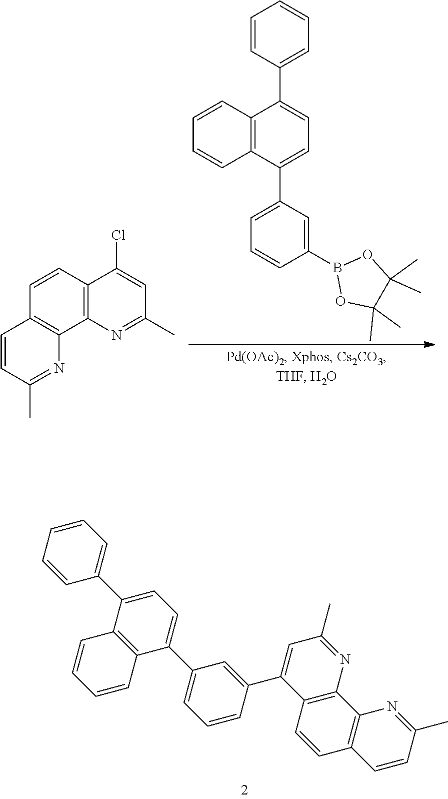 [SMILES: Cc1ccc2ccc3c(Cl)cc(C)nc3c2n1 ; CC1(C)OB(c2cccc(-c3ccc(-c4ccccc4)c4ccccc34)c2)OC1(C)C ; Cc1ccc2ccc3c(-c4cccc(-c5ccc(-c6ccccc6)c6ccccc56)c4)cc(C)nc3c2n1]

The same procedure as in [Synthesis Example 1], with the exception of using 4,4,5,5-tetramethyl-2-(4-(4-phenylnaphthalen-1-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.0 g, yield: 70%).

[LCMS]: 487

### [Synthesis Example 3] Synthesis of Compound 3

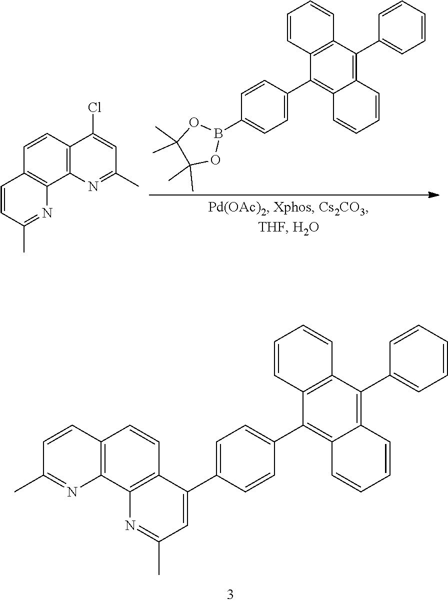

The same procedure as in [Synthesis Example 1], with the exception of using 4,4,5,5-tetramethyl-2-(4-(10-phenylanthracen-9-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.1 g, yield: 73%).

[LCMS]: 537

### [Synthesis Example 4] Synthesis of Compound 4

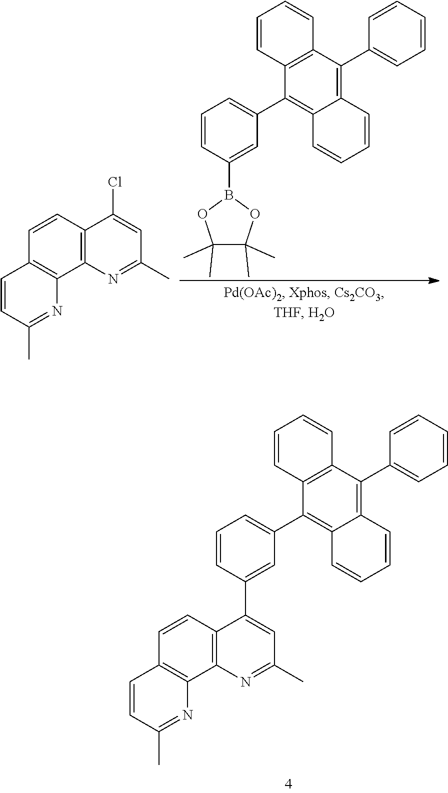

The same procedure as in [Synthesis Example 1], with the exception of using 4,4,5,5-tetramethyl-2-(3-(10-phenylanthracen-9-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.1 g, yield: 73%).

[LCMS]: 537

### [Synthesis Example 5] Synthesis of Compound 5

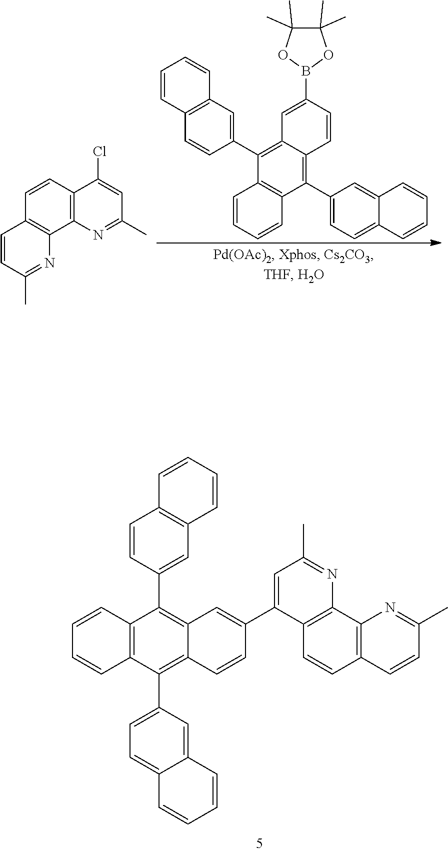 [SMILES: Cc1ccc2ccc3c(Cl)cc(C)nc3c2n1 ; CC1(C)OB(c2ccc3c(-c4ccc5ccccc5c4)c4ccccc4c(-c4ccc5ccccc5c4)c3c2)OC1(C)C ; Cc1ccc2ccc3c(-c4ccc5c(-c6ccc7ccccc7c6)c6ccccc6c(-c6ccc7ccccc7c6)c5c4)cc(C)nc3c2n1]

The same procedure as in [Synthesis Example 1], with the exception of using 2-(9,10-di(naphthalen-2-yl)anthracen-2-yl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (9.6 g, yield: 73%).

[LCMS]: 637

### [Synthesis Example 6] Synthesis of Compound 6

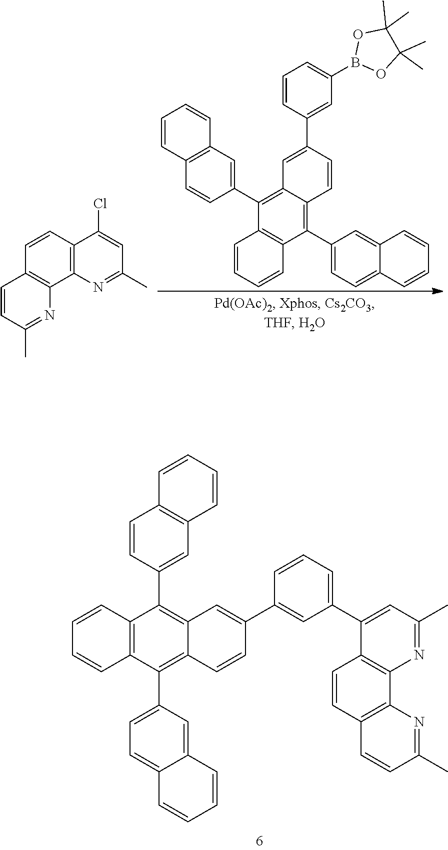 [SMILES: Cc1ccc2ccc3c(Cl)cc(C)nc3c2n1 ; CC1(C)OB(c2cccc(-c3ccc4c(-c5ccc6ccccc6c5)c5ccccc5c(-c5ccc6ccccc6c5)c4c3)c2)OC1(C)C ; Cc1ccc2ccc3c(-c4cccc(-c5ccc6c(-c7ccc8ccccc8c7)c7ccccc7c(-c7ccc8ccccc8c7)c6c5)c4)cc(C)nc3c2n1]

The same procedure as in [Synthesis Example 1], with the exception of using 2-(3-(9,10-di(naphthalen-2-yl)anthracen-2-yl)phenyl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (10.2 g, yield: 70%).

[LCMS]: 713

### [Synthesis Example 7] Synthesis of Compound 7

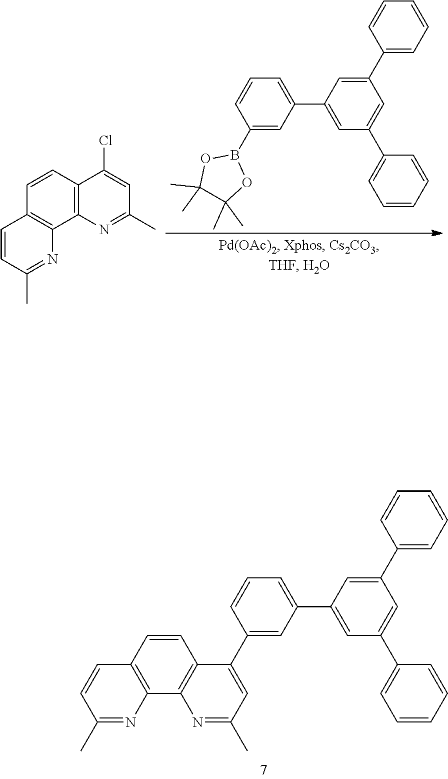

The same procedure as in [Synthesis Example 1], with the exception of using 4,4,5,5-tetramethyl-2-(5′-phenyl-[1,1′:3′,1′-terphenyl]-3-yl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.0 g, yield: 75%)

[LCMS]: 513

### [Synthesis Example 8] Synthesis of Compound 8

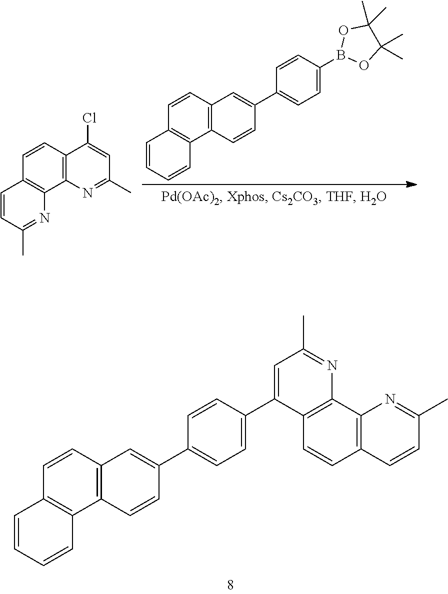

The same procedure as in [Synthesis Example 1], with the exception of using 4,4,5,5-tetramethyl-2-(4-(phenanthren-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.0 g, yield: 74%).

[LCMS]: 460

### [Synthesis Example 9] Synthesis of Compound 9

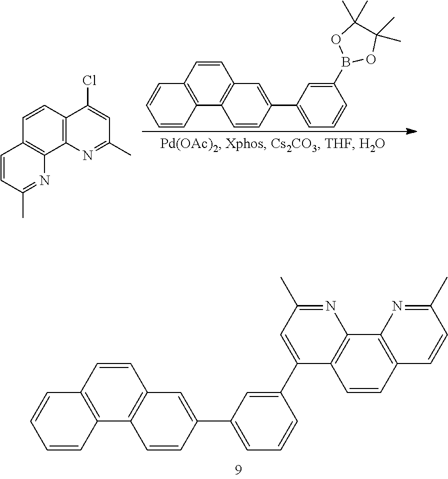 [SMILES: Cc1ccc2ccc3c(Cl)cc(C)nc3c2n1 ; CC1(C)OB(c2cccc(-c3ccc4c(ccc5ccccc54)c3)c2)OC1(C)C ; Cc1ccc2ccc3c(-c4cccc(-c5ccc6c(ccc7ccccc76)c5)c4)cc(C)nc3c2n1]

The same procedure as in [Synthesis Example 1], with the exception of using 4,4,5,5-tetramethyl-2-(3-(phenanthren-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.0 g, yield: 74%).

[LCMS]: 460

### [Synthesis Example 10] Synthesis of Compound 10

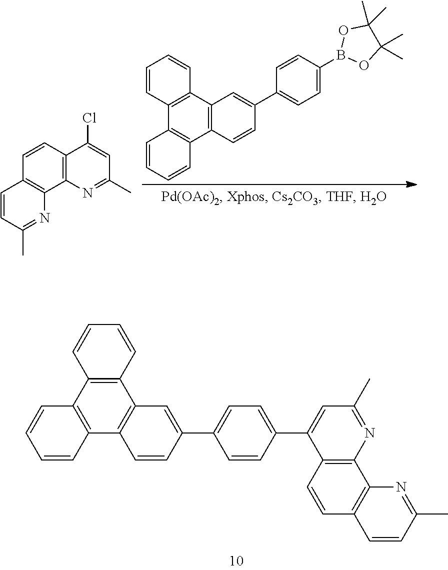

The same procedure as in [Synthesis Example 1], with the exception of using 4,4,5,5-tetramethyl-2-(3-(triphenylen-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.4 g, yield: 70%).

[LCMS]: 511

### [Synthesis Example 11] Synthesis of Compound 11

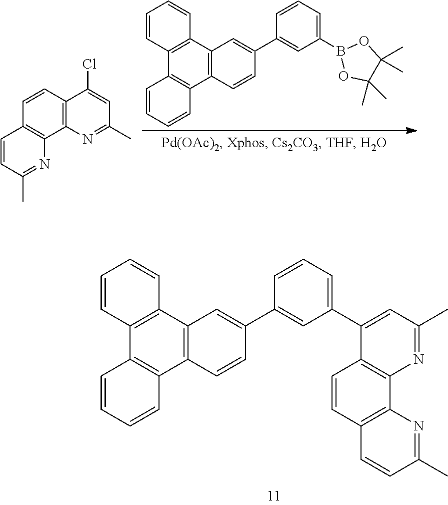

The same procedure as in [Synthesis Example 1], with the exception of using 4,4,5,5-tetramethyl-2-(4-(triphenylen-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.4 g, yield: 70%).

[LCMS]: 511

### [Synthesis Example 12] Synthesis of Compound 12

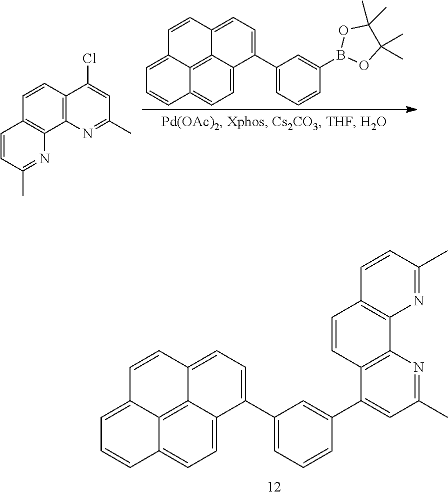 [SMILES: Cc1ccc2ccc3c(Cl)cc(C)nc3c2n1 ; CC1(C)OB(c2cccc(-c3ccc4ccc5cccc6ccc3c4c56)c2)OC1(C)C ; Cc1ccc2ccc3c(-c4cccc(-c5ccc6ccc7cccc8ccc5c6c78)c4)cc(C)nc3c2n1]

The same procedure as in [Synthesis Example 1], with the exception of using 4,4,5,5-tetramethyl-2-(3-(pyren-1-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.0 g, yield: 70%).

[LCMS]: 485

### [Synthesis Example 13] Synthesis of Compound 13

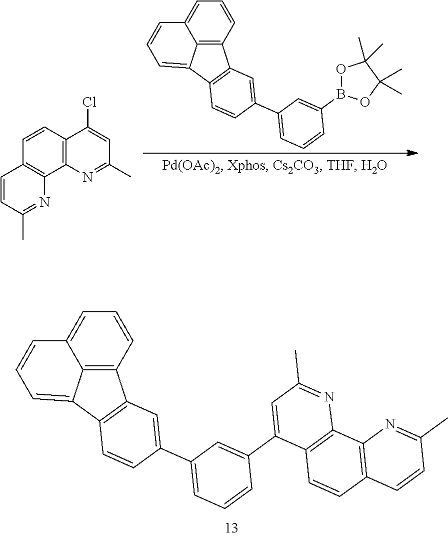 [SMILES: Cc1ccc2ccc3c(Cl)cc(C)nc3c2n1 ; CC1(C)OB(c2cccc(-c3ccc4c(c3)-c3cccc5cccc-4c35)c2)OC1(C)C ; Cc1ccc2ccc3c(-c4cccc(-c5ccc6c(c5)-c5cccc7cccc-6c57)c4)cc(C)nc3c2n1]

The same procedure as in [Synthesis Example 1], with the exception of using 2-(3-(fluoranthen-8-yl)phenyl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (6.8 g, yield: 68%).

[LCMS]: 485

### [Synthesis Example 14] Synthesis of Compound 14

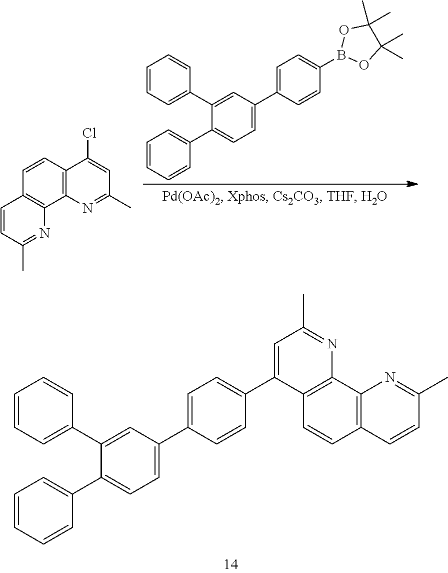 [SMILES: Cc1ccc2ccc3c(Cl)cc(C)nc3c2n1 ; CC1(C)OB(c2ccc(-c3ccc(-c4ccccc4)c(-c4ccccc4)c3)cc2)OC1(C)C ; Cc1ccc2ccc3c(-c4ccc(-c5ccc(-c6ccccc6)c(-c6ccccc6)c5)cc4)cc(C)nc3c2n1]

The same procedure as in [Synthesis Example 1], with the exception of using 4,4,5,5-tetramethyl-2-(4′-phenyl-[1,1′:3′,1″-terphenyl]-4-yl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.0 g, yield:68%)

[LCMS]: 513

### [Synthesis Example 15] Synthesis of Compound 15

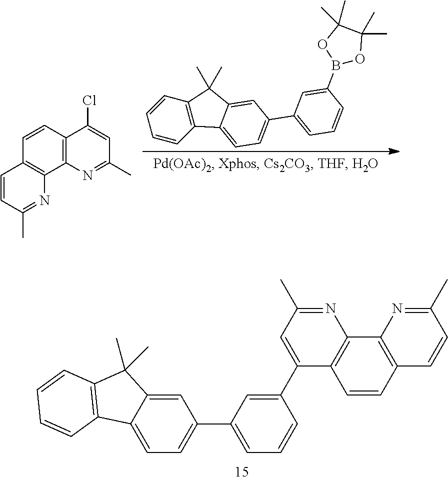 [SMILES: Cc1ccc2ccc3c(Cl)cc(C)nc3c2n1 ; CC1(C)c2ccccc2-c2ccc(-c3cccc(B4OC(C)(C)C(C)(C)O4)c3)cc21 ; Cc1ccc2ccc3c(-c4cccc(-c5ccc6c(c5)C(C)(C)c5ccccc5-6)c4)cc(C)nc3c2n1]

The same procedure as in [Synthesis Example 1], with the exception of using 2-(3-(9,9-dimethyl-9H-fluoren-2-yl)phenyl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.0 g, yield: 72%)

[LCMS]: 477

### [Synthesis Example 16] Synthesis of Compound 16

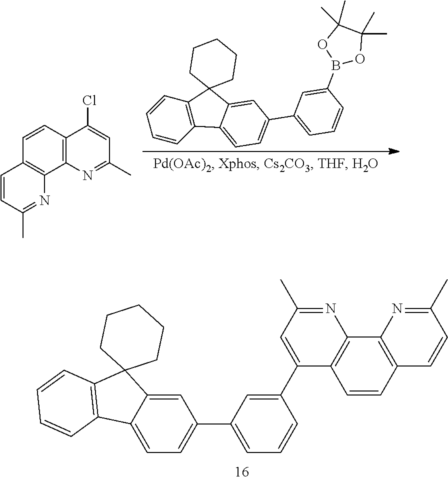 [SMILES: Cc1ccc2ccc3c(Cl)cc(C)nc3c2n1 ; CC1(C)OB(c2cccc(-c3ccc4c(c3)C3(CCCCC3)c3ccccc3-4)c2)OC1(C)C ; Cc1ccc2ccc3c(-c4cccc(-c5ccc6c(c5)C5(CCCCC5)c5ccccc5-6)c4)cc(C)nc3c2n1]

The same procedure as in [Synthesis Example 1], with the exception of using 4,4,5,5-tetramethyl-2-(3-(spiro[cyclohexane-1,9′-fluoren]-2′-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.8 g, yield: 74%).

[LCMS]: 517

### [Synthesis Example 17] Synthesis of Compound 17

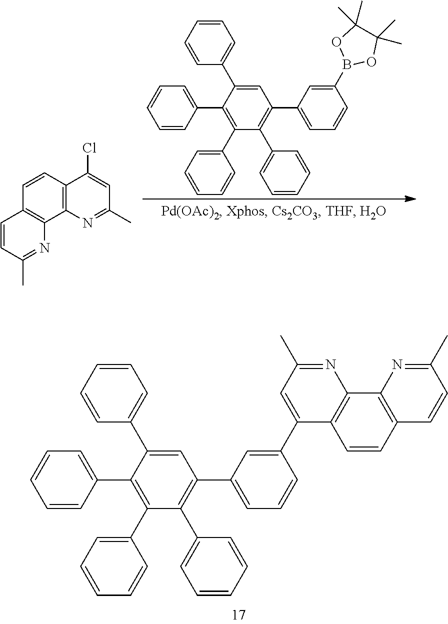 [SMILES: Cc1ccc2ccc3c(Cl)cc(C)nc3c2n1 ; CC1(C)OB(c2cccc(-c3cc(-c4ccccc4)c(-c4ccccc4)c(-c4ccccc4)c3-c3ccccc3)c2)OC1(C)C ; Cc1ccc2ccc3c(-c4cccc(-c5cc(-c6ccccc6)c(-c6ccccc6)c(-c6ccccc6)c5-c5ccccc5)c4)cc(C)nc3c2n1]

The same procedure as in [Synthesis Example 1], with the exception of using 4,4,5,5-tetramethyl-2-(3′,4′,5′-triphenyl-[1,1′:2′,1″-terphenyl]-3-yl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (9.5 g, yield: 70%).

[LCMS]: 665

### [Synthesis Example 18] Synthesis of Compound 18

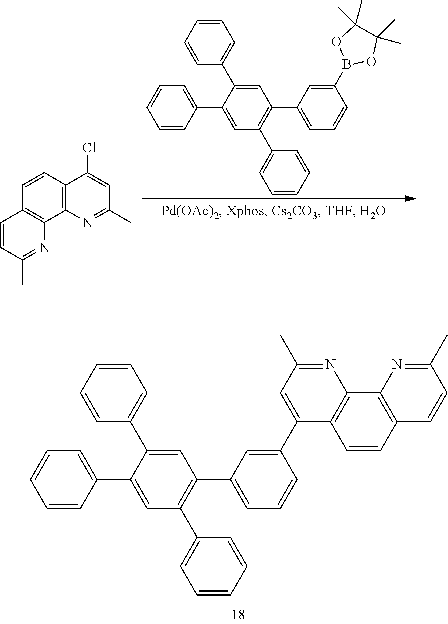 [SMILES: Cc1ccc2ccc3c(Cl)cc(C)nc3c2n1 ; CC1(C)OB(c2cccc(-c3cc(-c4ccccc4)c(-c4ccccc4)cc3-c3ccccc3)c2)OC1(C)C ; Cc1ccc2ccc3c(-c4cccc(-c5cc(-c6ccccc6)c(-c6ccccc6)cc5-c5ccccc5)c4)cc(C)nc3c2n1]

The same procedure as in [Synthesis Example 1], with the exception of using 2-(4′,5′-diphenyl-[1,1′:2′,1″-terphenyl]-3-yl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.3 g, yield: 69%)

[LCMS]: 589

### [Synthesis Example 19] Synthesis of Compound 19

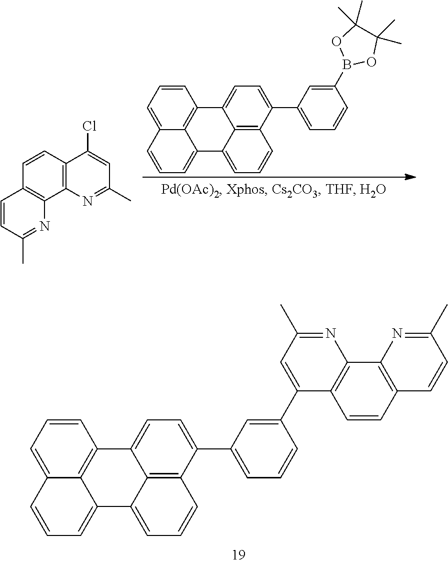 [SMILES: Cc1ccc2ccc3c(Cl)cc(C)nc3c2n1 ; CC1(C)OB(c2cccc(-c3ccc4c5cccc6cccc(c7cccc3c74)c65)c2)OC1(C)C ; Cc1ccc2ccc3c(-c4cccc(-c5ccc6c7cccc8cccc(c9cccc5c96)c87)c4)cc(C)nc3c2n1]

The same procedure as in [Synthesis Example 1], with the exception of using 4,4,5,5-tetramethyl-2-(3-(perylen-3-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.1 g, yield: 65%).

[LCMS]: 535

### [Synthesis Example 20] Synthesis of Compound 20

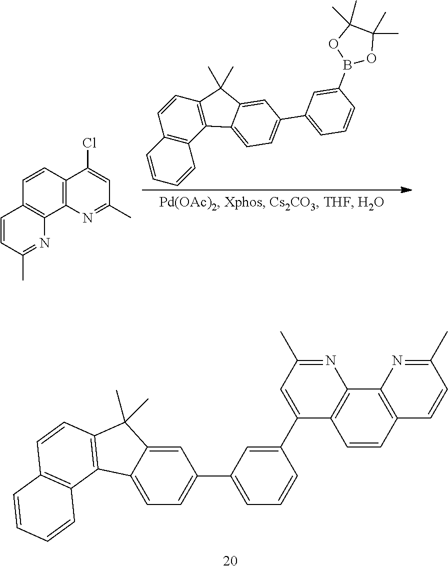 [SMILES: Cc1ccc2ccc3c(Cl)cc(C)nc3c2n1 ; CC1(C)c2cc(-c3cccc(B4OC(C)(C)C(C)(C)O4)c3)ccc2-c2c1ccc1ccccc21 ; Cc1ccc2ccc3c(-c4cccc(-c5ccc6c(c5)C(C)(C)c5ccc7ccccc7c5-6)c4)cc(C)nc3c2n1]

The same procedure as in [Synthesis Example 1], with the exception of using 2-(3-(7,7-dimethyl-7H-benzo[c]fluoren-9-yl)phenyl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.6 g, yield: 72%).

[LCMS]: 527

### [Synthesis Example 21] Synthesis of Compound 21

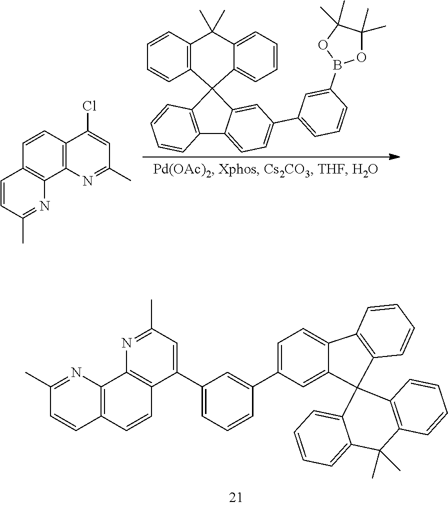 [SMILES: Cc1ccc2ccc3c(Cl)cc(C)nc3c2n1 ; CC1(C)c2ccccc2C2(c3ccccc3-c3ccc(-c4cccc(B5OC(C)(C)C(C)(C)O5)c4)cc32)c2ccccc21 ; Cc1ccc2ccc3c(-c4cccc(-c5ccc6c(c5)C5(c7ccccc7-6)c6ccccc6C(C)(C)c6ccccc65)c4)cc(C)nc3c2n1]

The same procedure as in [Synthesis Example 1], with the exception of using 2-(3-(7,7-dimethyl-7H-benzo[c]fluoren-9-yl)phenyl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.8 g, yield: 67%).

[LCMS]: 641

### [Synthesis Example 22] Synthesis of Compound 22

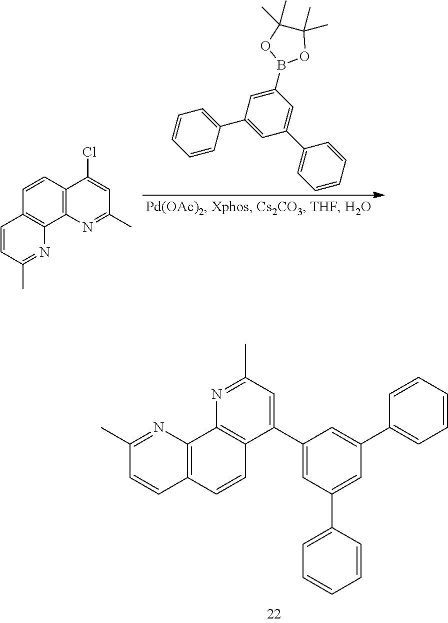 [SMILES: Cc1ccc2ccc3c(Cl)cc(C)nc3c2n1 ; CC1(C)OB(c2cc(-c3ccccc3)cc(-c3ccccc3)c2)OC1(C)C ; Cc1ccc2ccc3c(-c4cc(-c5ccccc5)cc(-c5ccccc5)c4)cc(C)nc3c2n1]

The same procedure as in [Synthesis Example 1], with the exception of using 2-([1,1′:3′,1″-terphenyl]-5′-yl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (6.2 g, yield: 70%).

[LCMS]: 437

### [Synthesis Example 23] Synthesis of Compound 23

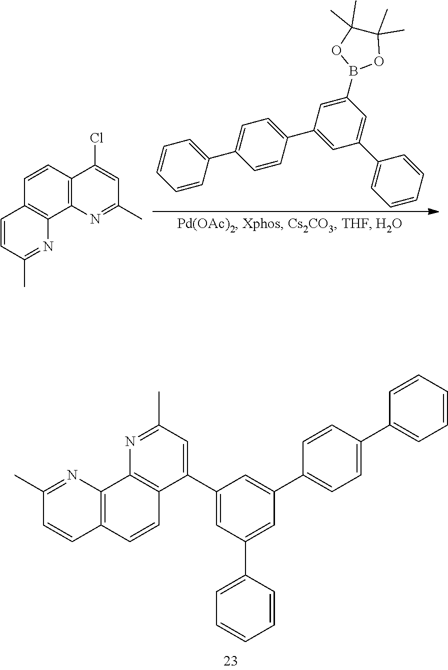 [SMILES: Cc1ccc2ccc3c(Cl)cc(C)nc3c2n1 ; CC1(C)OB(c2cc(-c3ccccc3)cc(-c3ccc(-c4ccccc4)cc3)c2)OC1(C)C ; Cc1ccc2ccc3c(-c4cc(-c5ccccc5)cc(-c5ccc(-c6ccccc6)cc5)c4)cc(C)nc3c2n1]

The same procedure as in [Synthesis Example 1], with the exception of using 2-([1,1′:3′,1″:4″,1′″-quaterphenyl]-5′-yl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.4 g, yield: 70%).

[LCMS]: 513

### [Synthesis Example 24] Synthesis of Compound 24

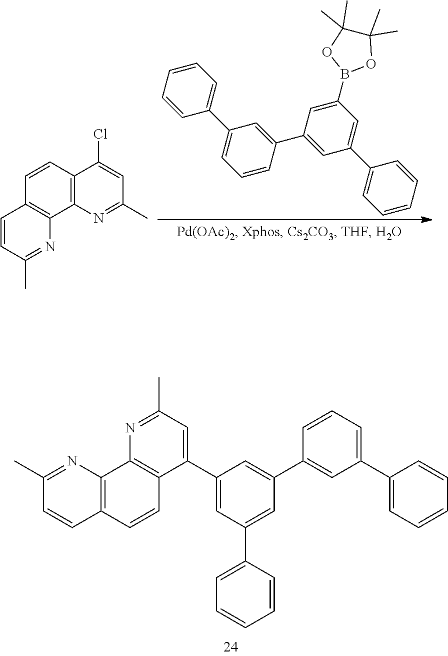 [SMILES: Cc1ccc2ccc3c(Cl)cc(C)nc3c2n1 ; CC1(C)OB(c2cc(-c3ccccc3)cc(-c3cccc(-c4ccccc4)c3)c2)OC1(C)C ; Cc1ccc2ccc3c(-c4cc(-c5ccccc5)cc(-c5cccc(-c6ccccc6)c5)c4)cc(C)nc3c2n1]

The same procedure as in [Synthesis Example 1], with the exception of using 2-([1,1′:3′,1″:4″,1′″-quaterphenyl]-5′-yl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (6.2 g, yield: 70%).

[LCMS]: 513

### [Synthesis Example 25] Synthesis of Compound 25

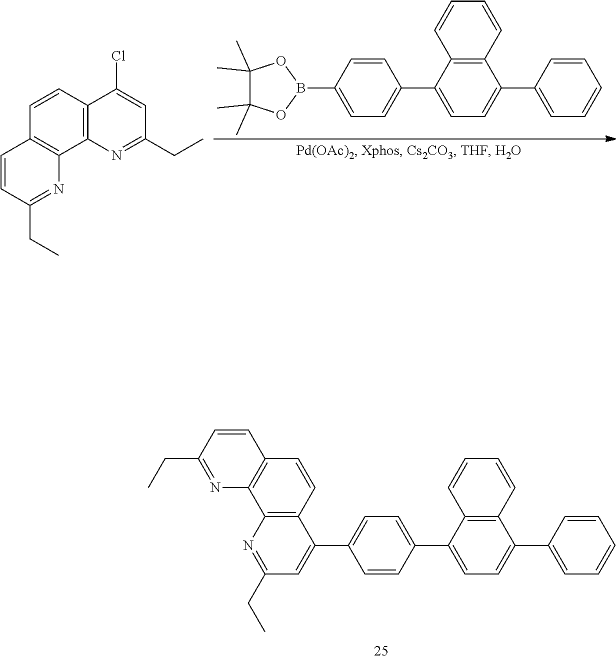

4-Chloro-2,9-diethyl-1,10-phenanthroline (5.6 g, 20.6 mmol), 4,4,5,5-tetramethyl-2-(4-(4-phenylnaphthalen-1-yl)phenyl)-1,3,2-dioxaborolane (8.3 g, 20.6 mmol), Pd(OAc)₂ (0.2 g, 1.0 mmol), Xphos (1.0 g, 2.1 mmol), and Cs₂CO₃ (13.5 g, 41.3 mmol) were added to toluene (50 ml), EtOH (10 ml), and H₂O (10 ml) and heated for 12 hours under reflux. After completion of the reaction, extraction was conducted with methylene chloride. The extract was added with MgSO₄ and then filtered. The solvent was removed from the filtered organic layer which was then purified by column chromatography to afford the target compound (7.4 g, yield: 70%).

[LCMS]: 515

### [Synthesis Example 26] Synthesis of Compound 26

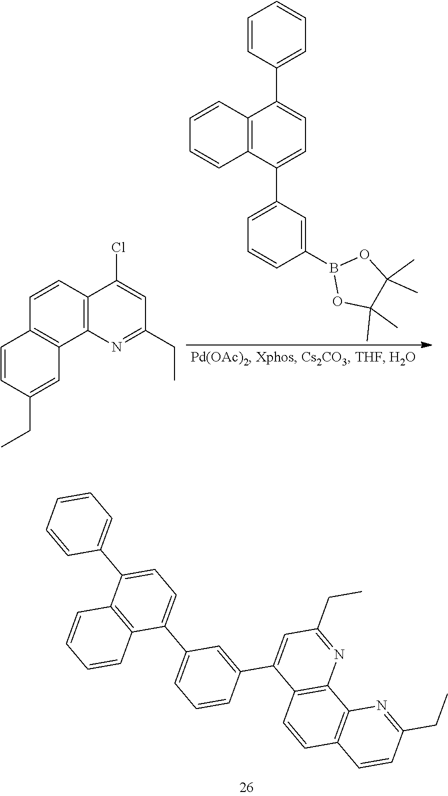 [SMILES: CCc1ccc2ccc3c(Cl)cc(CC)nc3c2c1 ; CC1(C)OB(c2cccc(-c3ccc(-c4ccccc4)c4ccccc34)c2)OC1(C)C ; CCc1ccc2ccc3c(-c4cccc(-c5ccc(-c6ccccc6)c6ccccc56)c4)cc(CC)nc3c2n1]

The same procedure as in [Synthesis Example 25], with the exception of using 4,4,5,5-tetramethyl-2-(3-(4-phenylnaphthalen-1-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.9 g, yield: 74%).

[LCMS]: 515

### [Synthesis Example 27] Synthesis of Compound 27

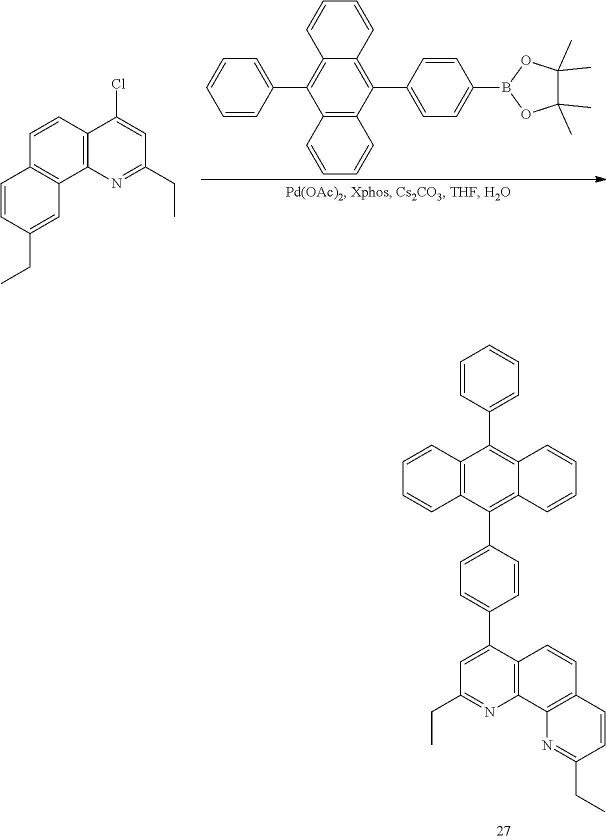 [SMILES: CCc1ccc2ccc3c(Cl)cc(CC)nc3c2c1 ; CC1(C)OB(c2ccc(-c3c4ccccc4c(-c4ccccc4)c4ccccc34)cc2)OC1(C)C ; CCc1ccc2ccc3c(-c4ccc(-c5c6ccccc6c(-c6ccccc6)c6ccccc56)cc4)cc(CC)nc3c2n1]

The same procedure as in [Synthesis Example 25], with the exception of using 4,4,5,5-tetramethyl-2-(4-(10-phenylanthracen-9-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.9 g, yield: 76%).

[LCMS]: 565

### [Synthesis Example 28] Synthesis of Compound 28

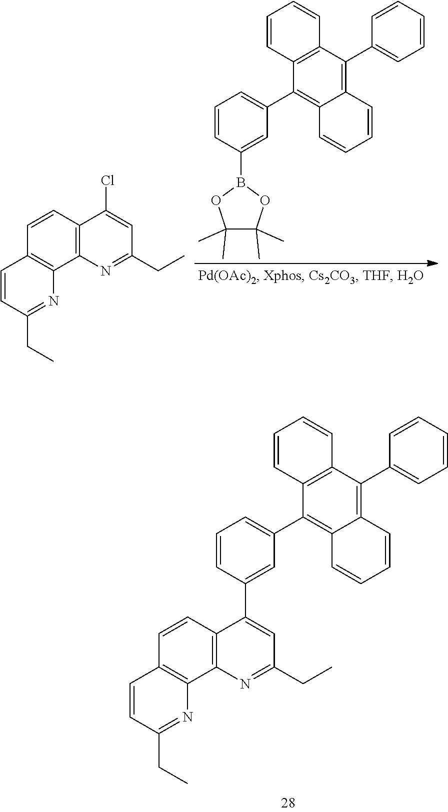

The same procedure as in [Synthesis Example 25], with the exception of using 4,4,5,5-tetramethyl-2-(3-(10-phenylanthracen-9-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.2 g, yield: 71).

[LCMS]: 565

### [Synthesis Example 29] Synthesis of Compound 29

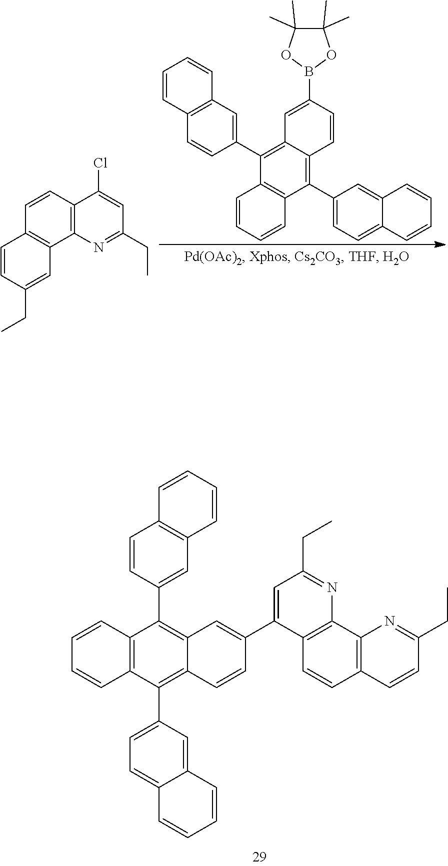 [SMILES: CCc1ccc2ccc3c(Cl)cc(CC)nc3c2c1 ; CC1(C)OB(c2ccc3c(-c4ccc5ccccc5c4)c4ccccc4c(-c4ccc5ccccc5c4)c3c2)OC1(C)C ; CCc1ccc2ccc3c(-c4ccc5c(-c6ccc7ccccc7c6)c6ccccc6c(-c6ccc7ccccc7c6)c5c4)cc(CC)nc3c2n1]

The same procedure as in [Synthesis Example 25], with the exception of using 2-(9,10-di(naphthalen-2-yl)anthracen-2-yl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (9.6 g, yield: 70%)

[LCMS]: 665

### [Synthesis Example 30] Synthesis of Compound 31

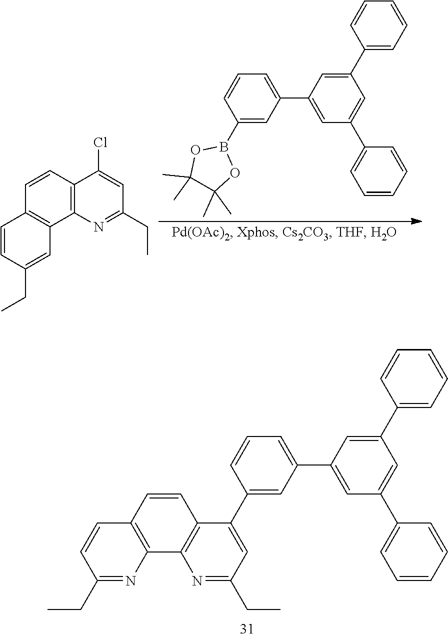

The same procedure as in [Synthesis Example 25], with the exception of using 4,4,5,5-tetramethyl-2-(5′-phenyl-[1,1′:3′,1′-terphenyl]-3-yl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.4 g, yield: 75%)

[LCMS]: 541

### [Synthesis Example 31] Synthesis of Compound 32

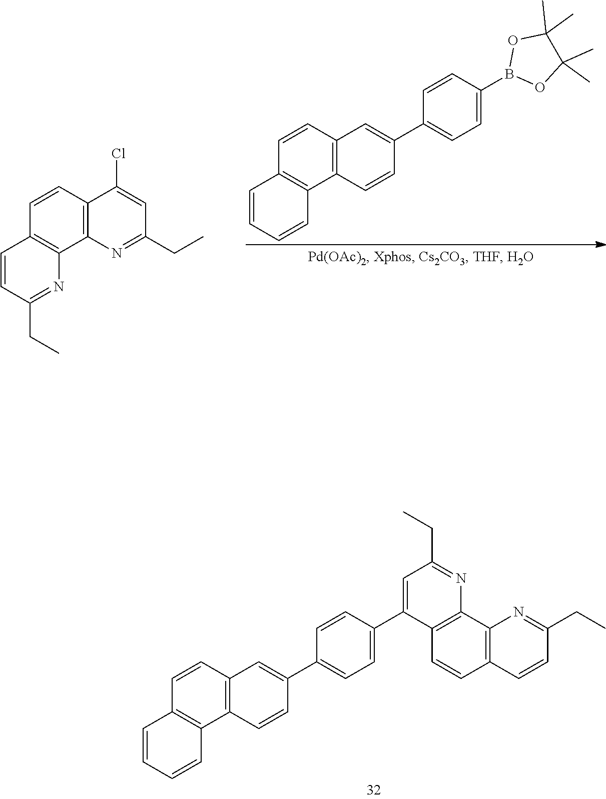 [SMILES: CCc1ccc2ccc3c(Cl)cc(CC)nc3c2n1 ; CC1(C)OB(c2ccc(-c3ccc4c(ccc5ccccc54)c3)cc2)OC1(C)C ; CCc1ccc2ccc3c(-c4ccc(-c5ccc6c(ccc7ccccc76)c5)cc4)cc(CC)nc3c2n1]

The same procedure as in [Synthesis Example 25], with the exception of using 4,4,5,5-tetramethyl-2-(4-(phenanthren-2-yl)phenyl)-1,3,2-dioxaborolane, instead of 4,4,5,5-tetramethyl-2-(4-(4-phenylnaphthalen-1-yl)phenyl)-1,3,2-dioxaborolane, as a reactant, was carried out to afford the target compound (7.0 g, yield: 70%)

[LCMS]: 489

### [Synthesis Example 32] Synthesis of Compound 33

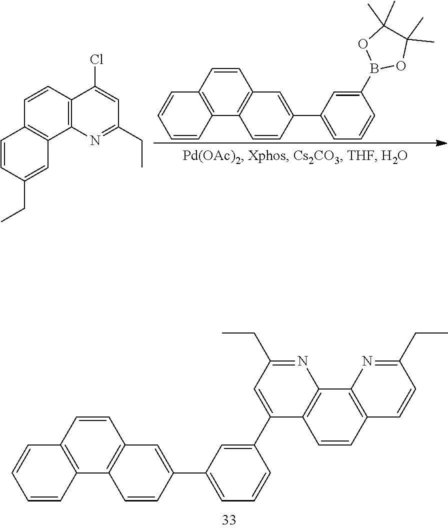

The same procedure as in [Synthesis Example 25], with the exception of using 4,4,5,5-tetramethyl-2-(3-(phenanthren-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.3 g, yield: 72%).

[LCMS]: 489

### [Synthesis Example 33] Synthesis of Compound 34

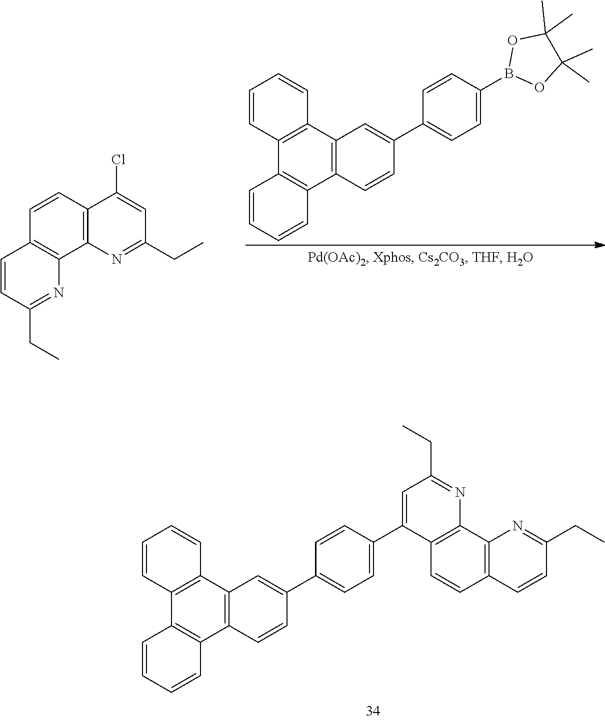 [SMILES: CCc1ccc2ccc3c(Cl)cc(CC)nc3c2n1 ; CC1(C)OB(c2ccc(-c3ccc4c5ccccc5c5ccccc5c4c3)cc2)OC1(C)C ; CCc1ccc2ccc3c(-c4ccc(-c5ccc6c7ccccc7c7ccccc7c6c5)cc4)cc(CC)nc3c2n1]

The same procedure as in [Synthesis Example 25], with the exception of using 4,4,5,5-tetramethyl-2-(4-(triphenylen-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.5 g, yield: 68%).

[LCMS]: 539

### [Synthesis Example 34] Synthesis of Compound 35

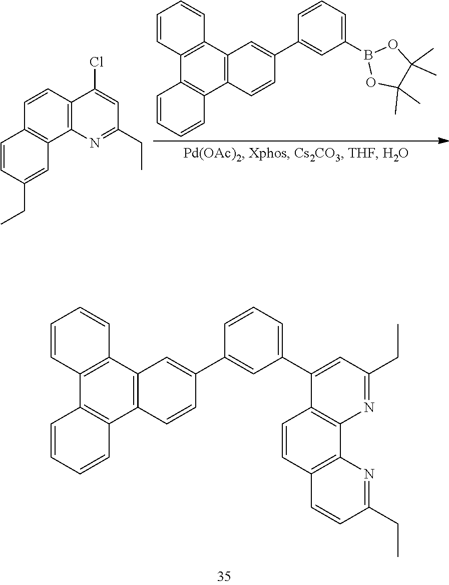

The same procedure as in [Synthesis Example 25], with the exception of using 4,4,5,5-tetramethyl-2-(3-(triphenylen-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.0 g, yield: 72%).

[LCMS]: 539

### [Synthesis Example 35] Synthesis of Compound 36

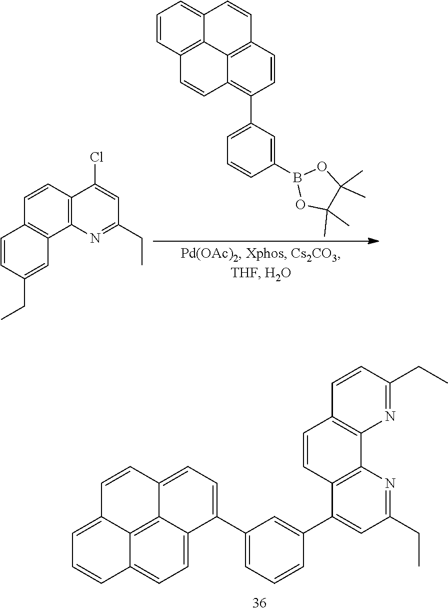 [SMILES: CCc1ccc2ccc3c(Cl)cc(CC)nc3c2c1 ; CC1(C)OB(c2cccc(-c3ccc4ccc5cccc6ccc3c4c56)c2)OC1(C)C ; CCc1ccc2ccc3c(-c4cccc(-c5ccc6ccc7cccc8ccc5c6c78)c4)cc(CC)nc3c2n1]

The same procedure as in [Synthesis Example 25], with the exception of using 4,4,5,5-tetramethyl-2-(3-(pyren-1-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.9 g, yield: 75%).

[LCMS]: 513

### [Synthesis Example 36] Synthesis of Compound 50

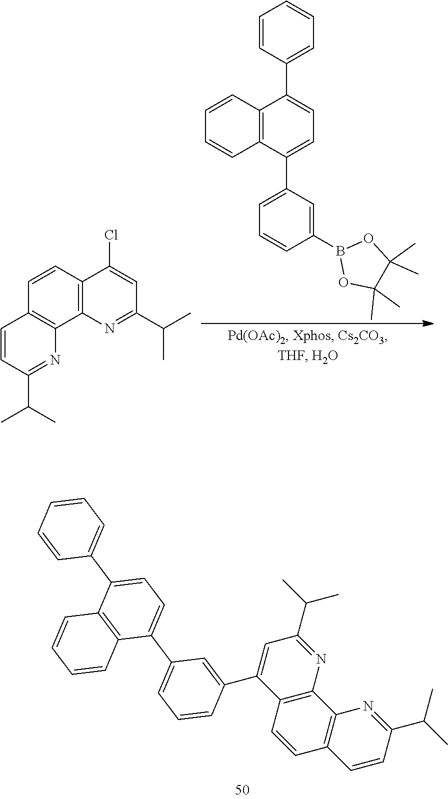 [SMILES: CC(C)c1ccc2ccc3c(Cl)cc(C(C)C)nc3c2n1 ; CC1(C)OB(c2cccc(-c3ccc(-c4ccccc4)c4ccccc34)c2)OC1(C)C ; CC(C)c1ccc2ccc3c(-c4cccc(-c5ccc(-c6ccccc6)c6ccccc56)c4)cc(C(C)C)nc3c2n1]

4-Chloro-2,9-diisopropyl-1,10-phenanthroline (6.15 g, 20.6 mmol), 4,4,5,5-tetramethyl-2-(4-(4-phenylnaphthalen-1-yl)phenyl)-1,3,2-dioxaborolane (8.3 g, 20.6 mmol), Pd(OAc)₂ (0.2 g, 1.0 mmol), Xphos (1.0 g, 2.1 mmol), and Cs₂CO₃ (13.5 g, 41.3 mmol) were added to toluene (50 ml), EtOH (10 ml), and H₂O (10 ml) and heated for 12 hours under reflux. After completion of the reaction, extraction was conducted with methylene chloride. The extract was added with MgSO₄ and then filtered. The solvent was removed from the filtered organic layer which was then purified by column chromatography to afford the target compound (7.7 g, yield: 69%).

[LCMS]: 543

### [Synthesis Example 37] Synthesis of Compound 51

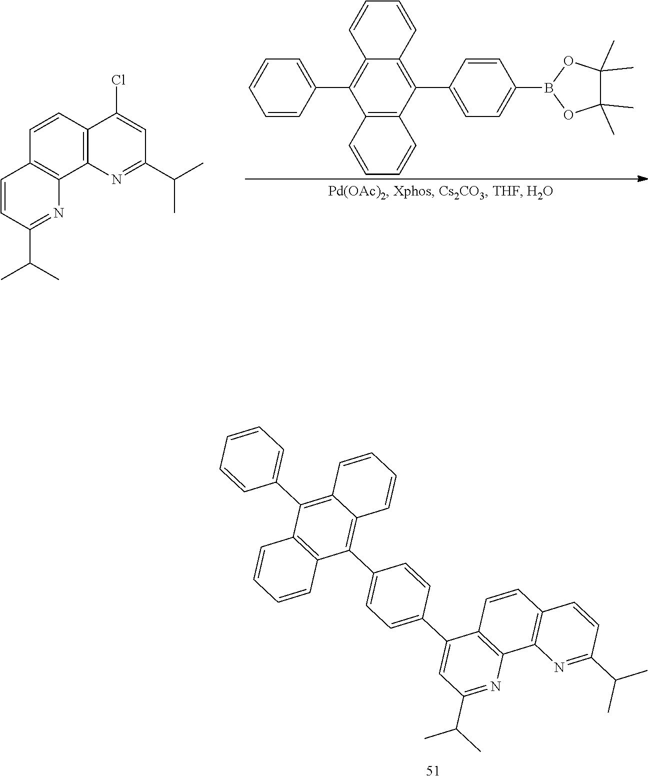 [SMILES: CC(C)c1ccc2ccc3c(Cl)cc(C(C)C)nc3c2n1 ; CC1(C)OB(c2ccc(-c3c4ccccc4c(-c4ccccc4)c4ccccc34)cc2)OC1(C)C ; CC(C)c1ccc2ccc3c(-c4ccc(-c5c6ccccc6c(-c6ccccc6)c6ccccc56)cc4)cc(C(C)C)nc3c2n1]

The same procedure as in [Synthesis Example 36], with the exception of using 4,4,5,5-tetramethyl-2-(4-(10-phenylanthracen-9-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.7 g, yield: 71%).

[LCMS]: 593

### [Synthesis Example 38] Synthesis of Compound 52

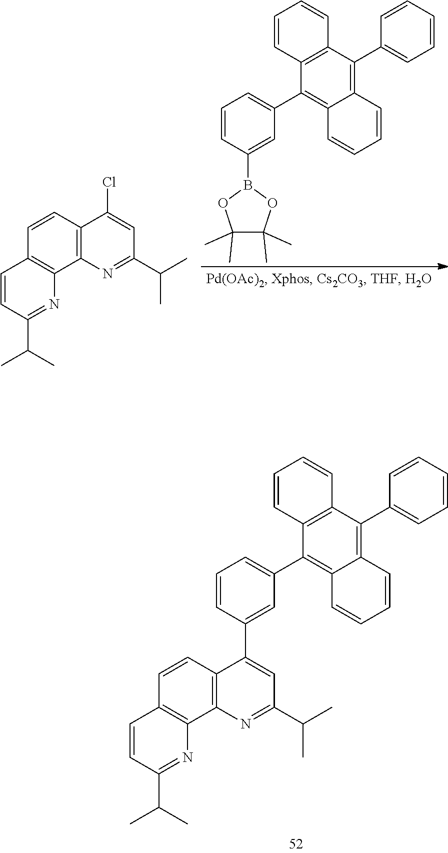

The same procedure as in [Synthesis Example 36], with the exception of using 4,4,5,5-tetramethyl-2-(3-(10-phenylanthracen-9-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.7 g, yield: 71%).

[LCMS]: 593

### [Synthesis Example 39] Synthesis of Compound 53

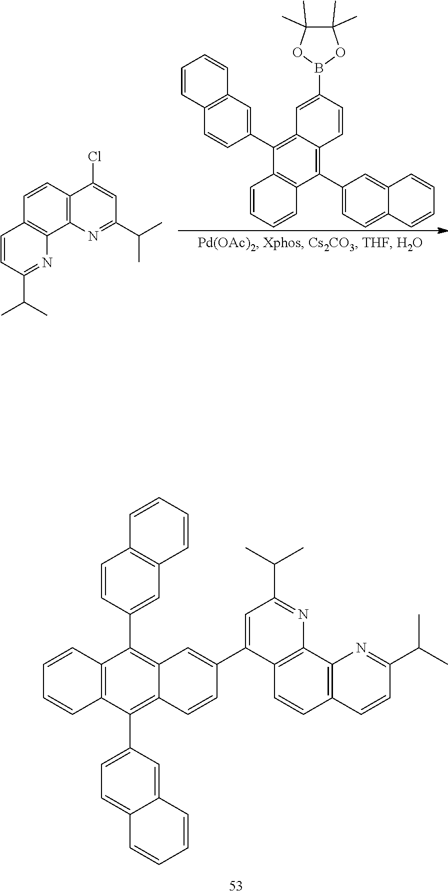 [SMILES: CC(C)c1ccc2ccc3c(Cl)cc(C(C)C)nc3c2n1 ; CC1(C)OB(c2ccc3c(-c4ccc5ccccc5c4)c4ccccc4c(-c4ccc5ccccc5c4)c3c2)OC1(C)C ; CC(C)c1ccc2ccc3c(-c4ccc5c(-c6ccc7ccccc7c6)c6ccccc6c(-c6ccc7ccccc7c6)c5c4)cc(C(C)C)nc3c2n1]

The same procedure as in [Synthesis Example 36], with the exception of using 2-(9,10-di(naphthalen-2-yl)anthracen-2-yl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (9.6 g, yield: 67%)

[LCMS]: 693

### [Synthesis Example 40] Synthesis of Compound 55

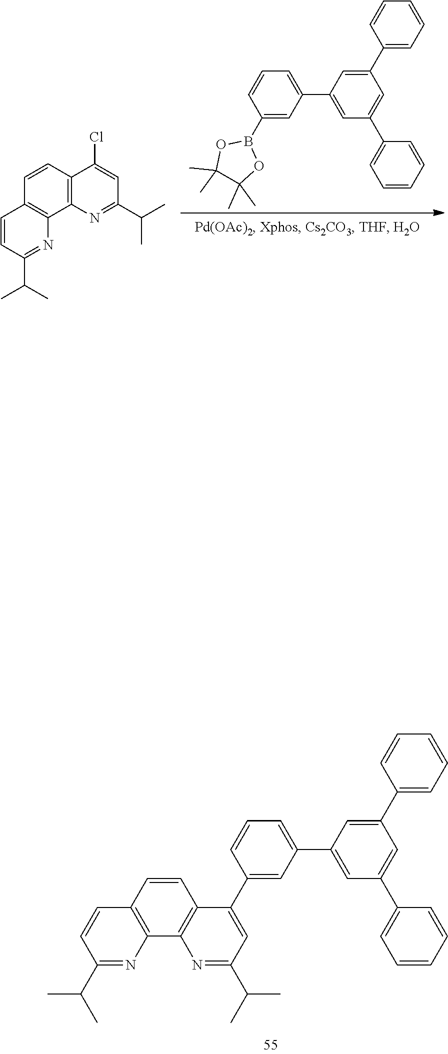

The same procedure as in [Synthesis Example 36], with the exception of using 4,4,5,5-tetramethyl-2-(5′-phenyl-[1,1′:3′,1′-terphenyl]-3-yl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.1 g, yield: 69%)

[LCMS]: 569

### [Synthesis Example 41] Synthesis of Compound 56

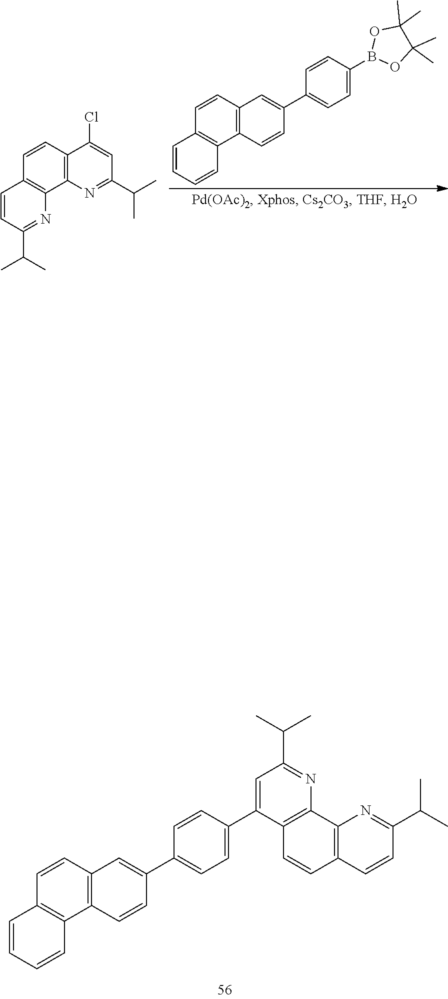

The same procedure as in [Synthesis Example 36], with the exception of using 4,4,5,5-tetramethyl-2-(4-(phenanthren-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.1 g, yield: 67%).

[LCMS]: 517

### [Synthesis Example 42] Synthesis of Compound 57

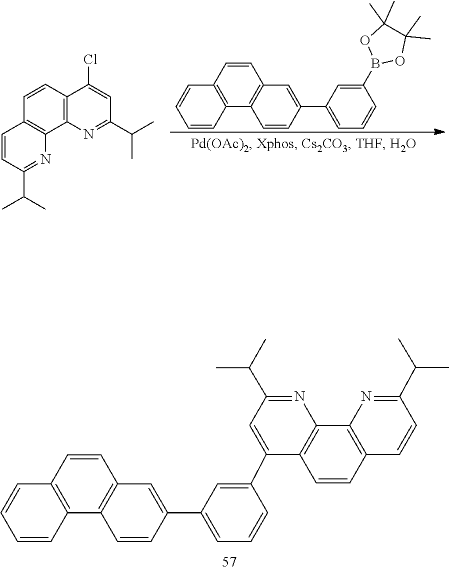

The same procedure as in [Synthesis Example 36], with the exception of using 4,4,5,5-tetramethyl-2-(3-(phenanthren-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.1 g, yield: 67%).

[LCMS]: 517

### [Synthesis Example 43] Synthesis of Compound 59

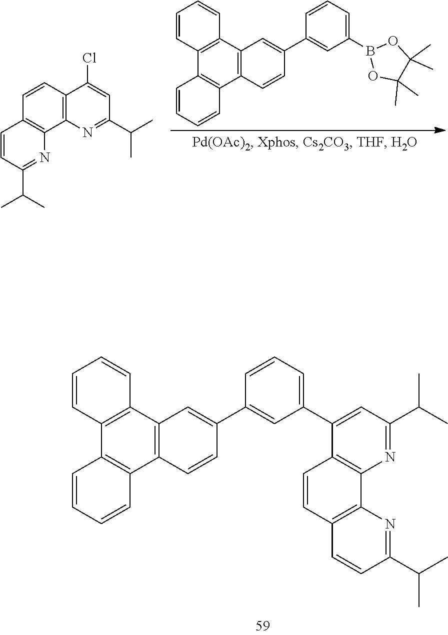

The same procedure as in [Synthesis Example 36], with the exception of using 4,4,5,5-tetramethyl-2-(3-(triphenylen-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.9 g, yield: 68%).

[LCMS]: 567

### [Synthesis Example 44] Synthesis of Compound 60

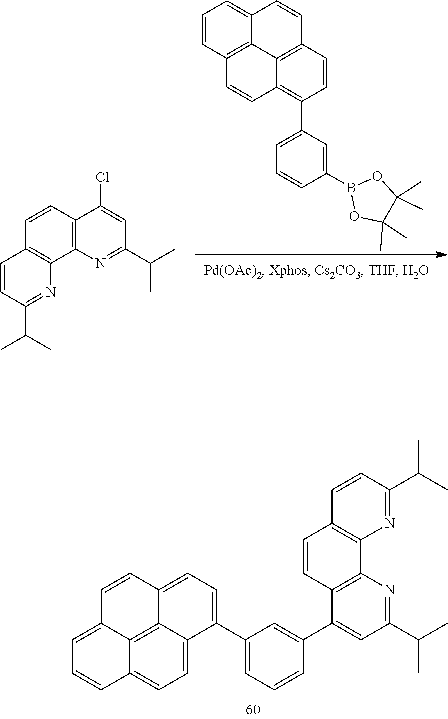 [SMILES: CC(C)c1ccc2ccc3c(Cl)cc(C(C)C)nc3c2n1 ; CC1(C)OB(c2cccc(-c3ccc4ccc5cccc6ccc3c4c56)c2)OC1(C)C ; CC(C)c1ccc2ccc3c(-c4cccc(-c5ccc6ccc7cccc8ccc5c6c78)c4)cc(C(C)C)nc3c2n1]

The same procedure as in [Synthesis Example 36], with the exception of using 4,4,5,5-tetramethyl-2-(3-(pyren-1-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.1 g, yield: 64%).

[LCMS]: 541

### [Synthesis Example 45] Synthesis of Compound 61

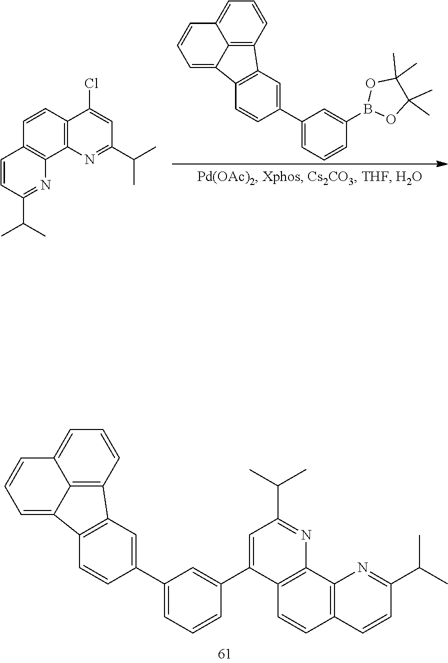 [SMILES: CC(C)c1ccc2ccc3c(Cl)cc(C(C)C)nc3c2n1 ; CC1(C)OB(c2cccc(-c3ccc4c(c3)-c3cccc5cccc-4c35)c2)OC1(C)C ; CC(C)c1ccc2ccc3c(-c4cccc(-c5ccc6c(c5)-c5cccc7cccc-6c57)c4)cc(C(C)C)nc3c2n1]

The same procedure as in [Synthesis Example 36], with the exception of using 2-(3-(fluoranthen-8-yl)phenyl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.9 g, yield: 71%).

[LCMS]: 541

### [Synthesis Example 46] Synthesis of Compound 63

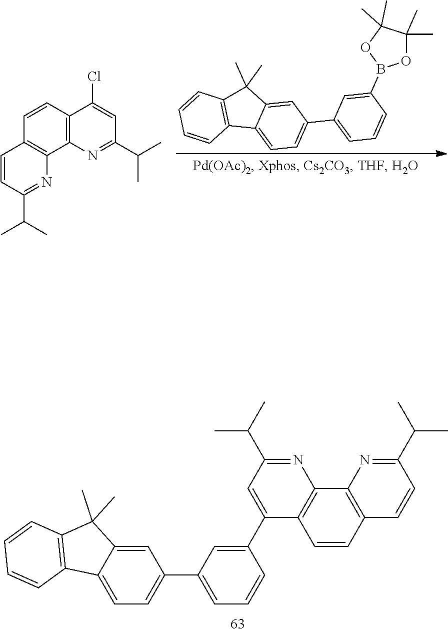

The same procedure as in [Synthesis Example 36], with the exception of using 2-(3-(9,9-dimethyl-9H-fluoren-2-yl)phenyl)-4,4,5,5-tetramethyl-1,3,2-dioxaborolane instead of 4,4,5,5-tetramethyl-2-(4-(4-phenylnaphthalen-1-yl)phenyl)-1,3,2-dioxaborolane, was carried out to afford the target compound (7.7 g, yield: 70%).

[LCMS]: 533

### [Synthesis Example 47] Synthesis of Compound 64

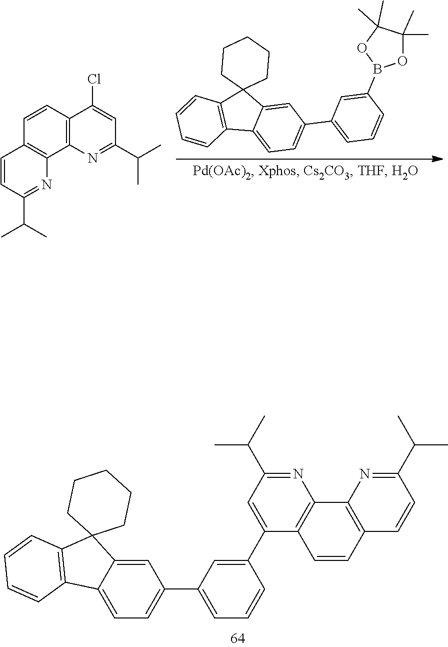

The same procedure as in [Synthesis Example 36], with the exception of using 4,4,5,5-tetramethyl-2-(3-(spiro[cyclohexane-1,9′-fluoren]-2′-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (8.0 g, yield: 68%).

[LCMS]: 573

### [Synthesis Example 48] Synthesis of Compound 74

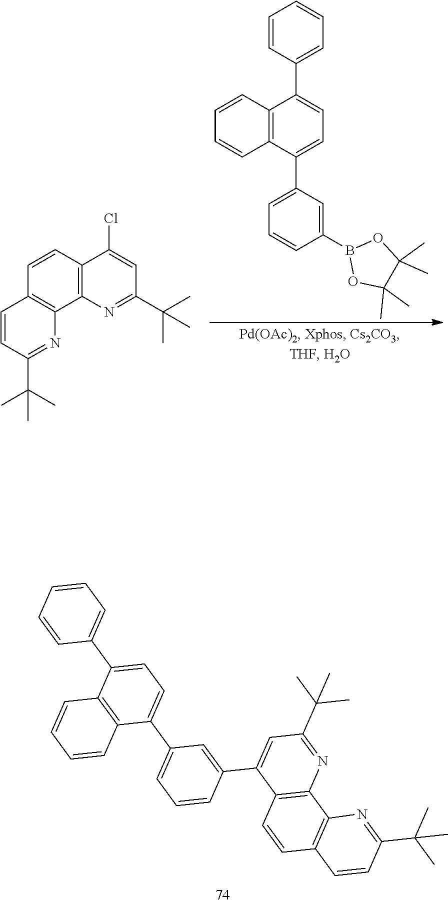 [SMILES: CC(C)(C)c1ccc2ccc3c(Cl)cc(C(C)(C)C)nc3c2n1 ; CC1(C)OB(c2cccc(-c3ccc(-c4ccccc4)c4ccccc34)c2)OC1(C)C ; CC(C)(C)c1ccc2ccc3c(-c4cccc(-c5ccc(-c6ccccc6)c6ccccc56)c4)cc(C(C)(C)C)nc3c2n1]

2,9-Di-tert-butyl-4-chloro-1,10-phenanthroline (6.75 g, 20.6 mmol), 4,4,5,5-tetramethyl-2-(3-(4-phenylnaphthalen-1-yl)phenyl)-1,3,2-dioxaborolane (8.3 g, 20.6 mmol), Pd(OAc)₂ (0.2 g, 1.0 mmol), Xphos (1.0 g, 2.1 mmol), and Cs₂CO₃ (13.5 g, 41.3 mmol) were added to toluene (50 ml), EtOH (10 ml), and H₂O (10 ml) and heated for 12 hours under reflux. After completion of the reaction, extraction was conducted with methylene chloride. The extract was added with MgSO₄ and then filtered. The solvent was removed from the filtered organic layer which was then purified by column chromatography to afford the target compound (7.6 g, yield: 65%).

[LCMS]: 571

### [Synthesis Example 49] Synthesis of Compound 75

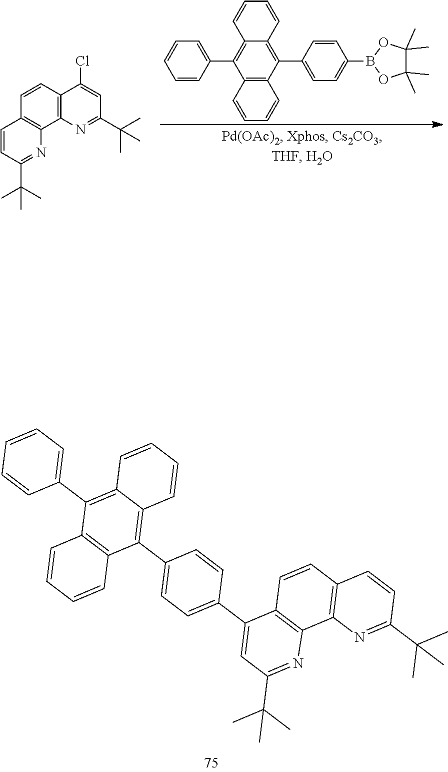 [SMILES: CC(C)(C)c1ccc2ccc3c(Cl)cc(C(C)(C)C)nc3c2n1 ; CC1(C)OB(c2ccc(-c3c4ccccc4c(-c4ccccc4)c4ccccc34)cc2)OC1(C)C ; CC(C)(C)c1ccc2ccc3c(-c4ccc(-c5c6ccccc6c(-c6ccccc6)c6ccccc56)cc4)cc(C(C)(C)C)nc3c2n1]

The same procedure as in [Synthesis Example 48], with the exception of using 4,4,5,5-tetramethyl-2-(4-(10-phenylanthracen-9-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (8.3 g, yield: 65%).

[LCMS]: 621

### [Synthesis Example 50] Synthesis of Compound 76

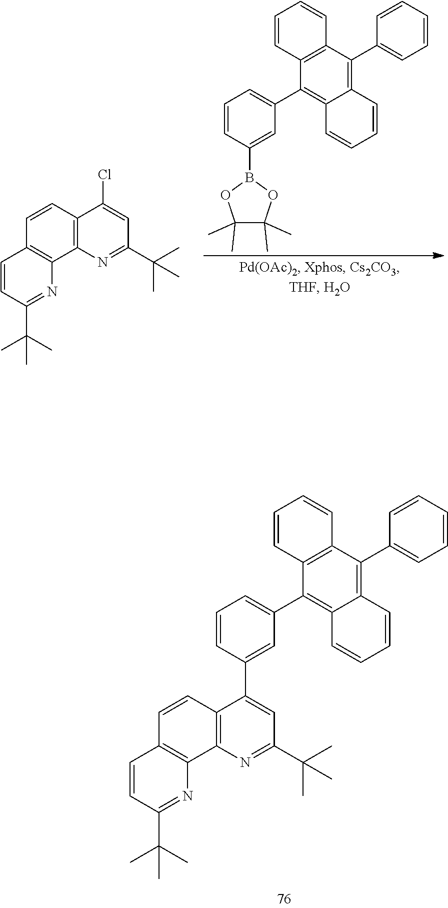

The same procedure as in [Synthesis Example 48], with the exception of using 4,4,5,5-tetramethyl-2-(3-(10-phenylanthracen-9-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (8.3 g, yield: 65%).

[LCMS]: 621

### [Synthesis Example 51] Synthesis of Compound 77

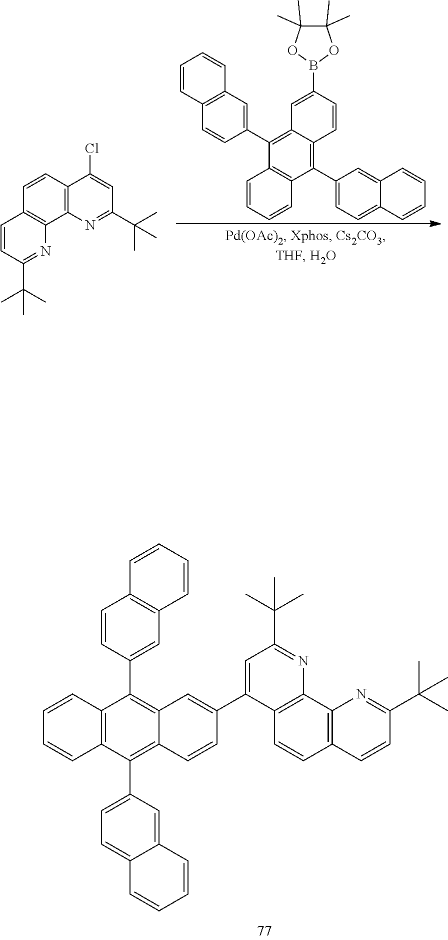 [SMILES: CC(C)(C)c1ccc2ccc3c(Cl)cc(C(C)(C)C)nc3c2n1 ; CC1(C)OB(c2ccc3c(-c4ccc5ccccc5c4)c4ccccc4c(-c4ccc5ccccc5c4)c3c2)OC1(C)C ; CC(C)(C)c1ccc2ccc3c(-c4ccc5c(-c6ccc7ccccc7c6)c6ccccc6c(-c6ccc7ccccc7c6)c5c4)cc(C(C)(C)C)nc3c2n1]

The same procedure as in [Synthesis Example 48], with the exception of using 2-(9,10-di(naphthalen-2-yl)anthracen-2-yl)-4,4,5,5-tetramethyl-1,3,2-dioxaborolane instead of 4,4,5,5-tetramethyl-2-(3-(4-phenylnaphthalen-1-yl)phenyl)-1,3,2-dioxaborolane, was carried out to afford the target compound (9.1 g, yield: 61%)

[LCMS]: 9.1

### [Synthesis Example 52] Synthesis of Compound 79

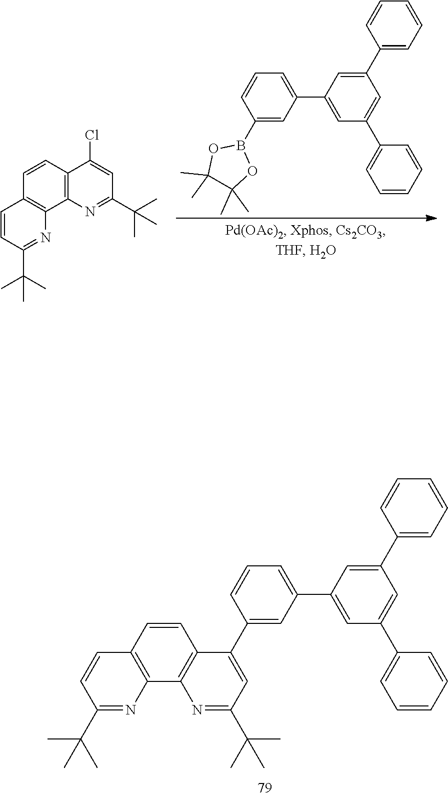

The same procedure as in [Synthesis Example 48], with the exception of using 4,4,5,5-tetramethyl-2-(5′-phenyl-[1,1′:3′,1′-terphenyl]-3-yl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (7.9 g, yield: 64%)

[LCMS]: 597

### [Synthesis Example 53] Synthesis of Compound 80

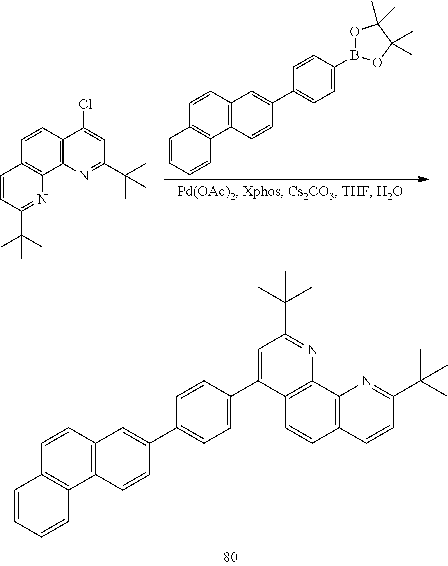

The same procedure as in [Synthesis Example 48], with the exception of using 4,4,5,5-tetramethyl-2-(4-(phenanthren-2-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (7.0 g, yield: 62%).

[LCMS]: 545

### [Synthesis Example 54] Synthesis of Compound 81

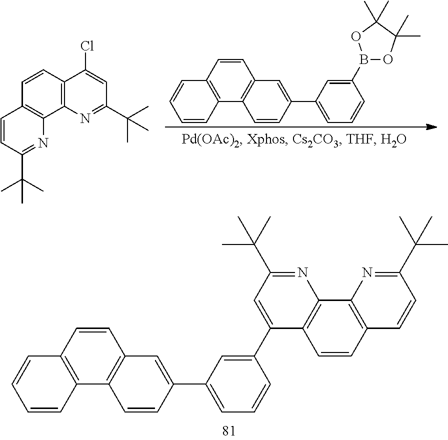

The same procedure as in [Synthesis Example 48], with the exception of using 4,4,5,5-tetramethyl-2-(3-(phenanthren-2-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (7.0 g, yield: 62%).

[LCMS]: 545

### [Synthesis Example 55] Synthesis of Compound 83

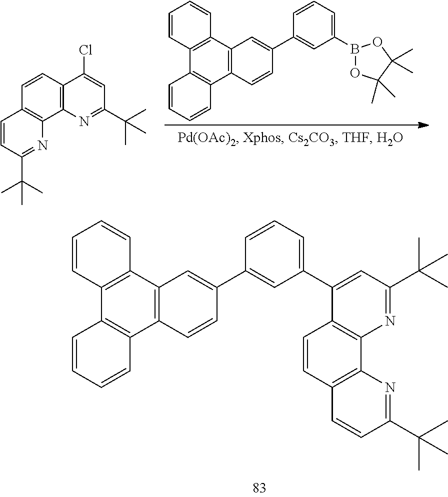

The same procedure as in [Synthesis Example 48], with the exception of using 4,4,5,5-tetramethyl-2-(3-(triphenylen-2-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (8.1 g, yield: 66%).

[LCMS]: 595

### [Synthesis Example 56] Synthesis of Compound 84

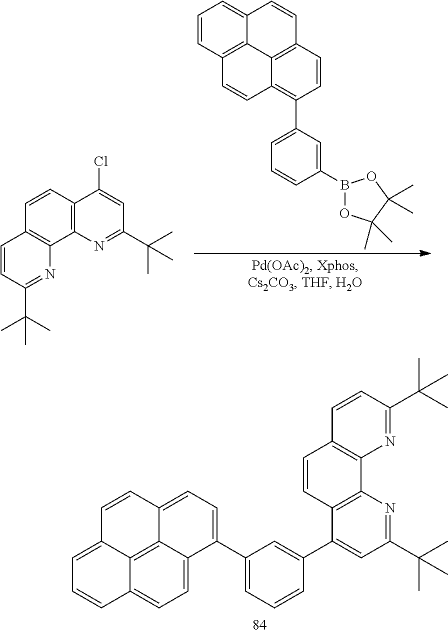 [SMILES: CC(C)(C)c1ccc2ccc3c(Cl)cc(C(C)(C)C)nc3c2n1 ; CC1(C)OB(c2cccc(-c3ccc4ccc5cccc6ccc3c4c56)c2)OC1(C)C ; CC(C)(C)c1ccc2ccc3c(-c4cccc(-c5ccc6ccc7cccc8ccc5c6c78)c4)cc(C(C)(C)C)nc3c2n1]

The same procedure as in [Synthesis Example 48], with the exception of using 4,4,5,5-tetramethyl-2-(3-(pyren-4-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (7.7 g, yield: 66%).

[LCMS]: 569

### [Synthesis Example 57] Synthesis of Compound 91

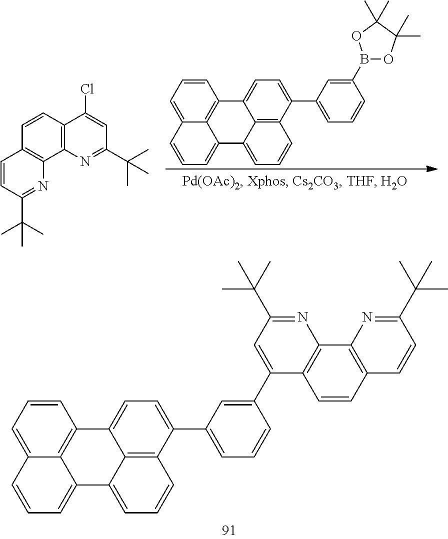

The same procedure as in [Synthesis Example 48], with the exception of using 4,4,5,5-tetramethyl-2-(3-(perylen-3-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (8.9 g, yield: 70%).

[LCMS]: 619

### [Synthesis Example 58] Synthesis of Compound 95

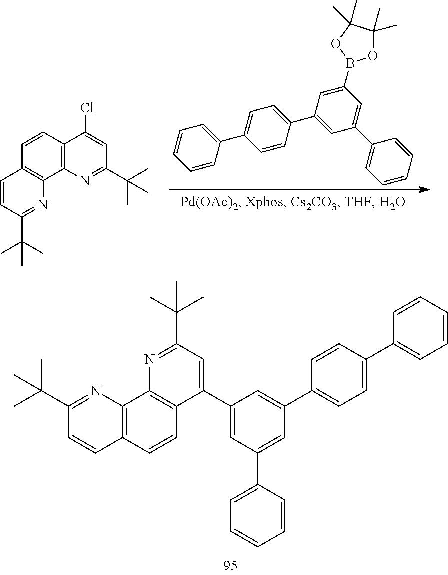

The same procedure as in [Synthesis Example 48], with the exception of using 2-([1,1′:3′,1″:4″,1′″-quaterphenyl]-5′-yl)-4,4,5,5-tetramethyl-1,3,2-dioxaborolane instead of 4,4,5,5-tetramethyl-2-(3-(4-phenylnaphthalen-1-yl)phenyl)-1,3,2-dioxaborolane, was carried out to afford the target compound (8.2 g, yield: 67%).

[LCMS]: 597

### [Synthesis Example 59] Synthesis of Compound 96

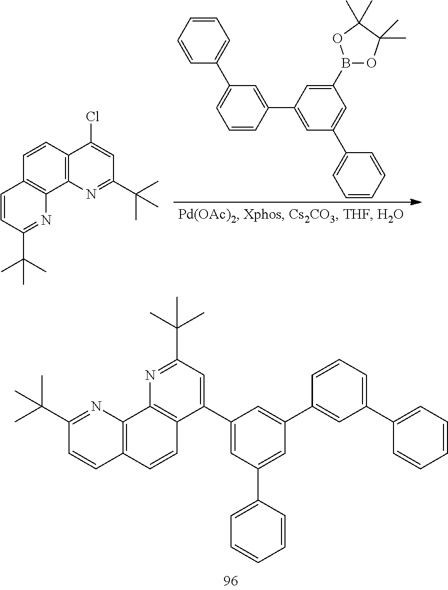 [SMILES: CC(C)(C)c1ccc2ccc3c(Cl)cc(C(C)(C)C)nc3c2n1 ; CC1(C)OB(c2cc(-c3ccccc3)cc(-c3cccc(-c4ccccc4)c3)c2)OC1(C)C ; CC(C)(C)c1ccc2ccc3c(-c4cc(-c5ccccc5)cc(-c5cccc(-c6ccccc6)c5)c4)cc(C(C)(C)C)nc3c2n1]

The same procedure as in [Synthesis Example 48], with the exception of using 2-([1,1′:3′,1″:4″,1′″-quaterphenyl]-5′-yl)-4,4,5,5-tetramethyl-1,3,2-dioxaborolane instead of 4,4,5,5-tetramethyl-2-(3-(4-phenylnaphthalen-1-yl)phenyl)-1,3,2-dioxaborolane, was carried out to afford the target compound (8.2 g, yield: 67%).

[LCMS]: 597

### [Synthesis Example 60] Synthesis of Compound 98

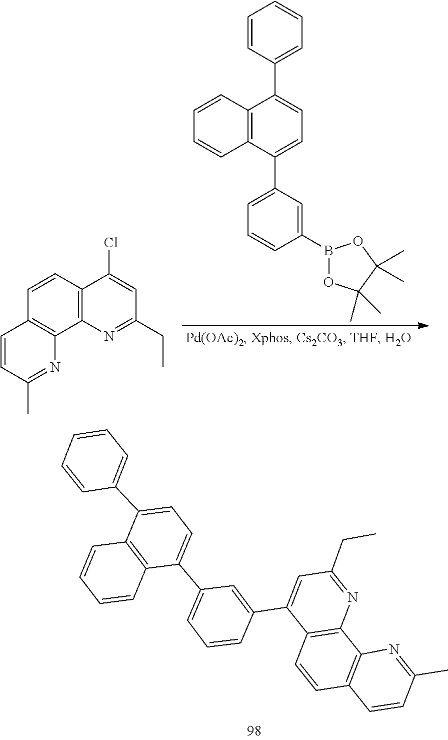 [SMILES: CCc1cc(Cl)c2ccc3ccc(C)nc3c2n1 ; CC1(C)OB(c2cccc(-c3ccc(-c4ccccc4)c4ccccc34)c2)OC1(C)C ; CCc1cc(-c2cccc(-c3ccc(-c4ccccc4)c4ccccc34)c2)c2ccc3ccc(C)nc3c2n1]

20.6 mmol), 4,4,5,5-tetramethyl-2-(3-(4-phenylnaphthalen-1-yl)phenyl)-1,3,2-dioxaborolane (8.3 g, 20.6 mmol), Pd(OAc)₂ (0.2 g, 1.0 mmol), Xphos (1.0 g, 2.1 mmol), and Cs₂CO₃ (13.5 g, 41.3 mmol) were added to toluene (50 ml), EtOH (10 ml), and H₂O (10 ml) and heated for 12 hours under reflux. After completion of the reaction, extraction was conducted with methylene chloride. The extract was added with MgSO₄ and then filtered. The solvent was removed from the filtered organic layer which was then purified by column chromatography to afford the target compound (6.6 g, yield: 64%).

[LCMS]: 501

### [Synthesis Example 61] Synthesis of Compound 99

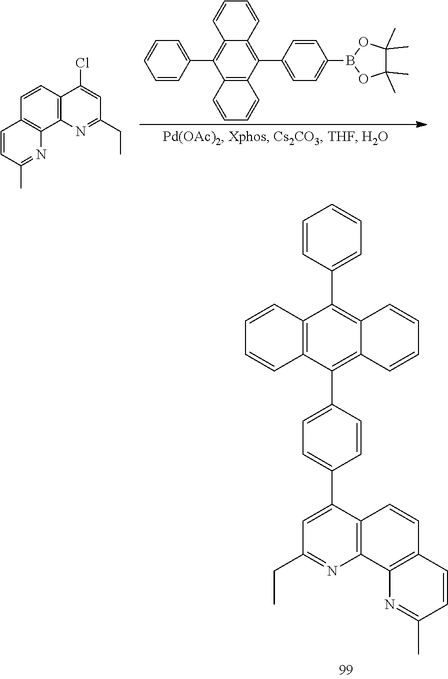 [SMILES: CCc1cc(Cl)c2ccc3ccc(C)nc3c2n1 ; CC1(C)OB(c2ccc(-c3c4ccccc4c(-c4ccccc4)c4ccccc34)cc2)OC1(C)C ; CCc1cc(-c2ccc(-c3c4ccccc4c(-c4ccccc4)c4ccccc34)cc2)c2ccc3ccc(C)nc3c2n1]

The same procedure as in [Synthesis Example 60], with the exception of using 4,4,5,5-tetramethyl-2-(4-(10-phenylanthracen-9-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (7.3 g, yield: 64%).

[LCMS]: 551

### [Synthesis Example 62] Synthesis of Compound 100

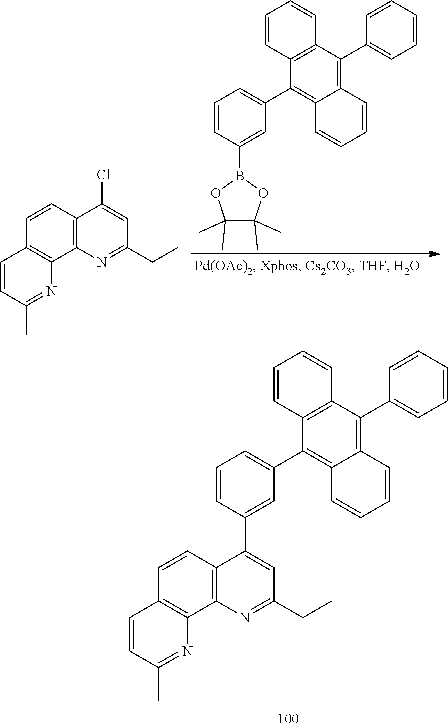

The same procedure as in [Synthesis Example 60], with the exception of using 4,4,5,5-tetramethyl-2-(3-(10-phenylanthracen-9-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (7.3 g, yield: 64%).

[LCMS]: 551

### [Synthesis Example 63] Synthesis of Compound 101

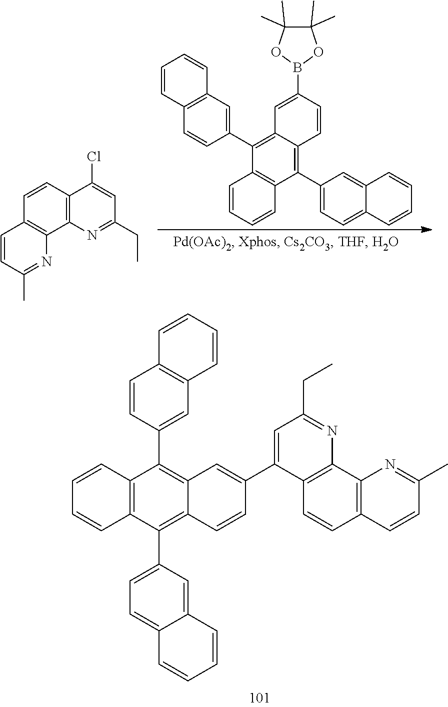 [SMILES: CCc1cc(Cl)c2ccc3ccc(C)nc3c2n1 ; CC1(C)OB(c2ccc3c(-c4ccc5ccccc5c4)c4ccccc4c(-c4ccc5ccccc5c4)c3c2)OC1(C)C ; CCc1cc(-c2ccc3c(-c4ccc5ccccc5c4)c4ccccc4c(-c4ccc5ccccc5c4)c3c2)c2ccc3ccc(C)nc3c2n1]

The same procedure as in [Synthesis Example 60], with the exception of using 2-(9,10-di(naphthalen-2-yl)anthracen-2-yl)-4,4,5,5-tetramethyl-1,3,2-dioxaborolane instead of 4,4,5,5-tetramethyl-2-(3-(4-phenylnaphthalen-1-yl)phenyl)-1,3,2-dioxaborolane, was carried out to afford the target compound (8.0 g, yield: 60%)

[LCMS]: 651

### [Synthesis Example 64] Synthesis of Compound 103

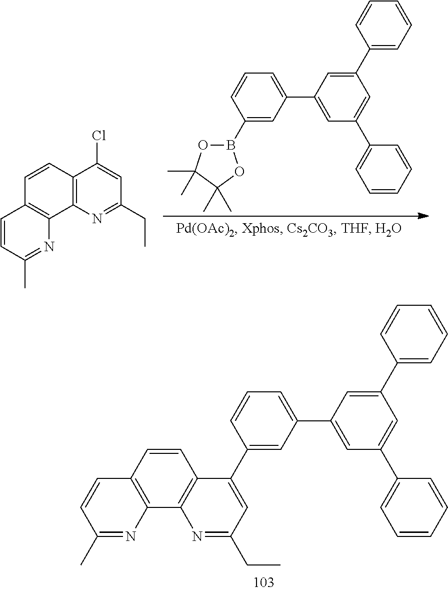 [SMILES: CCc1cc(Cl)c2ccc3ccc(C)nc3c2n1 ; CC1(C)OB(c2cccc(-c3cc(-c4ccccc4)cc(-c4ccccc4)c3)c2)OC1(C)C ; CCc1cc(-c2cccc(-c3cc(-c4ccccc4)cc(-c4ccccc4)c3)c2)c2ccc3ccc(C)nc3c2n1]

The same procedure as in [Synthesis Example 60], with the exception of using 4,4,5,5-tetramethyl-2-(5′-phenyl-[1,1′:3′,1′-terphenyl]-3-yl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (6.6 g, yield: 61%)

[LCMS]: 527

### [Synthesis Example 65] Synthesis of Compound 104

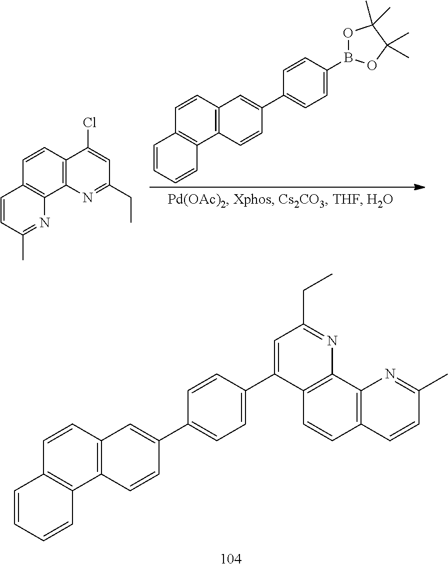 [SMILES: CCc1cc(Cl)c2ccc3ccc(C)nc3c2n1 ; CC1(C)OB(c2ccc(-c3ccc4c(ccc5ccccc54)c3)cc2)OC1(C)C ; CCc1cc(-c2ccc(-c3ccc4c(ccc5ccccc54)c3)cc2)c2ccc3ccc(C)nc3c2n1]

The same procedure as in [Synthesis Example 60], with the exception of using 4,4,5,5-tetramethyl-2-(4-(phenanthren-2-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (6.0 g, yield: 61%).

[LCMS]: 475

### [Synthesis Example 66] Synthesis of Compound 105

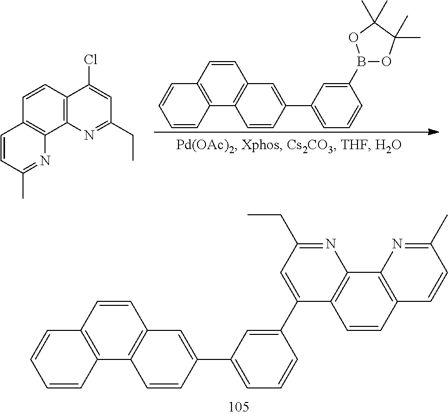 [SMILES: CCc1cc(Cl)c2ccc3ccc(C)nc3c2n1 ; CC1(C)OB(c2cccc(-c3ccc4c(ccc5ccccc54)c3)c2)OC1(C)C ; CCc1cc(-c2cccc(-c3ccc4c(ccc5ccccc54)c3)c2)c2ccc3ccc(C)nc3c2n1]

The same procedure as in [Synthesis Example 60], with the exception of using 4,4,5,5-tetramethyl-2-(3-(phenanthren-2-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (6.0 g, yield: 61%).

[LCMS]: 475

### [Synthesis Example 67] Synthesis of Compound 107

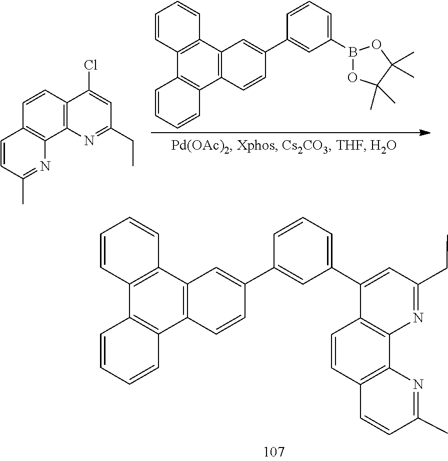

The same procedure as in [Synthesis Example 60], with the exception of using 4,4,5,5-tetramethyl-2-(3-(triphenylen-2-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (6.8 g, yield: 63%).

[LCMS]: 525

### [Synthesis Example 68] Synthesis of Compound 108

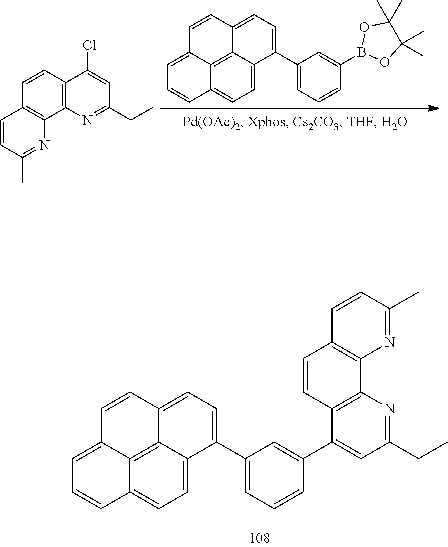

The same procedure as in [Synthesis Example 60], with the exception of using 4,4,5,5-tetramethyl-2-(3-(pyren-1-yl)phenyl)-1,3,2-dioxaborolane instead of 4,4,5,5-tetramethyl-2-(3-(4-phenylnaphthalen-1-yl)phenyl)-1,3,2-dioxaborolane, was carried out to afford the target compound (6.5 g, yield: 63%).

[LCMS]: 499

### [Synthesis Example 69] Synthesis of Compound 122

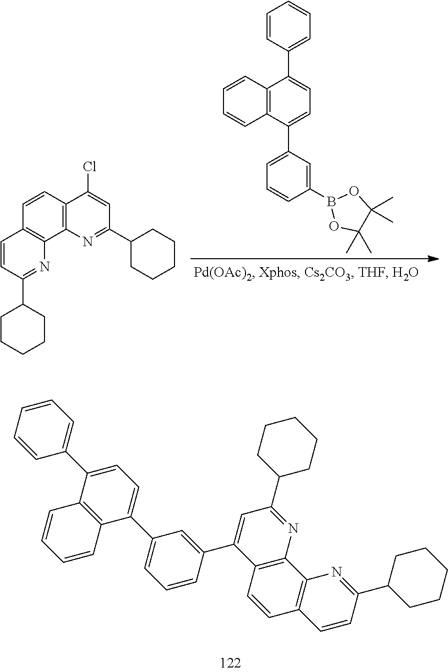 [SMILES: Clc1cc(C2CCCCC2)nc2c1ccc1ccc(C3CCCCC3)nc12 ; CC1(C)OB(c2cccc(-c3ccc(-c4ccccc4)c4ccccc34)c2)OC1(C)C ; c1ccc(-c2ccc(-c3cccc(-c4cc(C5CCCCC5)nc5c4ccc4ccc(C6CCCCC6)nc45)c3)c3ccccc23)cc1]

4-Chloro-2,9-dicyclohexyl-1,10-phenanthroline (7.8 g, 20.6 mmol) [symbol] 4,4,5,5-tetramethyl-2-(4-(4-phenylnaphthalen-1-yl)phenyl)-1,3,2-dioxaborolane (8.4 g, 20.6 mmol) [symbol] Pd(OAc)₂ (0.2 g, 1.0 mmol), Xphos (1.0 g, 2.1 mmol), and Cs₂CO₃ (13.5 g, 41.3 mmol) were added to a solvent mixture of toluene (50 ml), EtOH (10 ml), and H₂O (10 ml) and heated for 12 hours under reflux. After completion of the reaction, extraction was conducted with methylene chloride. The extract was added with MgSO₄ and then filtered. The solvent was removed from the filtered organic layer which was then purified by column chromatography to afford the target compound (9.0 g, yield: 70%)

[LCMS]:623

### [Synthesis Example 70] Synthesis of Compound 123

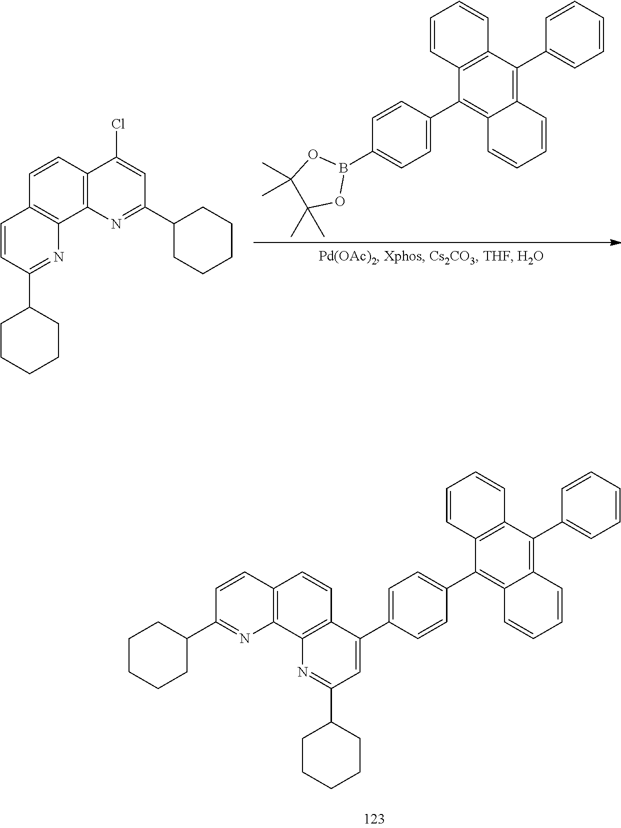

The same procedure as in [Synthesis Example 69], with the exception of using 4,4,5,5-tetramethyl-2-(4-(10-phenylanthracen-9-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (10.1 g, yield: 73%).

[LCMS]:673

### [Synthesis Example 71] Synthesis of Compound 124

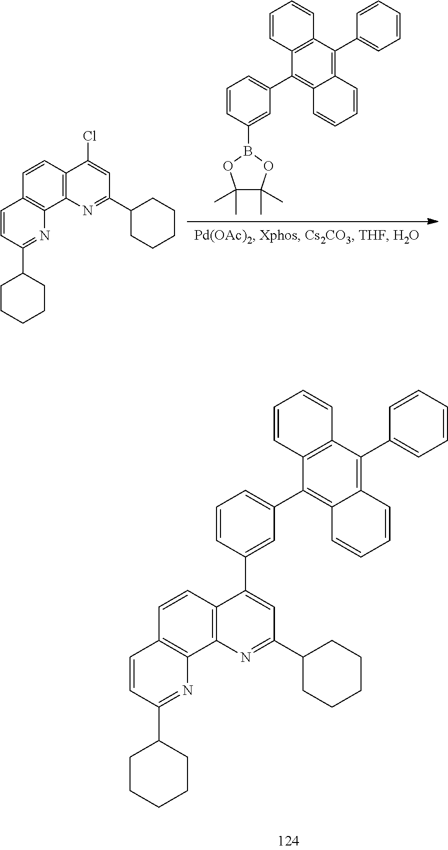 [SMILES: Clc1cc(C2CCCCC2)nc2c1ccc1ccc(C3CCCCC3)nc12 ; CC1(C)OB(c2cccc(-c3c4ccccc4c(-c4ccccc4)c4ccccc34)c2)OC1(C)C ; c1ccc(-c2c3ccccc3c(-c3cccc(-c4cc(C5CCCCC5)nc5c4ccc4ccc(C6CCCCC6)nc45)c3)c3ccccc23)cc1]

The same procedure as in [Synthesis Example 69], with the exception of using 4,4,5,5-tetramethyl-2-(3-(10-phenylanthracen-9-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (10.1 g, yield: 73%).

[LCMS]: 673

### [Synthesis Example 72] Synthesis of Compound 127

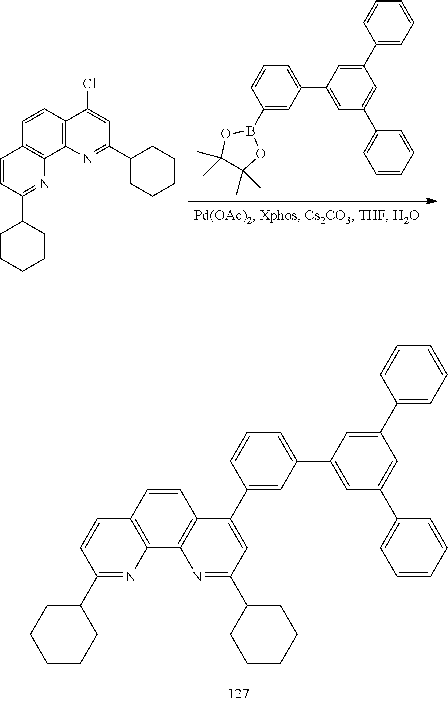

The same procedure as in [Synthesis Example 69], with the exception of using 4,4,5,5-tetramethyl-2-(5′-phenyl-[1,1′:3′,1′-terphenyl]-3-yl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (10.0 g, yield: 75%)

[LCMS]:649

### [Synthesis Example 73] Synthesis of Compound 128

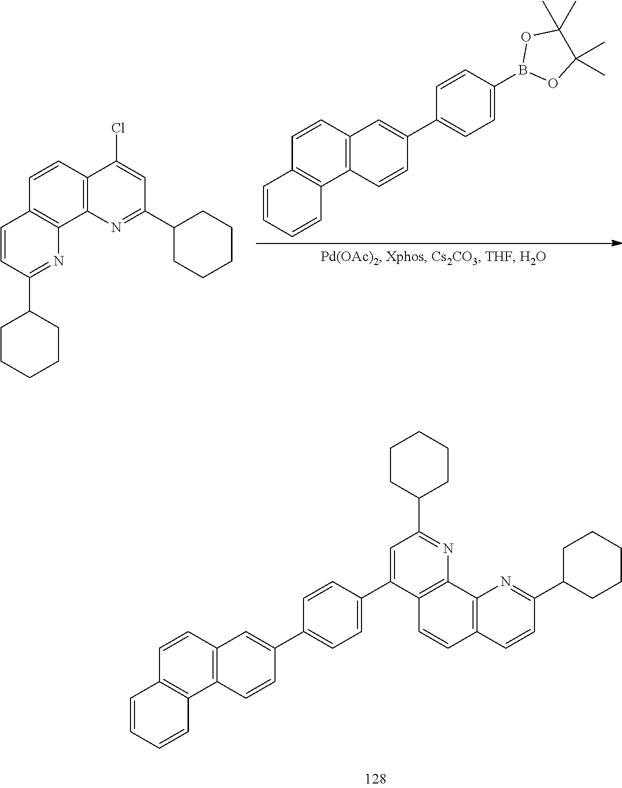

The same procedure as in [Synthesis Example 69], with the exception of using 4,4,5,5-tetramethyl-2-(4-(phenanthren-2-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (9.1 g, yield: 74%).

[LCMS]:597

### [Synthesis Example 74] Synthesis of Compound 132

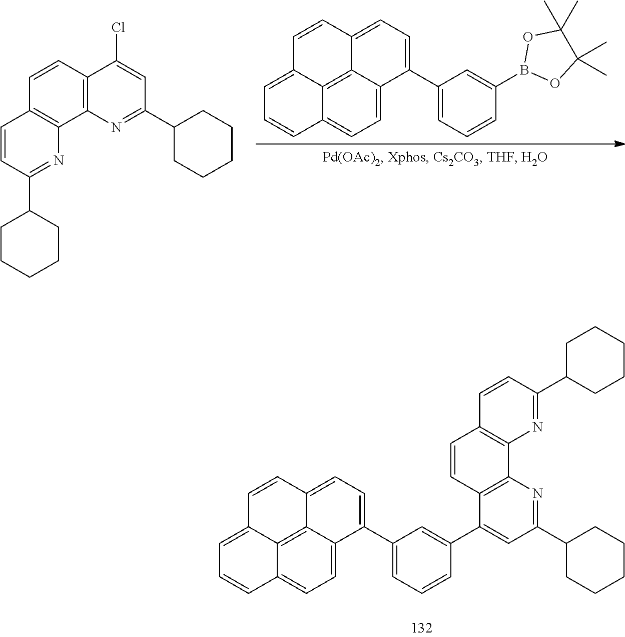

The same procedure as in [Synthesis Example 69], with the exception of using 4,4,5,5-tetramethyl-2-(3-(pyren-1-yl)phenyl)-1,3,2-dioxaborolane instead of 4,4,5,5-tetramethyl-2-(4-(4-phenylnaphthalen-1-yl)phenyl)-1,3,2-dioxaborolane, was carried out to afford the target compound (9.0 g, yield: 70%).

[LCMS]:621

### [Synthesis Example 75] Synthesis of Compound 169

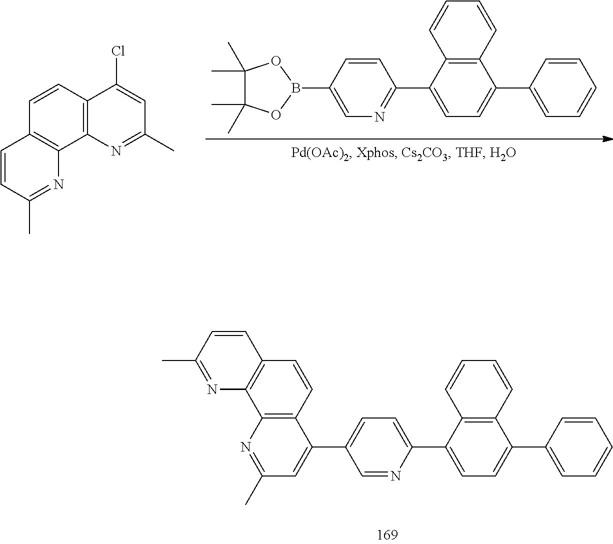 [SMILES: Cc1ccc2ccc3c(Cl)cc(C)nc3c2n1 ; CC1(C)OB(c2ccc(-c3ccc(-c4ccccc4)c4ccccc34)nc2)OC1(C)C ; Cc1ccc2ccc3c(-c4ccc(-c5ccc(-c6ccccc6)c6ccccc56)nc4)cc(C)nc3c2n1]

The same procedure as in [Synthesis Example 1], with the exception of using 2-(4-phenylnaphthalen-1-yl)-5-(4,4,5,5-tetramethyl-1,3,2-dioxaborolan-2-yl)pyridine instead of 4,4,5,5-tetramethyl-2-(4-(4-phenylnaphthalen-1-yl)phenyl)-1,3,2-dioxaborolane, was carried out to afford the target compound (6.3 g, yield: 63%)

[LCMS]:488

### [Synthesis Example 76] Synthesis of Compound 171

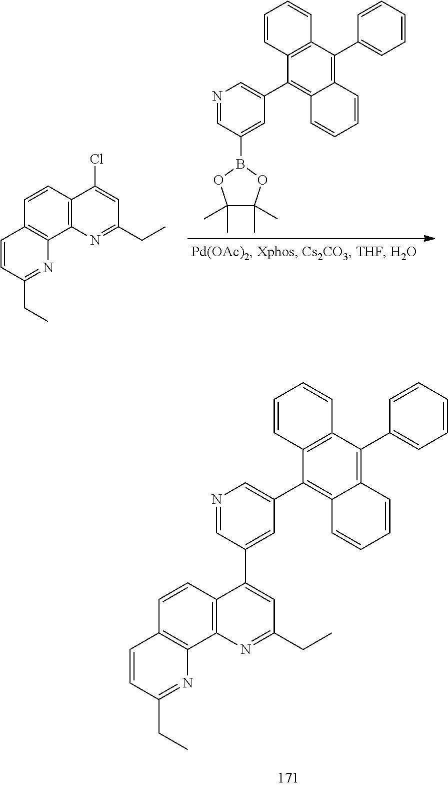 [SMILES: CCc1ccc2ccc3c(Cl)cc(CC)nc3c2n1 ; CC1(C)OB(c2cncc(-c3c4ccccc4c(-c4ccccc4)c4ccccc34)c2)OC1(C)C ; CCc1ccc2ccc3c(-c4cncc(-c5c6ccccc6c(-c6ccccc6)c6ccccc56)c4)cc(CC)nc3c2n1]

The same procedure as in [Synthesis Example 25], with the exception of using 3-(10-phenylanthracen-9-yl)-5-(4,4,5,5-tetramethyl-1,3,2-dioxaborolan-2-yl)pyridine instead of 4,4,5,5-tetramethyl-2-(4-(4-phenylnaphthalen-1-yl)phenyl)-1,3,2-dioxaborolane, was carried out to afford the target compound (7.5 g, yield: 64%)

[LCMS]:566

### [Synthesis Example 77] Synthesis of Compound 172

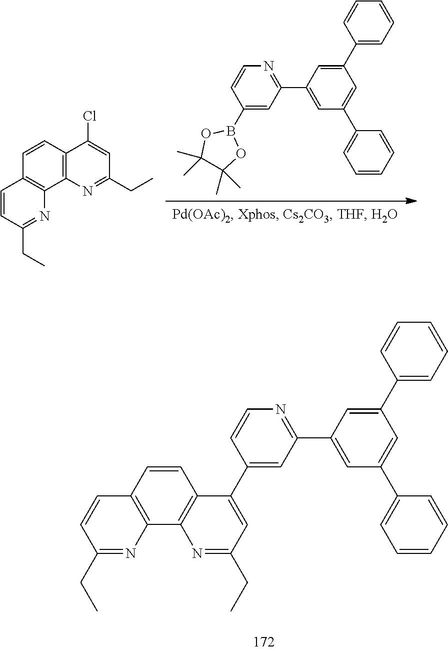

The same procedure as in [Synthesis Example 25], with the exception of using 2-([1,1′:3′,1″-terphenyl]-5′-yl)-4-(4,4,5,5-tetramethyl-1,3,2-dioxaborolan-2-yl)pyridine instead of 4,4,5,5-tetramethyl-2-(4-(4-phenylnaphthalen-1-yl)phenyl)-1,3,2-dioxaborolane, was carried out to afford the target compound (7.2 g, yield: 65%)

[LCMS]:542

### [Synthesis Example 78] Synthesis of Compound 173

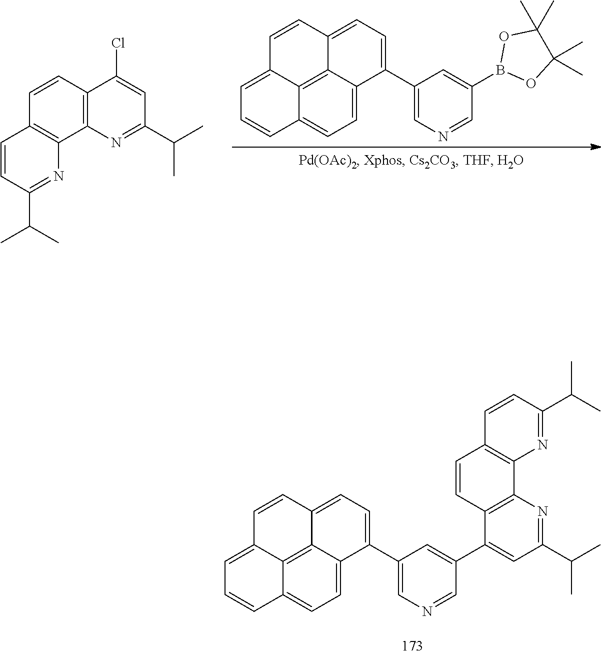

The same procedure as in [Synthesis Example 36], with the exception of using 3-(pyren-1-yl)-5-(4,4,5,5-tetramethyl-1,3,2-dioxaborolan-2-yl)pyridine instead of 4,4,5,5-tetramethyl-2-(4-(4-phenylnaphthalen-1-yl)phenyl)-1,3,2-dioxaborolane, was carried out to afford the target compound (6.7 g, yield: 60%).

[LCMS]:542

### [Synthesis Example 79] Synthesis of Compound 176

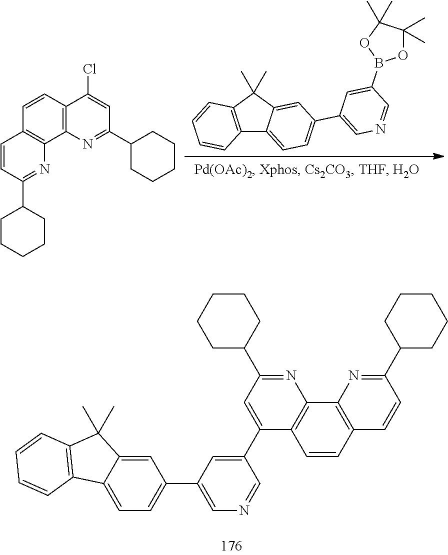

The same procedure as in [Synthesis Example 69], with the exception of using 3-(9,9-dimethyl-9H-fluoren-2-yl)-5-(4,4,5,5-tetramethyl-1,3,2-dioxaborolan-2-yl)pyridine instead of 4,4,5,5-tetramethyl-2-(4-(4-phenylnaphthalen-1-yl)phenyl)-1,3,2-dioxaborolane, was carried out to afford the target compound (7.6 g, yield: 60%)

[LCMS]:614

### [Synthesis Example 80] Synthesis of Compound 179

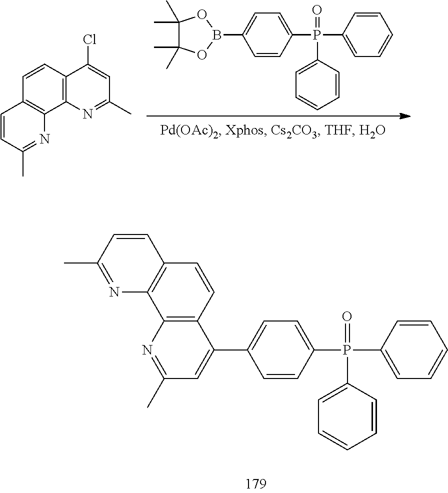 [SMILES: Cc1ccc2ccc3c(Cl)cc(C)nc3c2n1 ; CC1(C)OB(c2ccc(P(=O)(c3ccccc3)c3ccccc3)cc2)OC1(C)C ; Cc1ccc2ccc3c(-c4ccc(P(=O)(c5ccccc5)c5ccccc5)cc4)cc(C)nc3c2n1]

The same procedure as in [Synthesis Example 1], with the exception of using diphenyl(4-(4,4,5,5-tetramethyl-1,3,2-dioxaborolan-2-yl)phenyl)phosphine oxide instead of 4,4,5,5-tetramethyl-2-(4-(4-phenylnaphthalen-1-yl)phenyl)-1,3,2-dioxaborolane, was carried out to afford the target compound (7.0 g, yield: 70%).

[LCMS]: 485

### [Synthesis Example 81] Synthesis of Compound 180

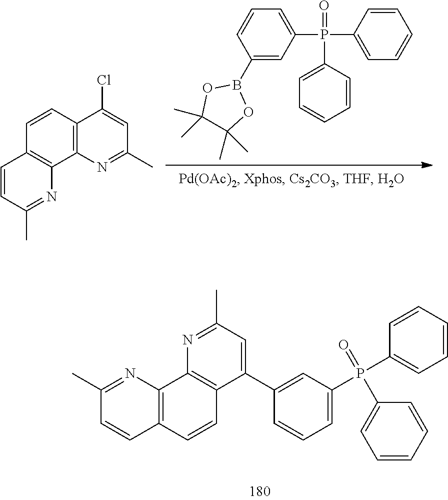 [SMILES: Cc1ccc2ccc3c(Cl)cc(C)nc3c2n1 ; CC1(C)OB(c2cccc(P(=O)(c3ccccc3)c3ccccc3)c2)OC1(C)C ; Cc1ccc2ccc3c(-c4cccc(P(=O)(c5ccccc5)c5ccccc5)c4)cc(C)nc3c2n1]

The same procedure as in [Synthesis Example 1], with the exception of using diphenyl(3-(4,4,5,5-tetramethyl-1,3,2-dioxaborolan-2-yl)phenyl)phosphine oxide instead of 4,4,5,5-tetramethyl-2-(4-(4-phenylnaphthalen-1-yl)phenyl)-1,3,2-dioxaborolane, was carried out to afford the target compound (7.0 g, yield: 70%).

[LCMS]: 485

### [Synthesis Example 82] Synthesis of Compound 181

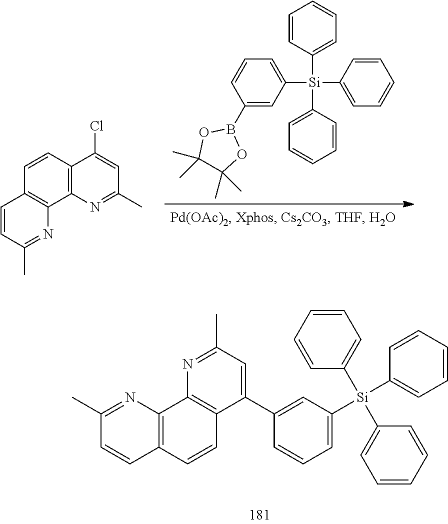 [SMILES: Cc1ccc2ccc3c(Cl)cc(C)nc3c2n1 ; CC1(C)OB(c2cccc([Si](c3ccccc3)(c3ccccc3)c3ccccc3)c2)OC1(C)C ; Cc1ccc2ccc3c(-c4cccc([Si](c5ccccc5)(c5ccccc5)c5ccccc5)c4)cc(C)nc3c2n1]

The same procedure as in [Synthesis Example 1], with the exception of using triphenyl(3-(4,4,5,5-tetramethyl-1,3,2-dioxaborolan-2-yl)phenyl)silane instead of 4,4,5,5-tetramethyl-2-(4-(4-phenylnaphthalen-1-yl)phenyl)-1,3,2-dioxaborolane, was carried out to afford the target compound (7.8 g, yield: 70%).

[LCMS]: 543

### [Example 1]—Fabrication of Blue Organic EL Device

Compound 1 synthesized in Synthesis Example 1 was subjected to highly pure sublimation purification by a typically known method, and then a blue organic EL device was fabricated as follows.

A glass substrate having a thin coat of indium tin oxide (ITO) 1,500 Å thick was ultrasonically washed with distilled water. After completion of the washing with distilled water, the substrate was again washed a solvent such as isopropyl alcohol, acetone, methanol, etc., under ultrasonication, and dried. The substrate was cleansed with UV for 5 minutes in a UV ozone cleaner (Power sonic 405, Hwashin Tech) and then transferred to a vacuum evaporator. DS-205 (Doosan Corporation Electronics, 80 nm)/NPB (15 nm)/ADN+5% DS-405 (Doosan Corporation Electronics, 30 nm)/Compound 1 (30 nm)/LiF (1 nm)/Al (200 nm) were laminated in that order on the ITO transparent electrode prepared above, to fabricate an organic EL device. The structures of NPB and ADN are as follows.

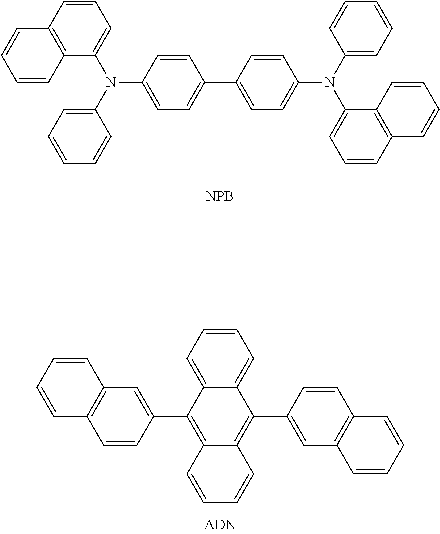

### Examples 2 to 82

Blue organic EL devices of Examples 2-82 were fabricated in the same manner as in Example 1, with the exception that the compounds listed in Table 1, instead of Compound 1, were used as respective electron transport layer materials.

### [Comparative Examples 1 to 8]-Fabrication of Blue Organic EL Devices

Blue organic EL devices of Comparative Examples 1-8 were fabricated in the same manner as in Example 1, with the exception that the following Compounds A to H, instead of Compound 1, were used as respective electron transport layer materials.

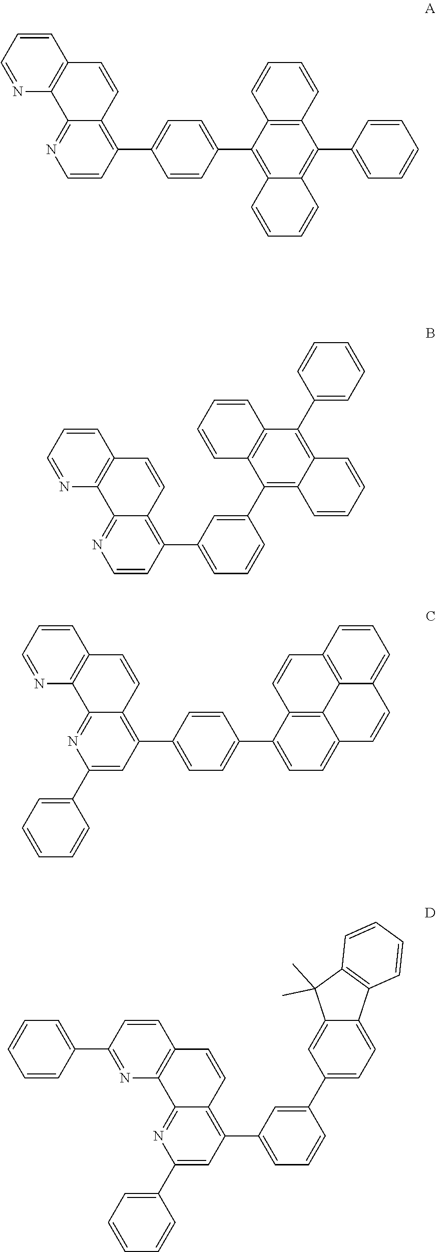 [SMILES: c1ccc(-c2c3ccccc3c(-c3ccc(-c4ccnc5c4ccc4cccnc45)cc3)c3ccccc23)cc1 ; c1ccc(-c2c3ccccc3c(-c3cccc(-c4ccnc5c4ccc4cccnc45)c3)c3ccccc23)cc1 ; c1ccc(-c2cc(-c3ccc(-c4ccc5ccc6cccc7ccc4c5c67)cc3)c3ccc4cccnc4c3n2)cc1 ; CC1(C)c2ccccc2-c2ccc(-c3cccc(-c4cc(-c5ccccc5)nc5c4ccc4ccc(-c6ccccc6)nc45)c3)cc21]

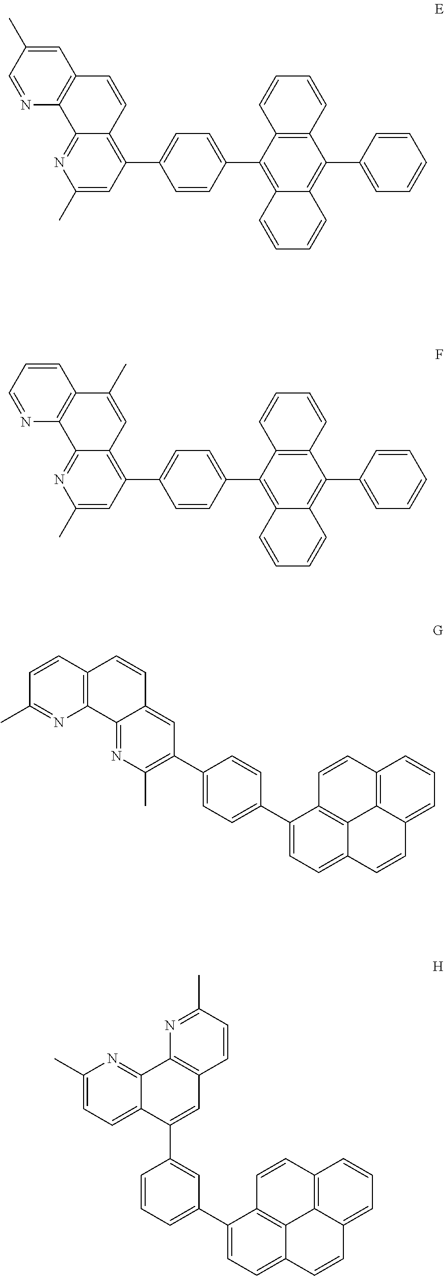 [SMILES: Cc1cnc2c(ccc3c(-c4ccc(-c5c6ccccc6c(-c6ccccc6)c6ccccc56)cc4)cc(C)nc32)c1 ; Cc1cc(-c2ccc(-c3c4ccccc4c(-c4ccccc4)c4ccccc34)cc2)c2cc(C)c3cccnc3c2n1 ; Cc1ccc2ccc3cc(-c4ccc(-c5ccc6ccc7cccc8ccc5c6c78)cc4)c(C)nc3c2n1 ; Cc1ccc2cc(-c3cccc(-c4ccc5ccc6cccc7ccc4c5c67)c3)c3ccc(C)nc3c2n1]

### Evaluation Example 1

The blue organic EL devices fabricated in Examples 1 to 82 and Comparative Examples 1 to 8 were measured for driving voltage, current efficiency, and light-emitting wavelength at a current density of 10 mA/cm2 and the measurements are summarized in Table 1, below.

TABLE 1

| | Electron | Driving | Luminescent | Current |
|---|---|---|---|---|
| | Transport | Voltage | Peak | Efficiency |
| Sample | Layer | (V) | (nm) | (Cd/A) |
| Ex. 1 | Compound 1 | 3.9 | 457 | 8.0 |
| Ex. 2 | Compound 2 | 4.1 | 455 | 8.0 |
| Ex. 3 | Compound 3 | 4.2 | 455 | 7.8 |
| Ex. 4 | Compound 4 | 4.1 | 457 | 8.1 |
| Ex. 5 | Compound 5 | 4.2 | 456 | 7.9 |
| Ex. 6 | Compound 6 | 3.9 | 457 | 8.0 |
| Ex. 7 | Compound 7 | 4.0 | 456 | 7.8 |
| Ex. 8 | Compound 8 | 4.1 | 456 | 8.1 |
| Ex. 9 | Compound 9 | 4.0 | 456 | 7.9 |
| Ex. 10 | Compound 10 | 4.2 | 458 | 8.1 |
| Ex. 11 | Compound 11 | 4.0 | 454 | 7.9 |
| Ex. 12 | Compound 12 | 3.9 | 454 | 8.1 |
| Ex. 13 | Compound 13 | 4.3 | 455 | 7.8 |
| Ex. 14 | Compound 14 | 4.0 | 455 | 8.0 |
| Ex. 15 | Compound 15 | 4.2 | 457 | 8.1 |
| Ex. 16 | Compound 16 | 4.2 | 457 | 7.8 |
| Ex. 17 | Compound 17 | 4.1 | 457 | 7.8 |
| Ex. 18 | Compound 18 | 4.3 | 455 | 7.9 |
| Ex. 19 | Compound 19 | 4.0 | 454 | 7.7 |
| Ex. 20 | Compound 20 | 4.2 | 455 | 7.7 |
| Ex. 21 | Compound 21 | 4.1 | 455 | 7.8 |
| Ex. 22 | Compound 22 | 4.3 | 457 | 7.9 |
| Ex. 23 | Compound 23 | 4.0 | 456 | 8.0 |
| Ex. 24 | Compound 24 | 4.3 | 457 | 7.9 |
| Ex. 25 | Compound 25 | 4.2 | 456 | 8.0 |
| Ex. 26 | Compound 26 | 4.1 | 456 | 7.7 |
| Ex. 27 | Compound 27 | 4.2 | 456 | 7.6 |
| Ex. 28 | Compound 28 | 4.1 | 456 | 7.7 |
| Ex. 29 | Compound 29 | 3.9 | 458 | 7.8 |
| Ex. 30 | Compound 31 | 4.1 | 454 | 7.6 |
| Ex. 31 | Compound 32 | 4.2 | 456 | 7.6 |
| Ex. 32 | Compound 33 | 4.2 | 458 | 7.8 |
| Ex. 33 | Compound 34 | 3.9 | 458 | 7.9 |
| Ex. 34 | Compound 35 | 4.1 | 457 | 7.8 |
| Ex. 35 | Compound 36 | 4.1 | 457 | 7.9 |
| Ex. 36 | Compound 50 | 4.0 | 457 | 8.0 |
| Ex. 37 | Compound 51 | 3.9 | 457 | 8.0 |
| Ex. 38 | Compound 52 | 4.1 | 455 | 8.0 |
| Ex. 39 | Compound 53 | 4.2 | 455 | 7.8 |
| Ex. 40 | Compound 55 | 4.1 | 457 | 7.9 |
| Ex. 41 | Compound 56 | 4.2 | 457 | 7.9 |
| Ex. 42 | Compound 57 | 3.9 | 457 | 8.0 |
| Ex. 43 | Compound 59 | 4.0 | 457 | 7.8 |
| Ex. 44 | Compound 60 | 4.1 | 456 | 8.1 |
| Ex. 45 | Compound 61 | 3.9 | 458 | 7.8 |
| Ex. 46 | Compound 63 | 4.1 | 454 | 7.6 |
| Ex. 47 | Compound 64 | 4.1 | 457 | 7.9 |
| Ex. 48 | Compound 74 | 3.9 | 454 | 8.1 |
| Ex. 49 | Compound 75 | 4.3 | 455 | 7.8 |
| Ex. 50 | Compound 76 | 4.0 | 454 | 8.0 |
| Ex. 51 | Compound 77 | 4.2 | 457 | 7.7 |
| Ex. 52 | Compound 79 | 4.2 | 457 | 7.8 |
| Ex. 53 | Compound 80 | 4.1 | 457 | 7.8 |
| Ex. 54 | Compound 81 | 4.3 | 455 | 7.9 |
| Ex. 55 | Compound 83 | 4.0 | 454 | 7.7 |
| Ex. 56 | Compound 84 | 4.2 | 455 | 7.7 |
| Ex. 57 | Compound 91 | 4.2 | 458 | 7.8 |
| Ex. 58 | Compound 95 | 4.0 | 454 | 7.9 |
| Ex. 59 | Compound 96 | 4.1 | 454 | 7.8 |
| Ex. 60 | Compound 98 | 4.1 | 457 | 8.1 |
| Ex. 61 | Compound 99 | 4.2 | 456 | 7.9 |
| Ex. 62 | Compound 100 | 4.1 | 455 | 7.9 |
| Ex. 63 | Compound 101 | 4.0 | 456 | 7.8 |
| Ex. 64 | Compound 103 | 4.1 | 456 | 8.1 |
| Ex. 65 | Compound 104 | 4.0 | 456 | 7.9 |
| Ex. 66 | Compound 105 | 4.2 | 458 | 8.1 |
| Ex. 67 | Compound 107 | 4.0 | 454 | 7.9 |
| Ex. 68 | Compound 108 | 4.0 | 455 | 8.0 |
| Ex. 69 | Compound 122 | 4.3 | 455 | 7.8 |
| Ex. 70 | Compound 123 | 4.0 | 455 | 8.0 |
| Ex. 71 | Compound 124 | 4.2 | 457 | 8.1 |
| Ex. 72 | Compound 127 | 4.2 | 457 | 7.8 |
| Ex. 73 | Compound 128 | 4.1 | 457 | 7.8 |
| Ex. 74 | Compound 132 | 4.3 | 455 | 7.9 |
| Ex. 75 | Compound 169 | 4.0 | 454 | 7.7 |
| Ex. 76 | Compound 171 | 4.2 | 455 | 7.7 |
| Ex. 77 | Compound 172 | 4.3 | 455 | 7.9 |
| Ex. 78 | Compound 173 | 4.3 | 455 | 7.8 |
| Ex. 79 | Compound 176 | 4.1 | 457 | 7.8 |
| Ex. 80 | Compound 179 | 4.1 | 457 | 7.9 |
| Ex. 81 | Compound 180 | 4.0 | 457 | 8.0 |
| Ex. 82 | Compound 181 | 4.0 | 457 | 8.0 |
| C. Ex. 1 | Compound A | 4.9 | 457 | 6.0 |
| C. Ex. 2 | Compound B | 4.9 | 457 | 6.1 |
| C. Ex. 3 | Compound C | 4.8 | 457 | 6.3 |
| C. Ex. 4 | Compound D | 4.7 | 456 | 6.5 |
| C. Ex. 5 | Compound E | 4.6 | 457 | 6.5 |
| C. Ex. 6 | Compound F | 4.6 | 458 | 6.7 |
| C. Ex. 7 | Compound G | 4.9 | 456 | 7.0 |
| C. Ex. 8 | Compound H | 4.8 | 459 | 7.0 |

As can be seen in Table 1, the blue EL devices (Examples 1 to 82) which employed the electron transport layers containing the compounds of the present disclosure (Compounds 1-181) in which the phenanthroline moiety has alkyl substituents at positions 2 and 9 therein were improved in driving voltage and current efficiency, compared to those that employed the electron transport layers containing unsubstituted phenanthroline moieties (Comparative Examples 1-2) and phenanthroline moieties substituted with aryls (Comparative Examples 3-4). In addition. the compounds of the present disclosure used in Examples 1-82 are lower in sublimation temperature than the compounds bearing the phenanthroline moiety substituted with aryl groups (Compounds C and D), and thus can prevent the devices from being deteriorated when applied to the fabrication of the devices.

Furthermore, a more improvement was brought about in device characteristics by the compounds of the present disclosure in which the phenanthroline moiety has alkyl substituents at positions 2 and 9 than compounds in which the phenanthroline moiety has alkyl substituents at other positions. Meanwhile, compared to the devices of Comparative Examples 1 to 4 employing the compounds in which the phenanthroline moiety is unsubstituted or substituted with aryl groups (i.e., Compounds A-D), the device of Comparative Example 5 employing the compound in which the phenanthroline moiety has alkyl substituents at positions 2 and 8 (i.e., Compound E), and the device of Comparative Example 6 employing the compound in which the phenanthroline moiety has alkyl substituents at positions 2 and 6 (i.e., Compound F) exhibited slightly improved current efficiency, but the characteristics of the device were not significantly improved because the active sites unique to phenanthroline was not blocked. The data indicate that even if the compound contains a phenanthroline derivative having an alkyl group introduced thereinto, the thermal stability of the material can be maintained only when the alkyl is substituted at positions 2 and 9, which are the active sites

In addition, the devices of Examples 1-82 employing the compounds of the present disclosure in which the phenanthroline moiety has alkyl substituents at position 2 and 9 and an aryl group substituent at position 4 were lower in driving voltage and higher in luminous efficiency than those of Comparative Examples 7-8 employing the compounds in which the phenanthroline moiety has alkyl substituents at positions 2 and 9 and an aryl group substituent at a different position from position 4 (e.g., position 3 or 5). From these data, it was understood that even if the compounds have the phenanthroline moiety in which alkyl groups are substituted at both positions 2 and 9, the position where aryl is introduced also has a great effect on device characteristics.

### [Example 83] Fabrication of Organic EL Device

Compound 1 synthesized in Synthesis Example 1 was subjected to highly pure sublimation purification by a typically known method, and then a blue organic EL device was fabricated as follows.

A glass substrate having a thin coat of indium tin oxide (ITO) 1,500 Å thick was ultrasonically washed with distilled water. After completion of the washing with distilled water, the substrate was again washed a solvent such as isopropyl alcohol, acetone, methanol, etc., under ultrasonication, and dried. The substrate was cleansed with UV for 5 minutes in a UV ozone cleaner (Power sonic 405, Hwashin Tech) and then transferred to a vacuum evaporator. DS-205 (Doosan Corporation Electronics, 80 nm)/NPB (15 nm)/ADN+5% DS-405 (Doosan Corporation Electronics, 30 nm)/Alq₃ (30 nm)/Compound 1 (30 nm)/DS-505 (Doosan Corporation Electronics, 15 nm/NPB (15 nm)/CBP+10% (piq)₂Ir(acac) (40 nm)/Alq₃ (30 nm)/LiF (1 nm)/Al (200 nm) were laminated in that order on the ITO transparent electrode prepared above, to fabricate an organic EL device. The structures of NPB and ADN are as shown above, and the structures of Ala₃, CBP, and (piq)₂Ir(acac) are as follows.

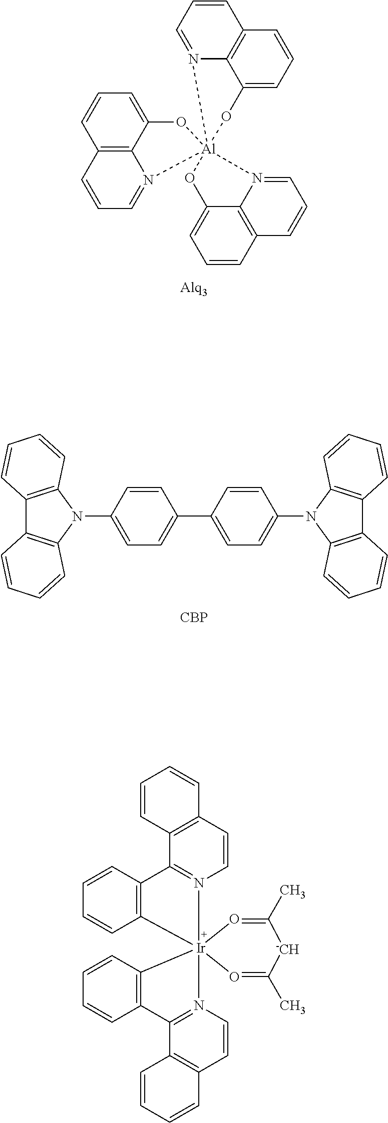

### Examples 84 to 164

Blue organic EL devices of Examples 84-164 were fabricated in the same manner as in Example 83, with the exception that the compounds listed in Table 2, instead of Compound 1, were used as respective N-type charge generation layer materials.

### [Comparative Examples 9 to 16] Fabrication of Organic EL Device

Blue organic EL devices of Comparative Examples 9-16 were fabricated in the same manner as in Example 83, with the exception that the following Compounds A to H, instead of Compound 1, were used as respective N-type charge generation layer materials. Structures of Compounds A to H are as shown in Comparative Examples 1 to 8.

### Evaluation Example 2

The blue organic EL devices fabricated in Examples 83 to 164 and Comparative Examples 9 to 16 were measured for driving voltage and current efficiency at a current density of 10 mA/cm2 and the measurements are summarized in Table 2, below.

TABLE 2

| | Electron | Driving | Current |
|---|---|---|---|
| | Transport | Voltage | Efficiency |
| Sample | Layer | (V) | (cd/A) |
| Ex. 83 | Compound 1 | 8.3 | 15.9 |
| Ex. 84 | Compound 2 | 8.3 | 15.7 |
| Ex. 85 | Compound 3 | 8.2 | 15.5 |
| Ex. 86 | Compound 4 | 8.3 | 15.9 |
| Ex. 87 | Compound 5 | 8.5 | 15.3 |
| Ex. 88 | Compound 6 | 8.3 | 15.3 |
| Ex. 89 | Compound 7 | 8.2 | 15.7 |
| Ex. 90 | Compound 8 | 8.3 | 15.8 |
| Ex. 91 | Compound 9 | 8.2 | 15.5 |
| Ex. 92 | Compound 10 | 8.5 | 15.3 |
| Ex. 93 | Compound 11 | 8.4 | 15.4 |
| Ex. 94 | Compound 12 | 8.2 | 15.9 |
| Ex. 95 | Compound 13 | 8.4 | 15.7 |
| Ex. 96 | Compound 14 | 8.3 | 15.7 |
| Ex. 97 | Compound 15 | 8.2 | 15.8 |
| Ex. 98 | Compound 16 | 8.4 | 15.5 |
| Ex. 99 | Compound 17 | 8.3 | 15.9 |
| Ex. 100 | Compound 18 | 8.4 | 15.6 |
| Ex. 101 | Compound 19 | 8.4 | 15.8 |
| Ex. 102 | Compound 20 | 8.4 | 15.6 |
| Ex. 103 | Compound 21 | 8.3 | 15.7 |
| Ex. 104 | Compound 22 | 8.3 | 15.5 |
| Ex. 105 | Compound 23 | 8.2 | 15.7 |
| Ex. 106 | Compound 24 | 8.3 | 15.7 |
| Ex. 107 | Compound 25 | 8.3 | 15.7 |
| Ex. 108 | Compound 26 | 8.3 | 15.7 |
| Ex. 109 | Compound 27 | 8.2 | 15.6 |
| Ex. 110 | Compound 28 | 8.2 | 15.6 |
| Ex. 111 | Compound 29 | 8.4 | 15.6 |
| Ex. 112 | Compound 31 | 8.3 | 15.5 |
| Ex. 113 | Compound 32 | 8.4 | 15.6 |
| Ex. 114 | Compound 33 | 8.3 | 15.5 |
| Ex. 115 | Compound 34 | 8.4 | 15.6 |
| Ex. 116 | Compound 35 | 8.3 | 15.5 |
| Ex. 117 | Compound 36 | 8.2 | 15.7 |
| Ex. 118 | Compound 50 | 8.3 | 15.8 |
| Ex. 119 | Compound 51 | 8.2 | 15.5 |
| Ex. 120 | Compound 52 | 8.5 | 15.3 |
| Ex. 121 | Compound 53 | 8.4 | 15.4 |
| Ex. 122 | Compound 55 | 8.3 | 15.5 |
| Ex. 123 | Compound 56 | 8.2 | 15.5 |
| Ex. 124 | Compound 57 | 8.2 | 15.5 |
| Ex. 125 | Compound 59 | 8.3 | 15.4 |
| Ex. 126 | Compound 60 | 8.2 | 15.5 |
| Ex. 127 | Compound 61 | 8.3 | 15.7 |
| Ex. 128 | Compound 63 | 8.4 | 15.2 |
| Ex. 129 | Compound 64 | 8.2 | 15.6 |
| Ex. 130 | Compound 74 | 8.3 | 15.5 |
| Ex. 131 | Compound 75 | 8.2 | 15.4 |
| Ex. 132 | Compound 76 | 8.3 | 15.5 |
| Ex. 133 | Compound 77 | 8.5 | 15.5 |
| Ex. 134 | Compound 79 | 8.4 | 15.4 |
| Ex. 135 | Compound 80 | 8.5 | 15.7 |
| Ex. 136 | Compound 81 | 8.4 | 15.8 |
| Ex. 137 | Compound 83 | 8.4 | 15.6 |
| Ex. 138 | Compound 84 | 8.3 | 15.7 |
| Ex. 139 | Compound 91 | 8.4 | 15.6 |
| Ex. 140 | Compound 95 | 8.4 | 15.7 |
| Ex. 141 | Compound 96 | 8.3 | 15.6 |
| Ex. 142 | Compound 98 | 8.5 | 15.5 |
| Ex. 143 | Compound 99 | 8.5 | 15.4 |
| Ex. 144 | Compound 100 | 8.4 | 15.6 |
| Ex. 145 | Compound 101 | 8.5 | 15.5 |
| Ex. 146 | Compound 103 | 8.4 | 15.4 |
| Ex. 147 | Compound 104 | 8.4 | 15.6 |
| Ex. 148 | Compound 105 | 8.4 | 15.5 |
| Ex. 149 | Compound 107 | 8.3 | 15.4 |
| Ex. 150 | Compound 108 | 8.2 | 15.6 |
| Ex. 151 | Compound 122 | 8.3 | 15.4 |
| Ex. 152 | Compound 123 | 8.2 | 15.5 |
| Ex. 153 | Compound 124 | 8.3 | 15.5 |
| Ex. 154 | Compound 127 | 8.3 | 15.4 |
| Ex. 155 | Compound 128 | 8.2 | 15.7 |
| Ex. 156 | Compound 132 | 8.3 | 15.8 |
| Ex. 157 | Compound 169 | 8.3 | 15.5 |
| Ex. 158 | Compound 171 | 8.4 | 15.4 |
| Ex. 159 | Compound 172 | 8.3 | 15.7 |
| Ex. 160 | Compound 173 | 8.5 | 15.8 |
| Ex. 161 | Compound 176 | 8.5 | 15.5 |
| Ex. 162 | Compound 179 | 8.4 | 15.4 |
| Ex. 163 | Compound 180 | 8.4 | 15.7 |
| Ex. 164 | Compound 181 | 8.2 | 15.8 |
| C. Ex. 9 | Compound A | 8.9 | 13.2 |
| C. Ex. 10 | Compound B | 8.9 | 13.3 |
| C. Ex. 11 | Compound C | 8.7 | 13.9 |
| C. Ex. 12 | Compound D | 8.8 | 13.7 |
| C. Ex. 13 | Compound E | 8.5 | 14.1 |
| C. Ex. 14 | Compound F | 8.6 | 14.4 |
| C. Ex. 15 | Compound G | 8.5 | 14.8 |
| C. Ex. 16 | Compound H | 8.5 | 14.7 |

As can be seen in Table 2, the blue EL devices (Examples 83-164) which employed N-type charge generation layers containing the compounds in which the phenanthroline moiety has alkyls as substituents at positions 2 and 9 were improved in driving voltage and current efficiency, compared to those that employed electron transport layers containing unsubstituted phenanthroline moieties (Comparative Examples 9-10) and phenanthroline moieties substituted with aryls (Comparative Examples 11-12). In addition. the compounds of the present disclosure used in Examples 83-164 are lower in sublimation temperature than the compounds bearing the phenanthroline moiety substituted with aryl groups (Compounds C and D), and thus can prevent the devices from being deteriorated when applied to the fabrication of the devices.

Furthermore, the devices of Examples 83-164 containing as N-type charge generation materials the compounds of the present disclosure in which the phenanthroline moiety has alkyl substituents at positions 2 and 9 were observed to exhibit higher performance in terms of driving voltage and current efficiency, compared to those of Comparative Examples 13-14 using the compounds in which the phenanthroline moiety has alkyl substituents at different positions. Meanwhile, compared to the devices of Comparative Examples 9 to 14 employing the compounds in which the phenanthroline moiety is unsubstituted or substituted with aryl groups, the device of Comparative Example 13 employing the compound in which the phenanthroline moiety has alkyl substituents at positions 2 and 8 (i.e., Compound E), and the device of Comparative Example 14 employing the compound in which the phenanthroline moiety has alkyl substituents at positions 2 and 6 (i.e., Compound F) exhibited slightly improved current efficiency, but the characteristics of the device were not significantly improved because the active sites unique to phenanthroline was not blocked. The data imply that even if the compound contains a phenanthroline derivative having an alkyl group introduced thereinto, the thermal stability of the material can be maintained only when the alkyl is substituted at positions 2 and 9, which are the active sites.

In addition, the devices of Examples 79-156 employing the compounds of the present disclosure in which the phenanthroline moiety has alkyl substituents at position 2 and 9 and an aryl group substituent at position 4 were lower in driving voltage and higher in luminous efficiency than those of Comparative Examples 15-16 employing the compounds in which the phenanthroline moiety has alkyl substituents at positions 2 and 9 and an aryl group substituent at a different position from position 4 (e.g., position 3 or 5).

1. An organic compound of the following Chemical Formula 1: 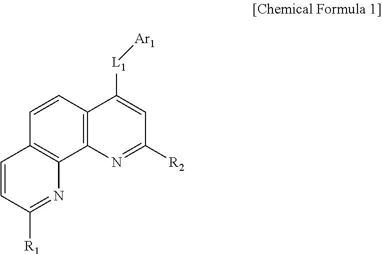
wherein,
R₁ and R₂, which are same or different, are each independently selected from the group consisting of a hydrogen atom, a deuterium atom (D), an alkyl group of C₁-C₆₀, a cycloalkyl group of C₃-C₆₀, and a heteroaryl group having 5 to 60 nuclear atoms, wherein a case where both of R₁ and R₂ are a hydrogen atom is excluded,
L₁ is selected from the group consisting of a single bond, an arylene group of C₆-C₆₀, and a heteroarylene group having 5 to 60 nuclear atoms, and
Ar₁ is selected from the group consisting of an aryl group of C₆-C₆₀, —P(═O)(R₃)(R₄), and —Si(R₅)(R₆)(R₇),
R₃ to R₇, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and
the alkyl group, the cycloalkyl group, and the heteroalkyl group of R₁ and R₂, the arylene group and the heteroarylene group of L₁, the arylene group of Ar₁, and the hydrazino group, the hydrazono group, the alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and the arylamine group of R₃ to R₇ are each independently substituted or unsubstituted with one or more substituents of: a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other).

2. The organic compound of claim 1, wherein at least one of R₁ and R₂ is an alkyl group of C₁-C₆₀ or a cycloalkyl group of C₃-C₆₀.

3. The organic compound of claim 1, wherein R₁ and R₂ are same or different and are each independently selected from the group consisting of a hydrogen atom, an alkyl group of C₁-C₂₀, and an alkyl group of C₃-C₂₀, with a proviso that at least one of R₁ and R₂ is an alkyl group of C₁-C₂₀ or a cycloalkyl group of C₃-C₂₀.

4. The organic compound of claim 1, wherein the compound of Chemical Formula 1 is a compound of any one of the following Chemical Formulas 2 to 6: 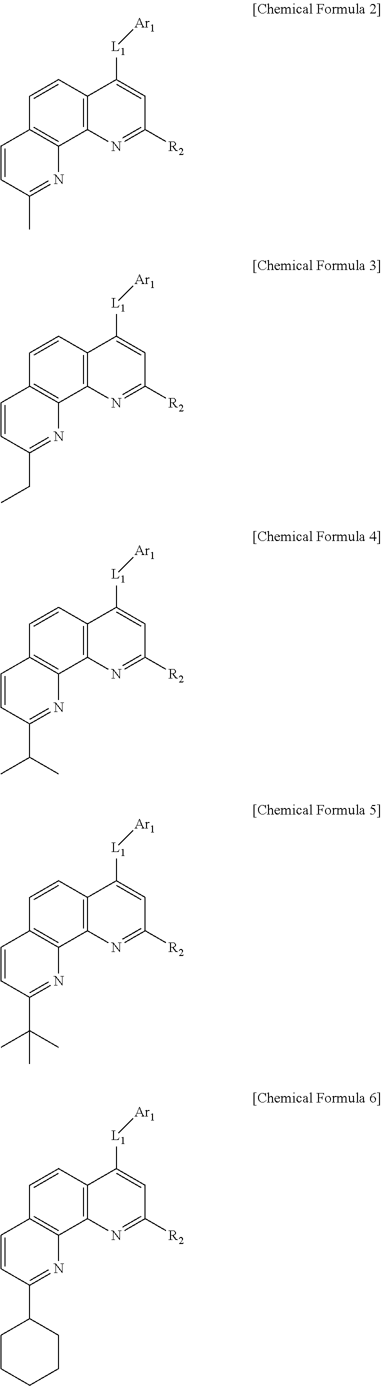
wherein,
L₁ and Ar₁ are as defined in claim 1, and
R₂ is an alkyl group of C₁-C₆ or a cycloalkyl group of C₃-C₆.

5. The organic compound of claim 1, wherein Ar₁ is a substituent selected from the group consisting of the following Chemical Formulas S1 to S8: 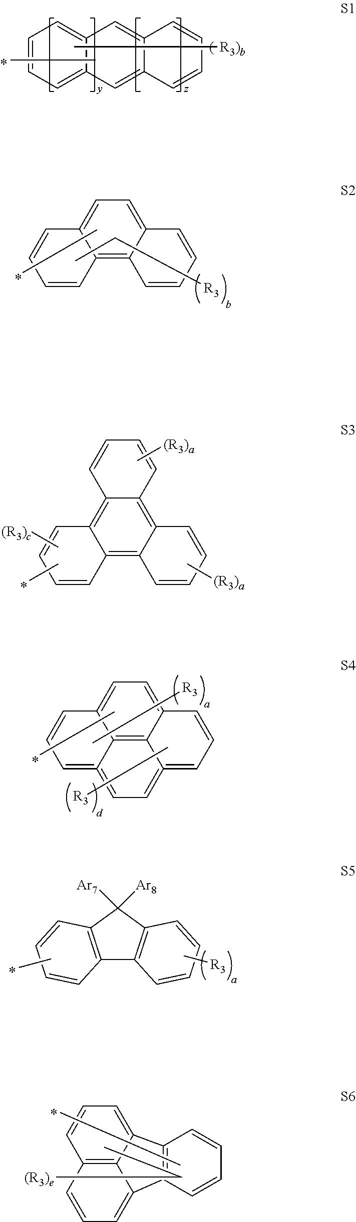 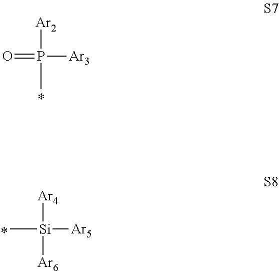
wherein,
a is an integer of 0 to 3,
d is an integer of 0 to 5,
e is an integer of 0 to 9,
the plurality of R₃ are the same as or different from each other,
R₃ or two or more R₃'s, if present, are each independently each independently selected from the group consisting of a hydrogen atom, a deuterium atom, a halogen group, a hydroxy group, a cyano group, a nitro group, an amino group, an amidino group, an hydrazine, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀,
y and z are each 0 or 1,
Ar₂ to Ar₈, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, or forming a fused ring with an adjacent group, and
the hydrazino group, the hydrazono group, the alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and the arylamine group of Ar₂ to Ar₈ are each independently substituted or unsubstituted with one or more substituents of: a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other.

6. The organic compound of claim 1, wherein L₁ is an arylene group of C₆-C₆₀ or an N-bearing heteroarylene group having 5 to 60 nuclear atoms.

7. The organic compound of claim 1, wherein L₁ is a linker of the following Chemical Formula L: 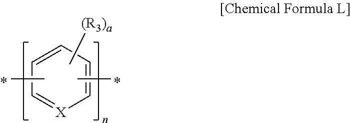
wherein,
n is an integer of 0 to 3,
a is an integer of 0 to 4,
X is C or N,
the plurality of R₃ are the same as or different from each other, and
R₃ or two or more R₃'s, if present, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀.

8. The organic compound of claim 1, wherein L₁ is any one of the following linkers L1 to L8:

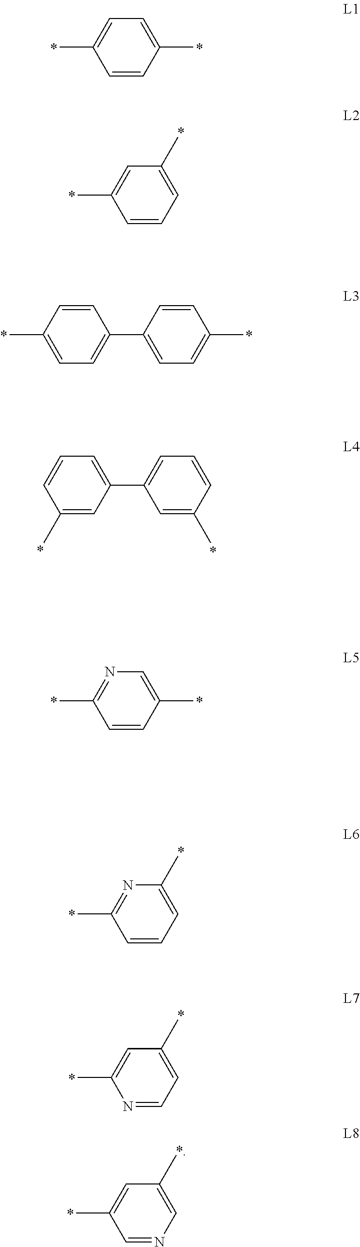

9. The organic compound of claim 1, wherein the compound of Chemical Formula 1 is a compound of any one of the following Chemical Formulas 7 to 12: 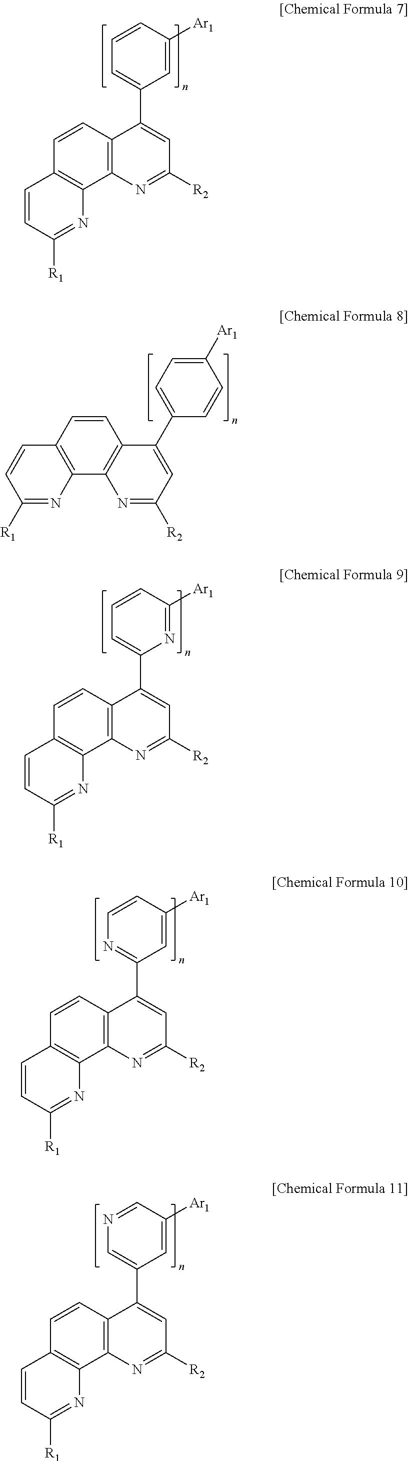 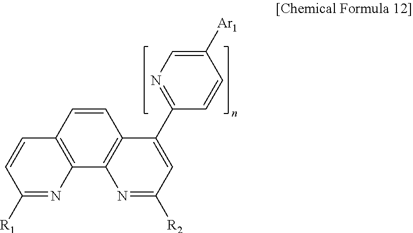
wherein,
R₁ and R₂, which are same or different, are each independently an alkyl group of C₁-C₆ or a cycloalkyl group of C₃-C₆,
n is 0 or 1, and
Ar₁ is selected from the group consisting of the following substituents S1 to S8: 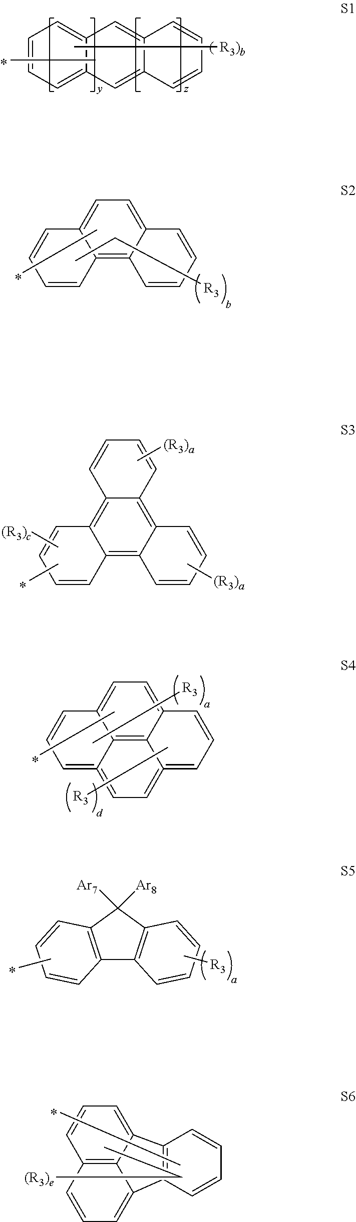 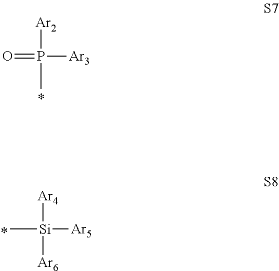
wherein,
a is an integer of 0 to 4,
b is an integer of 0 to 9,
c is an integer of 0 to 3,
d is an integer of 0 to 5,
e is an integer of 0 to 9,
the plurality of R₃ are the same as or different from each other,
R₃ or two or more R₃'s, if present, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀,
y and z are each 0 or 1,
Ar₂ to Ar₈, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, or forming a fused ring with an adjacent group, and
the hydrazino group, the hydrazono group, alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and the arylamine group of Ar₂ to Ar₈ are each independently substituted or unsubstituted with one or more substituents of: a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other.

10. The organic compound of claim 1, wherein the compound of Chemical Formula 1 is selected from the group consisting of the following Compounds 1 to 186:

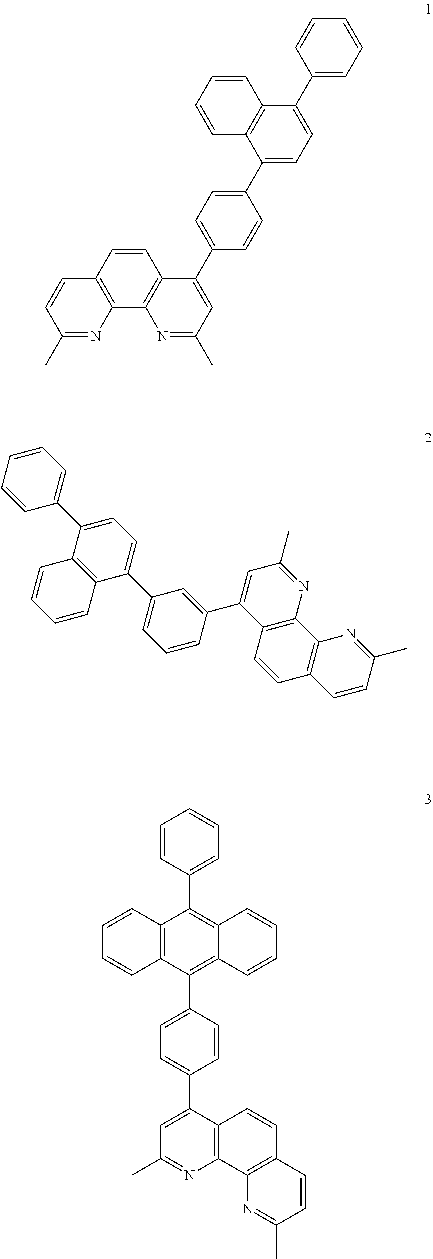 [SMILES: Cc1ccc2ccc3c(-c4ccc(-c5ccc(-c6ccccc6)c6ccccc56)cc4)cc(C)nc3c2n1 ; Cc1ccc2ccc3c(-c4cccc(-c5ccc(-c6ccccc6)c6ccccc56)c4)cc(C)nc3c2n1 ; Cc1ccc2ccc3c(-c4ccc(-c5c6ccccc6c(-c6ccccc6)c6ccccc56)cc4)cc(C)nc3c2n1]

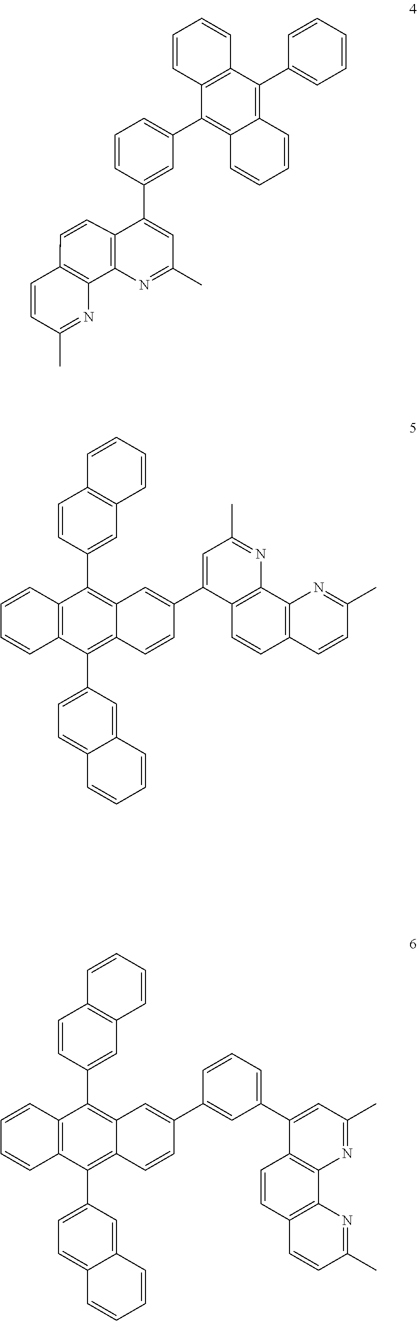 [SMILES: Cc1ccc2ccc3c(-c4cccc(-c5c6ccccc6c(-c6ccccc6)c6ccccc56)c4)cc(C)nc3c2n1 ; Cc1ccc2ccc3c(-c4ccc5c(-c6ccc7ccccc7c6)c6ccccc6c(-c6ccc7ccccc7c6)c5c4)cc(C)nc3c2n1 ; Cc1ccc2ccc3c(-c4cccc(-c5ccc6c(-c7ccc8ccccc8c7)c7ccccc7c(-c7ccc8ccccc8c7)c6c5)c4)cc(C)nc3c2n1]

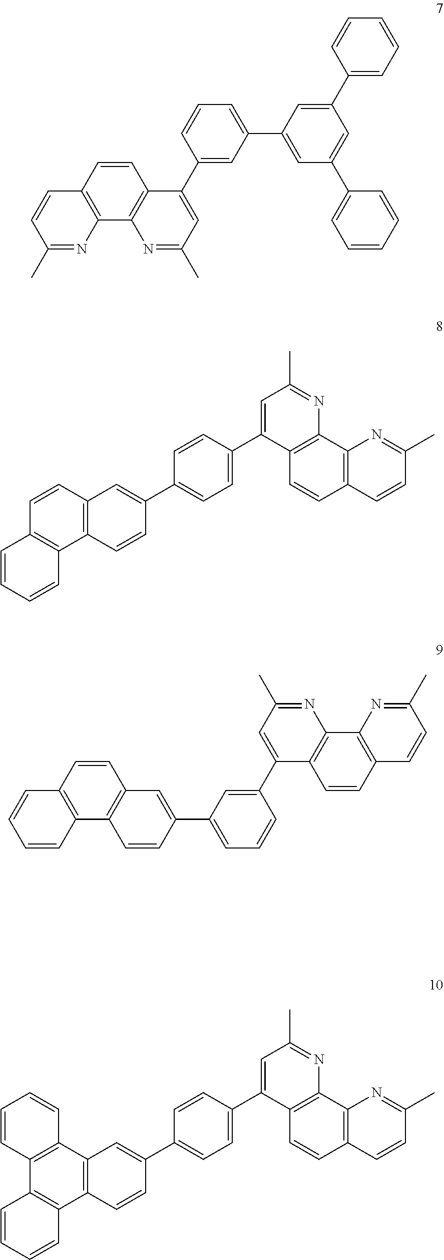 [SMILES: Cc1ccc2ccc3c(-c4cccc(-c5cc(-c6ccccc6)cc(-c6ccccc6)c5)c4)cc(C)nc3c2n1 ; Cc1ccc2ccc3c(-c4ccc(-c5ccc6c(ccc7ccccc76)c5)cc4)cc(C)nc3c2n1 ; Cc1ccc2ccc3c(-c4cccc(-c5ccc6c(ccc7ccccc76)c5)c4)cc(C)nc3c2n1 ; Cc1ccc2ccc3c(-c4ccc(-c5ccc6c7ccccc7c7ccccc7c6c5)cc4)cc(C)nc3c2n1]

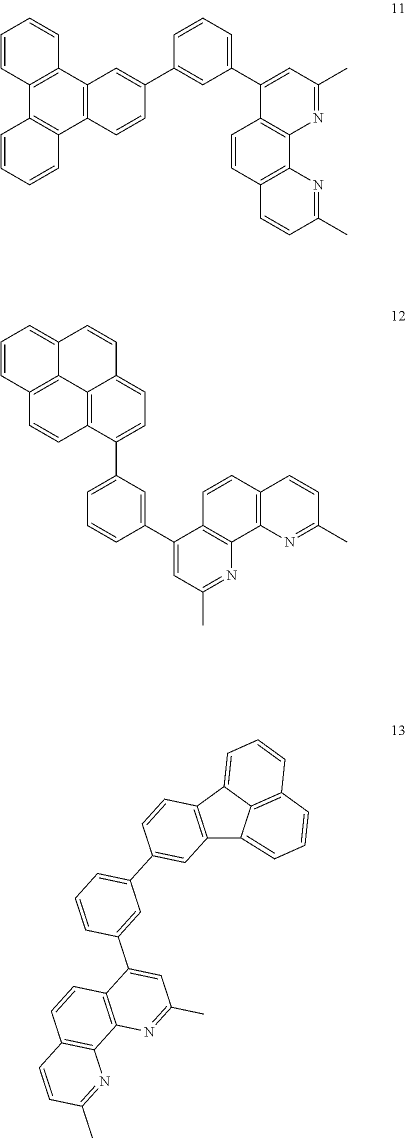 [SMILES: Cc1ccc2ccc3c(-c4cccc(-c5ccc6c7ccccc7c7ccccc7c6c5)c4)cc(C)nc3c2n1 ; Cc1ccc2ccc3c(-c4cccc(-c5ccc6ccc7cccc8ccc5c6c78)c4)cc(C)nc3c2n1 ; Cc1ccc2ccc3c(-c4cccc(-c5ccc6c(c5)-c5cccc7cccc-6c57)c4)cc(C)nc3c2n1]

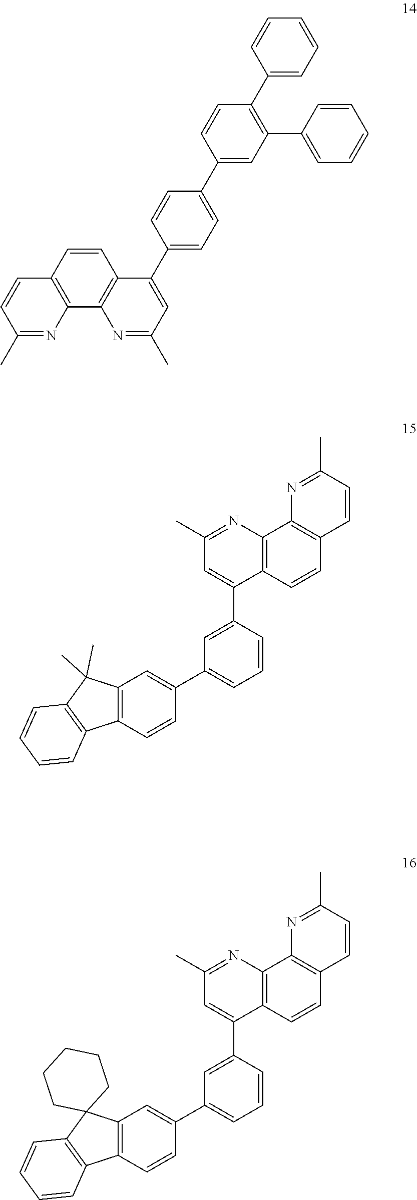 [SMILES: Cc1ccc2ccc3c(-c4ccc(-c5ccc(-c6ccccc6)c(-c6ccccc6)c5)cc4)cc(C)nc3c2n1 ; Cc1ccc2ccc3c(-c4cccc(-c5ccc6c(c5)C(C)(C)c5ccccc5-6)c4)cc(C)nc3c2n1 ; Cc1ccc2ccc3c(-c4cccc(-c5ccc6c(c5)C5(CCCCC5)c5ccccc5-6)c4)cc(C)nc3c2n1]

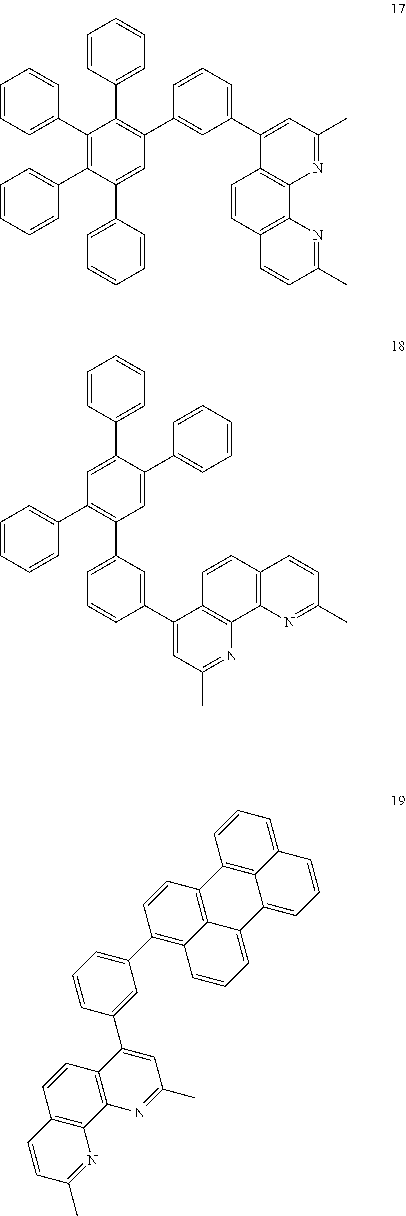 [SMILES: Cc1ccc2ccc3c(-c4cccc(-c5cc(-c6ccccc6)c(-c6ccccc6)c(-c6ccccc6)c5-c5ccccc5)c4)cc(C)nc3c2n1 ; Cc1ccc2ccc3c(-c4cccc(-c5cc(-c6ccccc6)c(-c6ccccc6)cc5-c5ccccc5)c4)cc(C)nc3c2n1 ; Cc1ccc2ccc3c(-c4cccc(-c5ccc6c7cccc8cccc(c9cccc5c96)c87)c4)cc(C)nc3c2n1]

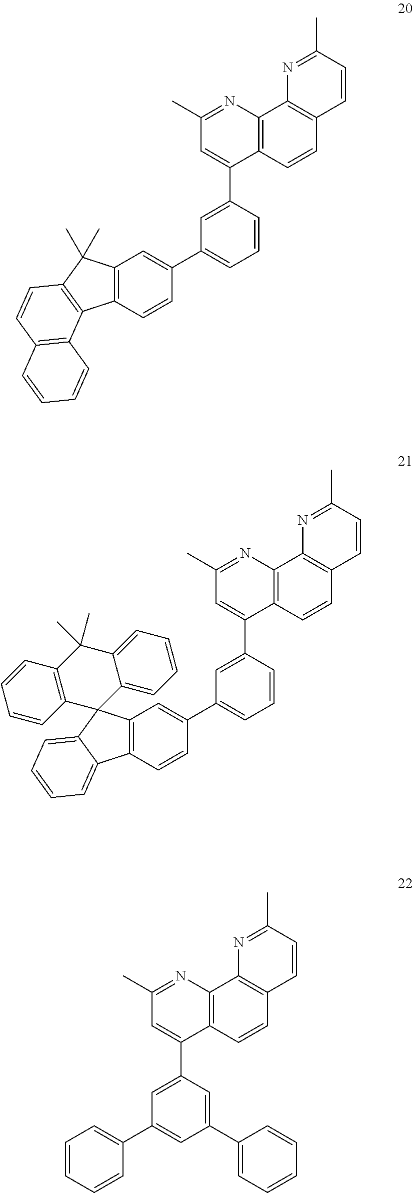 [SMILES: Cc1ccc2ccc3c(-c4cccc(-c5ccc6c(c5)C(C)(C)c5ccc7ccccc7c5-6)c4)cc(C)nc3c2n1 ; Cc1ccc2ccc3c(-c4cccc(-c5ccc6c(c5)C5(c7ccccc7-6)c6ccccc6C(C)(C)c6ccccc65)c4)cc(C)nc3c2n1 ; Cc1ccc2ccc3c(-c4cc(-c5ccccc5)cc(-c5ccccc5)c4)cc(C)nc3c2n1]

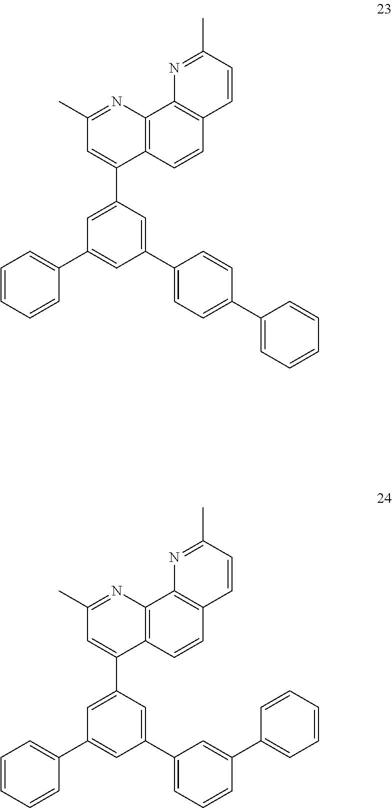 [SMILES: Cc1ccc2ccc3c(-c4cc(-c5ccccc5)cc(-c5ccc(-c6ccccc6)cc5)c4)cc(C)nc3c2n1 ; Cc1ccc2ccc3c(-c4cc(-c5ccccc5)cc(-c5cccc(-c6ccccc6)c5)c4)cc(C)nc3c2n1]

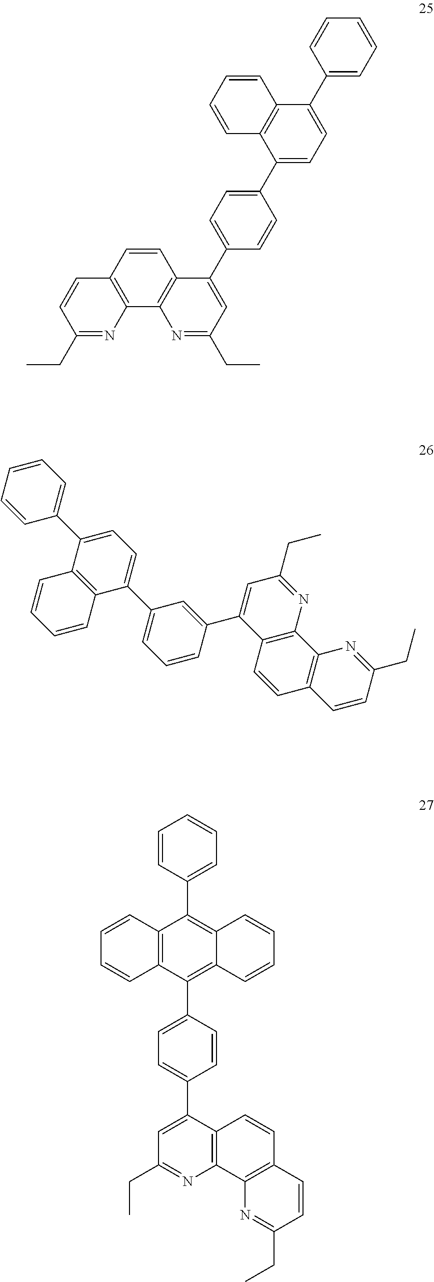 [SMILES: CCc1ccc2ccc3c(-c4ccc(-c5ccc(-c6ccccc6)c6ccccc56)cc4)cc(CC)nc3c2n1 ; CCc1ccc2ccc3c(-c4cccc(-c5ccc(-c6ccccc6)c6ccccc56)c4)cc(CC)nc3c2n1 ; CCc1ccc2ccc3c(-c4ccc(-c5c6ccccc6c(-c6ccccc6)c6ccccc56)cc4)cc(CC)nc3c2n1]

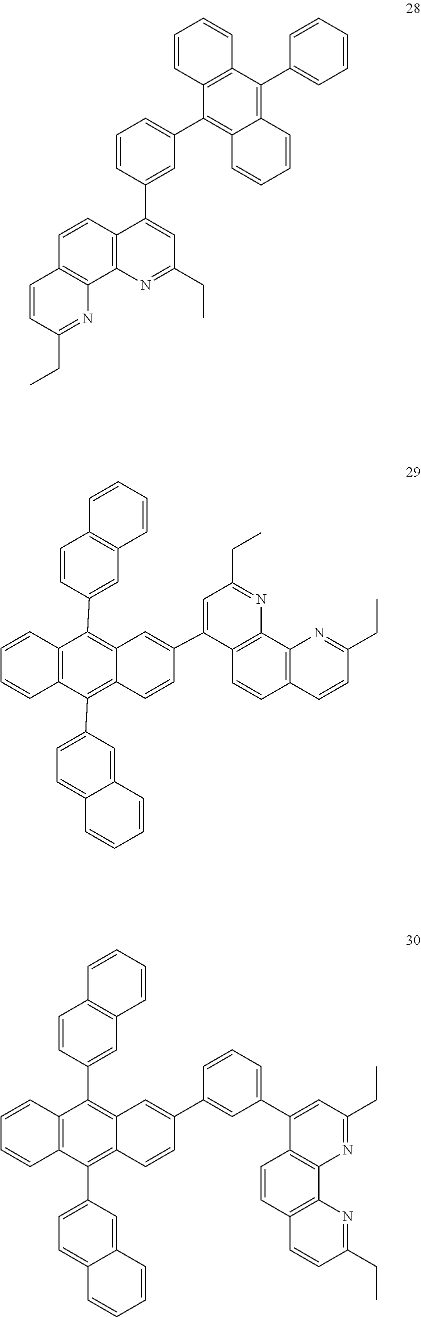 [SMILES: CCc1ccc2ccc3c(-c4cccc(-c5c6ccccc6c(-c6ccccc6)c6ccccc56)c4)cc(CC)nc3c2n1 ; CCc1ccc2ccc3c(-c4ccc5c(-c6ccc7ccccc7c6)c6ccccc6c(-c6ccc7ccccc7c6)c5c4)cc(CC)nc3c2n1 ; CCc1ccc2ccc3c(-c4cccc(-c5ccc6c(-c7ccc8ccccc8c7)c7ccccc7c(-c7ccc8ccccc8c7)c6c5)c4)cc(CC)nc3c2n1]

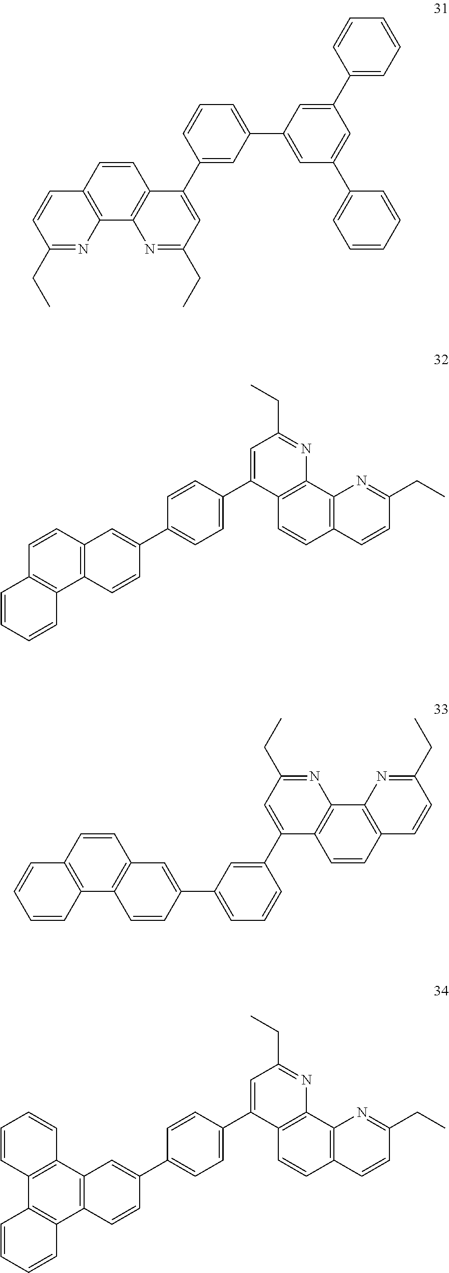 [SMILES: CCc1ccc2ccc3c(-c4cccc(-c5cc(-c6ccccc6)cc(-c6ccccc6)c5)c4)cc(CC)nc3c2n1 ; CCc1ccc2ccc3c(-c4ccc(-c5ccc6c(ccc7ccccc76)c5)cc4)cc(CC)nc3c2n1 ; CCc1ccc2ccc3c(-c4cccc(-c5ccc6c(ccc7ccccc76)c5)c4)cc(CC)nc3c2n1 ; CCc1ccc2ccc3c(-c4ccc(-c5ccc6c7ccccc7c7ccccc7c6c5)cc4)cc(CC)nc3c2n1]

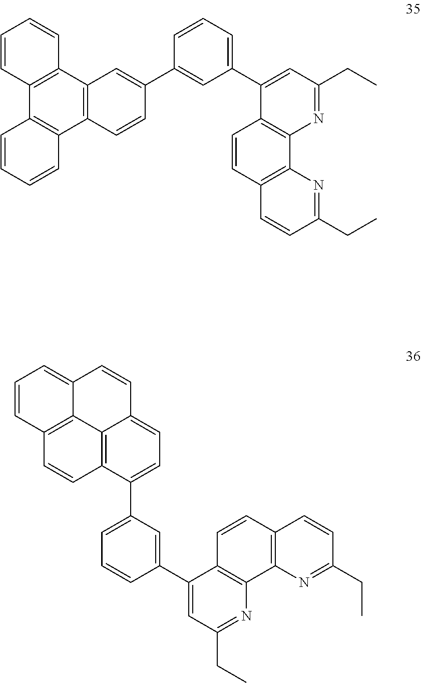 [SMILES: CCc1ccc2ccc3c(-c4cccc(-c5ccc6c7ccccc7c7ccccc7c6c5)c4)cc(CC)nc3c2n1 ; CCc1ccc2ccc3c(-c4cccc(-c5ccc6ccc7cccc8ccc5c6c78)c4)cc(CC)nc3c2n1]

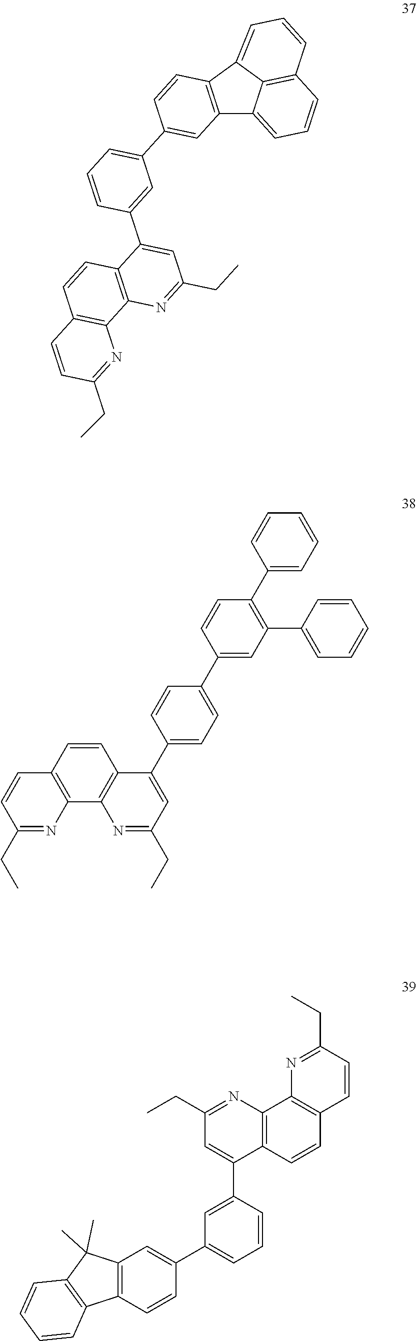 [SMILES: CCc1ccc2ccc3c(-c4cccc(-c5ccc6c(c5)-c5cccc7cccc-6c57)c4)cc(CC)nc3c2n1 ; CCc1ccc2ccc3c(-c4ccc(-c5ccc(-c6ccccc6)c(-c6ccccc6)c5)cc4)cc(CC)nc3c2n1 ; CCc1ccc2ccc3c(-c4cccc(-c5ccc6c(c5)C(C)(C)c5ccccc5-6)c4)cc(CC)nc3c2n1]

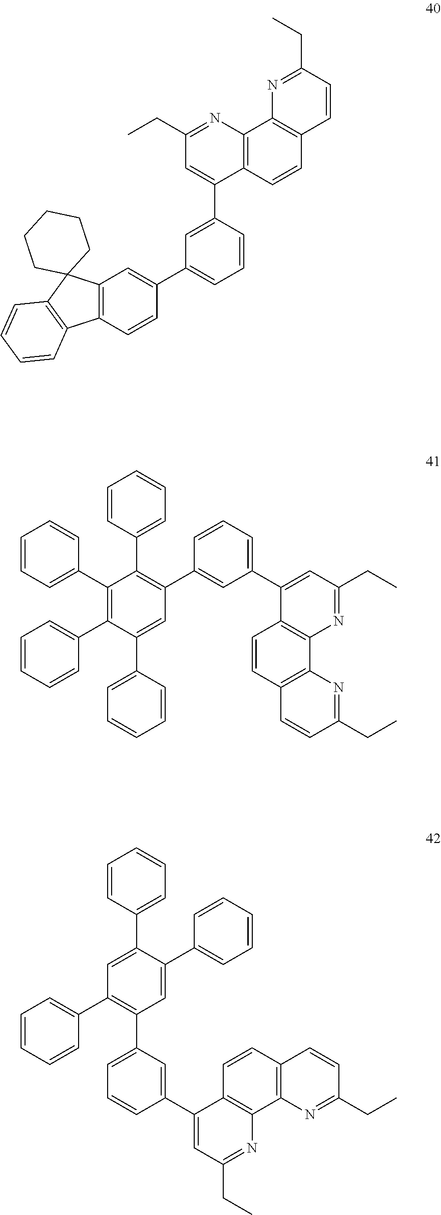 [SMILES: CCc1ccc2ccc3c(-c4cccc(-c5ccc6c(c5)C5(CCCCC5)c5ccccc5-6)c4)cc(CC)nc3c2n1 ; CCc1ccc2ccc3c(-c4cccc(-c5cc(-c6ccccc6)c(-c6ccccc6)c(-c6ccccc6)c5-c5ccccc5)c4)cc(CC)nc3c2n1 ; CCc1ccc2ccc3c(-c4cccc(-c5cc(-c6ccccc6)c(-c6ccccc6)cc5-c5ccccc5)c4)cc(CC)nc3c2n1]

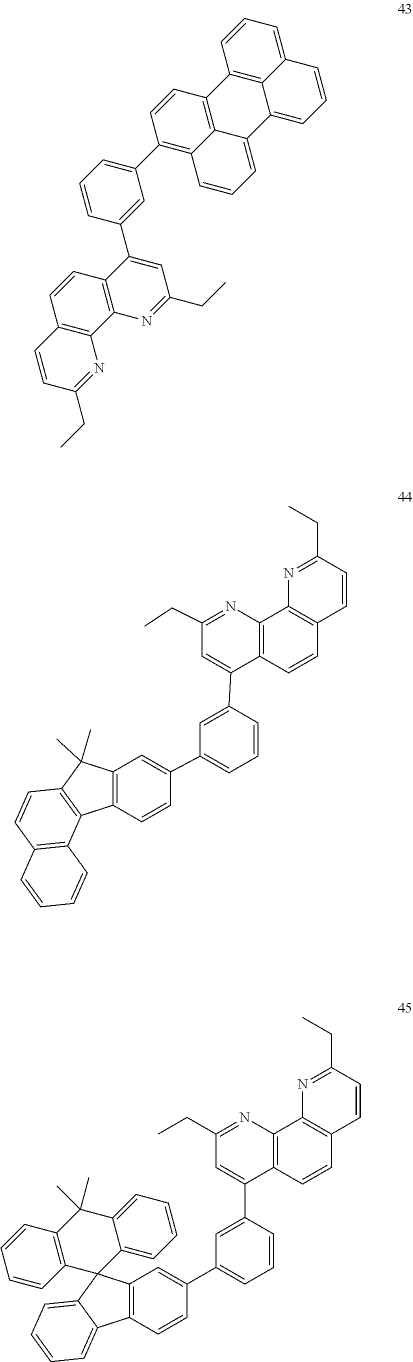 [SMILES: CCc1ccc2ccc3c(-c4cccc(-c5ccc6c7cccc8cccc(c9cccc5c96)c87)c4)cc(CC)nc3c2n1 ; CCc1ccc2ccc3c(-c4cccc(-c5ccc6c(c5)C(C)(C)c5ccc7ccccc7c5-6)c4)cc(CC)nc3c2n1 ; CCc1ccc2ccc3c(-c4cccc(-c5ccc6c(c5)C5(c7ccccc7-6)c6ccccc6C(C)(C)c6ccccc65)c4)cc(CC)nc3c2n1]

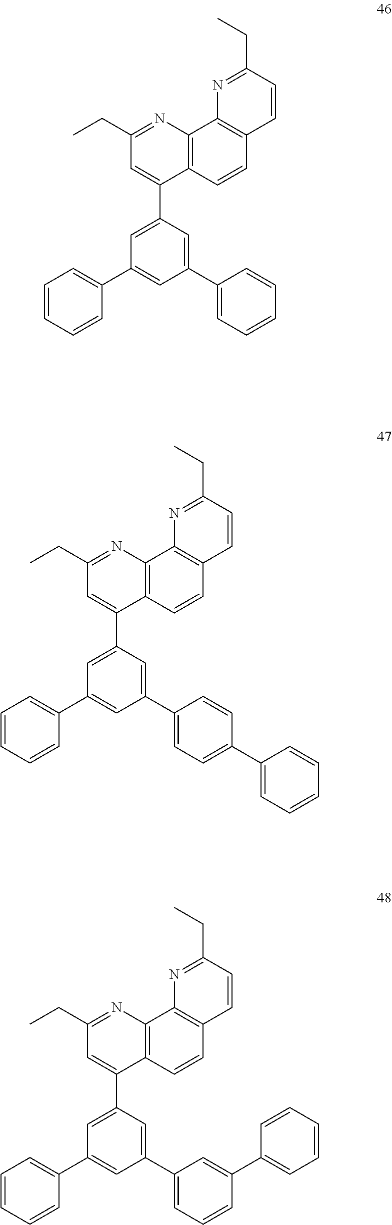 [SMILES: CCc1ccc2ccc3c(-c4cc(-c5ccccc5)cc(-c5ccccc5)c4)cc(CC)nc3c2n1 ; CCc1ccc2ccc3c(-c4cc(-c5ccccc5)cc(-c5ccc(-c6ccccc6)cc5)c4)cc(CC)nc3c2n1 ; CCc1ccc2ccc3c(-c4cc(-c5ccccc5)cc(-c5cccc(-c6ccccc6)c5)c4)cc(CC)nc3c2n1]

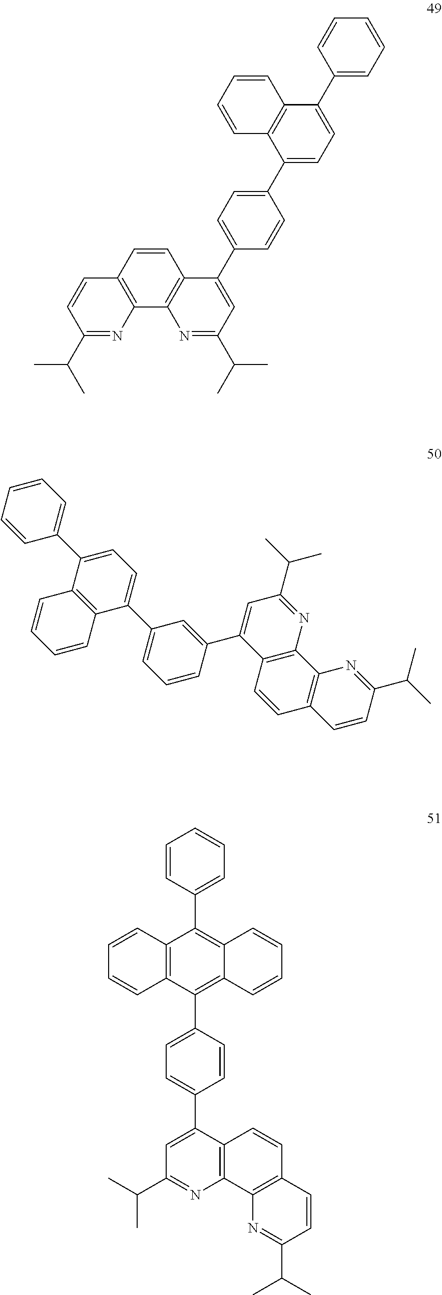 [SMILES: CC(C)c1ccc2ccc3c(-c4ccc(-c5ccc(-c6ccccc6)c6ccccc56)cc4)cc(C(C)C)nc3c2n1 ; CC(C)c1ccc2ccc3c(-c4cccc(-c5ccc(-c6ccccc6)c6ccccc56)c4)cc(C(C)C)nc3c2n1 ; CC(C)c1ccc2ccc3c(-c4ccc(-c5c6ccccc6c(-c6ccccc6)c6ccccc56)cc4)cc(C(C)C)nc3c2n1]

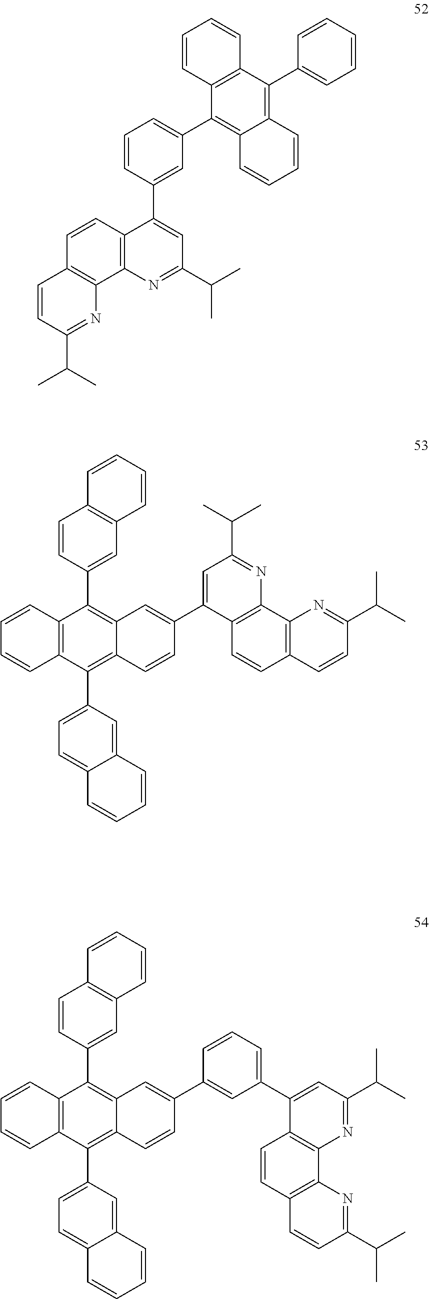 [SMILES: CC(C)c1ccc2ccc3c(-c4cccc(-c5c6ccccc6c(-c6ccccc6)c6ccccc56)c4)cc(C(C)C)nc3c2n1 ; CC(C)c1ccc2ccc3c(-c4ccc5c(-c6ccc7ccccc7c6)c6ccccc6c(-c6ccc7ccccc7c6)c5c4)cc(C(C)C)nc3c2n1 ; CC(C)c1ccc2ccc3c(-c4cccc(-c5ccc6c(-c7ccc8ccccc8c7)c7ccccc7c(-c7ccc8ccccc8c7)c6c5)c4)cc(C(C)C)nc3c2n1]

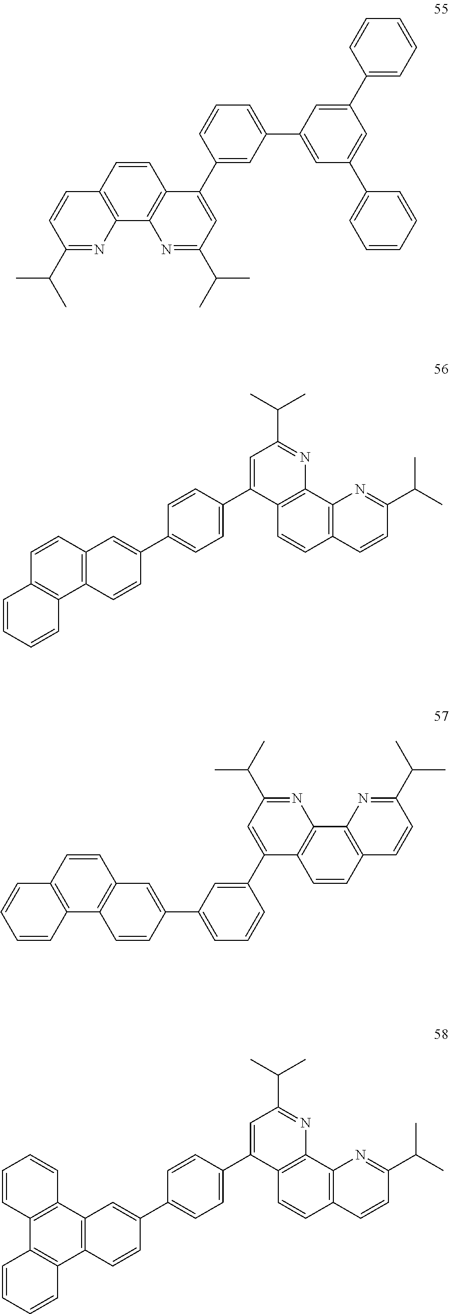 [SMILES: CC(C)c1ccc2ccc3c(-c4cccc(-c5cc(-c6ccccc6)cc(-c6ccccc6)c5)c4)cc(C(C)C)nc3c2n1 ; CC(C)c1ccc2ccc3c(-c4ccc(-c5ccc6c(ccc7ccccc76)c5)cc4)cc(C(C)C)nc3c2n1 ; CC(C)c1ccc2ccc3c(-c4cccc(-c5ccc6c(ccc7ccccc76)c5)c4)cc(C(C)C)nc3c2n1 ; CC(C)c1ccc2ccc3c(-c4ccc(-c5ccc6c7ccccc7c7ccccc7c6c5)cc4)cc(C(C)C)nc3c2n1]

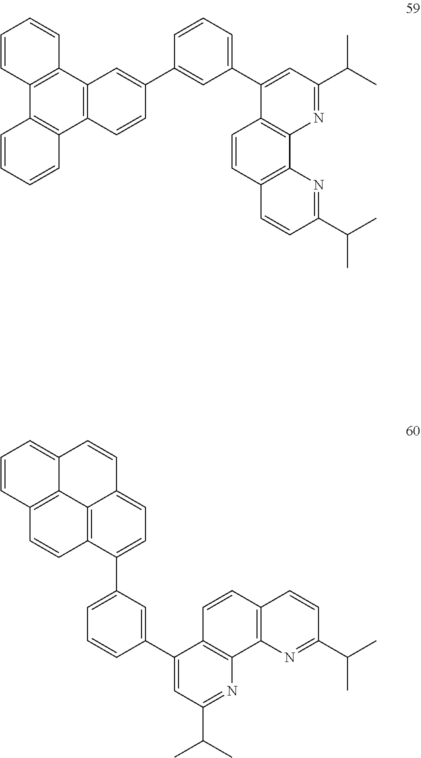 [SMILES: CC(C)c1ccc2ccc3c(-c4cccc(-c5ccc6c7ccccc7c7ccccc7c6c5)c4)cc(C(C)C)nc3c2n1 ; CC(C)c1ccc2ccc3c(-c4cccc(-c5ccc6ccc7cccc8ccc5c6c78)c4)cc(C(C)C)nc3c2n1]

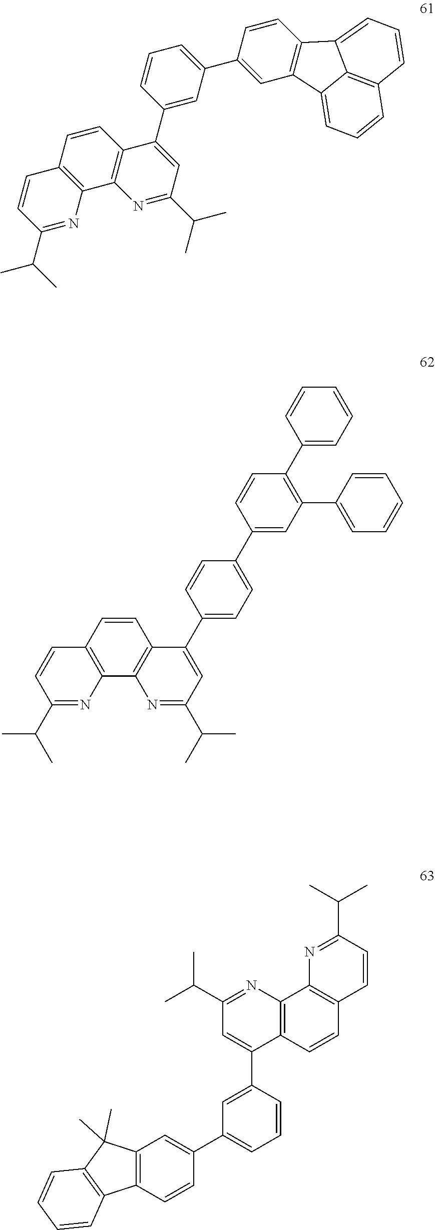 [SMILES: CC(C)c1ccc2ccc3c(-c4cccc(-c5ccc6c(c5)-c5cccc7cccc-6c57)c4)cc(C(C)C)nc3c2n1 ; CC(C)c1ccc2ccc3c(-c4ccc(-c5ccc(-c6ccccc6)c(-c6ccccc6)c5)cc4)cc(C(C)C)nc3c2n1 ; CC(C)c1ccc2ccc3c(-c4cccc(-c5ccc6c(c5)C(C)(C)c5ccccc5-6)c4)cc(C(C)C)nc3c2n1]

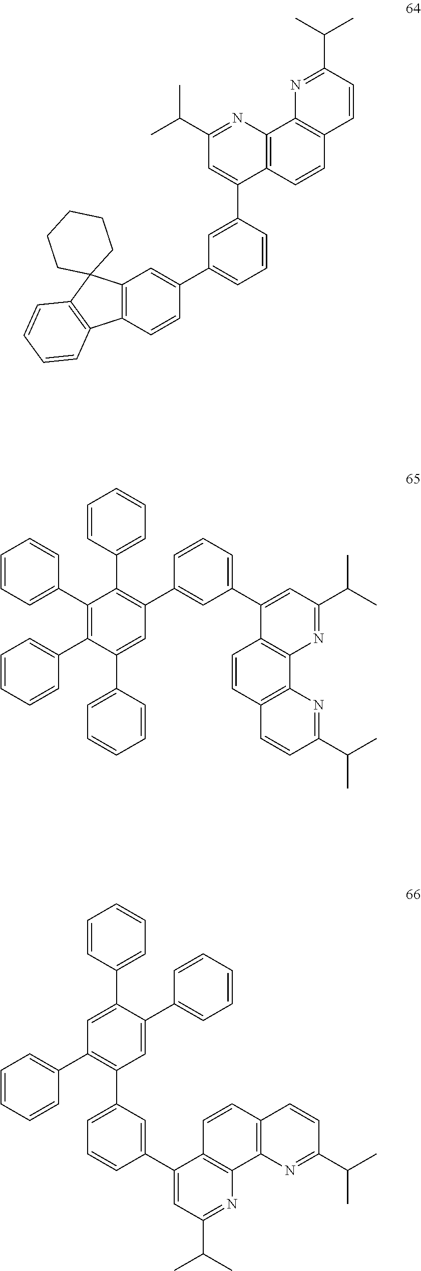 [SMILES: CC(C)c1ccc2ccc3c(-c4cccc(-c5ccc6c(c5)C5(CCCCC5)c5ccccc5-6)c4)cc(C(C)C)nc3c2n1 ; CC(C)c1ccc2ccc3c(-c4cccc(-c5cc(-c6ccccc6)c(-c6ccccc6)c(-c6ccccc6)c5-c5ccccc5)c4)cc(C(C)C)nc3c2n1 ; CC(C)c1ccc2ccc3c(-c4cccc(-c5cc(-c6ccccc6)c(-c6ccccc6)cc5-c5ccccc5)c4)cc(C(C)C)nc3c2n1]

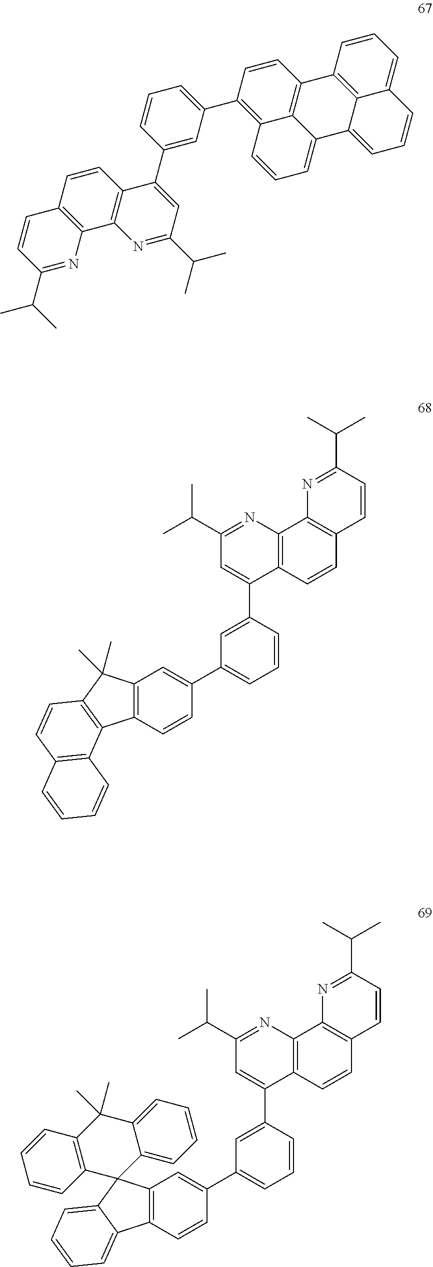 [SMILES: CC(C)c1ccc2ccc3c(-c4cccc(-c5ccc6c7cccc8cccc(c9cccc5c96)c87)c4)cc(C(C)C)nc3c2n1 ; CC(C)c1ccc2ccc3c(-c4cccc(-c5ccc6c(c5)C(C)(C)c5ccc7ccccc7c5-6)c4)cc(C(C)C)nc3c2n1 ; CC(C)c1ccc2ccc3c(-c4cccc(-c5ccc6c(c5)C5(c7ccccc7-6)c6ccccc6C(C)(C)c6ccccc65)c4)cc(C(C)C)nc3c2n1]

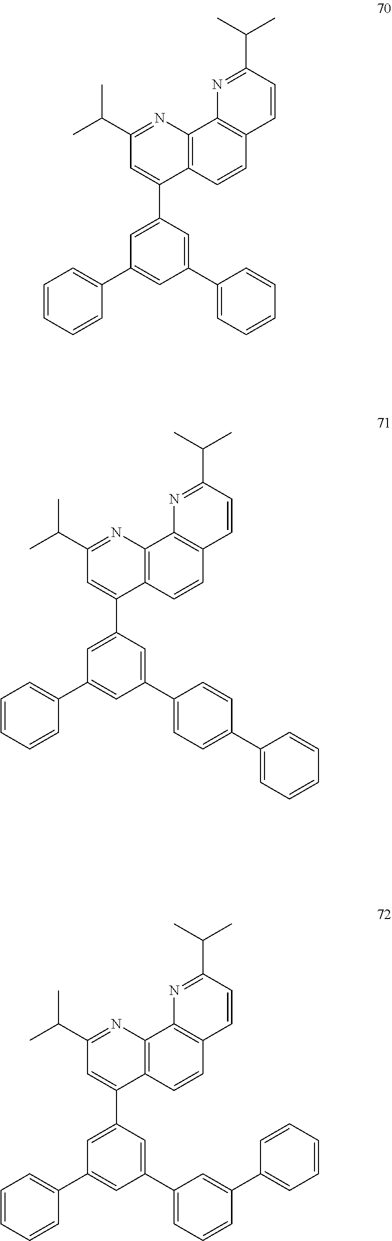 [SMILES: CC(C)c1ccc2ccc3c(-c4cc(-c5ccccc5)cc(-c5ccccc5)c4)cc(C(C)C)nc3c2n1 ; CC(C)c1ccc2ccc3c(-c4cc(-c5ccccc5)cc(-c5ccc(-c6ccccc6)cc5)c4)cc(C(C)C)nc3c2n1 ; CC(C)c1ccc2ccc3c(-c4cc(-c5ccccc5)cc(-c5cccc(-c6ccccc6)c5)c4)cc(C(C)C)nc3c2n1]

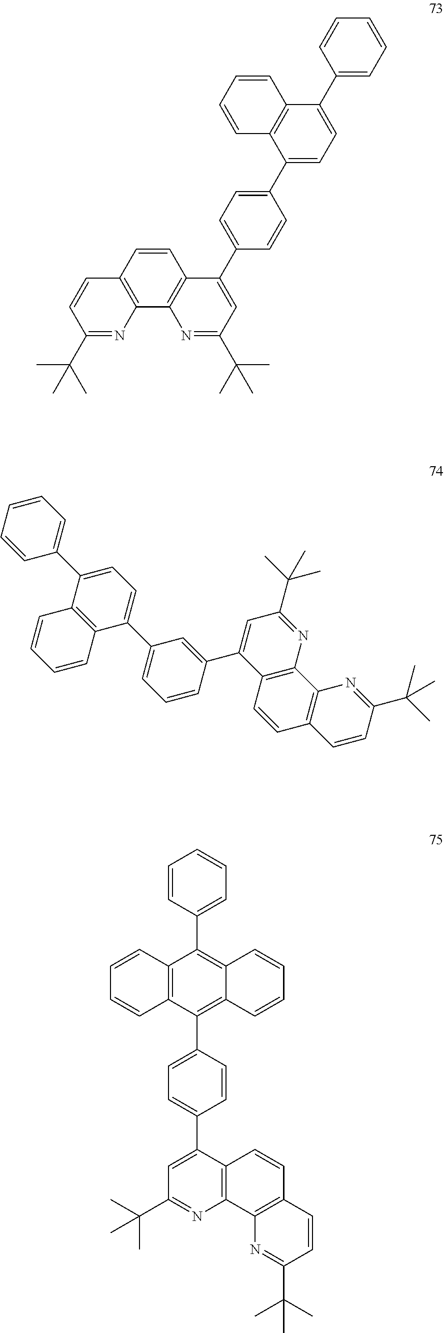 [SMILES: CC(C)(C)c1ccc2ccc3c(-c4ccc(-c5ccc(-c6ccccc6)c6ccccc56)cc4)cc(C(C)(C)C)nc3c2n1 ; CC(C)(C)c1ccc2ccc3c(-c4cccc(-c5ccc(-c6ccccc6)c6ccccc56)c4)cc(C(C)(C)C)nc3c2n1 ; CC(C)(C)c1ccc2ccc3c(-c4ccc(-c5c6ccccc6c(-c6ccccc6)c6ccccc56)cc4)cc(C(C)(C)C)nc3c2n1]

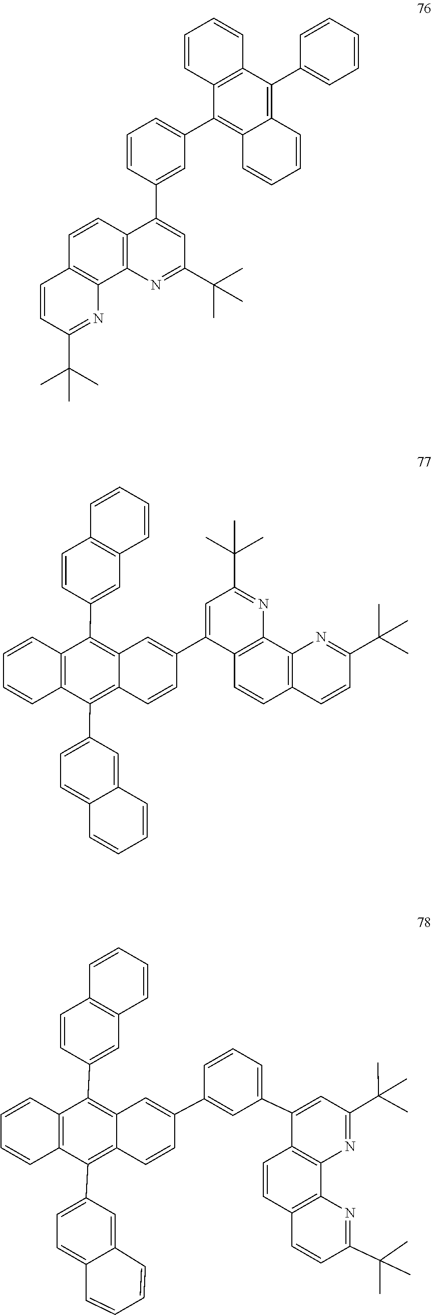 [SMILES: CC(C)(C)c1ccc2ccc3c(-c4cccc(-c5c6ccccc6c(-c6ccccc6)c6ccccc56)c4)cc(C(C)(C)C)nc3c2n1 ; CC(C)(C)c1ccc2ccc3c(-c4ccc5c(-c6ccc7ccccc7c6)c6ccccc6c(-c6ccc7ccccc7c6)c5c4)cc(C(C)(C)C)nc3c2n1 ; CC(C)(C)c1ccc2ccc3c(-c4cccc(-c5ccc6c(-c7ccc8ccccc8c7)c7ccccc7c(-c7ccc8ccccc8c7)c6c5)c4)cc(C(C)(C)C)nc3c2n1]

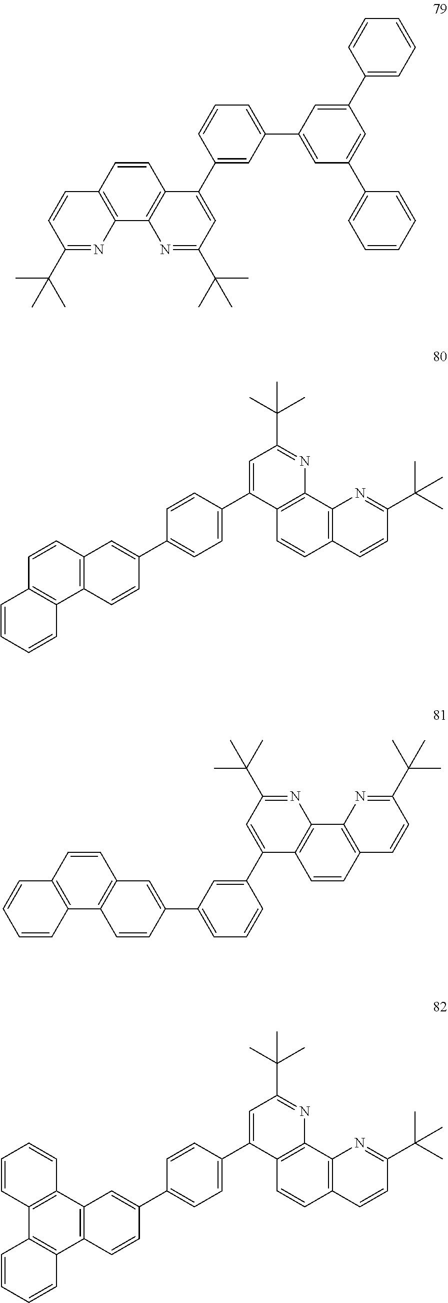 [SMILES: CC(C)(C)c1ccc2ccc3c(-c4cccc(-c5cc(-c6ccccc6)cc(-c6ccccc6)c5)c4)cc(C(C)(C)C)nc3c2n1 ; CC(C)(C)c1ccc2ccc3c(-c4ccc(-c5ccc6c(ccc7ccccc76)c5)cc4)cc(C(C)(C)C)nc3c2n1 ; CC(C)(C)c1ccc2ccc3c(-c4cccc(-c5ccc6c(ccc7ccccc76)c5)c4)cc(C(C)(C)C)nc3c2n1 ; CC(C)(C)c1ccc2ccc3c(-c4ccc(-c5ccc6c7ccccc7c7ccccc7c6c5)cc4)cc(C(C)(C)C)nc3c2n1]

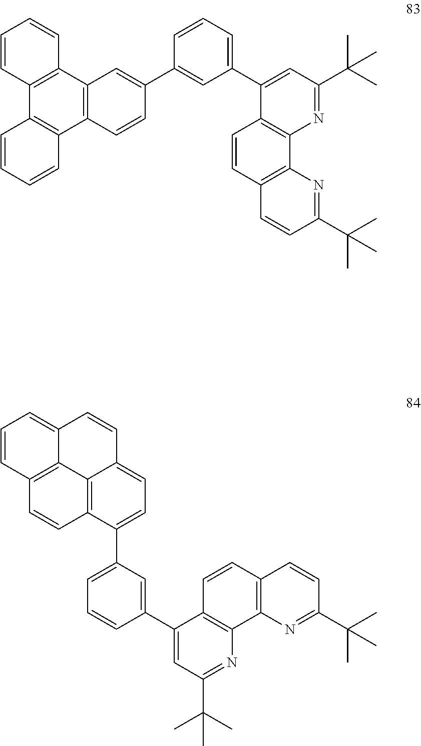 [SMILES: CC(C)(C)c1ccc2ccc3c(-c4cccc(-c5ccc6c7ccccc7c7ccccc7c6c5)c4)cc(C(C)(C)C)nc3c2n1 ; CC(C)(C)c1ccc2ccc3c(-c4cccc(-c5ccc6ccc7cccc8ccc5c6c78)c4)cc(C(C)(C)C)nc3c2n1]

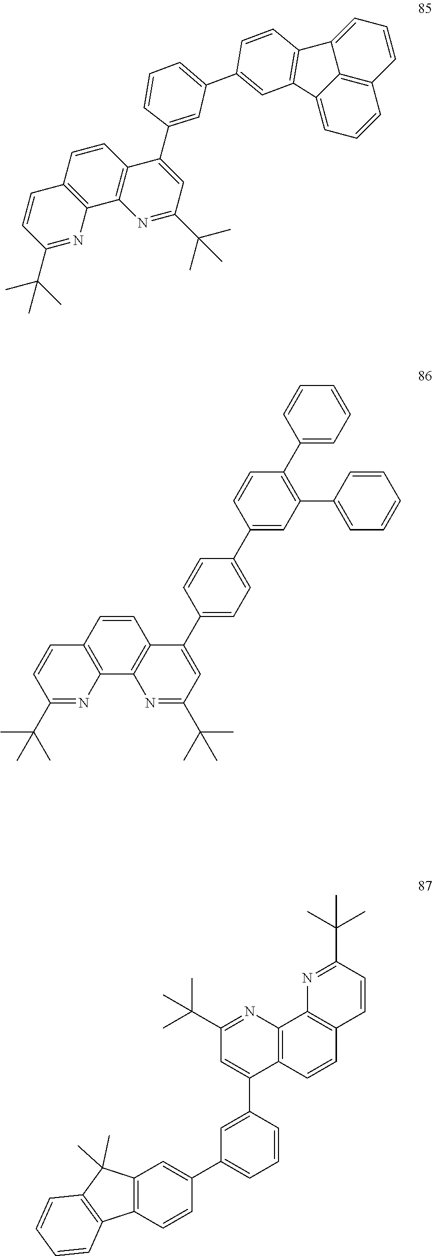 [SMILES: CC(C)(C)c1ccc2ccc3c(-c4cccc(-c5ccc6c(c5)-c5cccc7cccc-6c57)c4)cc(C(C)(C)C)nc3c2n1 ; CC(C)(C)c1ccc2ccc3c(-c4ccc(-c5ccc(-c6ccccc6)c(-c6ccccc6)c5)cc4)cc(C(C)(C)C)nc3c2n1 ; CC(C)(C)c1ccc2ccc3c(-c4cccc(-c5ccc6c(c5)C(C)(C)c5ccccc5-6)c4)cc(C(C)(C)C)nc3c2n1]

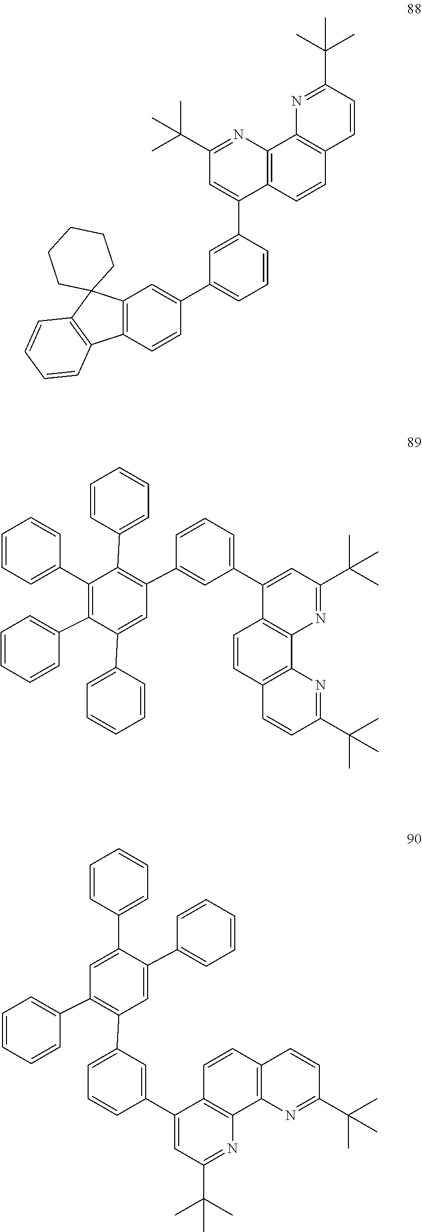 [SMILES: CC(C)(C)c1ccc2ccc3c(-c4cccc(-c5ccc6c(c5)C5(CCCCC5)c5ccccc5-6)c4)cc(C(C)(C)C)nc3c2n1 ; CC(C)(C)c1ccc2ccc3c(-c4cccc(-c5cc(-c6ccccc6)c(-c6ccccc6)c(-c6ccccc6)c5-c5ccccc5)c4)cc(C(C)(C)C)nc3c2n1 ; CC(C)(C)c1ccc2ccc3c(-c4cccc(-c5cc(-c6ccccc6)c(-c6ccccc6)cc5-c5ccccc5)c4)cc(C(C)(C)C)nc3c2n1]

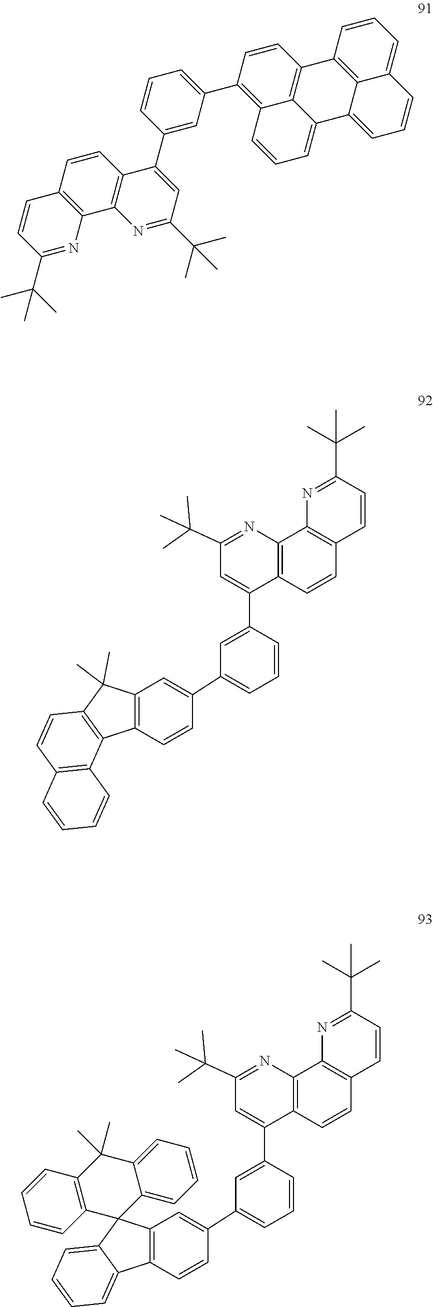 [SMILES: CC(C)(C)c1ccc2ccc3c(-c4cccc(-c5ccc6c7cccc8cccc(c9cccc5c96)c87)c4)cc(C(C)(C)C)nc3c2n1 ; CC(C)(C)c1ccc2ccc3c(-c4cccc(-c5ccc6c(c5)C(C)(C)c5ccc7ccccc7c5-6)c4)cc(C(C)(C)C)nc3c2n1 ; CC(C)(C)c1ccc2ccc3c(-c4cccc(-c5ccc6c(c5)C5(c7ccccc7-6)c6ccccc6C(C)(C)c6ccccc65)c4)cc(C(C)(C)C)nc3c2n1]

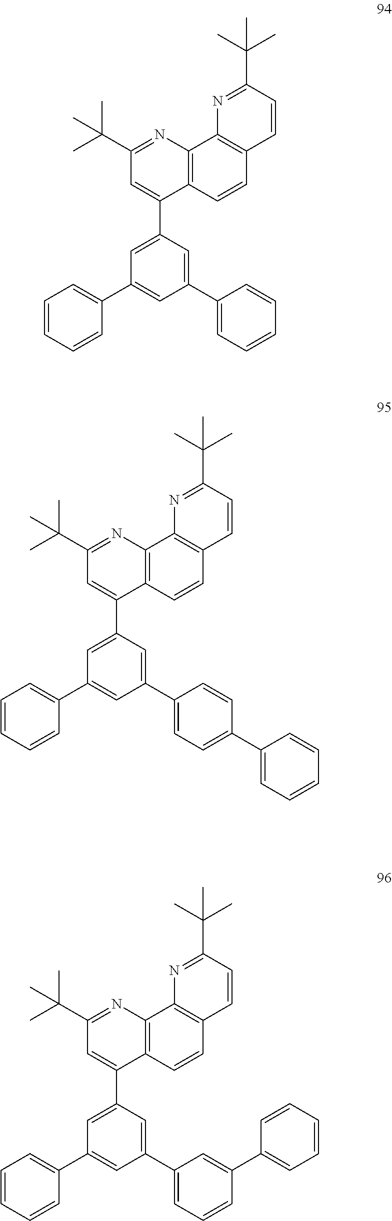 [SMILES: CC(C)(C)c1ccc2ccc3c(-c4cc(-c5ccccc5)cc(-c5ccccc5)c4)cc(C(C)(C)C)nc3c2n1 ; CC(C)(C)c1ccc2ccc3c(-c4cc(-c5ccccc5)cc(-c5ccc(-c6ccccc6)cc5)c4)cc(C(C)(C)C)nc3c2n1 ; CC(C)(C)c1ccc2ccc3c(-c4cc(-c5ccccc5)cc(-c5cccc(-c6ccccc6)c5)c4)cc(C(C)(C)C)nc3c2n1]

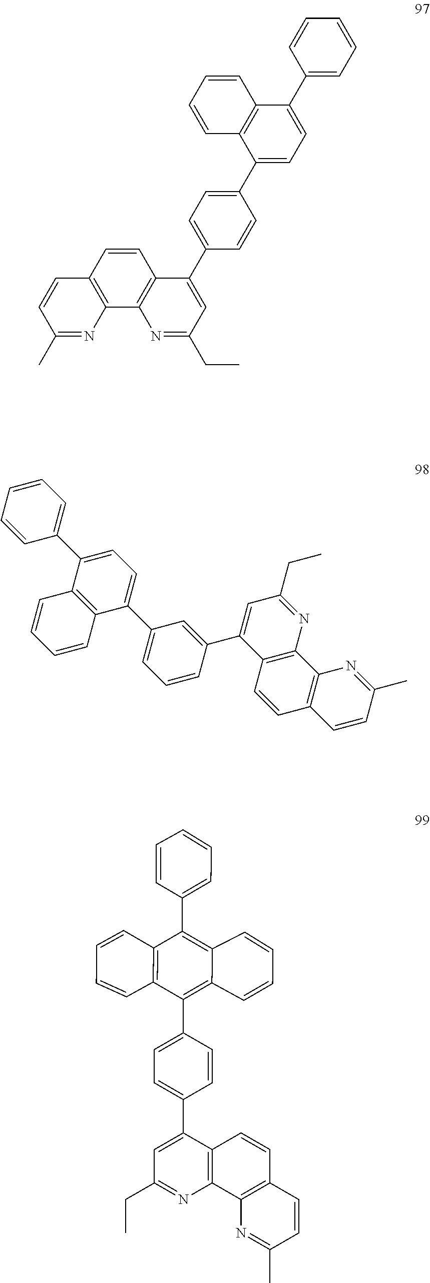 [SMILES: CCc1cc(-c2ccc(-c3ccc(-c4ccccc4)c4ccccc34)cc2)c2ccc3ccc(C)nc3c2n1 ; CCc1cc(-c2cccc(-c3ccc(-c4ccccc4)c4ccccc34)c2)c2ccc3ccc(C)nc3c2n1 ; CCc1cc(-c2ccc(-c3c4ccccc4c(-c4ccccc4)c4ccccc34)cc2)c2ccc3ccc(C)nc3c2n1]

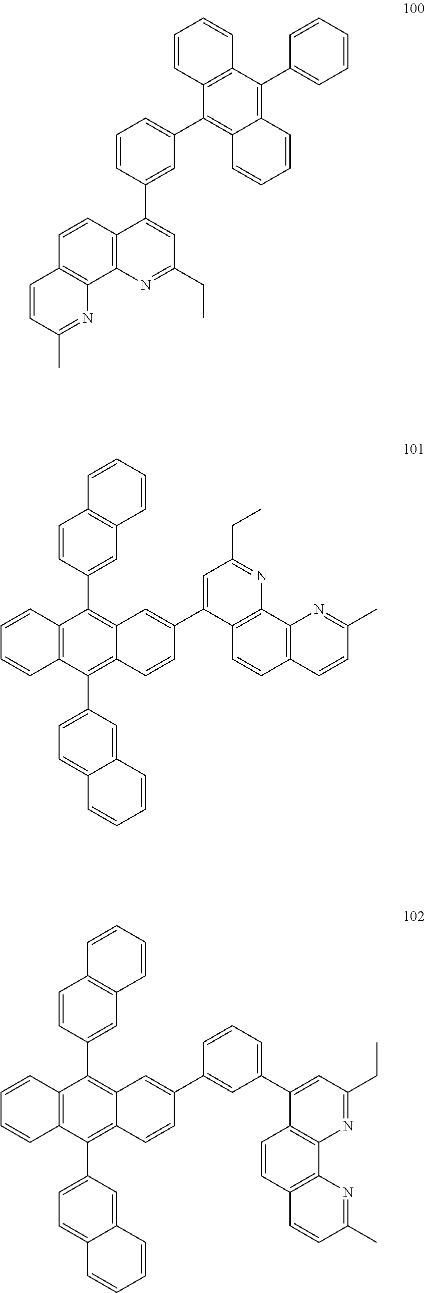 [SMILES: CCc1cc(-c2cccc(-c3c4ccccc4c(-c4ccccc4)c4ccccc34)c2)c2ccc3ccc(C)nc3c2n1 ; CCc1cc(-c2ccc3c(-c4ccc5ccccc5c4)c4ccccc4c(-c4ccc5ccccc5c4)c3c2)c2ccc3ccc(C)nc3c2n1 ; CCc1cc(-c2cccc(-c3ccc4c(-c5ccc6ccccc6c5)c5ccccc5c(-c5ccc6ccccc6c5)c4c3)c2)c2ccc3ccc(C)nc3c2n1]

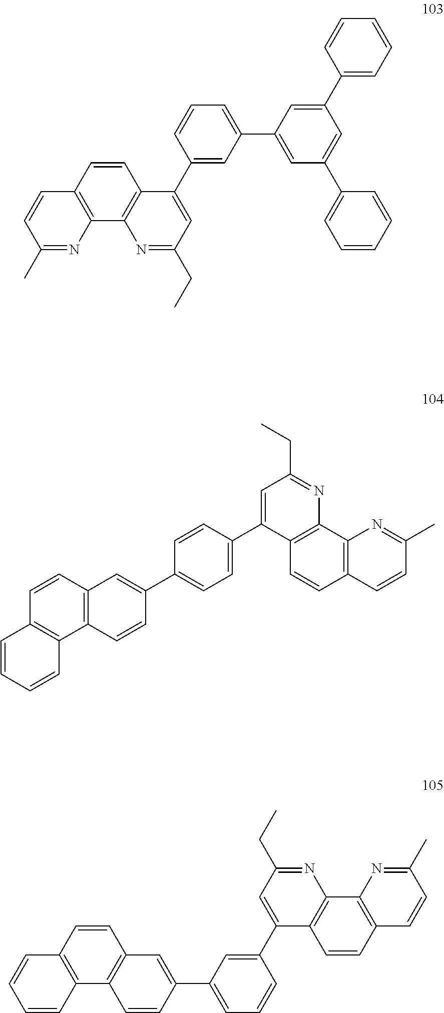 [SMILES: CCc1cc(-c2cccc(-c3cc(-c4ccccc4)cc(-c4ccccc4)c3)c2)c2ccc3ccc(C)nc3c2n1 ; CCc1cc(-c2ccc(-c3ccc4c(ccc5ccccc54)c3)cc2)c2ccc3ccc(C)nc3c2n1 ; CCc1cc(-c2cccc(-c3ccc4c(ccc5ccccc54)c3)c2)c2ccc3ccc(C)nc3c2n1]

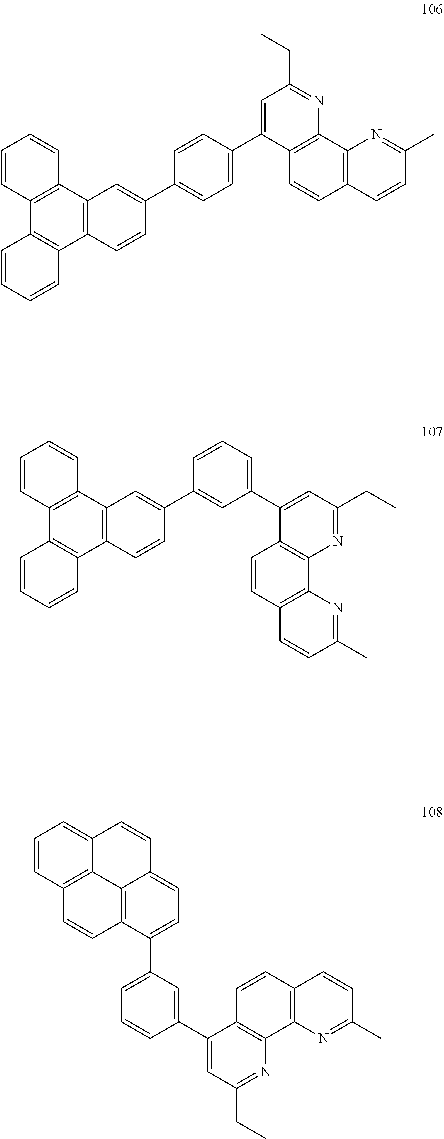 [SMILES: CCc1cc(-c2ccc(-c3ccc4c5ccccc5c5ccccc5c4c3)cc2)c2ccc3ccc(C)nc3c2n1 ; CCc1cc(-c2cccc(-c3ccc4c5ccccc5c5ccccc5c4c3)c2)c2ccc3ccc(C)nc3c2n1 ; CCc1cc(-c2cccc(-c3ccc4ccc5cccc6ccc3c4c56)c2)c2ccc3ccc(C)nc3c2n1]

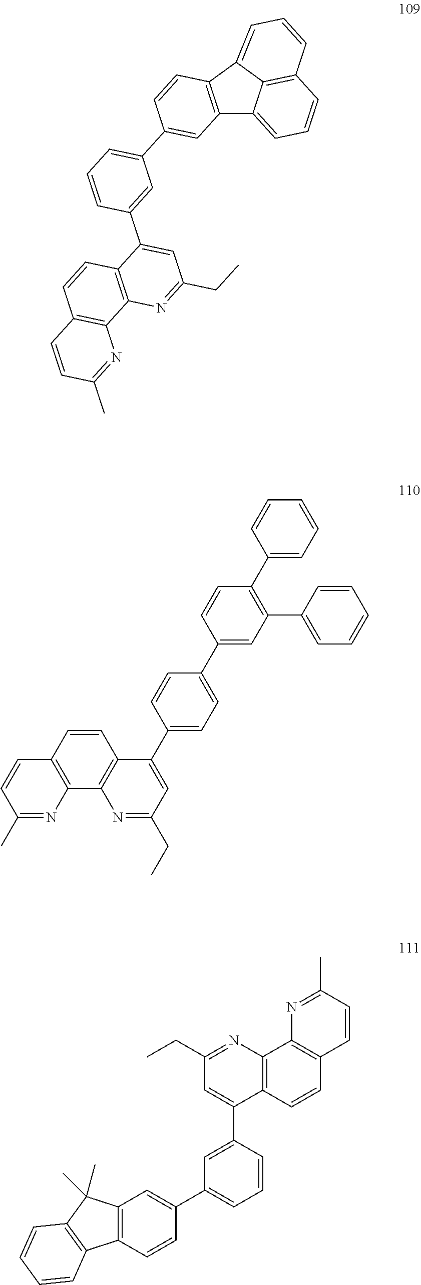 [SMILES: CCc1cc(-c2cccc(-c3ccc4c(c3)-c3cccc5cccc-4c35)c2)c2ccc3ccc(C)nc3c2n1 ; CCc1cc(-c2ccc(-c3ccc(-c4ccccc4)c(-c4ccccc4)c3)cc2)c2ccc3ccc(C)nc3c2n1 ; CCc1cc(-c2cccc(-c3ccc4c(c3)C(C)(C)c3ccccc3-4)c2)c2ccc3ccc(C)nc3c2n1]

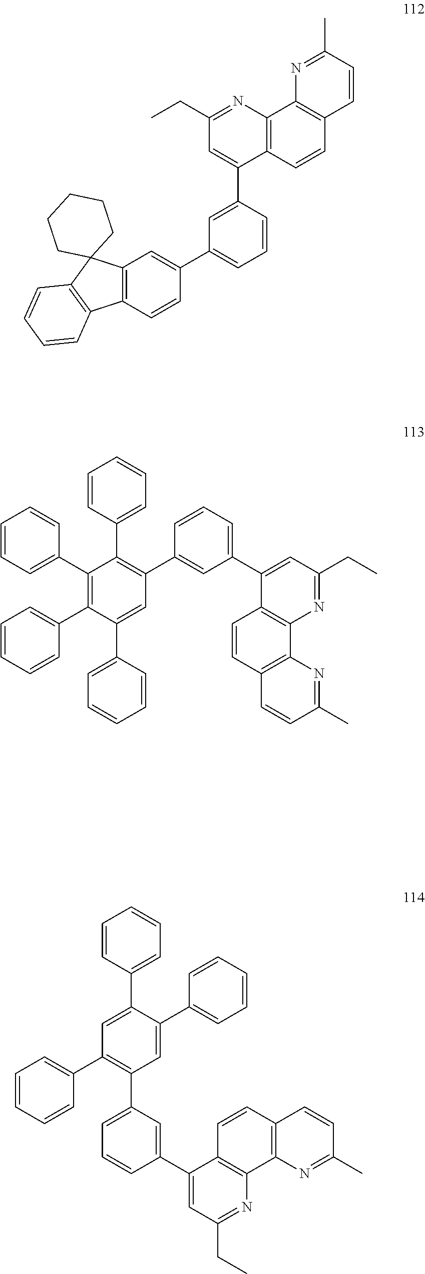 [SMILES: CCc1cc(-c2cccc(-c3ccc4c(c3)C3(CCCCC3)c3ccccc3-4)c2)c2ccc3ccc(C)nc3c2n1 ; CCc1cc(-c2cccc(-c3cc(-c4ccccc4)c(-c4ccccc4)c(-c4ccccc4)c3-c3ccccc3)c2)c2ccc3ccc(C)nc3c2n1 ; CCc1cc(-c2cccc(-c3cc(-c4ccccc4)c(-c4ccccc4)cc3-c3ccccc3)c2)c2ccc3ccc(C)nc3c2n1]

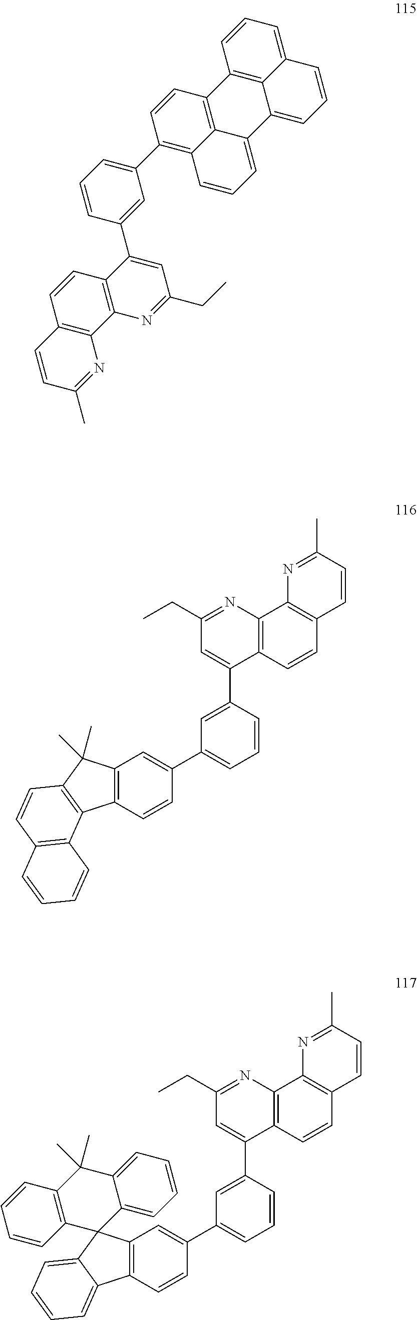 [SMILES: CCc1cc(-c2cccc(-c3ccc4c5cccc6cccc(c7cccc3c74)c65)c2)c2ccc3ccc(C)nc3c2n1 ; CCc1cc(-c2cccc(-c3ccc4c(c3)C(C)(C)c3ccc5ccccc5c3-4)c2)c2ccc3ccc(C)nc3c2n1 ; CCc1cc(-c2cccc(-c3ccc4c(c3)C3(c5ccccc5-4)c4ccccc4C(C)(C)c4ccccc43)c2)c2ccc3ccc(C)nc3c2n1]

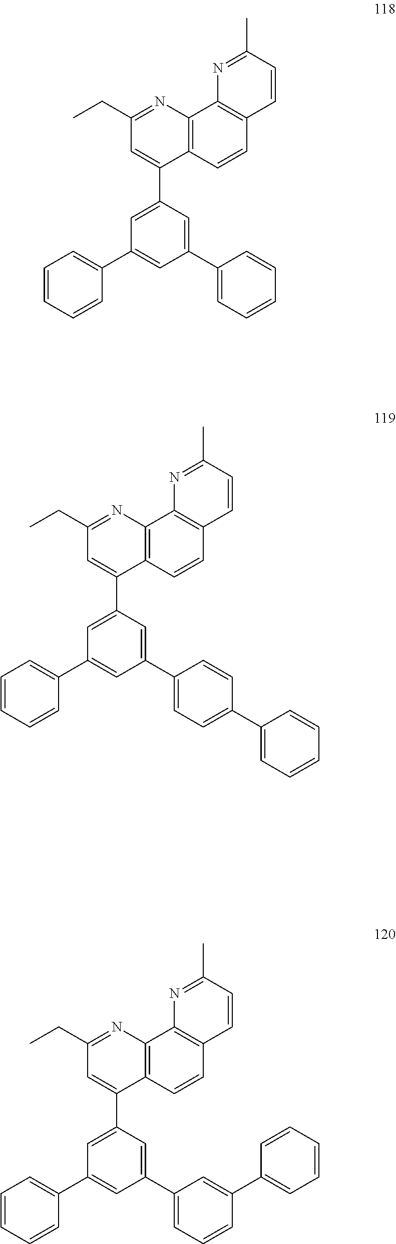 [SMILES: CCc1cc(-c2cc(-c3ccccc3)cc(-c3ccccc3)c2)c2ccc3ccc(C)nc3c2n1 ; CCc1cc(-c2cc(-c3ccccc3)cc(-c3ccc(-c4ccccc4)cc3)c2)c2ccc3ccc(C)nc3c2n1 ; CCc1cc(-c2cc(-c3ccccc3)cc(-c3cccc(-c4ccccc4)c3)c2)c2ccc3ccc(C)nc3c2n1]

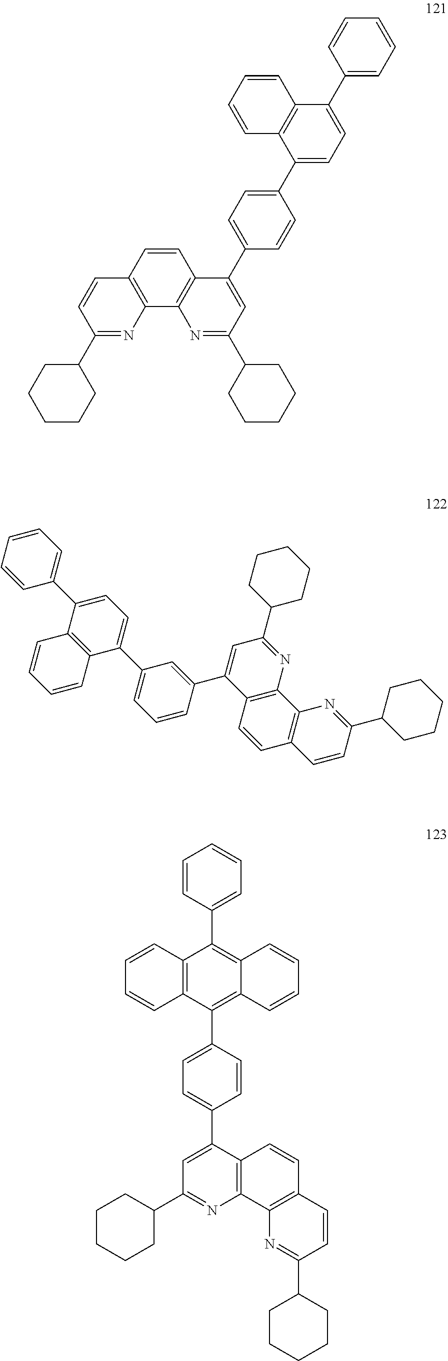 [SMILES: c1ccc(-c2ccc(-c3ccc(-c4cc(C5CCCCC5)nc5c4ccc4ccc(C6CCCCC6)nc45)cc3)c3ccccc23)cc1 ; c1ccc(-c2ccc(-c3cccc(-c4cc(C5CCCCC5)nc5c4ccc4ccc(C6CCCCC6)nc45)c3)c3ccccc23)cc1 ; c1ccc(-c2c3ccccc3c(-c3ccc(-c4cc(C5CCCCC5)nc5c4ccc4ccc(C6CCCCC6)nc45)cc3)c3ccccc23)cc1]

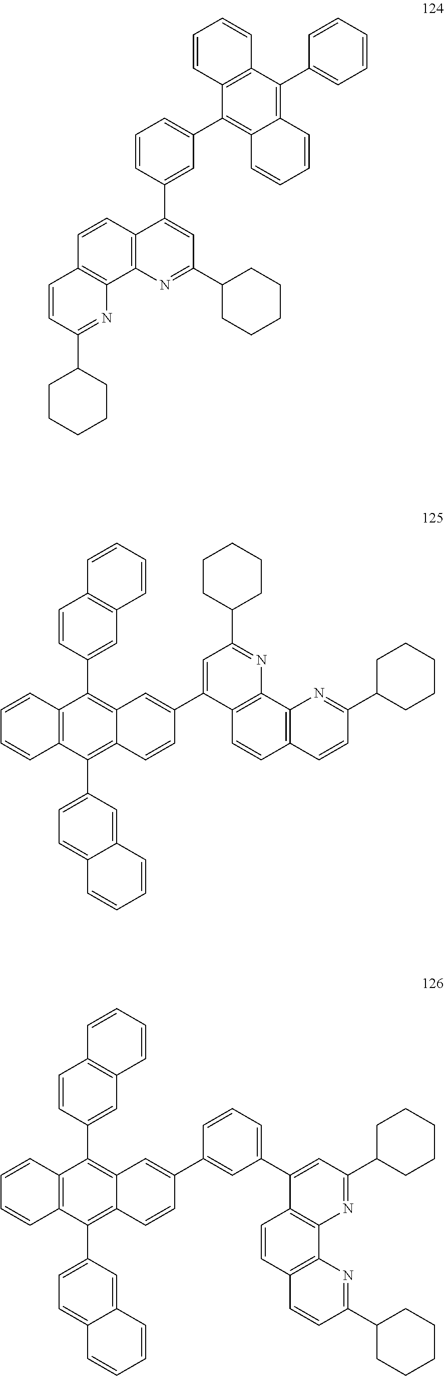 [SMILES: c1ccc(-c2c3ccccc3c(-c3cccc(-c4cc(C5CCCCC5)nc5c4ccc4ccc(C6CCCCC6)nc45)c3)c3ccccc23)cc1 ; c1ccc2cc(-c3c4ccccc4c(-c4ccc5ccccc5c4)c4cc(-c5cc(C6CCCCC6)nc6c5ccc5ccc(C7CCCCC7)nc56)ccc34)ccc2c1 ; c1cc(-c2ccc3c(-c4ccc5ccccc5c4)c4ccccc4c(-c4ccc5ccccc5c4)c3c2)cc(-c2cc(C3CCCCC3)nc3c2ccc2ccc(C4CCCCC4)nc23)c1]

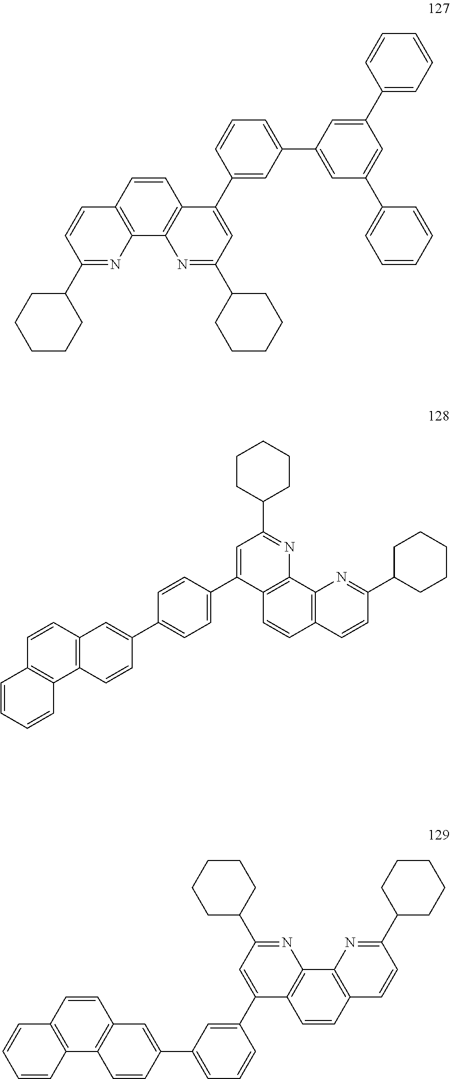 [SMILES: c1ccc(-c2cc(-c3ccccc3)cc(-c3cccc(-c4cc(C5CCCCC5)nc5c4ccc4ccc(C6CCCCC6)nc45)c3)c2)cc1 ; c1ccc2c(c1)ccc1cc(-c3ccc(-c4cc(C5CCCCC5)nc5c4ccc4ccc(C6CCCCC6)nc45)cc3)ccc12 ; c1cc(-c2ccc3c(ccc4ccccc43)c2)cc(-c2cc(C3CCCCC3)nc3c2ccc2ccc(C4CCCCC4)nc23)c1]

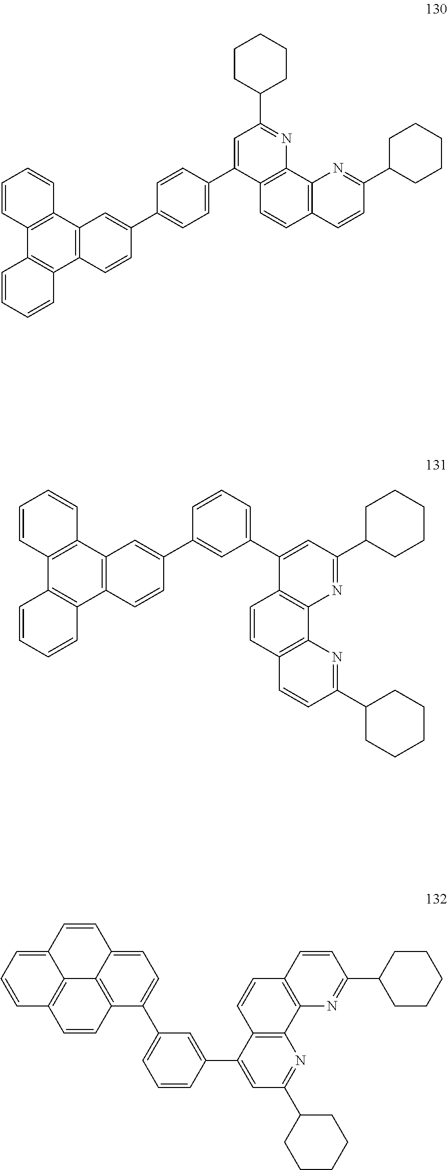 [SMILES: c1ccc2c(c1)c1ccccc1c1cc(-c3ccc(-c4cc(C5CCCCC5)nc5c4ccc4ccc(C6CCCCC6)nc45)cc3)ccc21 ; c1cc(-c2ccc3c4ccccc4c4ccccc4c3c2)cc(-c2cc(C3CCCCC3)nc3c2ccc2ccc(C4CCCCC4)nc23)c1 ; c1cc(-c2cc(C3CCCCC3)nc3c2ccc2ccc(C4CCCCC4)nc23)cc(-c2ccc3ccc4cccc5ccc2c3c45)c1]

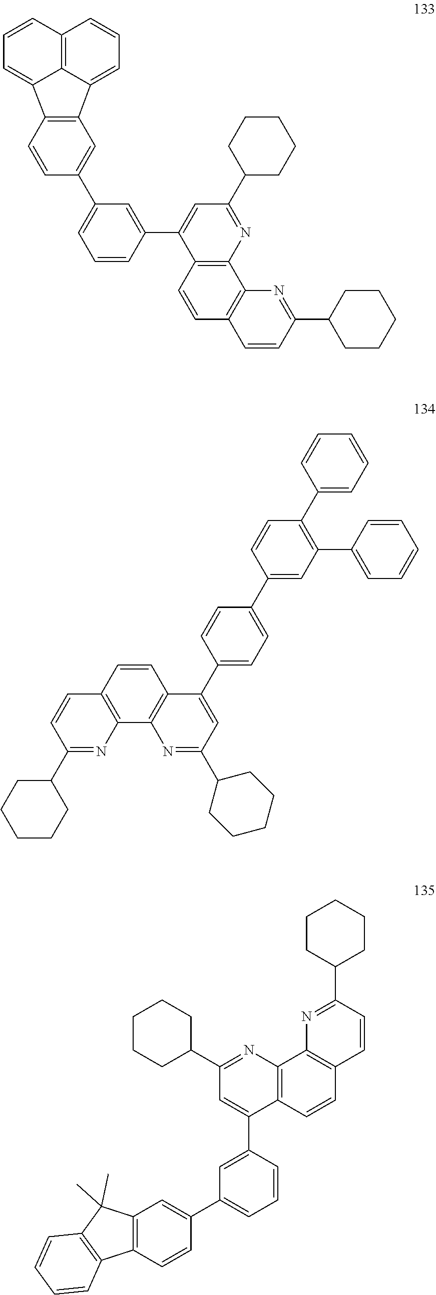 [SMILES: c1cc(-c2ccc3c(c2)-c2cccc4cccc-3c24)cc(-c2cc(C3CCCCC3)nc3c2ccc2ccc(C4CCCCC4)nc23)c1 ; c1ccc(-c2ccc(-c3ccc(-c4cc(C5CCCCC5)nc5c4ccc4ccc(C6CCCCC6)nc45)cc3)cc2-c2ccccc2)cc1 ; CC1(C)c2ccccc2-c2ccc(-c3cccc(-c4cc(C5CCCCC5)nc5c4ccc4ccc(C6CCCCC6)nc45)c3)cc21]

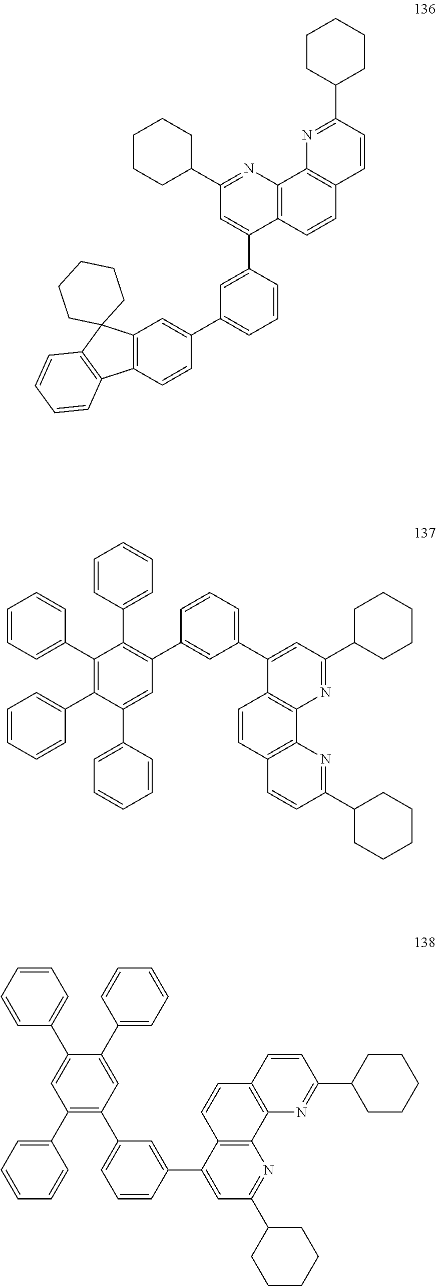 [SMILES: c1cc(-c2ccc3c(c2)C2(CCCCC2)c2ccccc2-3)cc(-c2cc(C3CCCCC3)nc3c2ccc2ccc(C4CCCCC4)nc23)c1 ; c1ccc(-c2cc(-c3cccc(-c4cc(C5CCCCC5)nc5c4ccc4ccc(C6CCCCC6)nc45)c3)c(-c3ccccc3)c(-c3ccccc3)c2-c2ccccc2)cc1 ; c1ccc(-c2cc(-c3ccccc3)c(-c3cccc(-c4cc(C5CCCCC5)nc5c4ccc4ccc(C6CCCCC6)nc45)c3)cc2-c2ccccc2)cc1]

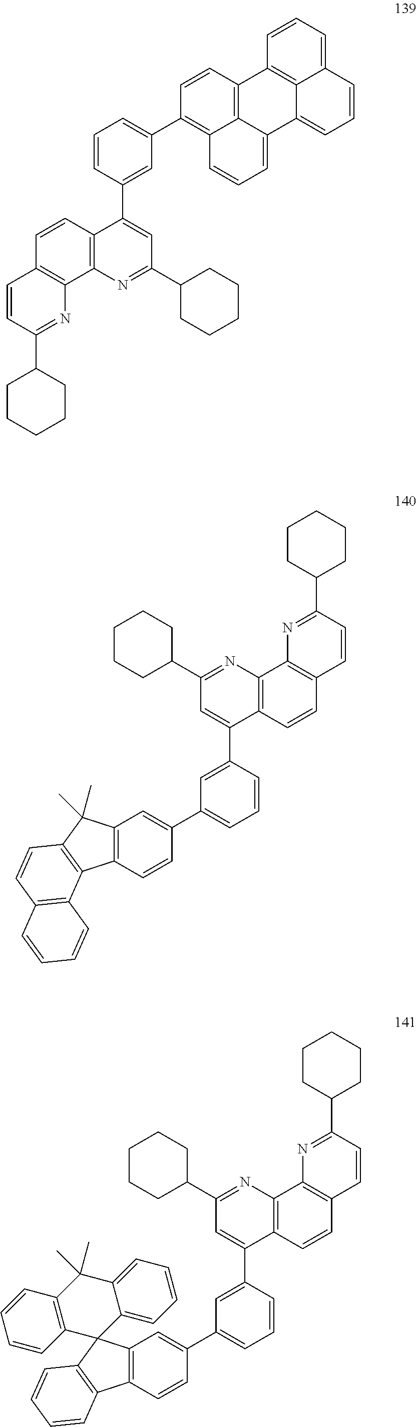 [SMILES: c1cc(-c2cc(C3CCCCC3)nc3c2ccc2ccc(C4CCCCC4)nc23)cc(-c2ccc3c4cccc5cccc(c6cccc2c63)c54)c1 ; CC1(C)c2cc(-c3cccc(-c4cc(C5CCCCC5)nc5c4ccc4ccc(C6CCCCC6)nc45)c3)ccc2-c2c1ccc1ccccc21 ; CC1(C)c2ccccc2C2(c3ccccc3-c3ccc(-c4cccc(-c5cc(C6CCCCC6)nc6c5ccc5ccc(C7CCCCC7)nc56)c4)cc32)c2ccccc21]

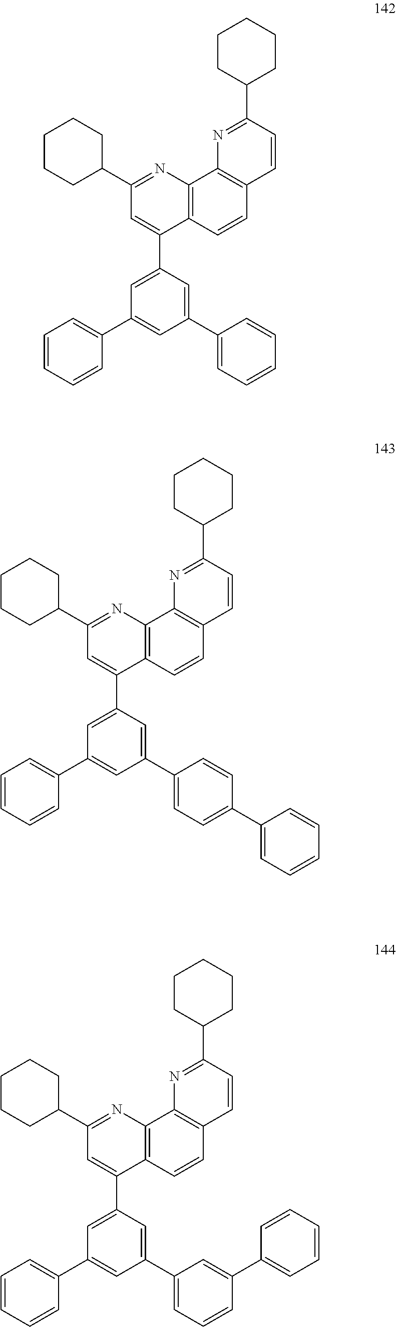 [SMILES: c1ccc(-c2cc(-c3ccccc3)cc(-c3cc(C4CCCCC4)nc4c3ccc3ccc(C5CCCCC5)nc34)c2)cc1 ; c1ccc(-c2ccc(-c3cc(-c4ccccc4)cc(-c4cc(C5CCCCC5)nc5c4ccc4ccc(C6CCCCC6)nc45)c3)cc2)cc1 ; c1ccc(-c2cccc(-c3cc(-c4ccccc4)cc(-c4cc(C5CCCCC5)nc5c4ccc4ccc(C6CCCCC6)nc45)c3)c2)cc1]

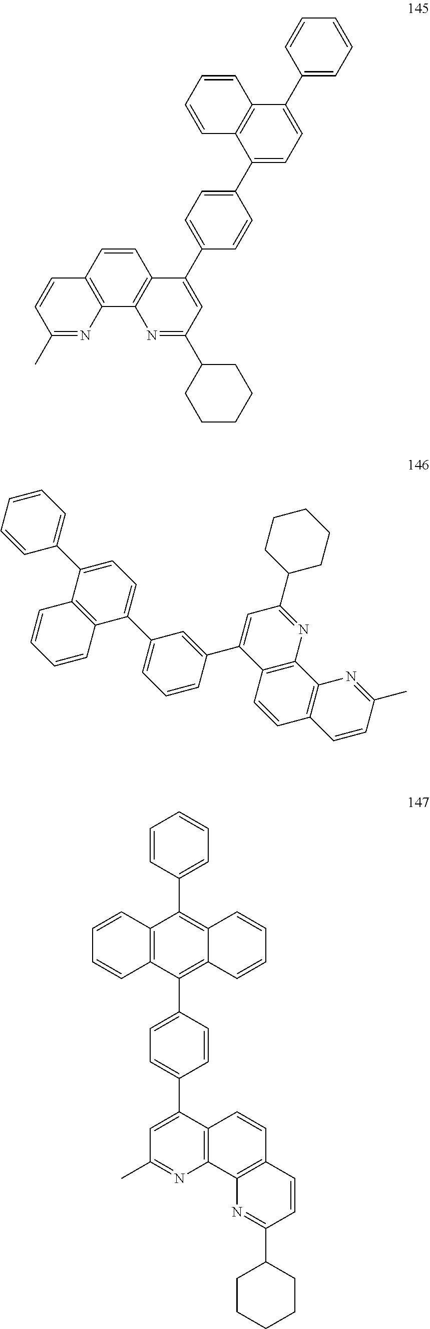 [SMILES: Cc1ccc2ccc3c(-c4ccc(-c5ccc(-c6ccccc6)c6ccccc56)cc4)cc(C4CCCCC4)nc3c2n1 ; Cc1ccc2ccc3c(-c4cccc(-c5ccc(-c6ccccc6)c6ccccc56)c4)cc(C4CCCCC4)nc3c2n1 ; Cc1cc(-c2ccc(-c3c4ccccc4c(-c4ccccc4)c4ccccc34)cc2)c2ccc3ccc(C4CCCCC4)nc3c2n1]

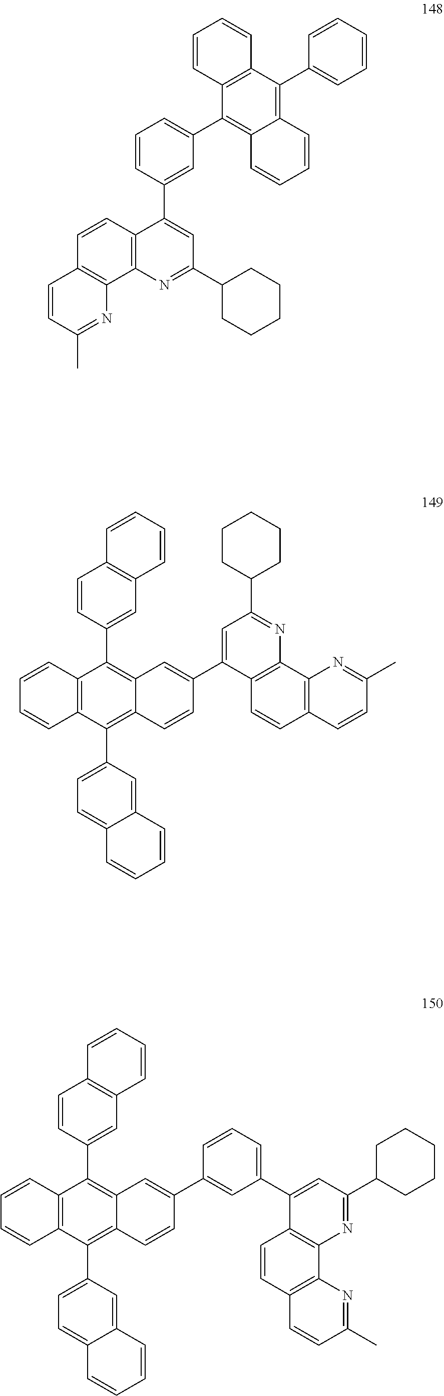 [SMILES: Cc1ccc2ccc3c(-c4cccc(-c5c6ccccc6c(-c6ccccc6)c6ccccc56)c4)cc(C4CCCCC4)nc3c2n1 ; Cc1ccc2ccc3c(-c4ccc5c(-c6ccc7ccccc7c6)c6ccccc6c(-c6ccc7ccccc7c6)c5c4)cc(C4CCCCC4)nc3c2n1 ; Cc1ccc2ccc3c(-c4cccc(-c5ccc6c(-c7ccc8ccccc8c7)c7ccccc7c(-c7ccc8ccccc8c7)c6c5)c4)cc(C4CCCCC4)nc3c2n1]

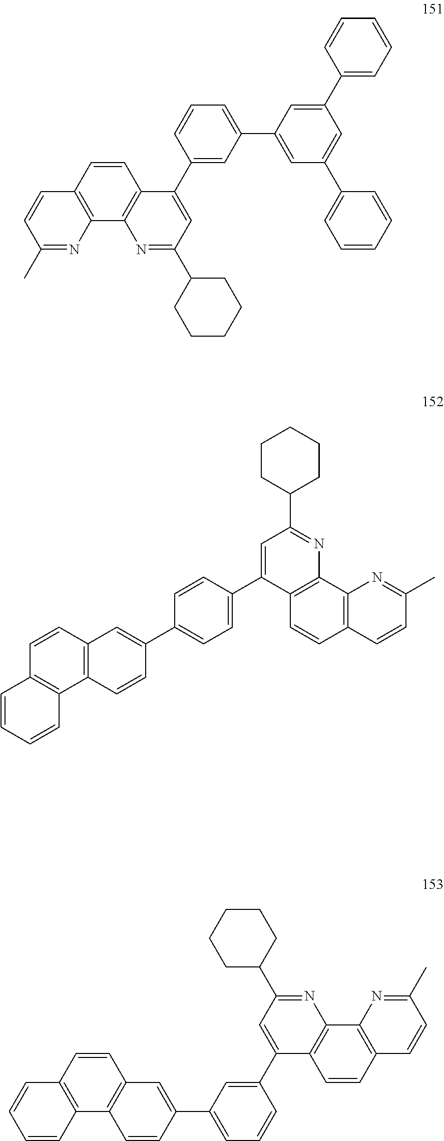 [SMILES: Cc1ccc2ccc3c(-c4cccc(-c5cc(-c6ccccc6)cc(-c6ccccc6)c5)c4)cc(C4CCCCC4)nc3c2n1 ; Cc1ccc2ccc3c(-c4ccc(-c5ccc6c(ccc7ccccc76)c5)cc4)cc(C4CCCCC4)nc3c2n1 ; Cc1ccc2ccc3c(-c4cccc(-c5ccc6c(ccc7ccccc76)c5)c4)cc(C4CCCCC4)nc3c2n1]

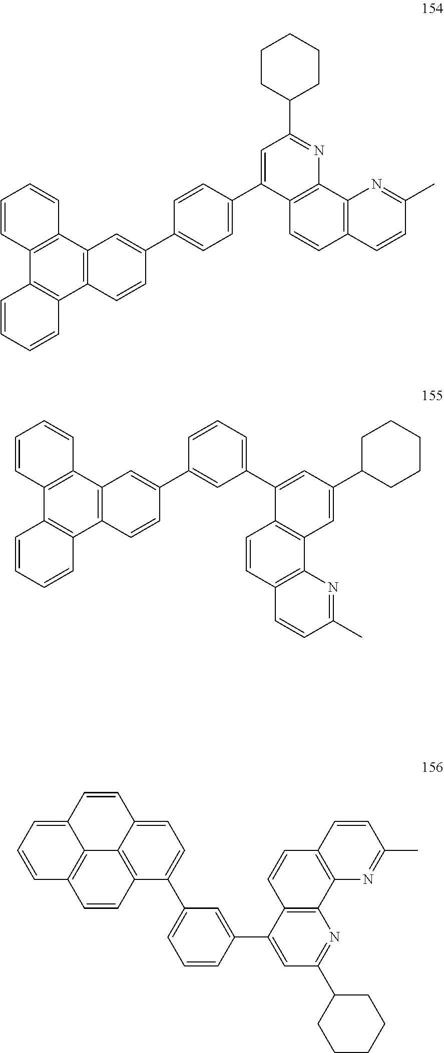 [SMILES: Cc1ccc2ccc3c(-c4ccc(-c5ccc6c7ccccc7c7ccccc7c6c5)cc4)cc(C4CCCCC4)nc3c2n1 ; Cc1ccc2ccc3c(-c4cccc(-c5ccc6c7ccccc7c7ccccc7c6c5)c4)cc(C4CCCCC4)cc3c2n1 ; Cc1ccc2ccc3c(-c4cccc(-c5ccc6ccc7cccc8ccc5c6c78)c4)cc(C4CCCCC4)nc3c2n1]

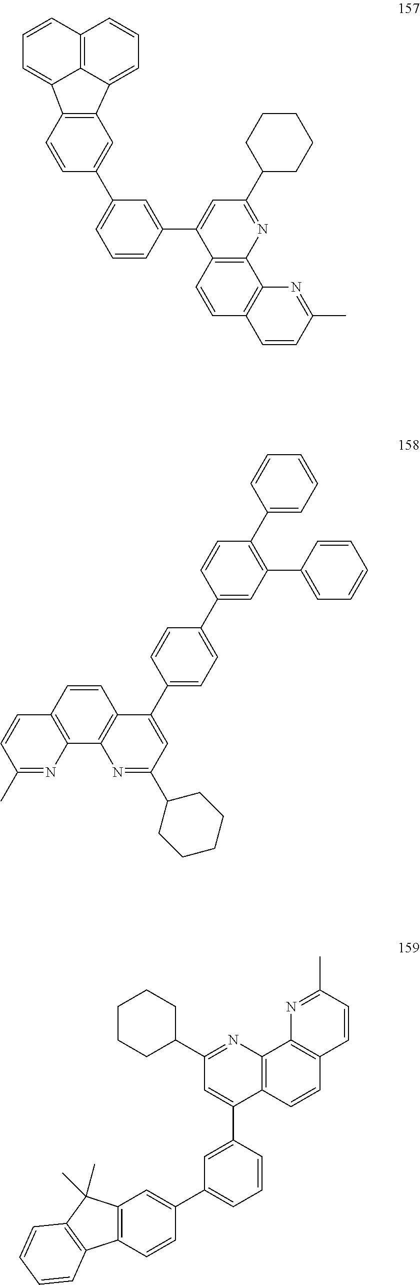 [SMILES: Cc1ccc2ccc3c(-c4cccc(-c5ccc6c(c5)-c5cccc7cccc-6c57)c4)cc(C4CCCCC4)nc3c2n1 ; Cc1ccc2ccc3c(-c4ccc(-c5ccc(-c6ccccc6)c(-c6ccccc6)c5)cc4)cc(C4CCCCC4)nc3c2n1 ; Cc1ccc2ccc3c(-c4cccc(-c5ccc6c(c5)C(C)(C)c5ccccc5-6)c4)cc(C4CCCCC4)nc3c2n1]

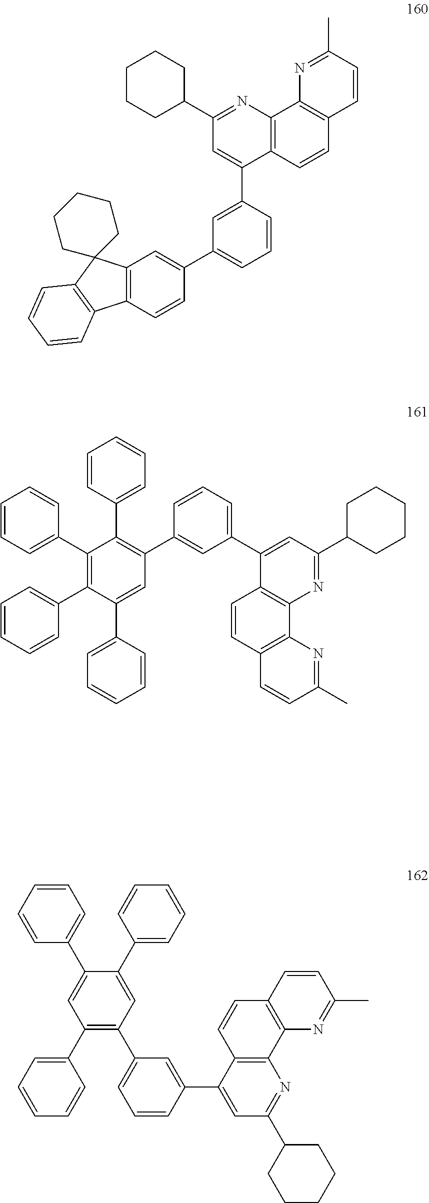 [SMILES: Cc1ccc2ccc3c(-c4cccc(-c5ccc6c(c5)C5(CCCCC5)c5ccccc5-6)c4)cc(C4CCCCC4)nc3c2n1 ; Cc1ccc2ccc3c(-c4cccc(-c5cc(-c6ccccc6)c(-c6ccccc6)c(-c6ccccc6)c5-c5ccccc5)c4)cc(C4CCCCC4)nc3c2n1 ; Cc1ccc2ccc3c(-c4cccc(-c5cc(-c6ccccc6)c(-c6ccccc6)cc5-c5ccccc5)c4)cc(C4CCCCC4)nc3c2n1]

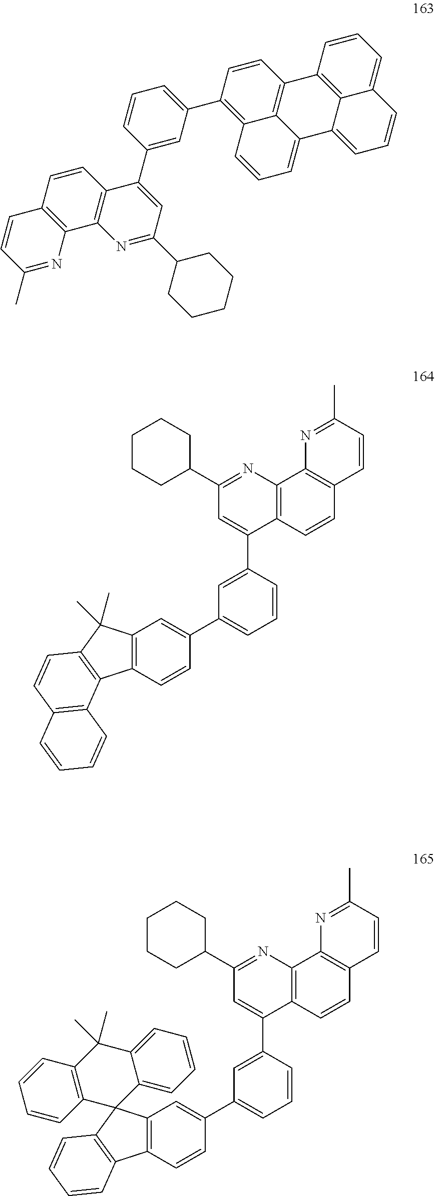 [SMILES: Cc1ccc2ccc3c(-c4cccc(-c5ccc6c7cccc8cccc(c9cccc5c96)c87)c4)cc(C4CCCCC4)nc3c2n1 ; Cc1ccc2ccc3c(-c4cccc(-c5ccc6c(c5)C(C)(C)c5ccc7ccccc7c5-6)c4)cc(C4CCCCC4)nc3c2n1 ; Cc1ccc2ccc3c(-c4cccc(-c5ccc6c(c5)C5(c7ccccc7-6)c6ccccc6C(C)(C)c6ccccc65)c4)cc(C4CCCCC4)nc3c2n1]

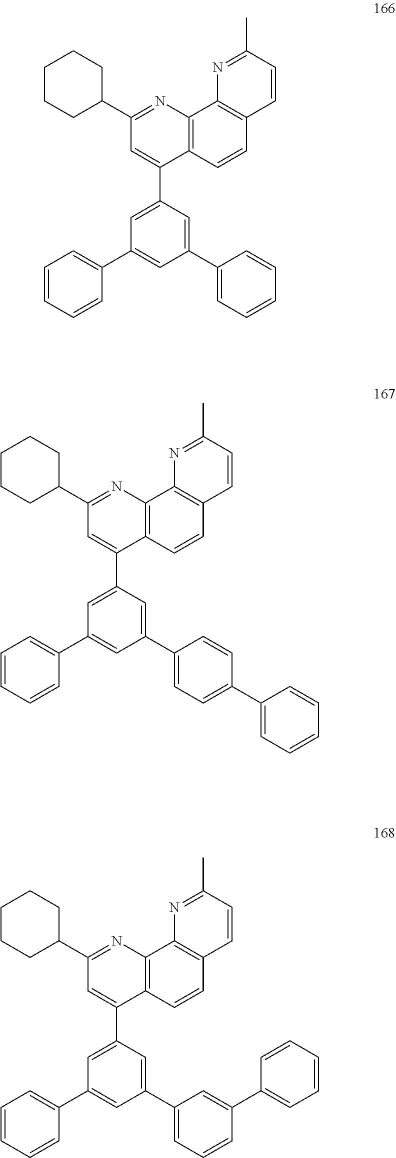 [SMILES: Cc1ccc2ccc3c(-c4cc(-c5ccccc5)cc(-c5ccccc5)c4)cc(C4CCCCC4)nc3c2n1 ; Cc1ccc2ccc3c(-c4cc(-c5ccccc5)cc(-c5ccc(-c6ccccc6)cc5)c4)cc(C4CCCCC4)nc3c2n1 ; Cc1ccc2ccc3c(-c4cc(-c5ccccc5)cc(-c5cccc(-c6ccccc6)c5)c4)cc(C4CCCCC4)nc3c2n1]

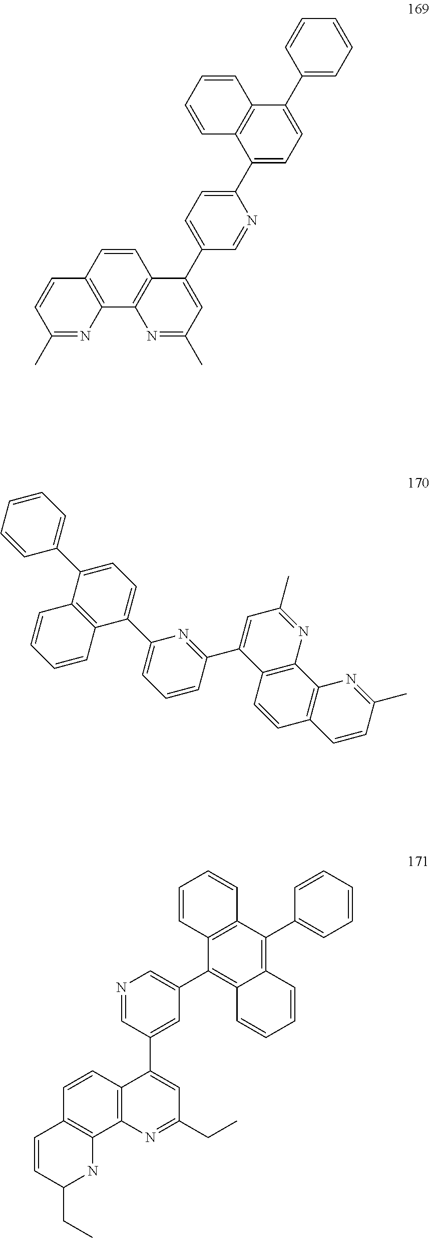

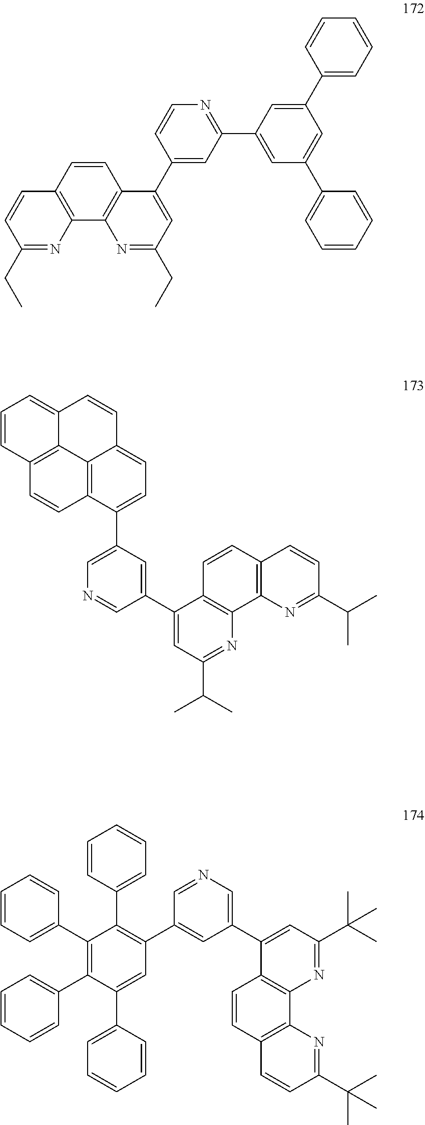 [SMILES: CCc1ccc2ccc3c(-c4ccnc(-c5cc(-c6ccccc6)cc(-c6ccccc6)c5)c4)cc(CC)nc3c2n1 ; CC(C)c1ccc2ccc3c(-c4cncc(-c5ccc6ccc7cccc8ccc5c6c78)c4)cc(C(C)C)nc3c2n1 ; CC(C)(C)c1ccc2ccc3c(-c4cncc(-c5cc(-c6ccccc6)c(-c6ccccc6)c(-c6ccccc6)c5-c5ccccc5)c4)cc(C(C)(C)C)nc3c2n1]

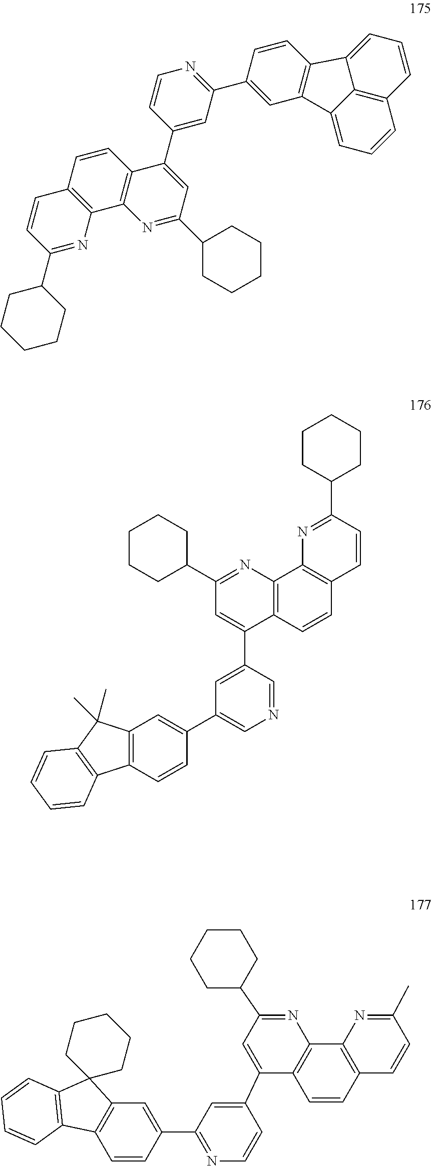 [SMILES: c1cc2c3c(cccc3c1)-c1cc(-c3cc(-c4cc(C5CCCCC5)nc5c4ccc4ccc(C6CCCCC6)nc45)ccn3)ccc1-2 ; CC1(C)c2ccccc2-c2ccc(-c3cncc(-c4cc(C5CCCCC5)nc5c4ccc4ccc(C6CCCCC6)nc45)c3)cc21 ; Cc1ccc2ccc3c(-c4ccnc(-c5ccc6c(c5)C5(CCCCC5)c5ccccc5-6)c4)cc(C4CCCCC4)nc3c2n1]

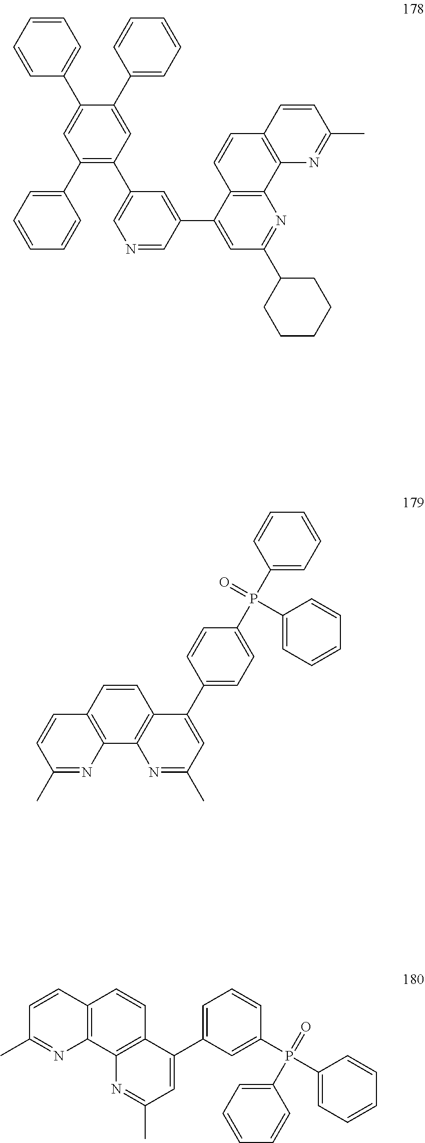

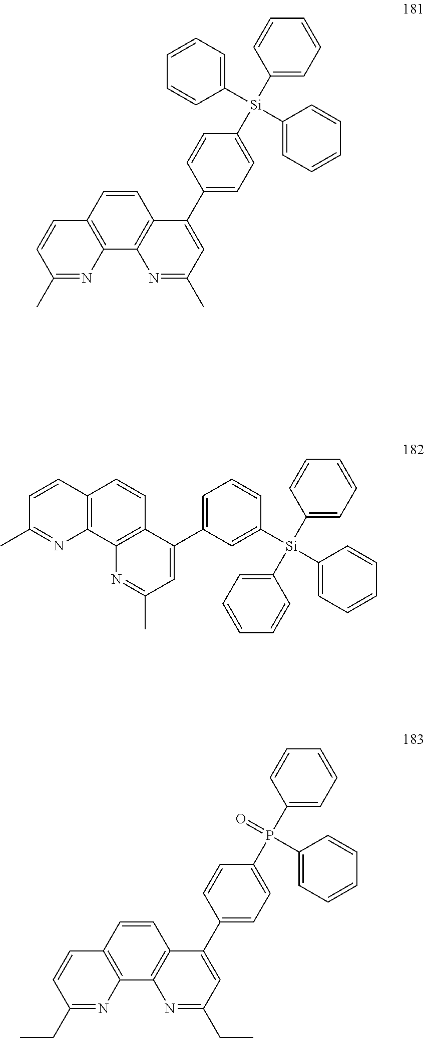 [SMILES: Cc1ccc2ccc3c(-c4ccc([Si](c5ccccc5)(c5ccccc5)c5ccccc5)cc4)cc(C)nc3c2n1 ; Cc1ccc2ccc3c(-c4cccc([Si](c5ccccc5)(c5ccccc5)c5ccccc5)c4)cc(C)nc3c2n1 ; CCc1ccc2ccc3c(-c4ccc(P(=O)(c5ccccc5)c5ccccc5)cc4)cc(CC)nc3c2n1]

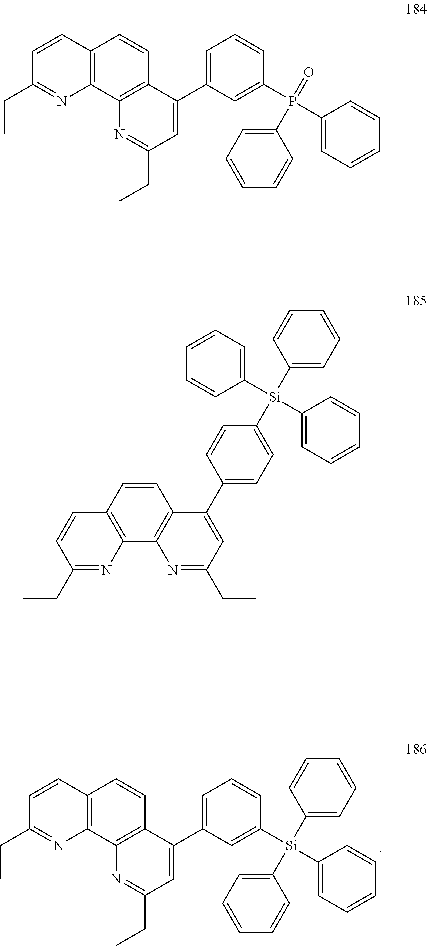 [SMILES: CCc1ccc2ccc3c(-c4cccc(P(=O)(c5ccccc5)c5ccccc5)c4)cc(CC)nc3c2n1 ; CCc1ccc2ccc3c(-c4ccc([Si](c5ccccc5)(c5ccccc5)c5ccccc5)cc4)cc(CC)nc3c2n1 ; CCc1ccc2ccc3c(-c4cccc([Si](c5ccccc5)(c5ccccc5)c5ccccc5)c4)cc(CC)nc3c2n1]

11. An organic electroluminescent device, comprising:
an anode; a cathode, and one or more organic layers disposed between the anode and the cathode,
wherein the at least one organic layer contains the organic compound of the following Chemical Formula 1 according to claim 1: 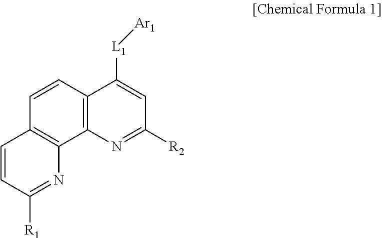
wherein,
R₁ and R₂, which are same or different, are each independently selected from the group consisting of a hydrogen atom, a deuterium atom (D), an alkyl group of C₁-C₆₀, a cycloalkyl group of C₃-C₆₀, and a heteroaryl group having 5 to 60 nuclear atoms, wherein a case where both of R₁ and R₂ are a hydrogen atom is excluded,
L₁ is selected from the group consisting of a single bond, an arylene group of C₆-C₆₀, and a heteroarylene group having 5 to 60 nuclear atoms, and
Ar₁ is selected from the group consisting of an aryl group of C₆-C₆₀, —P(═O)(R₃)(R₄), and —Si(R₅)(R₆)(R₇),
R₃ to R₇, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and
the alkyl group, the cycloalkyl group, and the heteroalkyl group of R₁ and R₂, the arylene group and the heteroarylene group of L₁, the arylene group of Ar₁, and the hydrazino group, the hydrazono group, the alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and the arylamine group of R₃ to R₇ are each independently substituted or unsubstituted with one or more substituents of: a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other.

12. The organic electroluminescent device of claim 11, wherein the organic layer containing the organic compound is an electron transport layer.

13. An organic electroluminescent device, comprising:
an anode and a cathode, spaced apart from each other;
a plurality of light-emitting units interposed between the anode and the cathode; and
an N- and a P-type charge generation layer disposed between adjacent light-emitting units,
wherein each of the light-emitting units comprises a hole transport layer, a light-emitting layer, and an electron transport layer and the N-type charge generation layer contains the compound of the following Chemical Formula 1 according to claim 1: 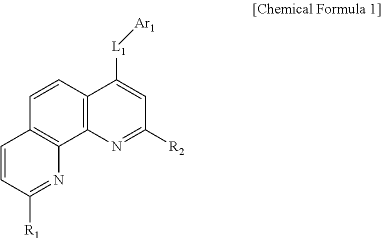
wherein,
R₁ and R₂, which are same or different, are each independently selected from the group consisting of a hydrogen atom, a deuterium atom (D), an alkyl group of C₁-C₆₀, a cycloalkyl group of C₃-C₆₀, and a heteroaryl group having 5 to 60 nuclear atoms, wherein a case where both of R₁ and R₂ are a hydrogen atom is excluded,
L₁ is selected from the group consisting of a single bond, an arylene group of C₆-C₆₀, and a heteroarylene group having 5 to 60 nuclear atoms, and
Ar₁ is selected from the group consisting of an aryl group of C₆-C₆₀, —P(═O)(R₃)(R₄), and —Si(R₅)(R₆)(R₇),
R₃ to R₇, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and
the alkyl group, the cycloalkyl group, and the heteroalkyl group of R₁ and R₂, the arylene group and the heteroarylene group of L₁, the arylene group of Ar₁, and the hydrazino group, the hydrazono group, the alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and the arylamine group of R₃ to R₇ are each independently substituted or unsubstituted with one or more substituents of: a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other.

14. The organic electroluminescent device of claim 13, wherein the N-type charge generation layer contains one host having an electron transport property, and the host is the compound of the Chemical Formula 1.

15. The organic electroluminescent device of claim 14, wherein the N-type charge generation layer further contains an N-type dopant.

16. The organic electroluminescent device of claim 11, wherein at least one of R₁ and R₂ is an alkyl group of C₁-C₆₀ or a cycloalkyl group of C₃-C₆₀.

17. The organic electroluminescent device of claim 11, wherein the compound of Chemical Formula 1 is a compound of any one of the following Chemical Formulas 2 to 6: 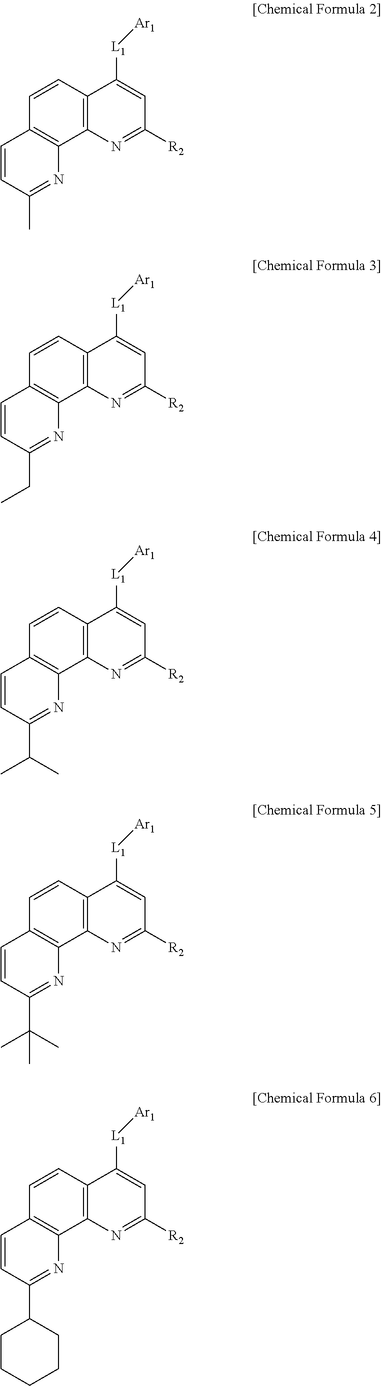
wherein,
L₁ and Ar₁ are as defined in claim 11, and
R₂ is an alkyl group of C₁-C₆ or a cycloalkyl group of C₃-C₆.

18. The organic electroluminescent device of claim 11, wherein the compound of Chemical Formula 1 is a compound of any one of the following Chemical Formulas 7 to 12: 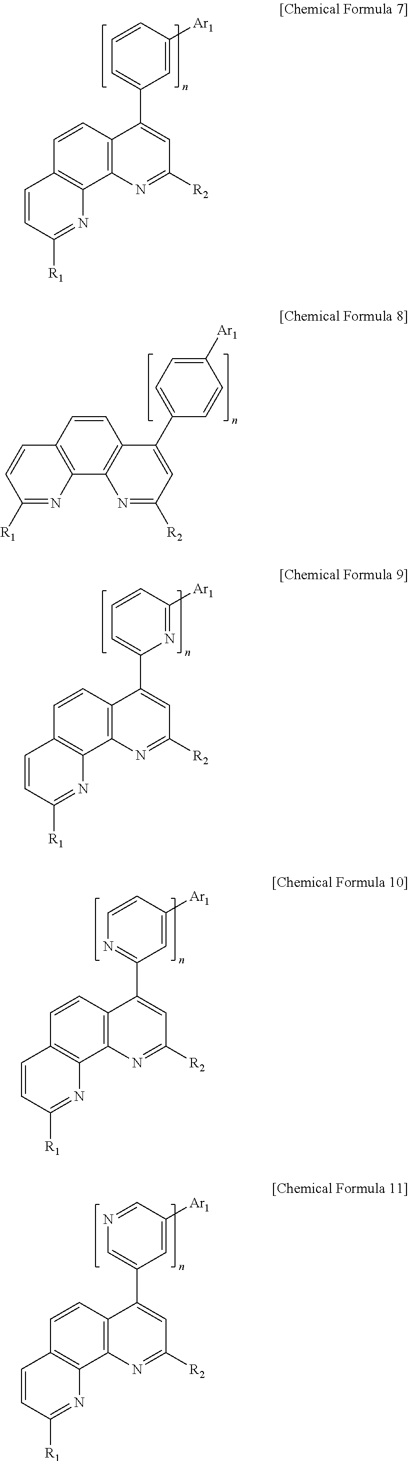 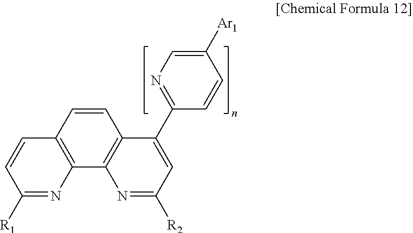
wherein,
R₁ and R₂, which are same or different, are each independently an alkyl group of C₁-C₆ or a cycloalkyl group of C₃-C₆,
n is 0 or 1, and
Ar₁ is selected from the group consisting of the following substituents S1 to S8: 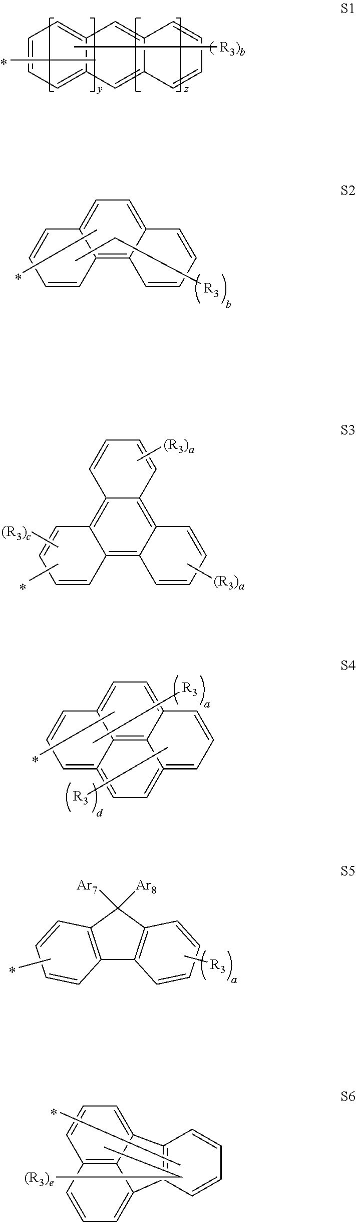 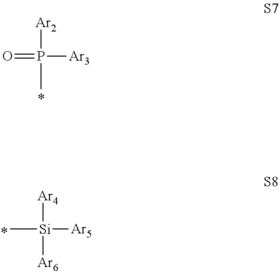
wherein,
a is an integer of 0 to 4,
b is an integer of 0 to 9,
c is an integer of 0 to 3,
d is an integer of 0 to 5,
e is an integer of 0 to 9,
the plurality of R₃ are the same as or different from each other,
R₃ or two or more R₃'s, if present, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀,
y and z are each 0 or 1,
Ar₂ to Ar₈, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, or forming a fused ring with an adjacent group, and
the hydrazino group, the hydrazono group, alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and the arylamine group of Ar₂ to Ar₈ are each independently substituted or unsubstituted with one or more substituents of: a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other.

19. The organic electroluminescent device of claim 11, wherein the compound of Chemical Formula 1 is selected from the group consisting of the following Compounds 1 to 186:

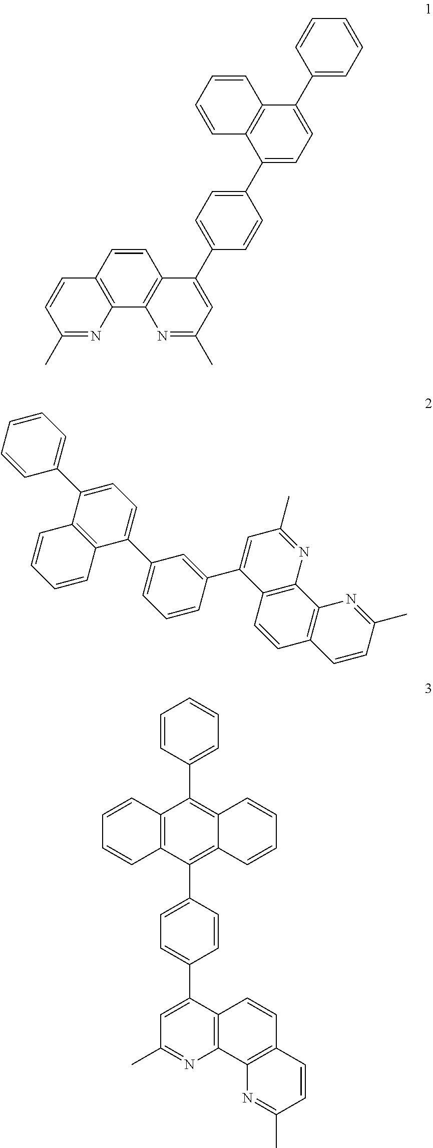 [SMILES: Cc1ccc2ccc3c(-c4ccc(-c5ccc(-c6ccccc6)c6ccccc56)cc4)cc(C)nc3c2n1 ; Cc1ccc2ccc3c(-c4cccc(-c5ccc(-c6ccccc6)c6ccccc56)c4)cc(C)nc3c2n1 ; Cc1ccc2ccc3c(-c4ccc(-c5c6ccccc6c(-c6ccccc6)c6ccccc56)cc4)cc(C)nc3c2n1]

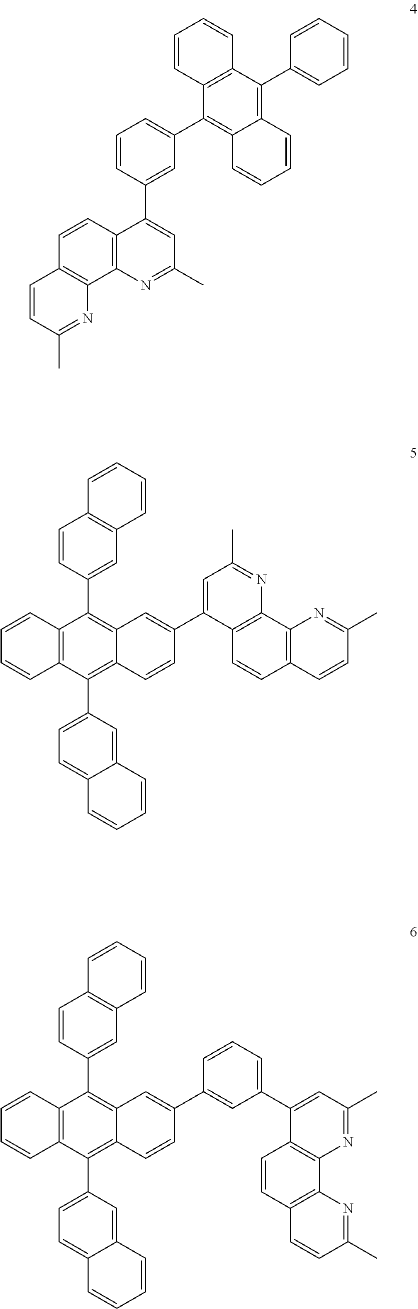 [SMILES: Cc1ccc2ccc3c(-c4cccc(-c5c6ccccc6c(-c6ccccc6)c6ccccc56)c4)cc(C)nc3c2n1 ; Cc1ccc2ccc3c(-c4ccc5c(-c6ccc7ccccc7c6)c6ccccc6c(-c6ccc7ccccc7c6)c5c4)cc(C)nc3c2n1 ; Cc1ccc2ccc3c(-c4cccc(-c5ccc6c(-c7ccc8ccccc8c7)c7ccccc7c(-c7ccc8ccccc8c7)c6c5)c4)cc(C)nc3c2n1]

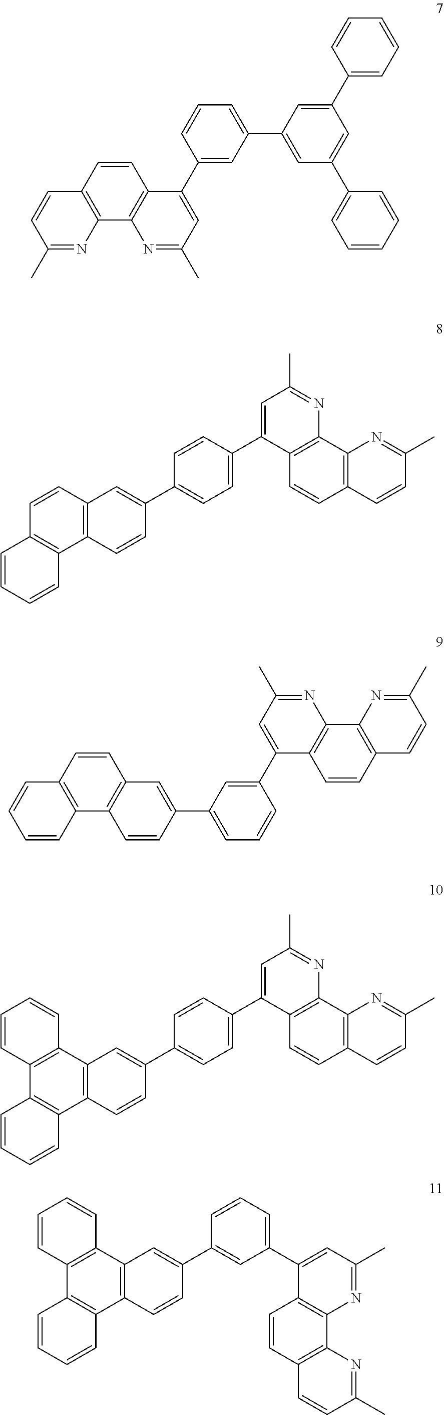 [SMILES: Cc1ccc2ccc3c(-c4cccc(-c5cc(-c6ccccc6)cc(-c6ccccc6)c5)c4)cc(C)nc3c2n1 ; Cc1ccc2ccc3c(-c4ccc(-c5ccc6c(ccc7ccccc76)c5)cc4)cc(C)nc3c2n1 ; Cc1ccc2ccc3c(-c4cccc(-c5ccc6c(ccc7ccccc76)c5)c4)cc(C)nc3c2n1 ; Cc1ccc2ccc3c(-c4ccc(-c5ccc6c7ccccc7c7ccccc7c6c5)cc4)cc(C)nc3c2n1 ; Cc1ccc2ccc3c(-c4cccc(-c5ccc6c7ccccc7c7ccccc7c6c5)c4)cc(C)nc3c2n1]

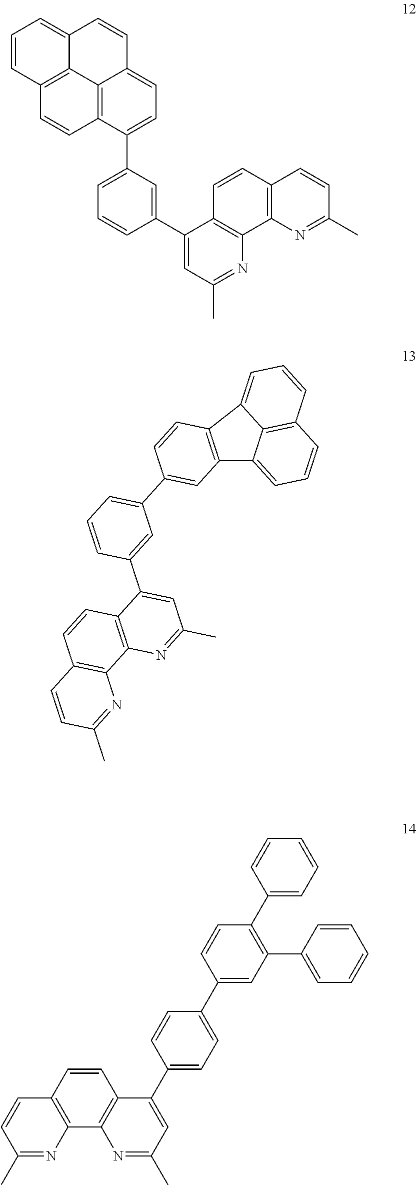 [SMILES: Cc1ccc2ccc3c(-c4cccc(-c5ccc6ccc7cccc8ccc5c6c78)c4)cc(C)nc3c2n1 ; Cc1ccc2ccc3c(-c4cccc(-c5ccc6c(c5)-c5cccc7cccc-6c57)c4)cc(C)nc3c2n1 ; Cc1ccc2ccc3c(-c4ccc(-c5ccc(-c6ccccc6)c(-c6ccccc6)c5)cc4)cc(C)nc3c2n1]

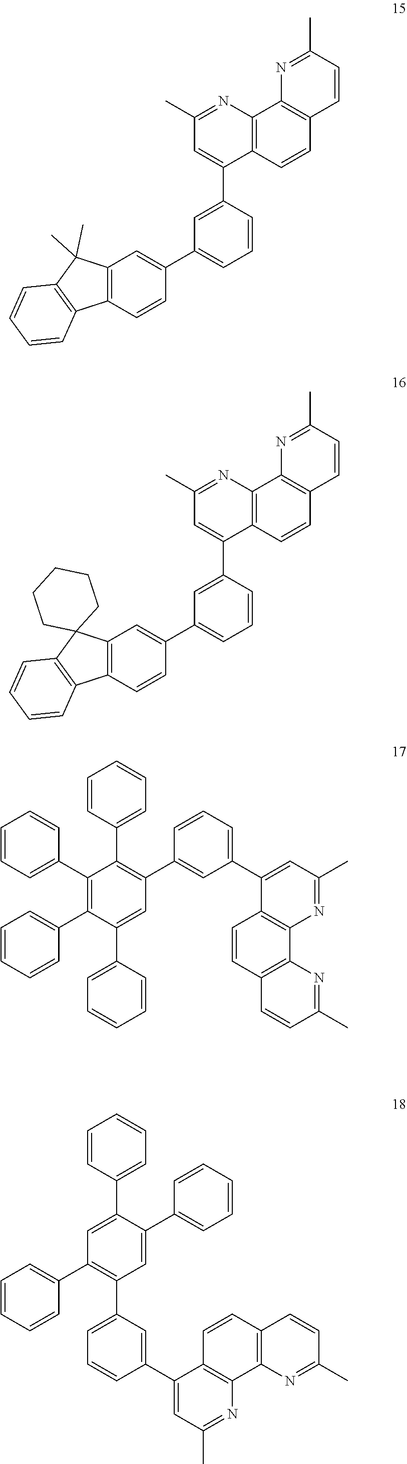 [SMILES: Cc1ccc2ccc3c(-c4cccc(-c5ccc6c(c5)C(C)(C)c5ccccc5-6)c4)cc(C)nc3c2n1 ; Cc1ccc2ccc3c(-c4cccc(-c5ccc6c(c5)C5(CCCCC5)c5ccccc5-6)c4)cc(C)nc3c2n1 ; Cc1ccc2ccc3c(-c4cccc(-c5cc(-c6ccccc6)c(-c6ccccc6)c(-c6ccccc6)c5-c5ccccc5)c4)cc(C)nc3c2n1 ; Cc1ccc2ccc3c(-c4cccc(-c5cc(-c6ccccc6)c(-c6ccccc6)cc5-c5ccccc5)c4)cc(C)nc3c2n1]

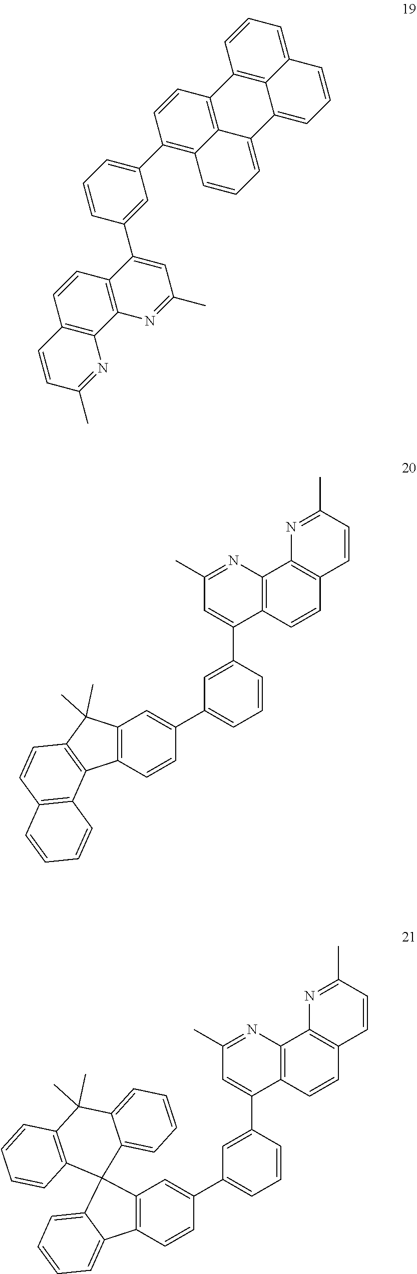 [SMILES: Cc1ccc2ccc3c(-c4cccc(-c5ccc6c7cccc8cccc(c9cccc5c96)c87)c4)cc(C)nc3c2n1 ; Cc1ccc2ccc3c(-c4cccc(-c5ccc6c(c5)C(C)(C)c5ccc7ccccc7c5-6)c4)cc(C)nc3c2n1 ; Cc1ccc2ccc3c(-c4cccc(-c5ccc6c(c5)C5(c7ccccc7-6)c6ccccc6C(C)(C)c6ccccc65)c4)cc(C)nc3c2n1]

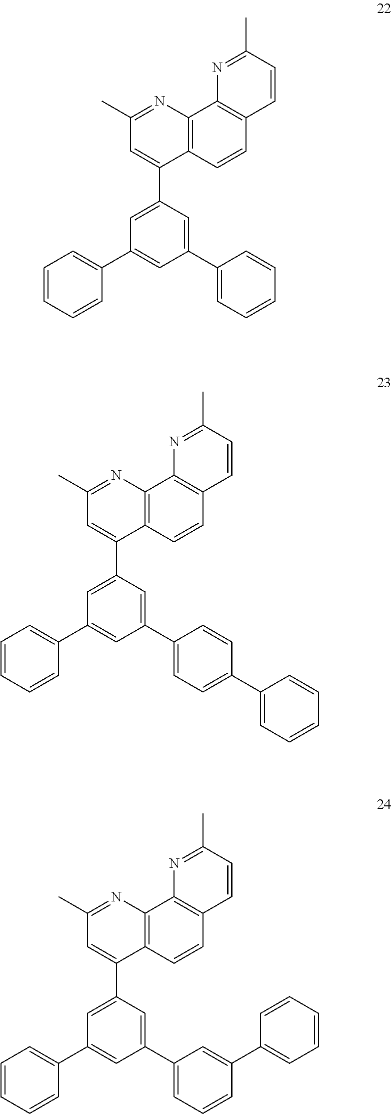 [SMILES: Cc1ccc2ccc3c(-c4cc(-c5ccccc5)cc(-c5ccccc5)c4)cc(C)nc3c2n1 ; Cc1ccc2ccc3c(-c4cc(-c5ccccc5)cc(-c5ccc(-c6ccccc6)cc5)c4)cc(C)nc3c2n1 ; Cc1ccc2ccc3c(-c4cc(-c5ccccc5)cc(-c5cccc(-c6ccccc6)c5)c4)cc(C)nc3c2n1]

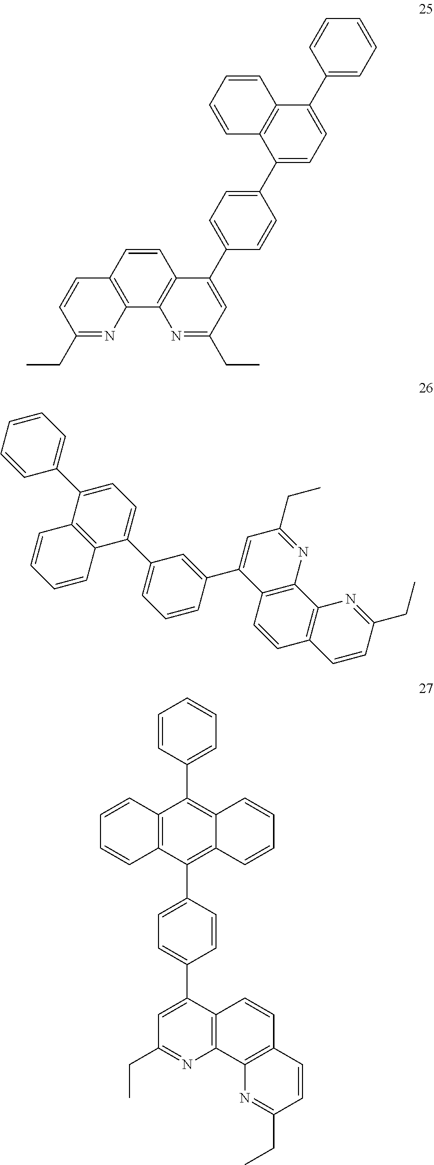 [SMILES: CCc1ccc2ccc3c(-c4ccc(-c5ccc(-c6ccccc6)c6ccccc56)cc4)cc(CC)nc3c2n1 ; CCc1ccc2ccc3c(-c4cccc(-c5ccc(-c6ccccc6)c6ccccc56)c4)cc(CC)nc3c2n1 ; CCc1ccc2ccc3c(-c4ccc(-c5c6ccccc6c(-c6ccccc6)c6ccccc56)cc4)cc(CC)nc3c2n1]

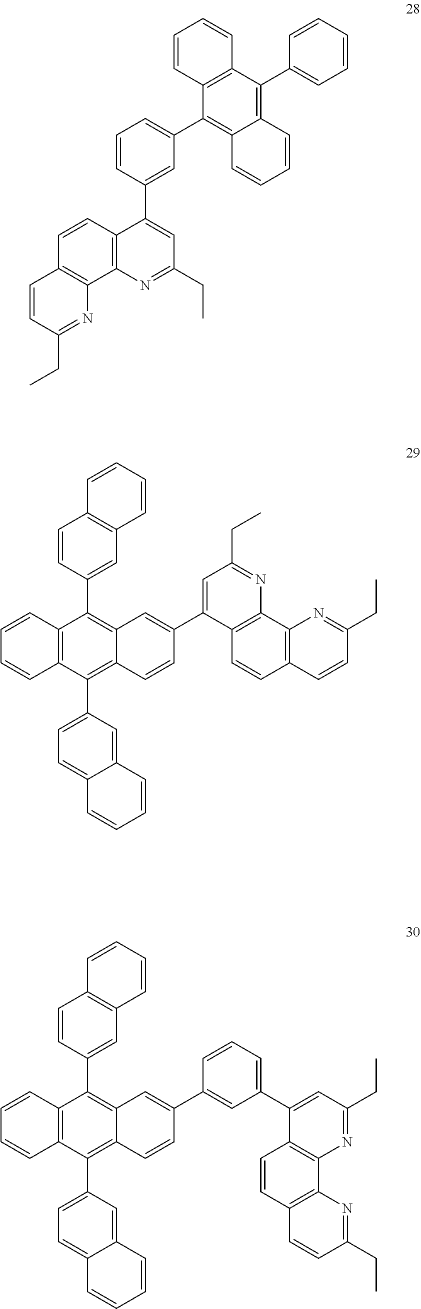 [SMILES: CCc1ccc2ccc3c(-c4cccc(-c5c6ccccc6c(-c6ccccc6)c6ccccc56)c4)cc(CC)nc3c2n1 ; CCc1ccc2ccc3c(-c4ccc5c(-c6ccc7ccccc7c6)c6ccccc6c(-c6ccc7ccccc7c6)c5c4)cc(CC)nc3c2n1 ; CCc1ccc2ccc3c(-c4cccc(-c5ccc6c(-c7ccc8ccccc8c7)c7ccccc7c(-c7ccc8ccccc8c7)c6c5)c4)cc(CC)nc3c2n1]

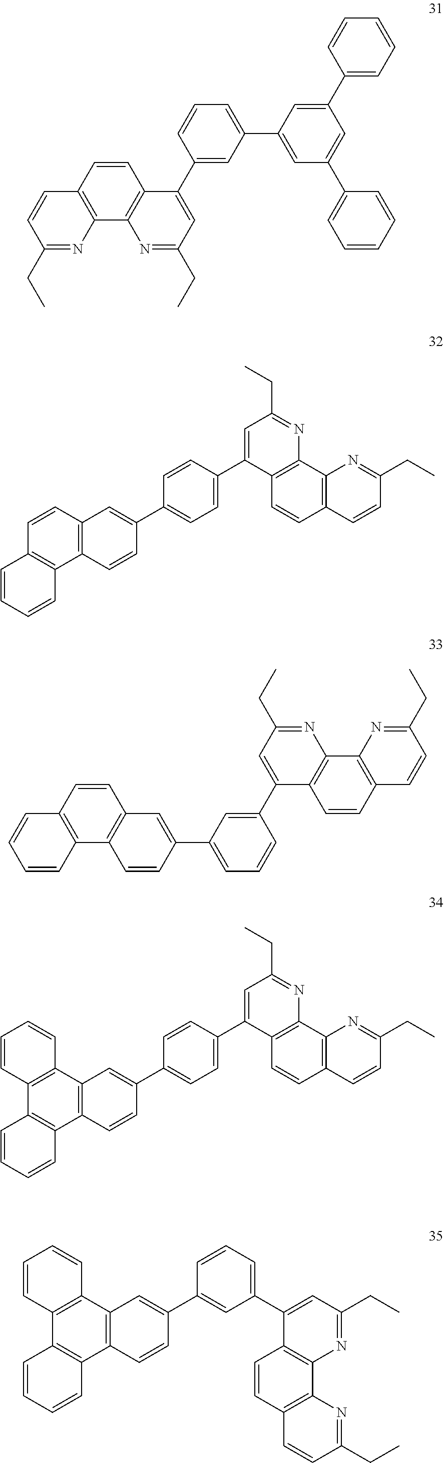 [SMILES: CCc1ccc2ccc3c(-c4cccc(-c5cc(-c6ccccc6)cc(-c6ccccc6)c5)c4)cc(CC)nc3c2n1 ; CCc1ccc2ccc3c(-c4ccc(-c5ccc6c(ccc7ccccc76)c5)cc4)cc(CC)nc3c2n1 ; CCc1ccc2ccc3c(-c4cccc(-c5ccc6c(ccc7ccccc76)c5)c4)cc(CC)nc3c2n1 ; CCc1ccc2ccc3c(-c4ccc(-c5ccc6c7ccccc7c7ccccc7c6c5)cc4)cc(CC)nc3c2n1 ; CCc1ccc2ccc3c(-c4cccc(-c5ccc6c7ccccc7c7ccccc7c6c5)c4)cc(CC)nc3c2n1]

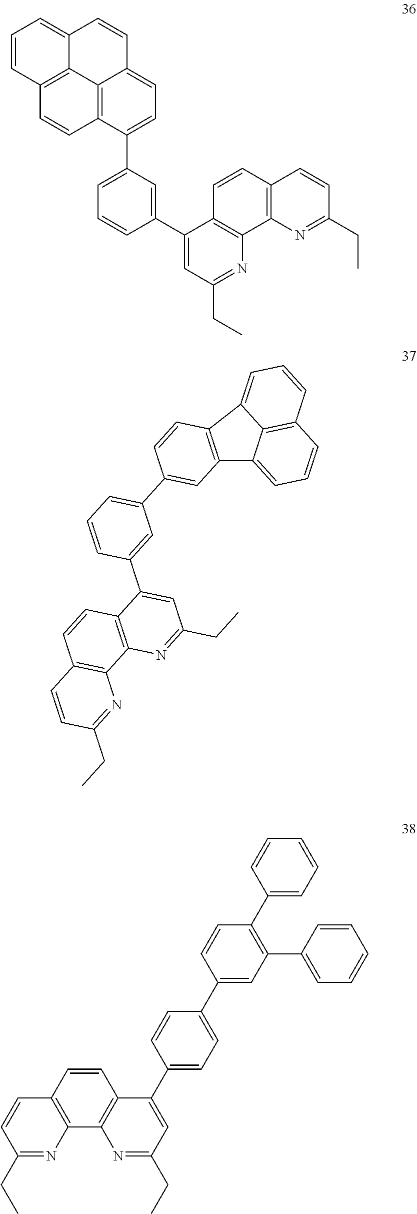

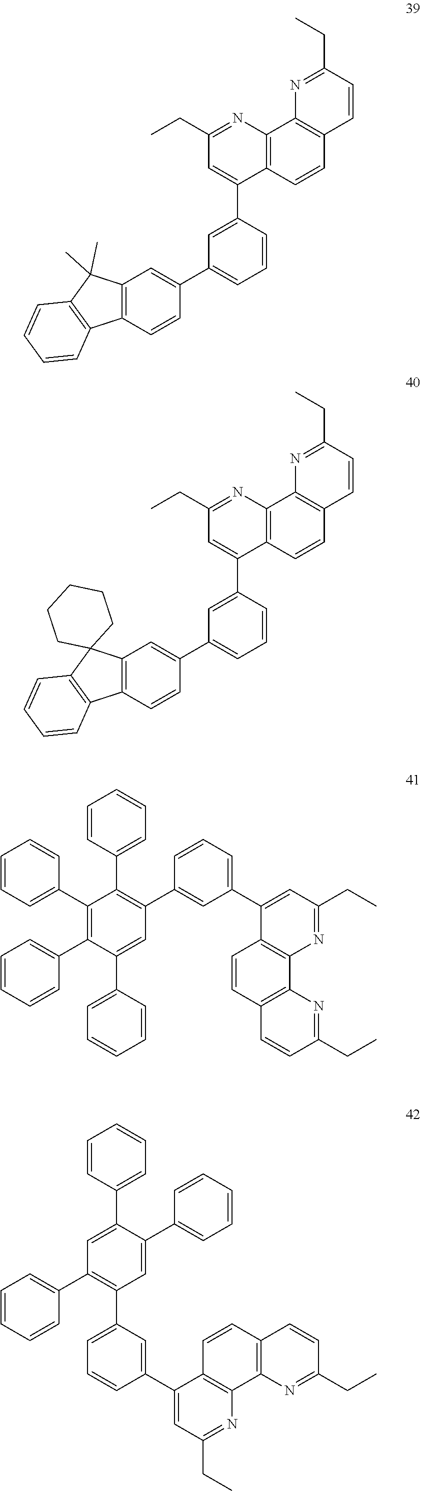 [SMILES: CCc1ccc2ccc3c(-c4cccc(-c5ccc6c(c5)C(C)(C)c5ccccc5-6)c4)cc(CC)nc3c2n1 ; CCc1ccc2ccc3c(-c4cccc(-c5ccc6c(c5)C5(CCCCC5)c5ccccc5-6)c4)cc(CC)nc3c2n1 ; CCc1ccc2ccc3c(-c4cccc(-c5cc(-c6ccccc6)c(-c6ccccc6)c(-c6ccccc6)c5-c5ccccc5)c4)cc(CC)nc3c2n1 ; CCc1ccc2ccc3c(-c4cccc(-c5cc(-c6ccccc6)c(-c6ccccc6)cc5-c5ccccc5)c4)cc(CC)nc3c2n1]

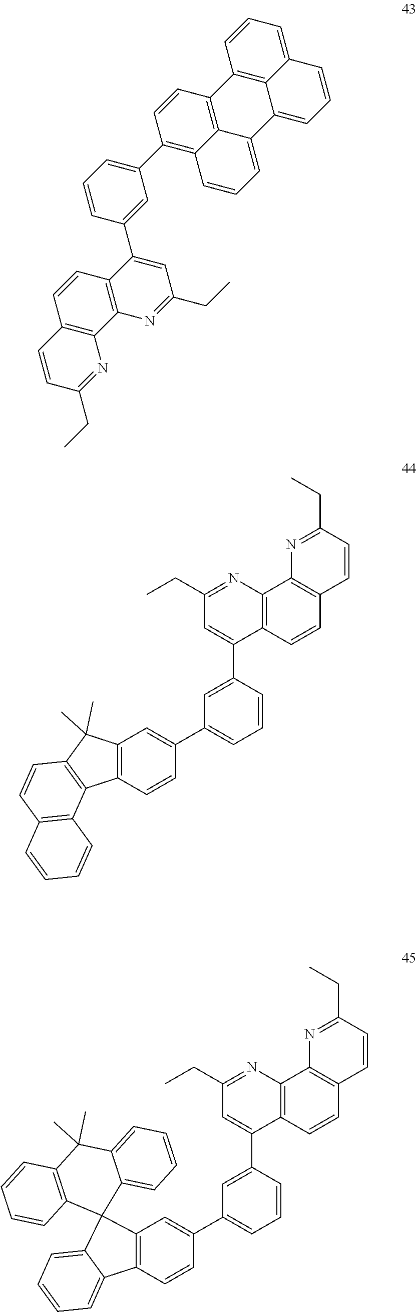 [SMILES: CCc1ccc2ccc3c(-c4cccc(-c5ccc6c7cccc8cccc(c9cccc5c96)c87)c4)cc(CC)nc3c2n1 ; CCc1ccc2ccc3c(-c4cccc(-c5ccc6c(c5)C(C)(C)c5ccc7ccccc7c5-6)c4)cc(CC)nc3c2n1 ; CCc1ccc2ccc3c(-c4cccc(-c5ccc6c(c5)C5(c7ccccc7-6)c6ccccc6C(C)(C)c6ccccc65)c4)cc(CC)nc3c2n1]

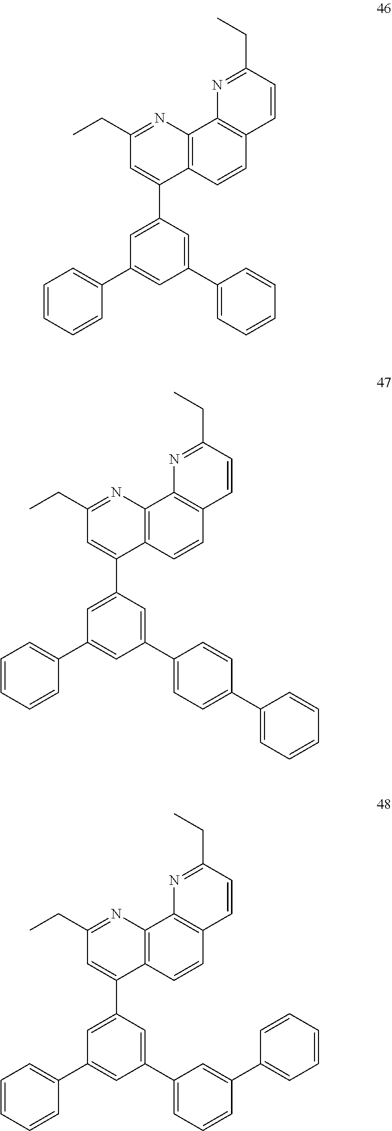 [SMILES: CCc1ccc2ccc3c(-c4cc(-c5ccccc5)cc(-c5ccccc5)c4)cc(CC)nc3c2n1 ; CCc1ccc2ccc3c(-c4cc(-c5ccccc5)cc(-c5ccc(-c6ccccc6)cc5)c4)cc(CC)nc3c2n1 ; CCc1ccc2ccc3c(-c4cc(-c5ccccc5)cc(-c5cccc(-c6ccccc6)c5)c4)cc(CC)nc3c2n1]

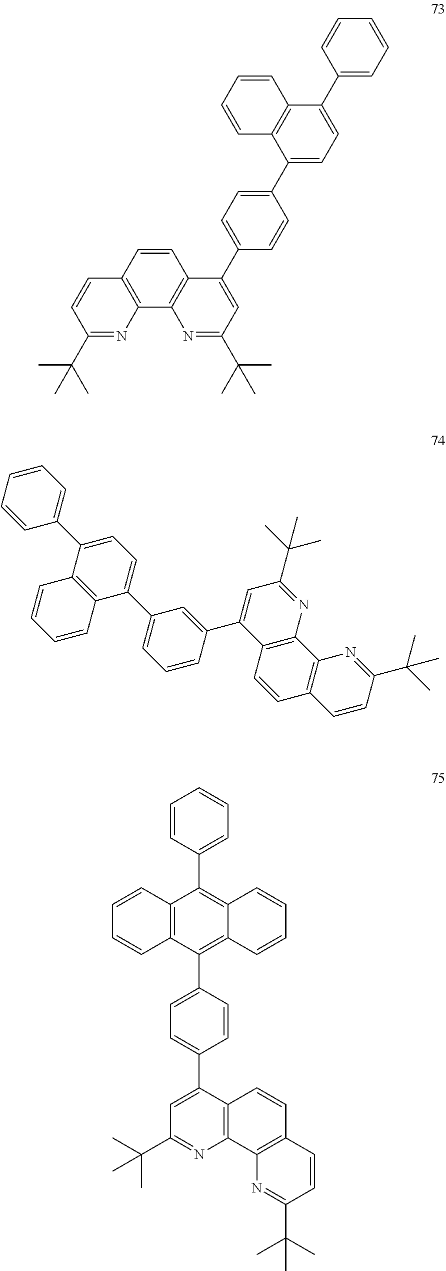 [SMILES: CC(C)(C)c1ccc2ccc3c(-c4ccc(-c5ccc(-c6ccccc6)c6ccccc56)cc4)cc(C(C)(C)C)nc3c2n1 ; CC(C)(C)c1ccc2ccc3c(-c4cccc(-c5ccc(-c6ccccc6)c6ccccc56)c4)cc(C(C)(C)C)nc3c2n1 ; CC(C)(C)c1ccc2ccc3c(-c4ccc(-c5c6ccccc6c(-c6ccccc6)c6ccccc56)cc4)cc(C(C)(C)C)nc3c2n1]

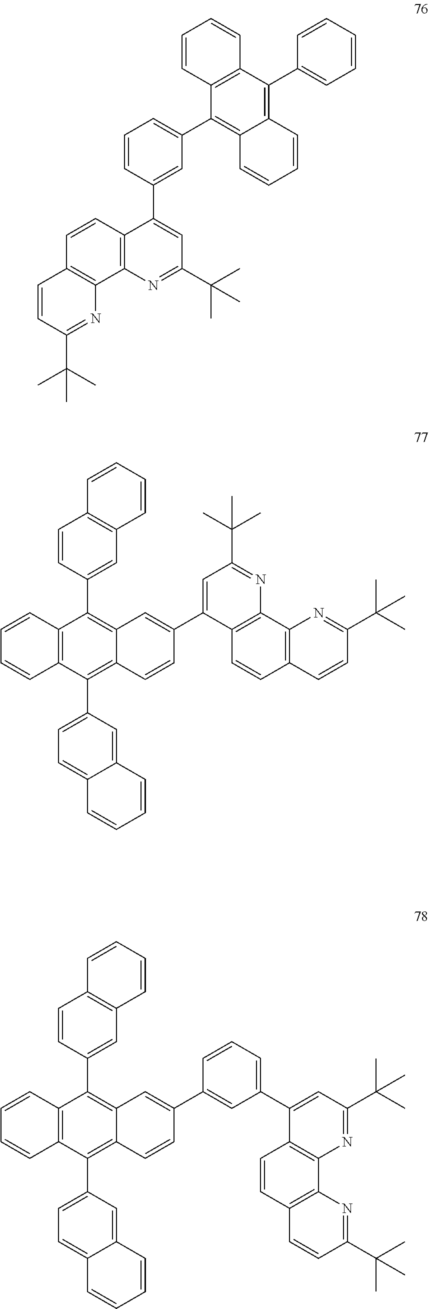 [SMILES: CC(C)(C)c1ccc2ccc3c(-c4cccc(-c5c6ccccc6c(-c6ccccc6)c6ccccc56)c4)cc(C(C)(C)C)nc3c2n1 ; CC(C)(C)c1ccc2ccc3c(-c4ccc5c(-c6ccc7ccccc7c6)c6ccccc6c(-c6ccc7ccccc7c6)c5c4)cc(C(C)(C)C)nc3c2n1 ; CC(C)(C)c1ccc2ccc3c(-c4cccc(-c5ccc6c(-c7ccc8ccccc8c7)c7ccccc7c(-c7ccc8ccccc8c7)c6c5)c4)cc(C(C)(C)C)nc3c2n1]

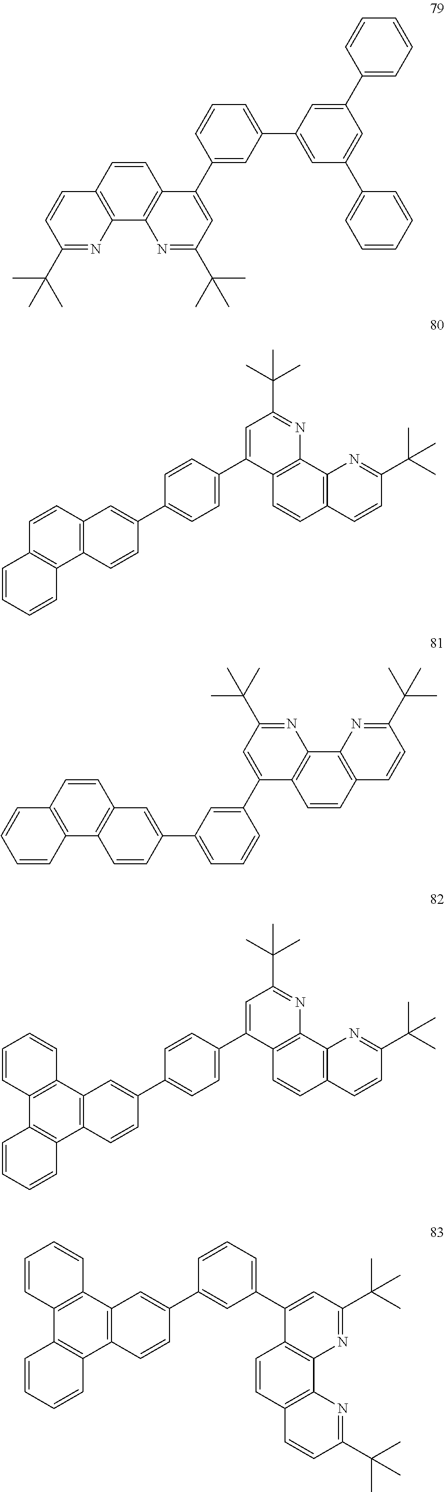

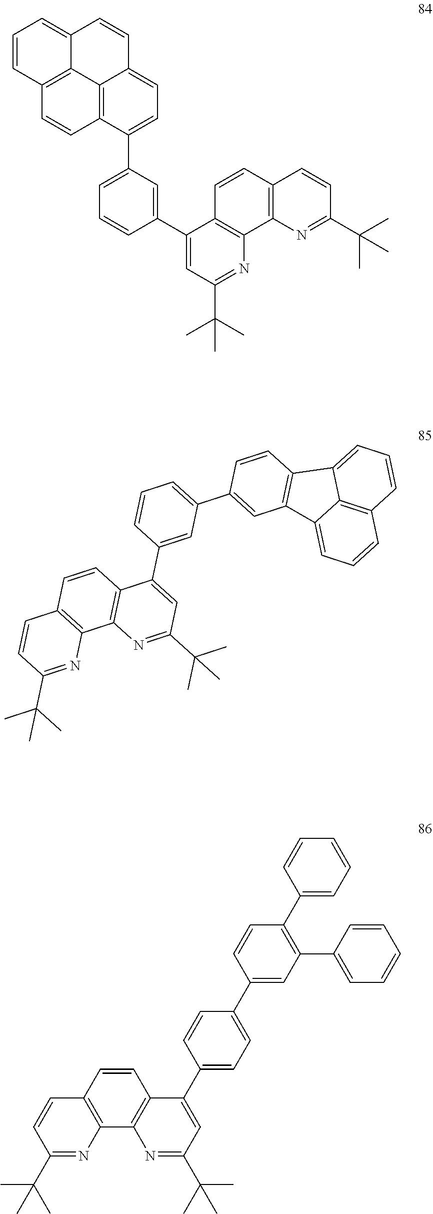

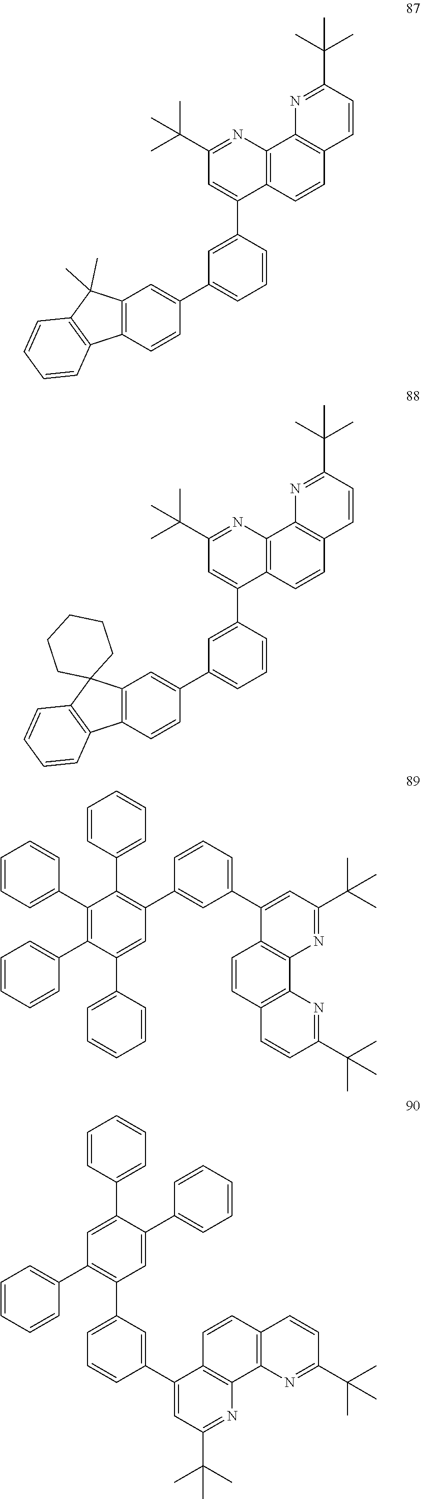

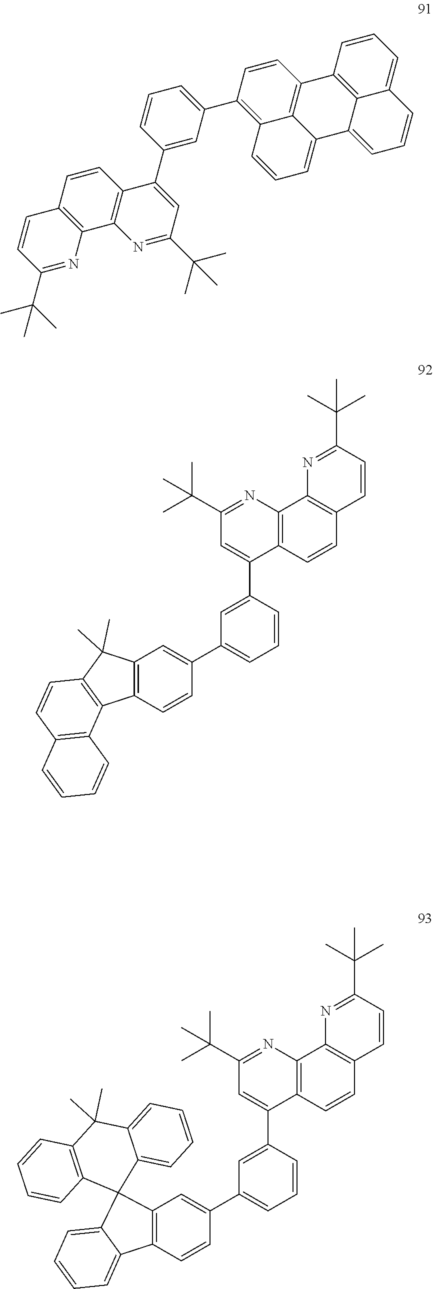 [SMILES: CC(C)(C)c1ccc2ccc3c(-c4cccc(-c5ccc6c7cccc8cccc(c9cccc5c96)c87)c4)cc(C(C)(C)C)nc3c2n1 ; CC(C)(C)c1ccc2ccc3c(-c4cccc(-c5ccc6c(c5)C(C)(C)c5ccc7ccccc7c5-6)c4)cc(C(C)(C)C)nc3c2n1 ; CC(C)(C)c1ccc2ccc3c(-c4cccc(-c5ccc6c(c5)C5(c7ccccc7-6)c6ccccc6C(C)(C)c6ccccc65)c4)cc(C(C)(C)C)nc3c2n1]

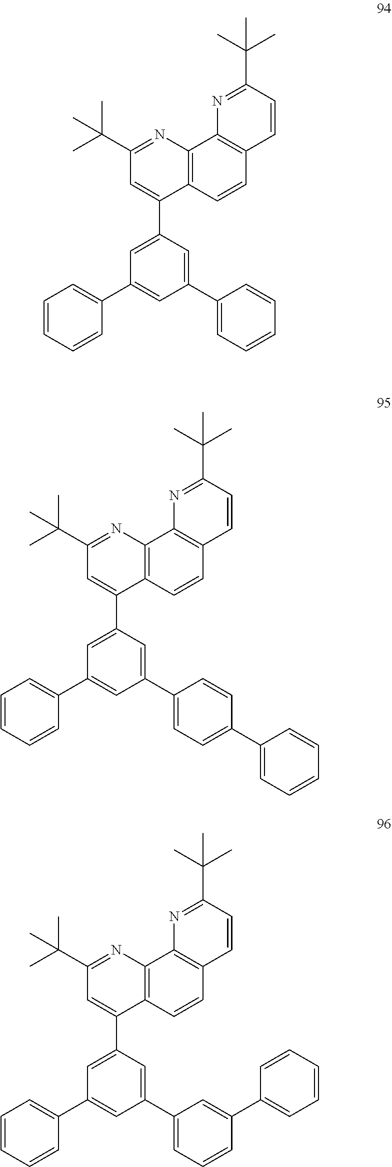 [SMILES: CC(C)(C)c1ccc2ccc3c(-c4cc(-c5ccccc5)cc(-c5ccccc5)c4)cc(C(C)(C)C)nc3c2n1 ; CC(C)(C)c1ccc2ccc3c(-c4cc(-c5ccccc5)cc(-c5ccc(-c6ccccc6)cc5)c4)cc(C(C)(C)C)nc3c2n1 ; CC(C)(C)c1ccc2ccc3c(-c4cc(-c5ccccc5)cc(-c5cccc(-c6ccccc6)c5)c4)cc(C(C)(C)C)nc3c2n1]

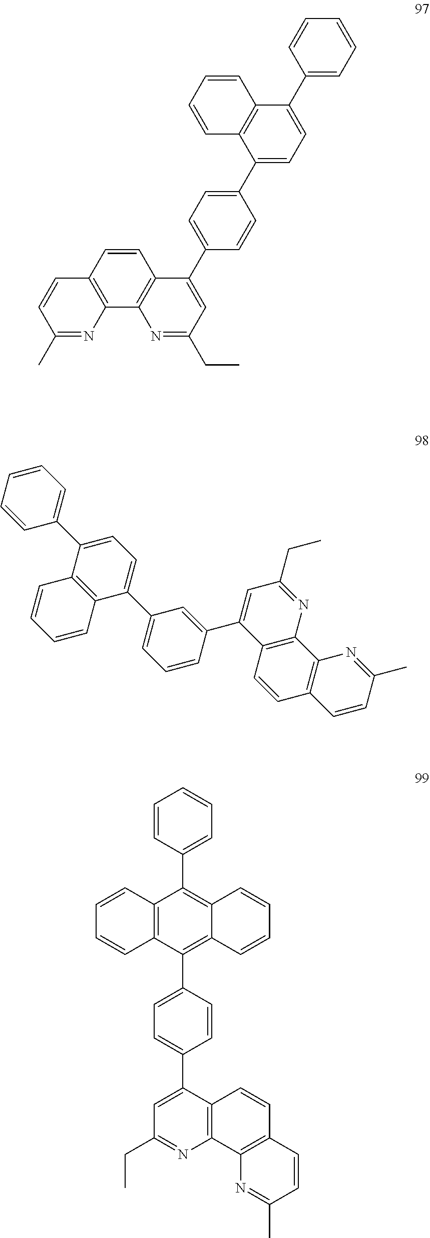 [SMILES: CCc1cc(-c2ccc(-c3ccc(-c4ccccc4)c4ccccc34)cc2)c2ccc3ccc(C)nc3c2n1 ; CCc1cc(-c2cccc(-c3ccc(-c4ccccc4)c4ccccc34)c2)c2ccc3ccc(C)nc3c2n1 ; CCc1cc(-c2ccc(-c3c4ccccc4c(-c4ccccc4)c4ccccc34)cc2)c2ccc3ccc(C)nc3c2n1]

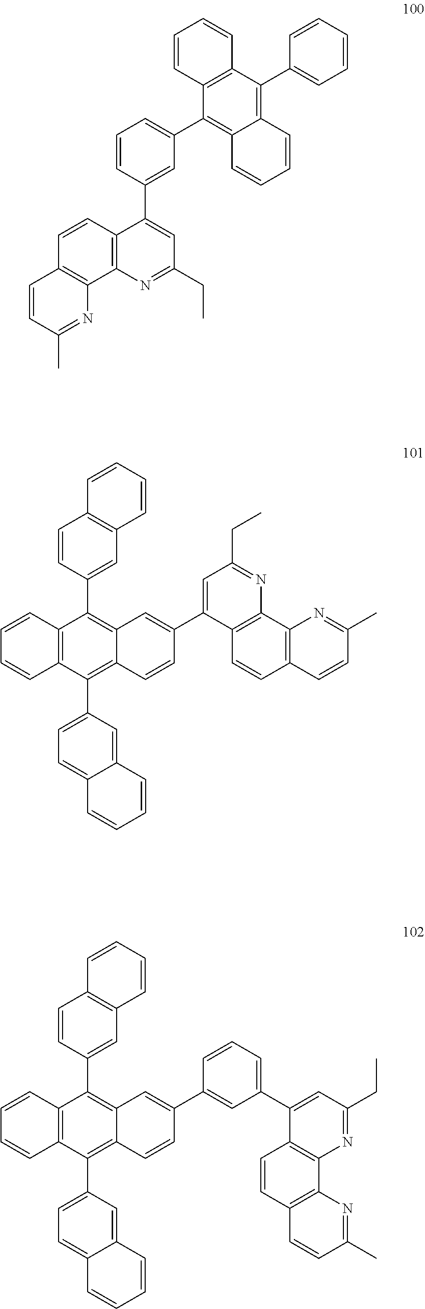 [SMILES: CCc1cc(-c2cccc(-c3c4ccccc4c(-c4ccccc4)c4ccccc34)c2)c2ccc3ccc(C)nc3c2n1 ; CCc1cc(-c2ccc3c(-c4ccc5ccccc5c4)c4ccccc4c(-c4ccc5ccccc5c4)c3c2)c2ccc3ccc(C)nc3c2n1 ; CCc1cc(-c2cccc(-c3ccc4c(-c5ccc6ccccc6c5)c5ccccc5c(-c5ccc6ccccc6c5)c4c3)c2)c2ccc3ccc(C)nc3c2n1]

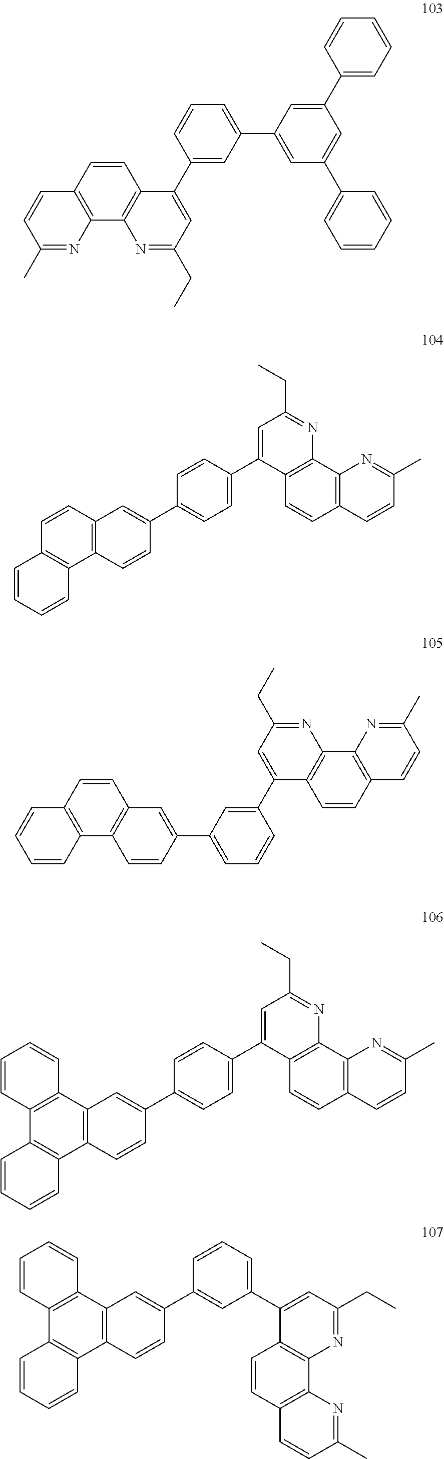

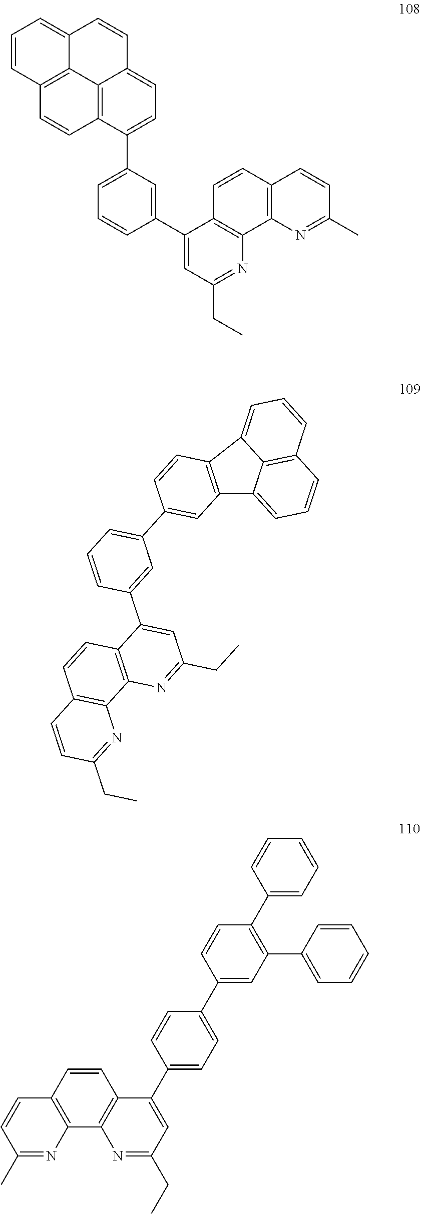 [SMILES: CCc1cc(-c2cccc(-c3ccc4ccc5cccc6ccc3c4c56)c2)c2ccc3ccc(C)nc3c2n1 ; CCc1ccc2ccc3c(-c4cccc(-c5ccc6c(c5)-c5cccc7cccc-6c57)c4)cc(CC)nc3c2n1 ; CCc1cc(-c2ccc(-c3ccc(-c4ccccc4)c(-c4ccccc4)c3)cc2)c2ccc3ccc(C)nc3c2n1]

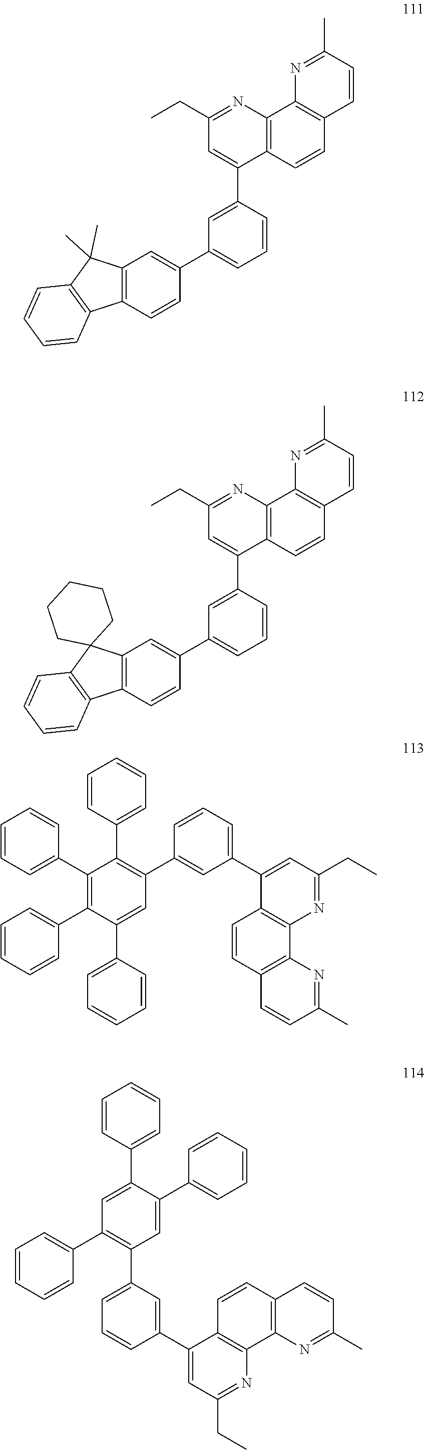

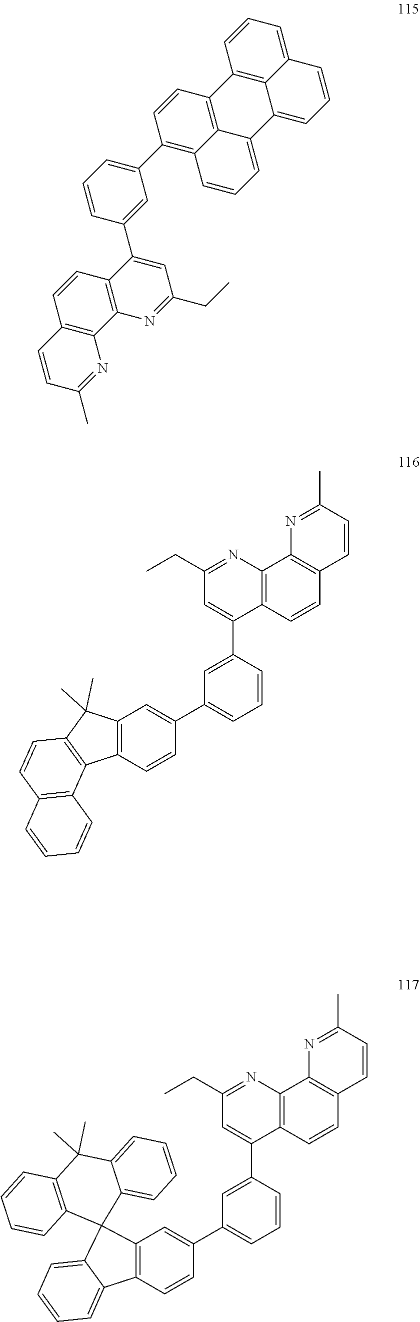 [SMILES: CCc1cc(-c2cccc(-c3ccc4c5cccc6cccc(c7cccc3c74)c65)c2)c2ccc3ccc(C)nc3c2n1 ; CCc1cc(-c2cccc(-c3ccc4c(c3)C(C)(C)c3ccc5ccccc5c3-4)c2)c2ccc3ccc(C)nc3c2n1 ; CCc1cc(-c2cccc(-c3ccc4c(c3)C3(c5ccccc5-4)c4ccccc4C(C)(C)c4ccccc43)c2)c2ccc3ccc(C)nc3c2n1]

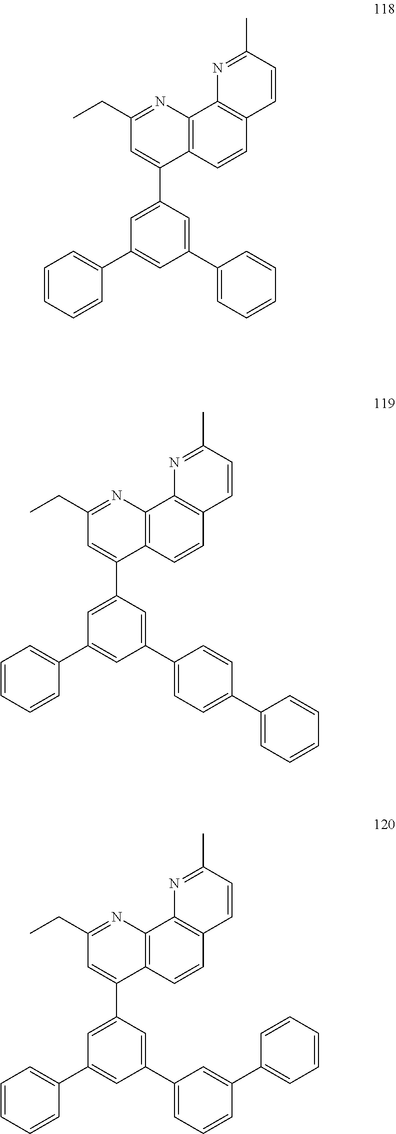 [SMILES: CCc1cc(-c2cc(-c3ccccc3)cc(-c3ccccc3)c2)c2ccc3ccc(C)nc3c2n1 ; CCc1cc(-c2cc(-c3ccccc3)cc(-c3ccc(-c4ccccc4)cc3)c2)c2ccc3ccc(C)nc3c2n1 ; CCc1cc(-c2cc(-c3ccccc3)cc(-c3cccc(-c4ccccc4)c3)c2)c2ccc3ccc(C)nc3c2n1]

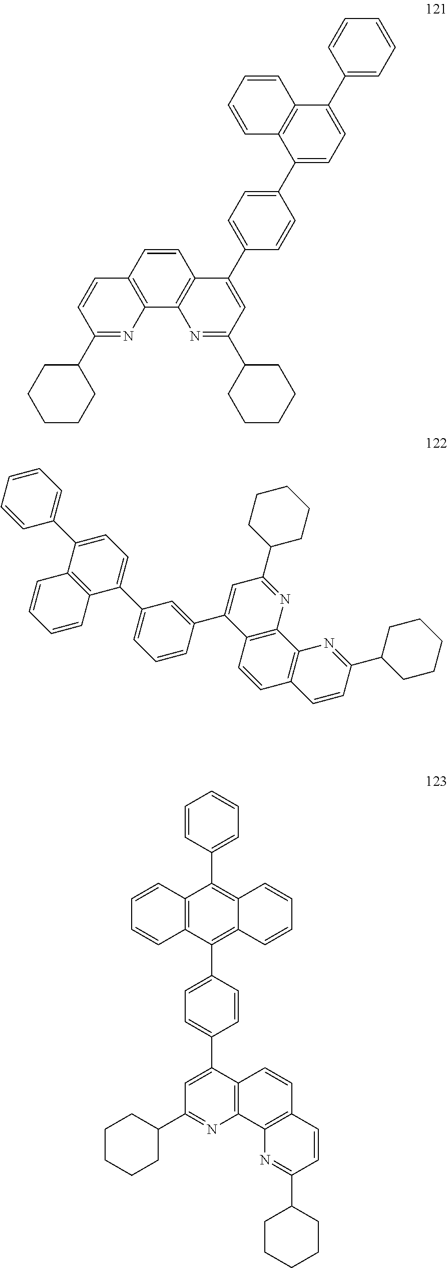 [SMILES: c1ccc(-c2ccc(-c3ccc(-c4cc(C5CCCCC5)nc5c4ccc4ccc(C6CCCCC6)nc45)cc3)c3ccccc23)cc1 ; c1ccc(-c2ccc(-c3cccc(-c4cc(C5CCCCC5)nc5c4ccc4ccc(C6CCCCC6)nc45)c3)c3ccccc23)cc1 ; c1ccc(-c2c3ccccc3c(-c3ccc(-c4cc(C5CCCCC5)nc5c4ccc4ccc(C6CCCCC6)nc45)cc3)c3ccccc23)cc1]

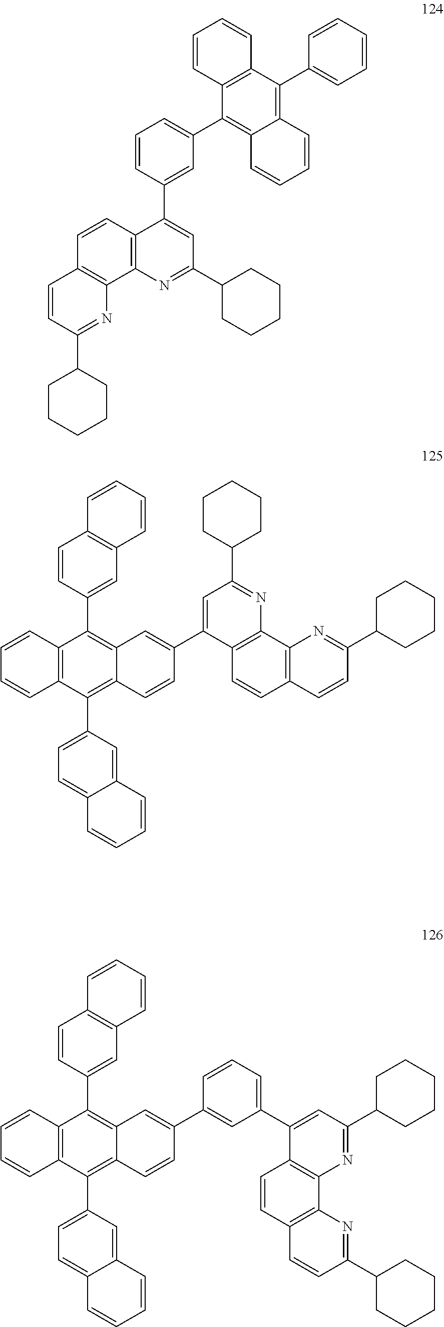 [SMILES: c1ccc(-c2c3ccccc3c(-c3cccc(-c4cc(C5CCCCC5)nc5c4ccc4ccc(C6CCCCC6)nc45)c3)c3ccccc23)cc1 ; c1ccc2cc(-c3c4ccccc4c(-c4ccc5ccccc5c4)c4cc(-c5cc(C6CCCCC6)nc6c5ccc5ccc(C7CCCCC7)nc56)ccc34)ccc2c1 ; c1cc(-c2ccc3c(-c4ccc5ccccc5c4)c4ccccc4c(-c4ccc5ccccc5c4)c3c2)cc(-c2cc(C3CCCCC3)nc3c2ccc2ccc(C4CCCCC4)nc23)c1]

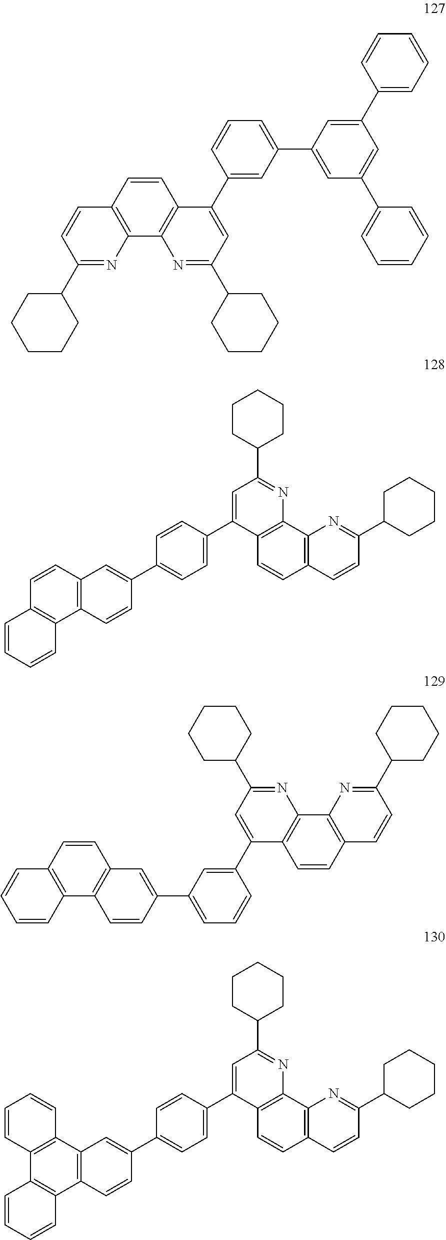

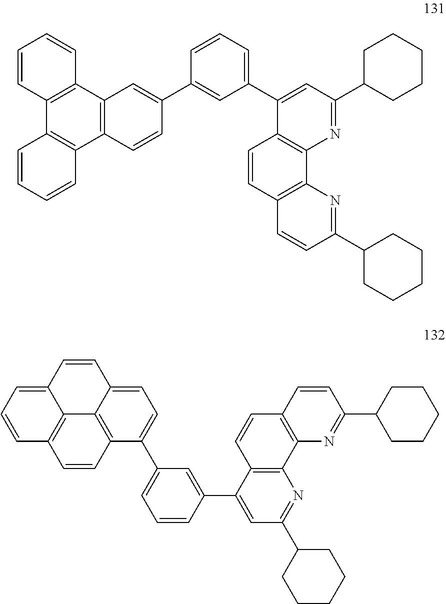 [SMILES: c1cc(-c2ccc3c4ccccc4c4ccccc4c3c2)cc(-c2cc(C3CCCCC3)nc3c2ccc2ccc(C4CCCCC4)nc23)c1 ; c1cc(-c2cc(C3CCCCC3)nc3c2ccc2ccc(C4CCCCC4)nc23)cc(-c2ccc3ccc4cccc5ccc2c3c45)c1]

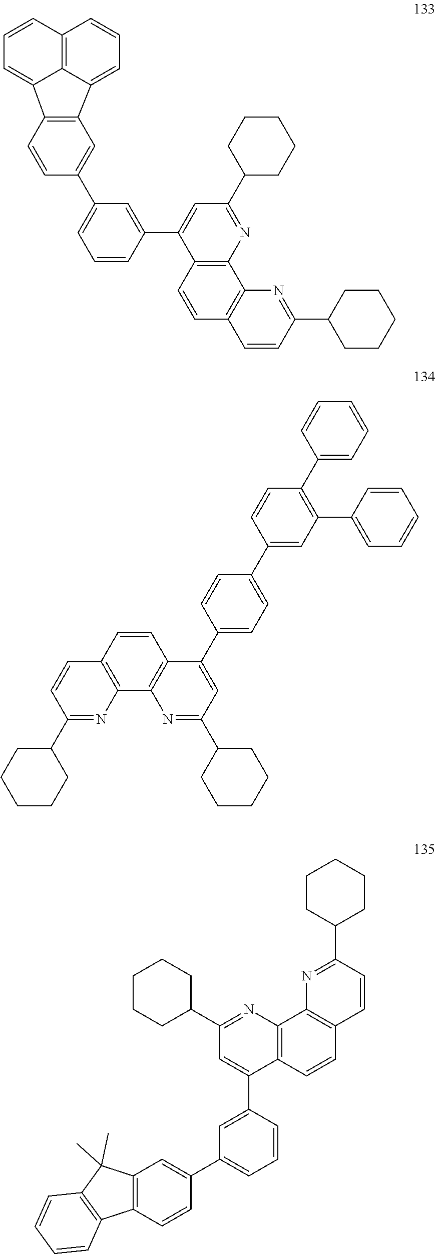 [SMILES: c1cc(-c2ccc3c(c2)-c2cccc4cccc-3c24)cc(-c2cc(C3CCCCC3)nc3c2ccc2ccc(C4CCCCC4)nc23)c1 ; c1ccc(-c2ccc(-c3ccc(-c4cc(C5CCCCC5)nc5c4ccc4ccc(C6CCCCC6)nc45)cc3)cc2-c2ccccc2)cc1 ; CC1(C)c2ccccc2-c2ccc(-c3cccc(-c4cc(C5CCCCC5)nc5c4ccc4ccc(C6CCCCC6)nc45)c3)cc21]

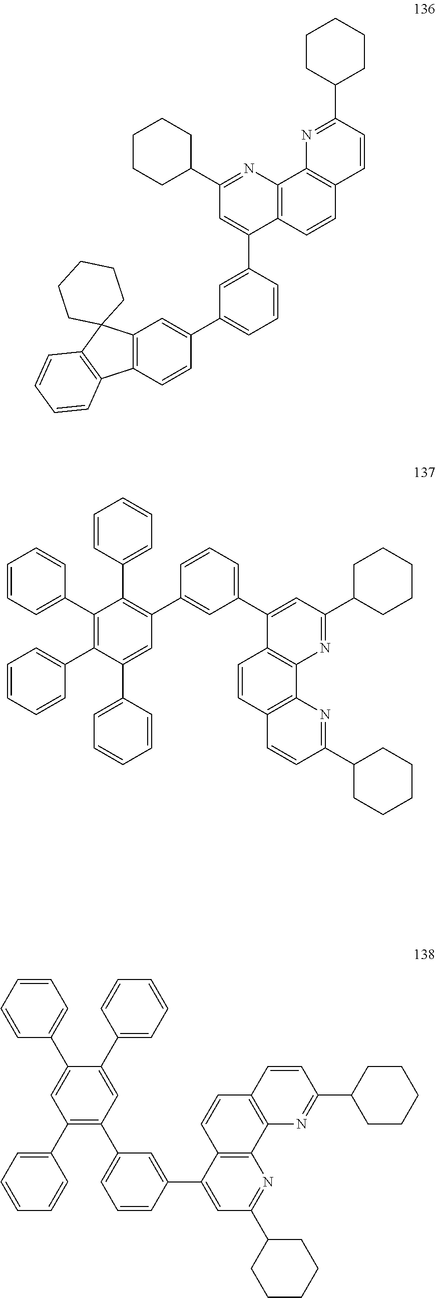 [SMILES: c1cc(-c2ccc3c(c2)C2(CCCCC2)c2ccccc2-3)cc(-c2cc(C3CCCCC3)nc3c2ccc2ccc(C4CCCCC4)nc23)c1 ; c1ccc(-c2cc(-c3cccc(-c4cc(C5CCCCC5)nc5c4ccc4ccc(C6CCCCC6)nc45)c3)c(-c3ccccc3)c(-c3ccccc3)c2-c2ccccc2)cc1 ; c1ccc(-c2cc(-c3ccccc3)c(-c3cccc(-c4cc(C5CCCCC5)nc5c4ccc4ccc(C6CCCCC6)nc45)c3)cc2-c2ccccc2)cc1]

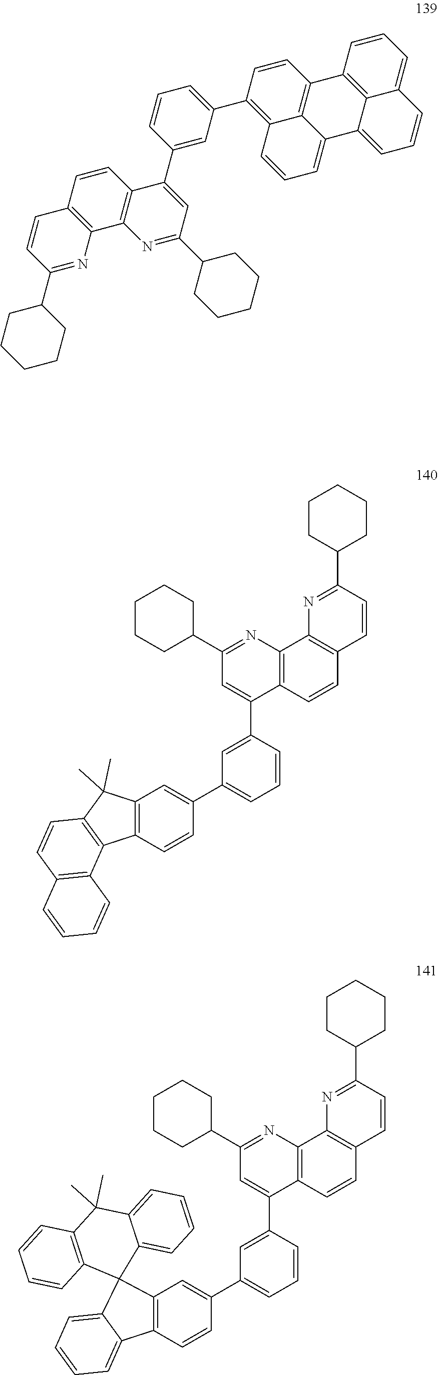 [SMILES: c1cc(-c2cc(C3CCCCC3)nc3c2ccc2ccc(C4CCCCC4)nc23)cc(-c2ccc3c4cccc5cccc(c6cccc2c63)c54)c1 ; CC1(C)c2cc(-c3cccc(-c4cc(C5CCCCC5)nc5c4ccc4ccc(C6CCCCC6)nc45)c3)ccc2-c2c1ccc1ccccc21 ; CC1(C)c2ccccc2C2(c3ccccc3-c3ccc(-c4cccc(-c5cc(C6CCCCC6)nc6c5ccc5ccc(C7CCCCC7)nc56)c4)cc32)c2ccccc21]

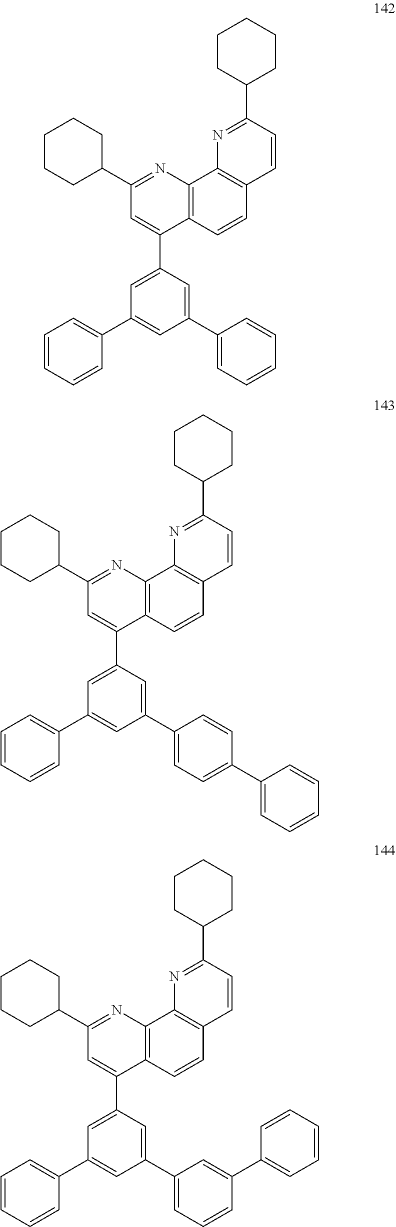 [SMILES: c1ccc(-c2cc(-c3ccccc3)cc(-c3cc(C4CCCCC4)nc4c3ccc3ccc(C5CCCCC5)nc34)c2)cc1 ; c1ccc(-c2ccc(-c3cc(-c4ccccc4)cc(-c4cc(C5CCCCC5)nc5c4ccc4ccc(C6CCCCC6)nc45)c3)cc2)cc1 ; c1ccc(-c2cccc(-c3cc(-c4ccccc4)cc(-c4cc(C5CCCCC5)nc5c4ccc4ccc(C6CCCCC6)nc45)c3)c2)cc1]

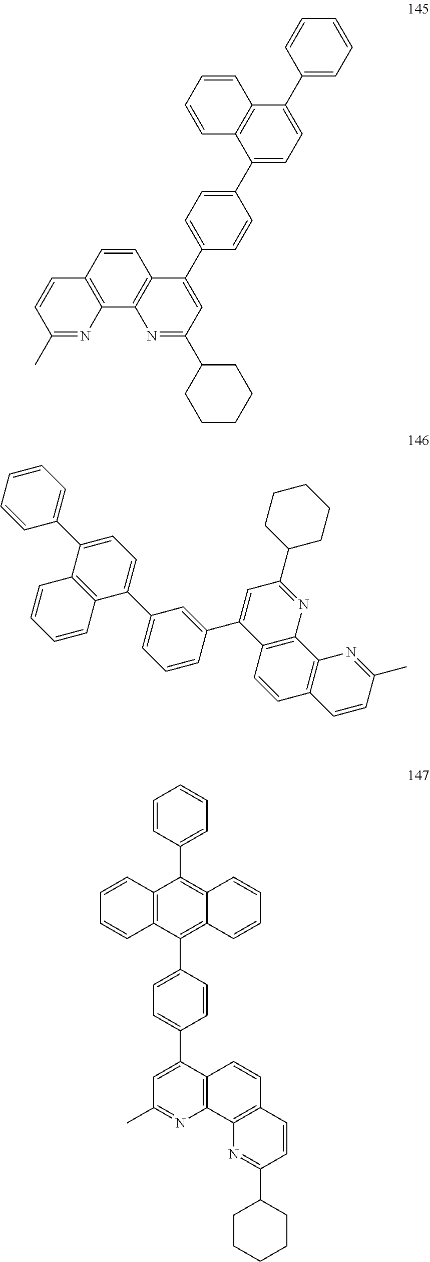 [SMILES: Cc1ccc2ccc3c(-c4ccc(-c5ccc(-c6ccccc6)c6ccccc56)cc4)cc(C4CCCCC4)nc3c2n1 ; Cc1ccc2ccc3c(-c4cccc(-c5ccc(-c6ccccc6)c6ccccc56)c4)cc(C4CCCCC4)nc3c2n1 ; Cc1cc(-c2ccc(-c3c4ccccc4c(-c4ccccc4)c4ccccc34)cc2)c2ccc3ccc(C4CCCCC4)nc3c2n1]

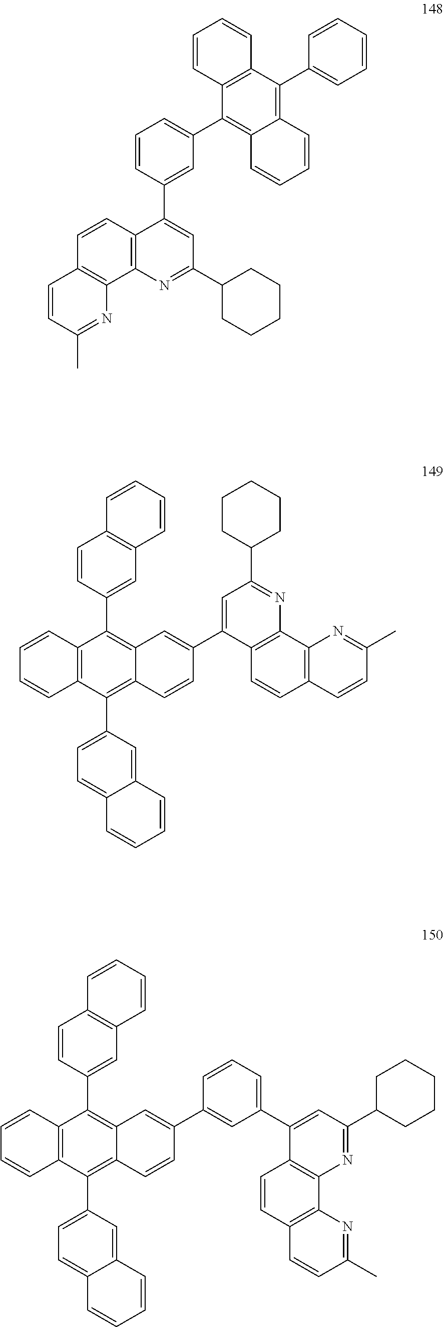 [SMILES: Cc1ccc2ccc3c(-c4cccc(-c5c6ccccc6c(-c6ccccc6)c6ccccc56)c4)cc(C4CCCCC4)nc3c2n1 ; Cc1ccc2ccc3c(-c4ccc5c(-c6ccc7ccccc7c6)c6ccccc6c(-c6ccc7ccccc7c6)c5c4)cc(C4CCCCC4)nc3c2n1 ; Cc1ccc2ccc3c(-c4cccc(-c5ccc6c(-c7ccc8ccccc8c7)c7ccccc7c(-c7ccc8ccccc8c7)c6c5)c4)cc(C4CCCCC4)nc3c2n1]

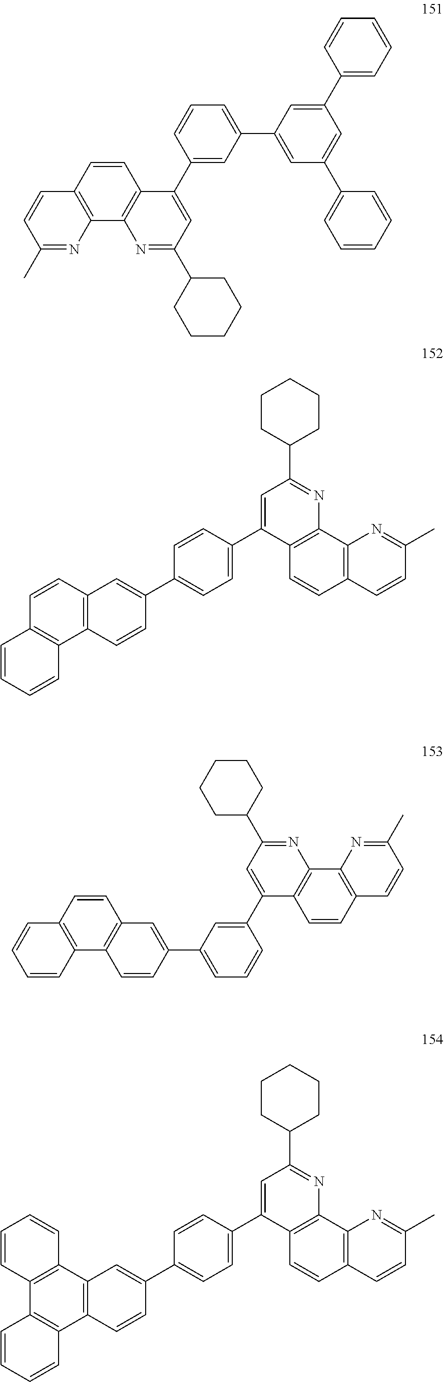 [SMILES: Cc1ccc2ccc3c(-c4cccc(-c5cc(-c6ccccc6)cc(-c6ccccc6)c5)c4)cc(C4CCCCC4)nc3c2n1 ; Cc1ccc2ccc3c(-c4ccc(-c5ccc6c(ccc7ccccc76)c5)cc4)cc(C4CCCCC4)nc3c2n1 ; Cc1ccc2ccc3c(-c4cccc(-c5ccc6c(ccc7ccccc76)c5)c4)cc(C4CCCCC4)nc3c2n1 ; Cc1ccc2ccc3c(-c4ccc(-c5ccc6c7ccccc7c7ccccc7c6c5)cc4)cc(C4CCCCC4)nc3c2n1]

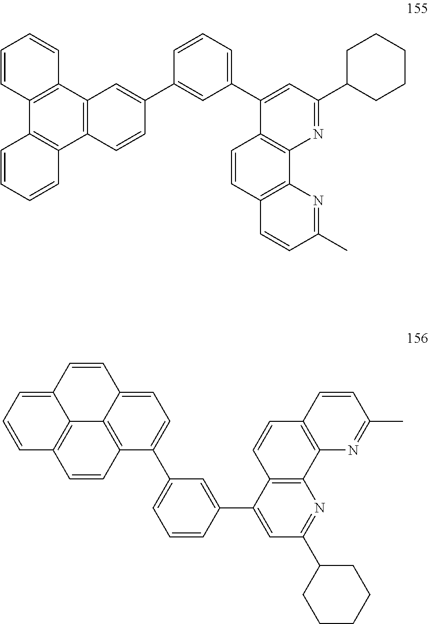 [SMILES: Cc1ccc2ccc3c(-c4cccc(-c5ccc6c7ccccc7c7ccccc7c6c5)c4)cc(C4CCCCC4)nc3c2n1 ; Cc1ccc2ccc3c(-c4cccc(-c5ccc6ccc7cccc8ccc5c6c78)c4)cc(C4CCCCC4)nc3c2n1]

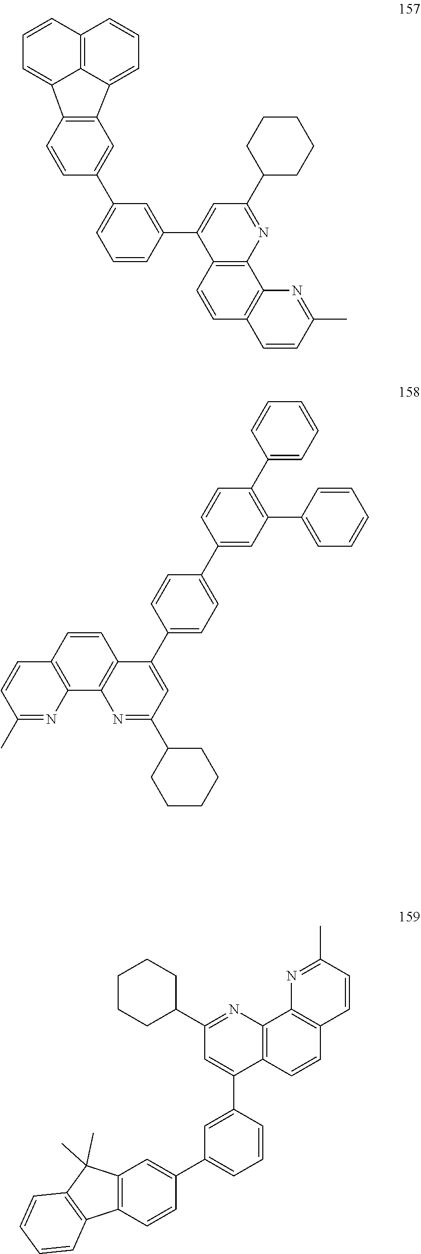 [SMILES: Cc1ccc2ccc3c(-c4cccc(-c5ccc6c(c5)-c5cccc7cccc-6c57)c4)cc(C4CCCCC4)nc3c2n1 ; Cc1ccc2ccc3c(-c4ccc(-c5ccc(-c6ccccc6)c(-c6ccccc6)c5)cc4)cc(C4CCCCC4)nc3c2n1 ; Cc1ccc2ccc3c(-c4cccc(-c5ccc6c(c5)C(C)(C)c5ccccc5-6)c4)cc(C4CCCCC4)nc3c2n1]

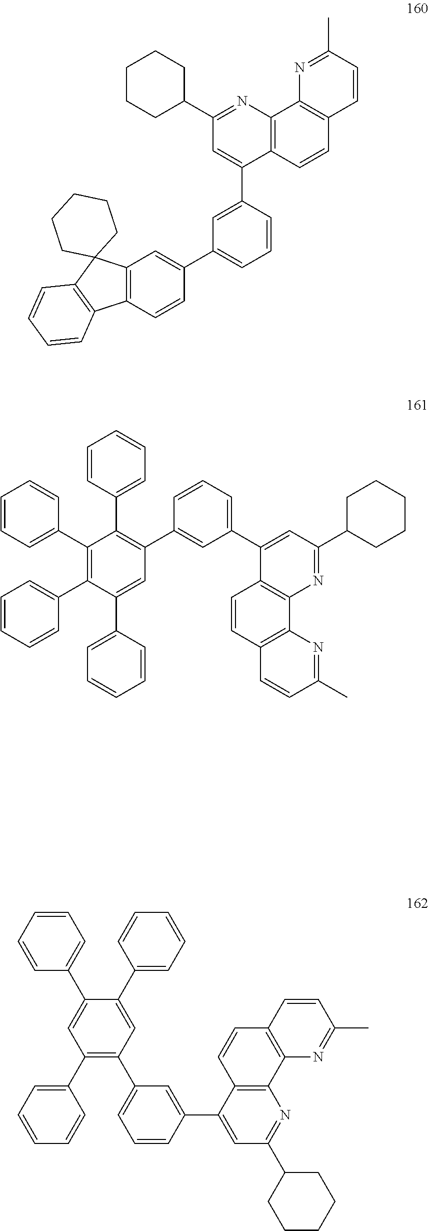 [SMILES: Cc1ccc2ccc3c(-c4cccc(-c5ccc6c(c5)C5(CCCCC5)c5ccccc5-6)c4)cc(C4CCCCC4)nc3c2n1 ; Cc1ccc2ccc3c(-c4cccc(-c5cc(-c6ccccc6)c(-c6ccccc6)c(-c6ccccc6)c5-c5ccccc5)c4)cc(C4CCCCC4)nc3c2n1 ; Cc1ccc2ccc3c(-c4cccc(-c5cc(-c6ccccc6)c(-c6ccccc6)cc5-c5ccccc5)c4)cc(C4CCCCC4)nc3c2n1]

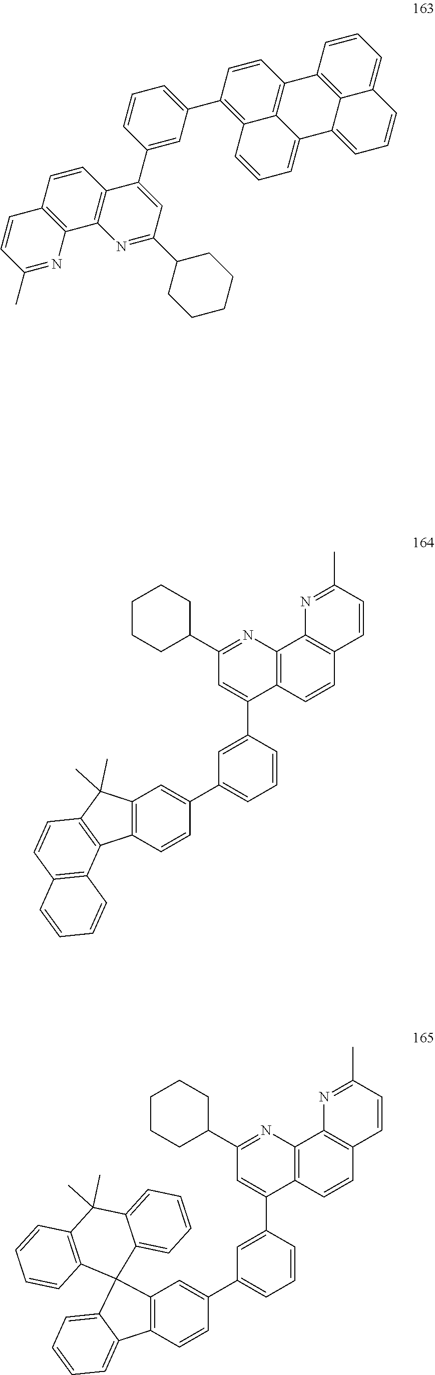 [SMILES: Cc1ccc2ccc3c(-c4cccc(-c5ccc6c7cccc8cccc(c9cccc5c96)c87)c4)cc(C4CCCCC4)nc3c2n1 ; Cc1ccc2ccc3c(-c4cccc(-c5ccc6c(c5)C(C)(C)c5ccc7ccccc7c5-6)c4)cc(C4CCCCC4)nc3c2n1 ; Cc1ccc2ccc3c(-c4cccc(-c5ccc6c(c5)C5(c7ccccc7-6)c6ccccc6C(C)(C)c6ccccc65)c4)cc(C4CCCCC4)nc3c2n1]

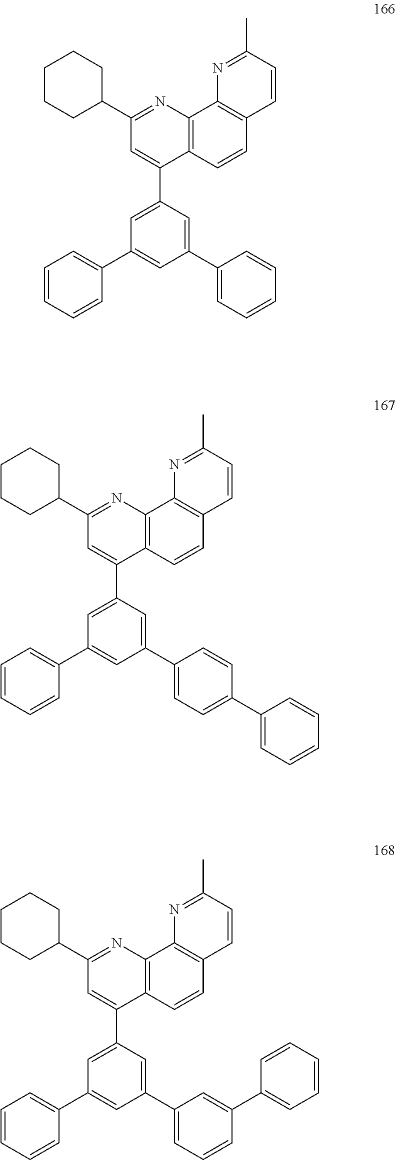 [SMILES: Cc1ccc2ccc3c(-c4cc(-c5ccccc5)cc(-c5ccccc5)c4)cc(C4CCCCC4)nc3c2n1 ; Cc1ccc2ccc3c(-c4cc(-c5ccccc5)cc(-c5ccc(-c6ccccc6)cc5)c4)cc(C4CCCCC4)nc3c2n1 ; Cc1ccc2ccc3c(-c4cc(-c5ccccc5)cc(-c5cccc(-c6ccccc6)c5)c4)cc(C4CCCCC4)nc3c2n1]

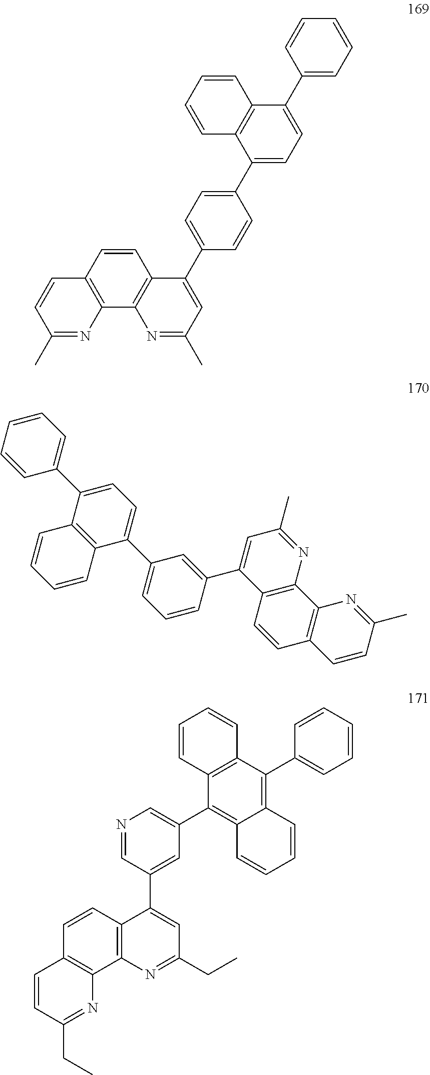

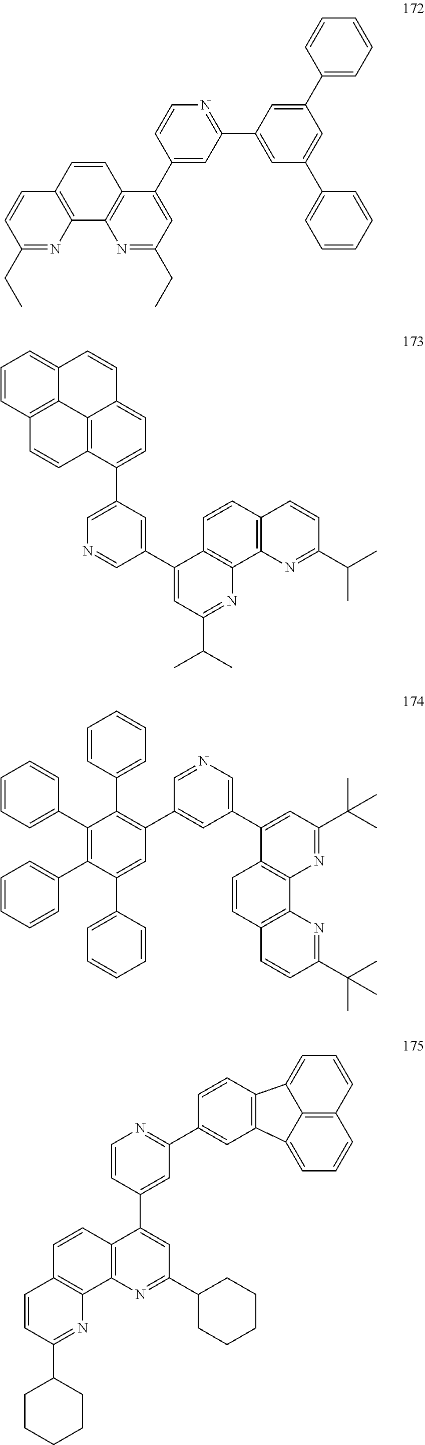

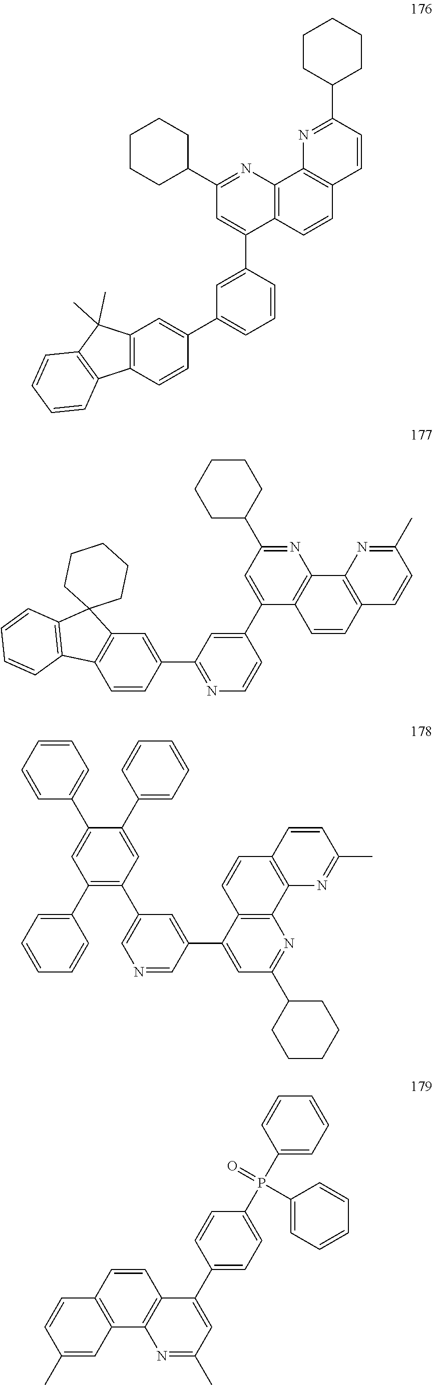

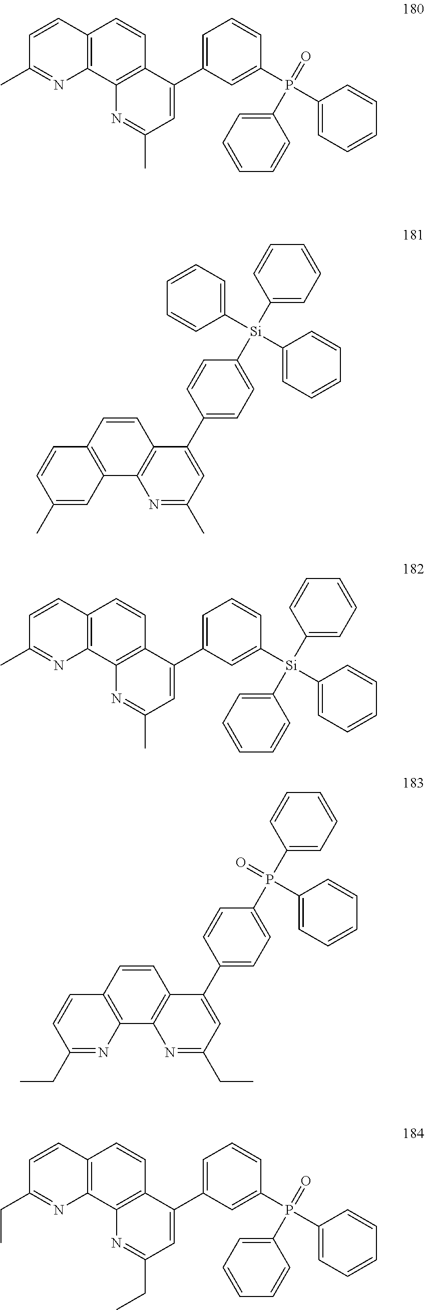

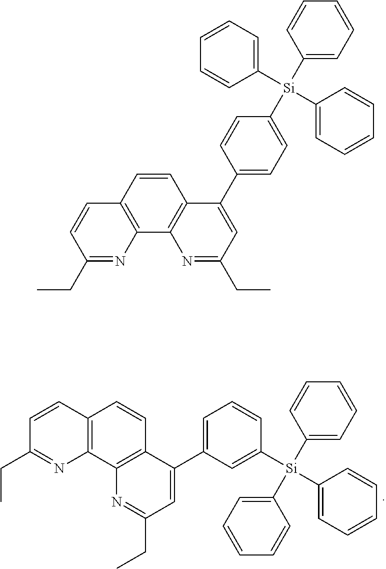 [SMILES: CCc1ccc2ccc3c(-c4ccc([Si](c5ccccc5)(c5ccccc5)c5ccccc5)cc4)cc(CC)nc3c2n1 ; CCc1ccc2ccc3c(-c4cccc([Si](c5ccccc5)(c5ccccc5)c5ccccc5)c4)cc(CC)nc3c2n1]

20. The organic electroluminescent device of claim 13, wherein at least one of R₁ and R₂ is an alkyl group of C₁-C₆₀ or a cycloalkyl group of C₃-C₆₀.

21. The organic electroluminescent device of claim 13, wherein the compound of Chemical Formula 1 is a compound of any one of the following Chemical Formulas 2 to 6: 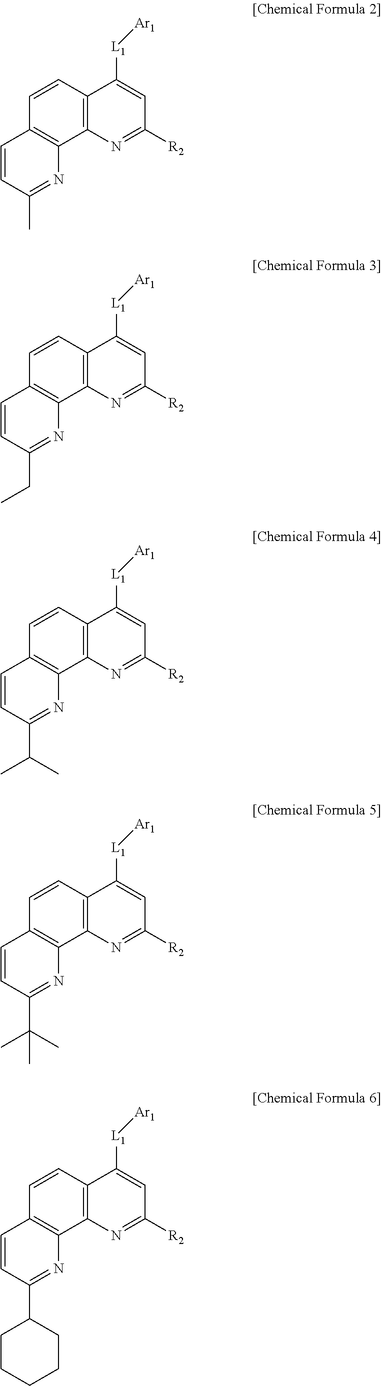
wherein,
L₁ and Ar₁ are as defined in claim 13, and
R₂ is an alkyl group of C₁-C₆ or a cycloalkyl group of C₃-C₆.

22. The organic electroluminescent device of claim 13, wherein the compound of Chemical Formula 1 is a compound of any one of the following Chemical Formulas 7 to 12: 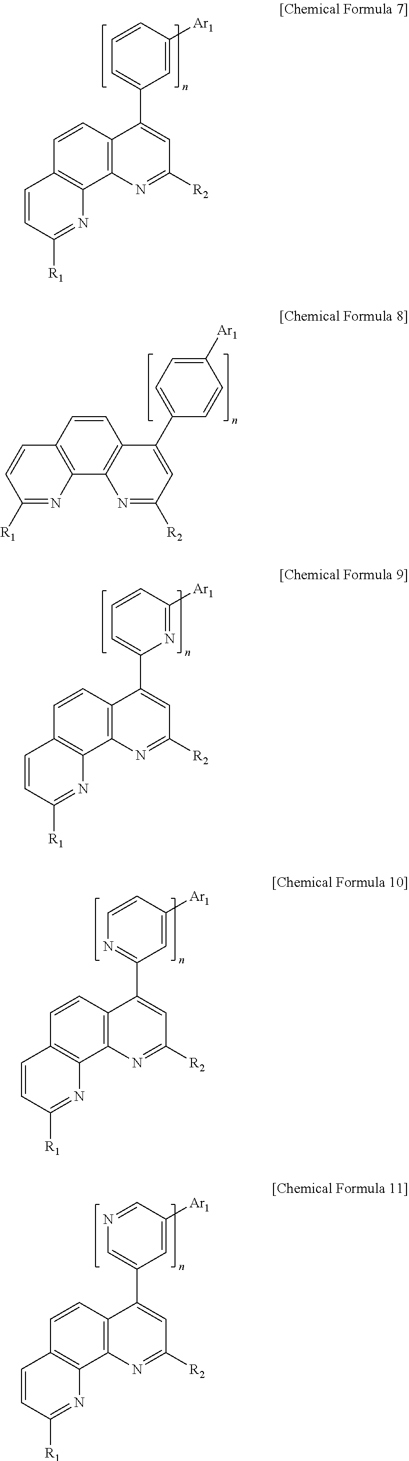 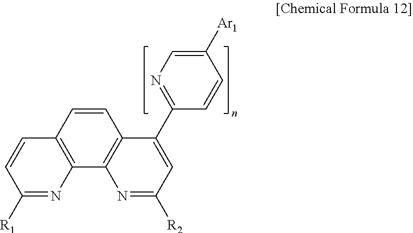
wherein,
R₁ and R₂, which are same or different, are each independently an alkyl group of C₁-C₆ or a cycloalkyl group of C₃-C₆,
n is 0 or 1, and
Ar₁ is selected from the group consisting of the following substituents S1 to S8: 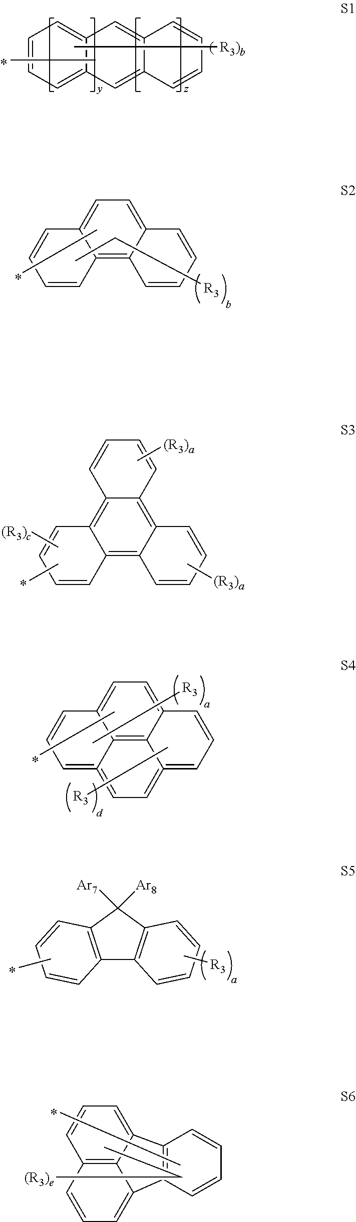 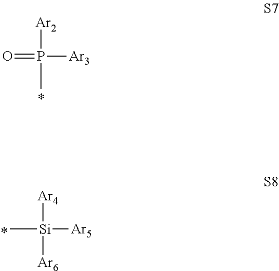
wherein,
a is an integer of 0 to 4,
b is an integer of 0 to 9,
c is an integer of 0 to 3,
d is an integer of 0 to 5,
e is an integer of 0 to 9,
the plurality of R₃ are the same as or different from each other,
R₃ or two or more R₃'s, if present, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀,
y and z are each 0 or 1,
Ar₂ to Ar₈, which are same or different, are each independently selected from the group consisting of a hydrogen atom, a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, or forming a fused ring with an adjacent group, and
the hydrazino group, the hydrazono group, alkyl group, the alkenyl group, the alkynyl group, the cycloalkyl group, the heterocycloalkyl group, the cycloalkenyl group, the heterocycloalkenyl group, the aryl group, the heteroaryl group, the alkyloxy group, the aryloxy group, the alkylsilyl group, the arylsilyl group, the alkylboron group, the arylboron group, the aryl phosphine group, the aryl phosphine oxide group, and the arylamine group of Ar₂ to Ar₈ are each independently substituted or unsubstituted with one or more substituents of: a deuterium atom, a halogen group, a hydroxy group, a cyano group, a nitro group, an amino group, an amidino group, a hydrazino group, a hydrazono group, an alkyl group of C₁-C₆₀, an alkenyl group of C₂-C₆₀, an alkynyl group of C₂-C₆₀, a cycloalkyl group of C₃-C₆₀, a heterocycloalkyl group having 3 to 60 nuclear atoms, a cycloalkenyl group of C₃-C₆₀, a heterocycloalkenyl group having 3 to 60 nuclear atoms, an aryl group of C₆-C₆₀, a heteroaryl group having 5 to 60 nuclear atoms, an alkyloxy group of C₁-C₆₀, an aryloxy group of C₆-C₆₀, an alkylsilyl group of C₁-C₆₀, an arylsilyl group of C₆-C₆₀, an alkylboron group of C₁-C₄₀, an arylboron group of C₆-C₆₀, an arylphosphine group of C₆-C₆₀, an arylphosphine oxide group of C₆-C₆₀, and an arylamine group of C₆-C₆₀, and when the substituents are plural in number, the substituents are the same as or different from each other.

23. The organic electroluminescent device of claim 13, wherein the compound of Chemical Formula 1 is selected from the group consisting of the following Compounds 1 to 186: 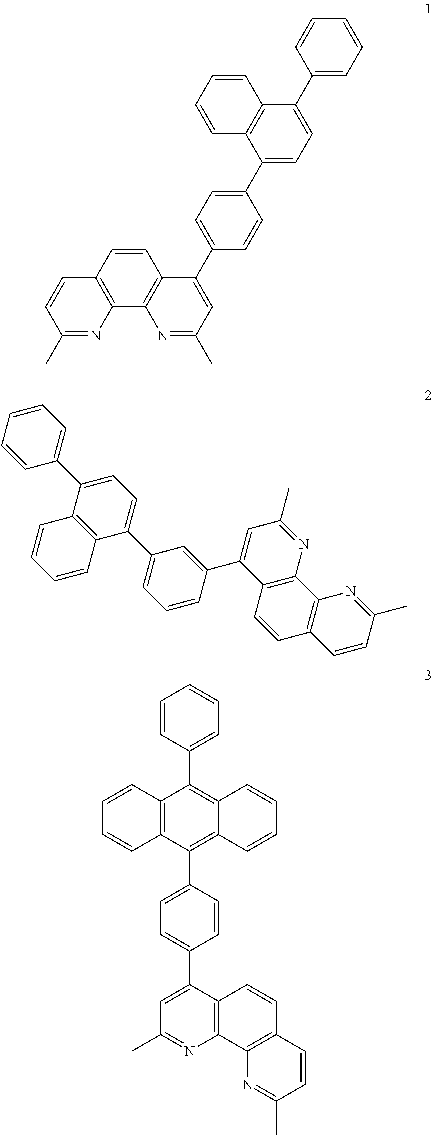 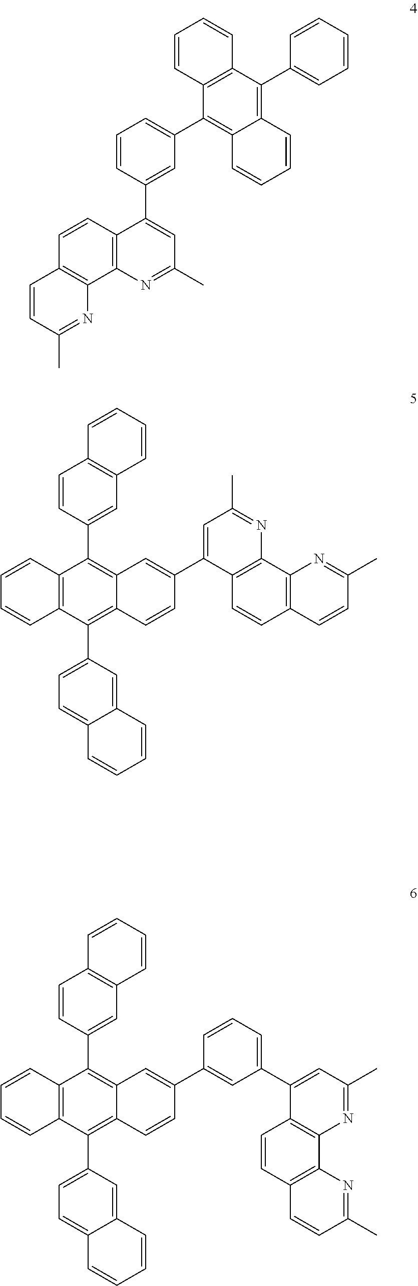 [SMILES: Cc1ccc2ccc3c(-c4cccc(-c5c6ccccc6c(-c6ccccc6)c6ccccc56)c4)cc(C)nc3c2n1 ; Cc1ccc2ccc3c(-c4ccc5c(-c6ccc7ccccc7c6)c6ccccc6c(-c6ccc7ccccc7c6)c5c4)cc(C)nc3c2n1 ; Cc1ccc2ccc3c(-c4cccc(-c5ccc6c(-c7ccc8ccccc8c7)c7ccccc7c(-c7ccc8ccccc8c7)c6c5)c4)cc(C)nc3c2n1] 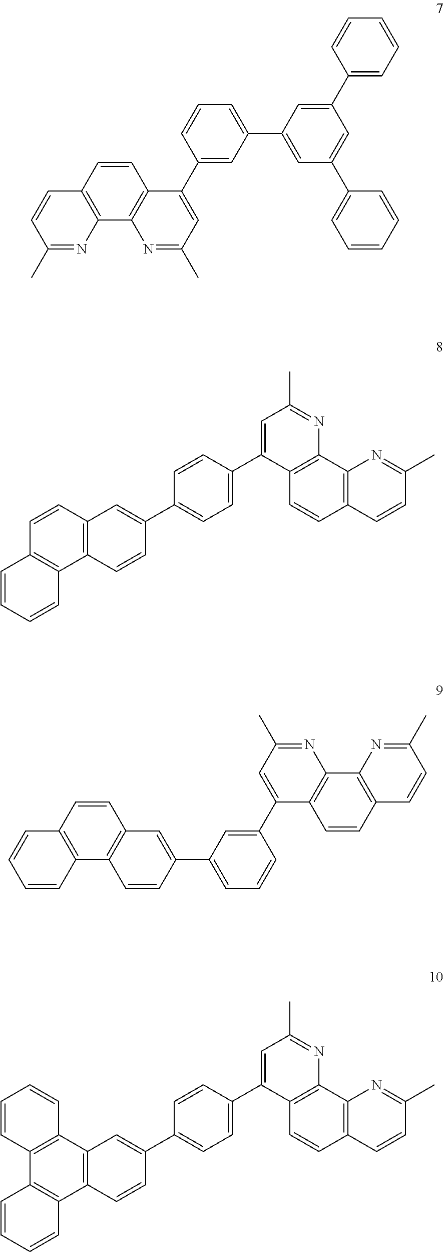 [SMILES: Cc1ccc2ccc3c(-c4cccc(-c5cc(-c6ccccc6)cc(-c6ccccc6)c5)c4)cc(C)nc3c2n1 ; Cc1ccc2ccc3c(-c4ccc(-c5ccc6c(ccc7ccccc76)c5)cc4)cc(C)nc3c2n1 ; Cc1ccc2ccc3c(-c4cccc(-c5ccc6c(ccc7ccccc76)c5)c4)cc(C)nc3c2n1 ; Cc1ccc2ccc3c(-c4ccc(-c5ccc6c7ccccc7c7ccccc7c6c5)cc4)cc(C)nc3c2n1] 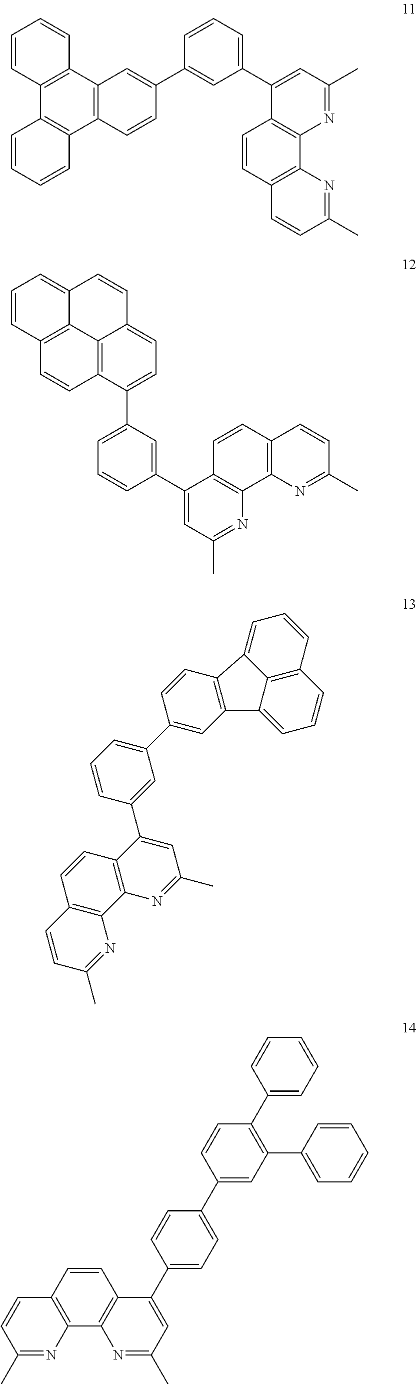 [SMILES: Cc1ccc2ccc3c(-c4cccc(-c5ccc6c7ccccc7c7ccccc7c6c5)c4)cc(C)nc3c2n1 ; Cc1ccc2ccc3c(-c4cccc(-c5ccc6ccc7cccc8ccc5c6c78)c4)cc(C)nc3c2n1 ; Cc1ccc2ccc3c(-c4cccc(-c5ccc6c(c5)-c5cccc7cccc-6c57)c4)cc(C)nc3c2n1 ; Cc1ccc2ccc3c(-c4ccc(-c5ccc(-c6ccccc6)c(-c6ccccc6)c5)cc4)cc(C)nc3c2n1] 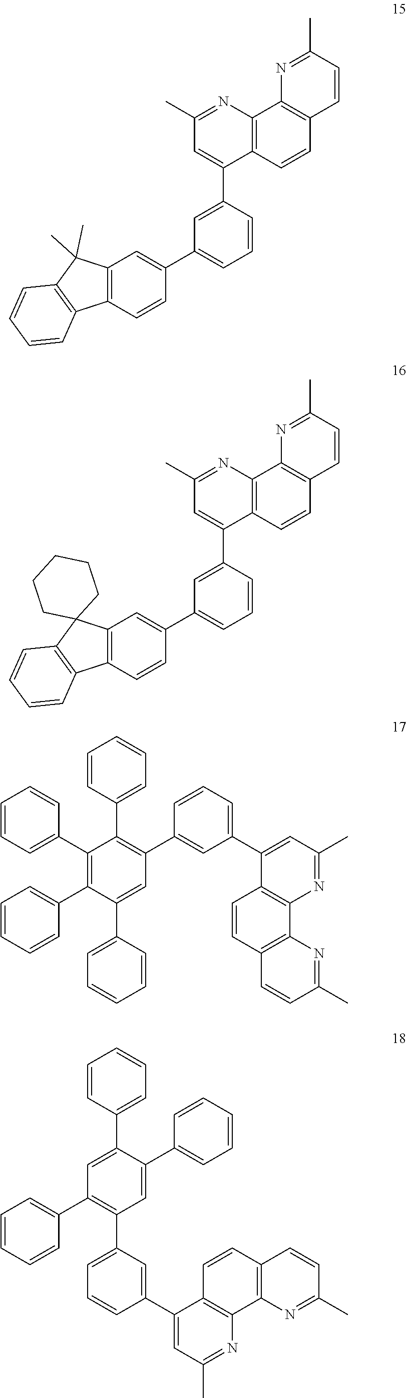 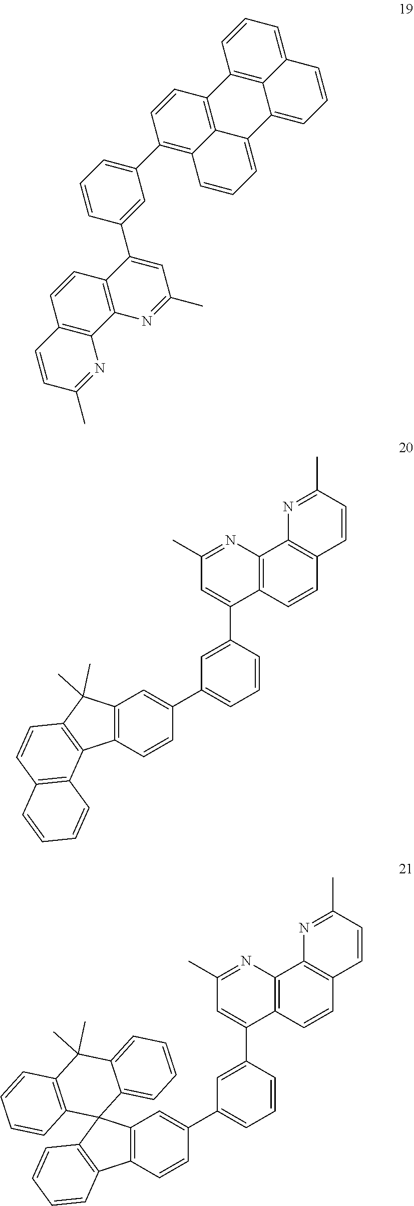 [SMILES: Cc1ccc2ccc3c(-c4cccc(-c5ccc6c7cccc8cccc(c9cccc5c96)c87)c4)cc(C)nc3c2n1 ; Cc1ccc2ccc3c(-c4cccc(-c5ccc6c(c5)C(C)(C)c5ccc7ccccc7c5-6)c4)cc(C)nc3c2n1 ; Cc1ccc2ccc3c(-c4cccc(-c5ccc6c(c5)C5(c7ccccc7-6)c6ccccc6C(C)(C)c6ccccc65)c4)cc(C)nc3c2n1] 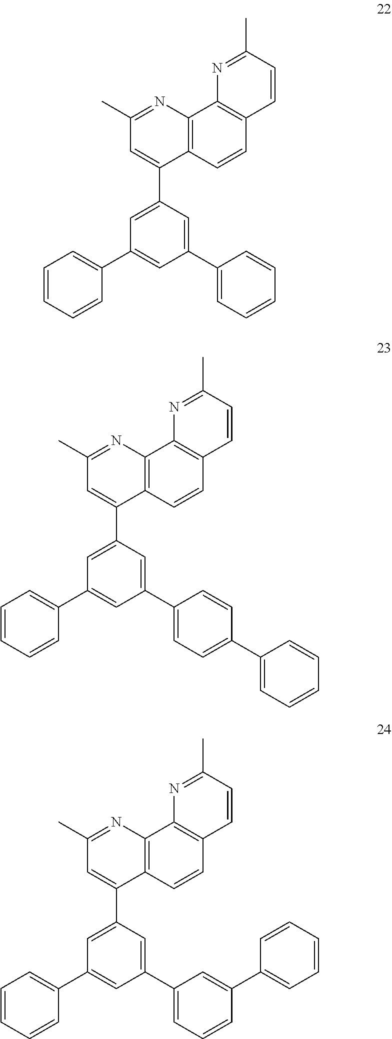 [SMILES: Cc1ccc2ccc3c(-c4cc(-c5ccccc5)cc(-c5ccccc5)c4)cc(C)nc3c2n1 ; Cc1ccc2ccc3c(-c4cc(-c5ccccc5)cc(-c5ccc(-c6ccccc6)cc5)c4)cc(C)nc3c2n1 ; Cc1ccc2ccc3c(-c4cc(-c5ccccc5)cc(-c5cccc(-c6ccccc6)c5)c4)cc(C)nc3c2n1] 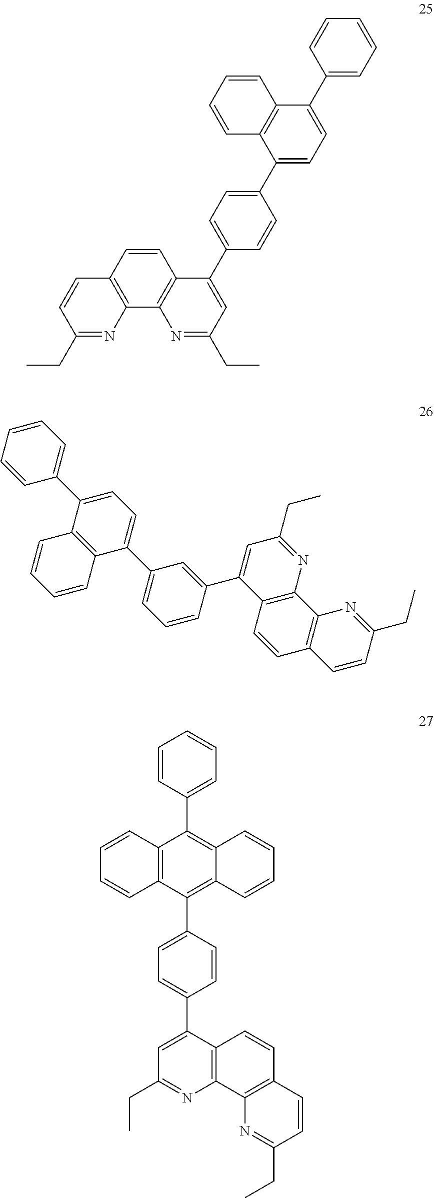 [SMILES: CCc1ccc2ccc3c(-c4ccc(-c5ccc(-c6ccccc6)c6ccccc56)cc4)cc(CC)nc3c2n1 ; CCc1ccc2ccc3c(-c4cccc(-c5ccc(-c6ccccc6)c6ccccc56)c4)cc(CC)nc3c2n1 ; CCc1ccc2ccc3c(-c4ccc(-c5c6ccccc6c(-c6ccccc6)c6ccccc56)cc4)cc(CC)nc3c2n1] 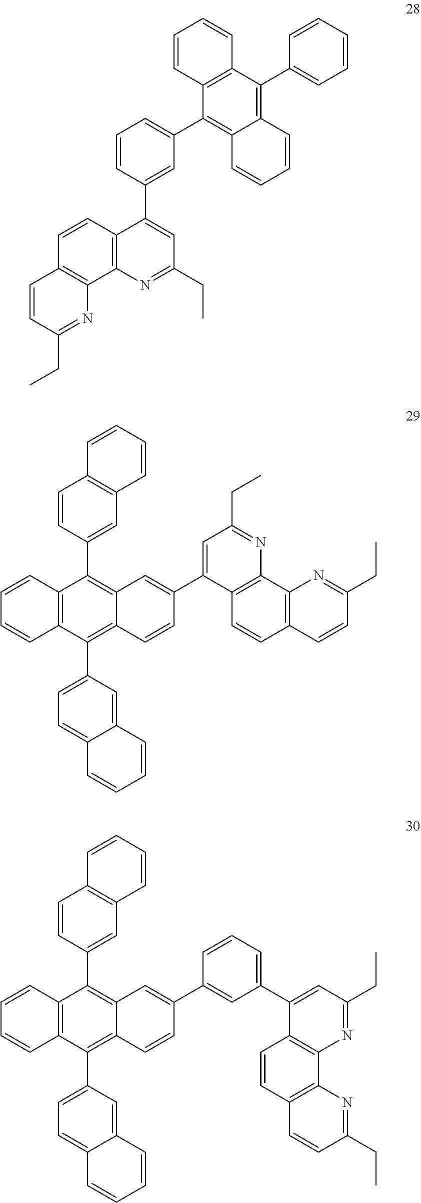 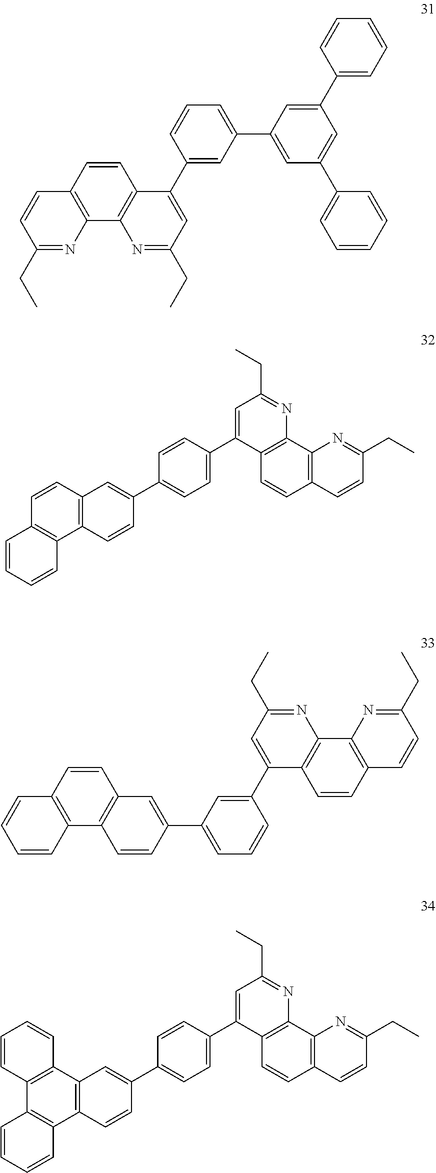 [SMILES: CCc1ccc2ccc3c(-c4cccc(-c5cc(-c6ccccc6)cc(-c6ccccc6)c5)c4)cc(CC)nc3c2n1 ; CCc1ccc2ccc3c(-c4ccc(-c5ccc6c(ccc7ccccc76)c5)cc4)cc(CC)nc3c2n1 ; CCc1ccc2ccc3c(-c4cccc(-c5ccc6c(ccc7ccccc76)c5)c4)cc(CC)nc3c2n1 ; CCc1ccc2ccc3c(-c4ccc(-c5ccc6c7ccccc7c7ccccc7c6c5)cc4)cc(CC)nc3c2n1] 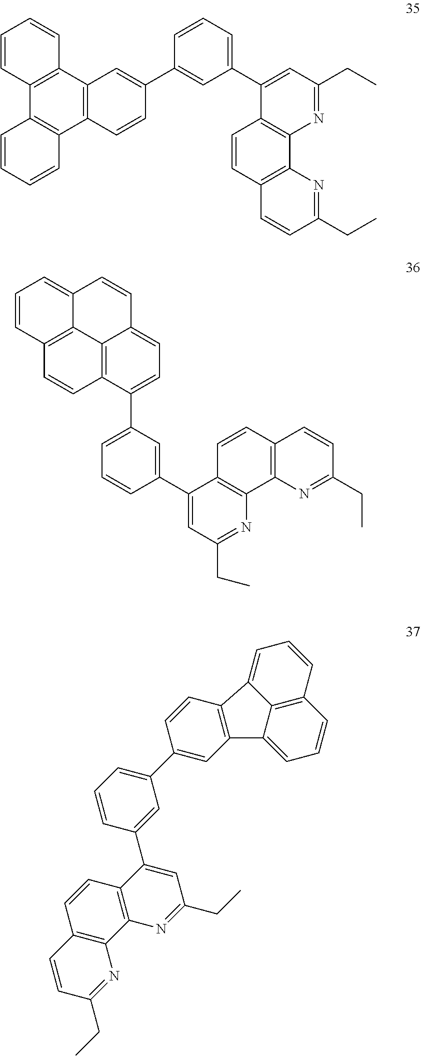 [SMILES: CCc1ccc2ccc3c(-c4cccc(-c5ccc6c7ccccc7c7ccccc7c6c5)c4)cc(CC)nc3c2n1 ; CCc1ccc2ccc3c(-c4cccc(-c5ccc6ccc7cccc8ccc5c6c78)c4)cc(CC)nc3c2n1 ; CCc1ccc2ccc3c(-c4cccc(-c5ccc6c(c5)-c5cccc7cccc-6c57)c4)cc(CC)nc3c2n1] 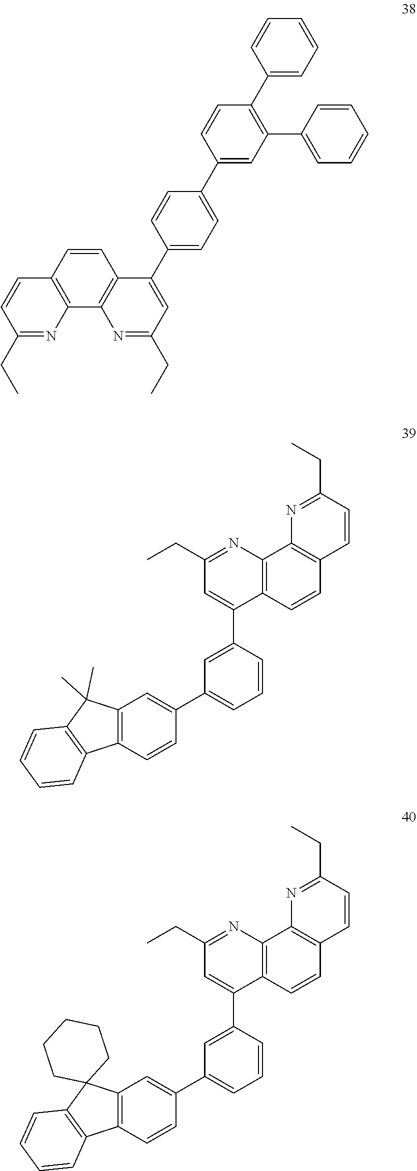 [SMILES: CCc1ccc2ccc3c(-c4ccc(-c5ccc(-c6ccccc6)c(-c6ccccc6)c5)cc4)cc(CC)nc3c2n1 ; CCc1ccc2ccc3c(-c4cccc(-c5ccc6c(c5)C(C)(C)c5ccccc5-6)c4)cc(CC)nc3c2n1 ; CCc1ccc2ccc3c(-c4cccc(-c5ccc6c(c5)C5(CCCCC5)c5ccccc5-6)c4)cc(CC)nc3c2n1] 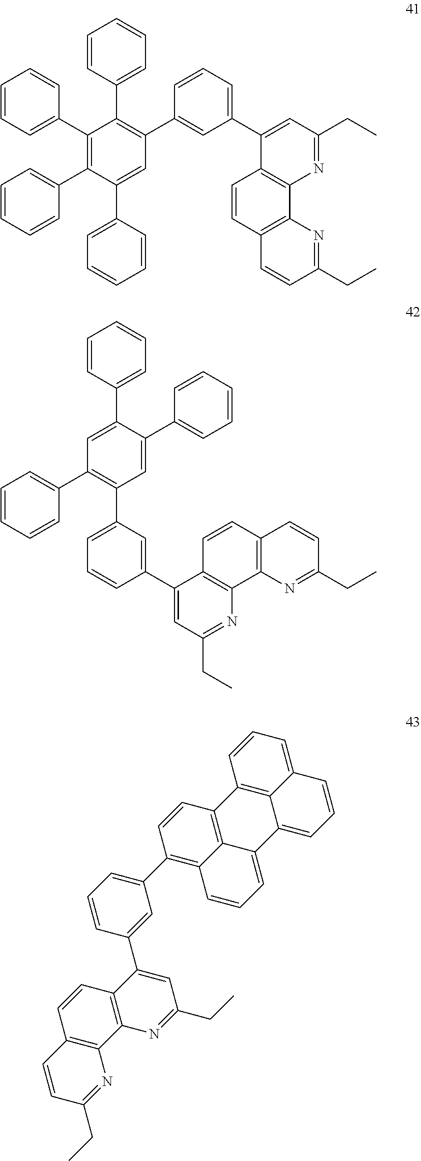 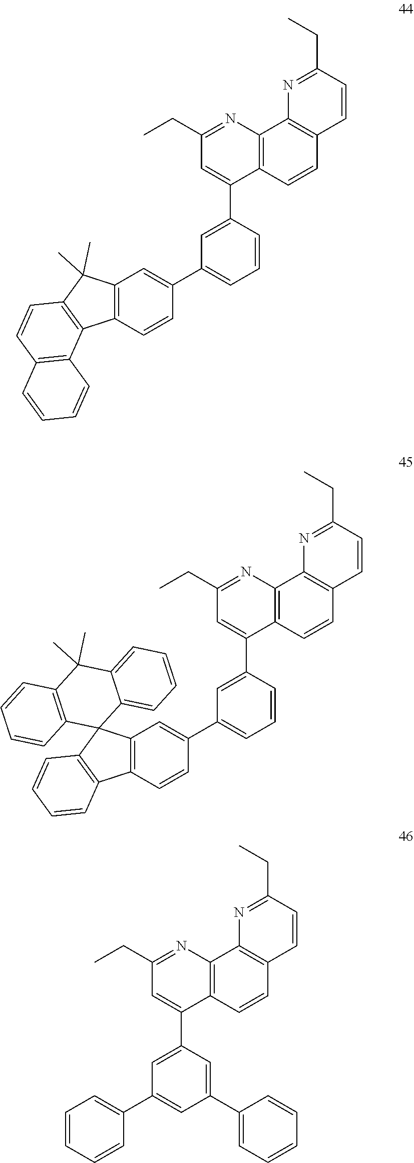 [SMILES: CCc1ccc2ccc3c(-c4cccc(-c5ccc6c(c5)C(C)(C)c5ccc7ccccc7c5-6)c4)cc(CC)nc3c2n1 ; CCc1ccc2ccc3c(-c4cccc(-c5ccc6c(c5)C5(c7ccccc7-6)c6ccccc6C(C)(C)c6ccccc65)c4)cc(CC)nc3c2n1 ; CCc1ccc2ccc3c(-c4cc(-c5ccccc5)cc(-c5ccccc5)c4)cc(CC)nc3c2n1] 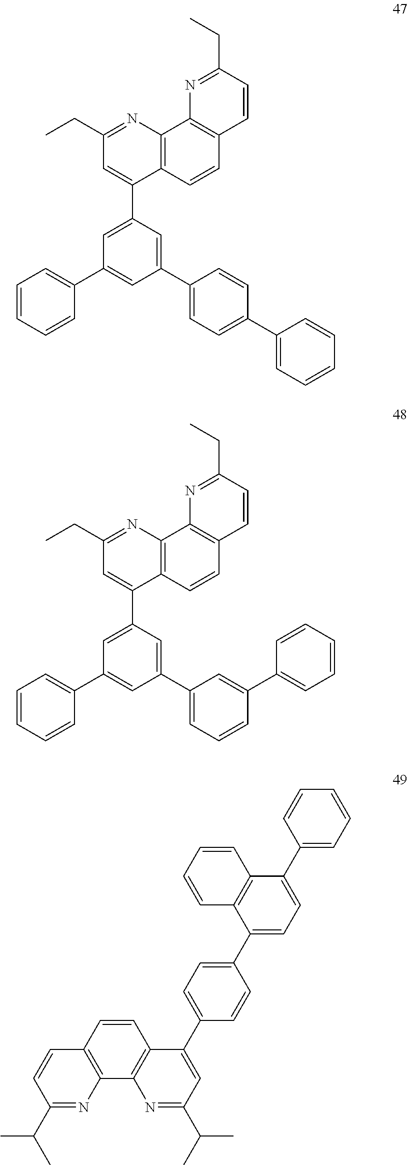 [SMILES: CCc1ccc2ccc3c(-c4cc(-c5ccccc5)cc(-c5ccc(-c6ccccc6)cc5)c4)cc(CC)nc3c2n1 ; CCc1ccc2ccc3c(-c4cc(-c5ccccc5)cc(-c5cccc(-c6ccccc6)c5)c4)cc(CC)nc3c2n1 ; CC(C)c1ccc2ccc3c(-c4ccc(-c5ccc(-c6ccccc6)c6ccccc56)cc4)cc(C(C)C)nc3c2n1] 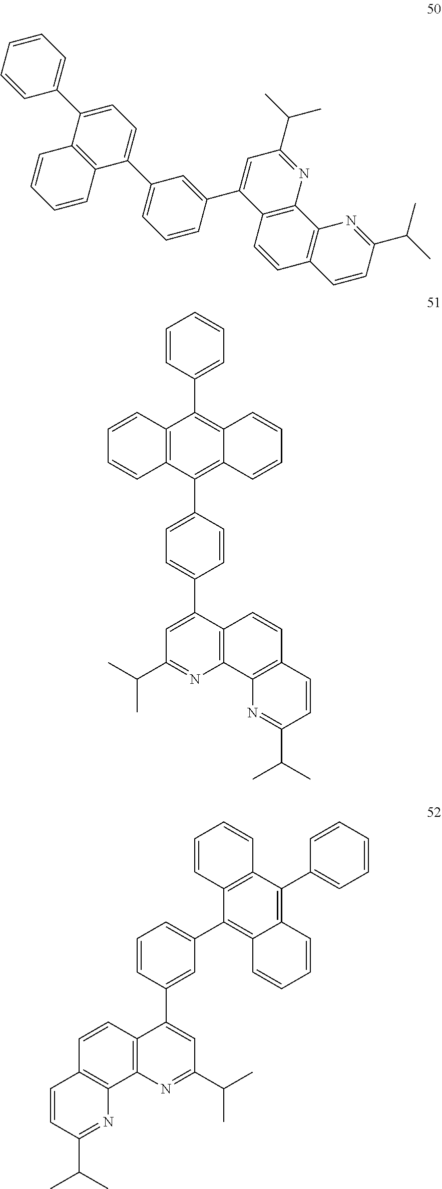 [SMILES: CC(C)c1ccc2ccc3c(-c4cccc(-c5ccc(-c6ccccc6)c6ccccc56)c4)cc(C(C)C)nc3c2n1 ; CC(C)c1ccc2ccc3c(-c4ccc(-c5c6ccccc6c(-c6ccccc6)c6ccccc56)cc4)cc(C(C)C)nc3c2n1 ; CC(C)c1ccc2ccc3c(-c4cccc(-c5c6ccccc6c(-c6ccccc6)c6ccccc56)c4)cc(C(C)C)nc3c2n1] 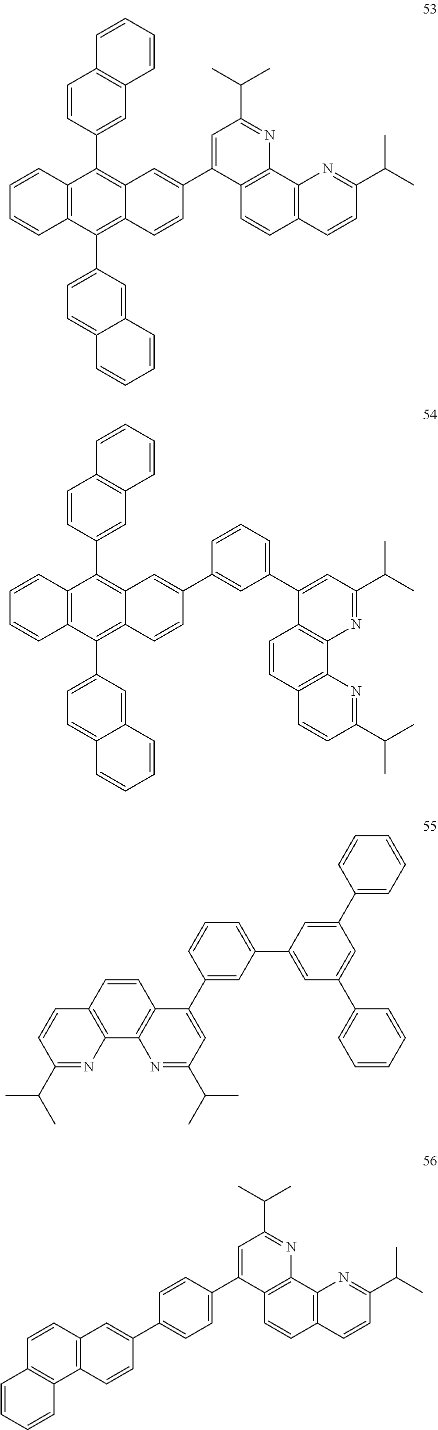 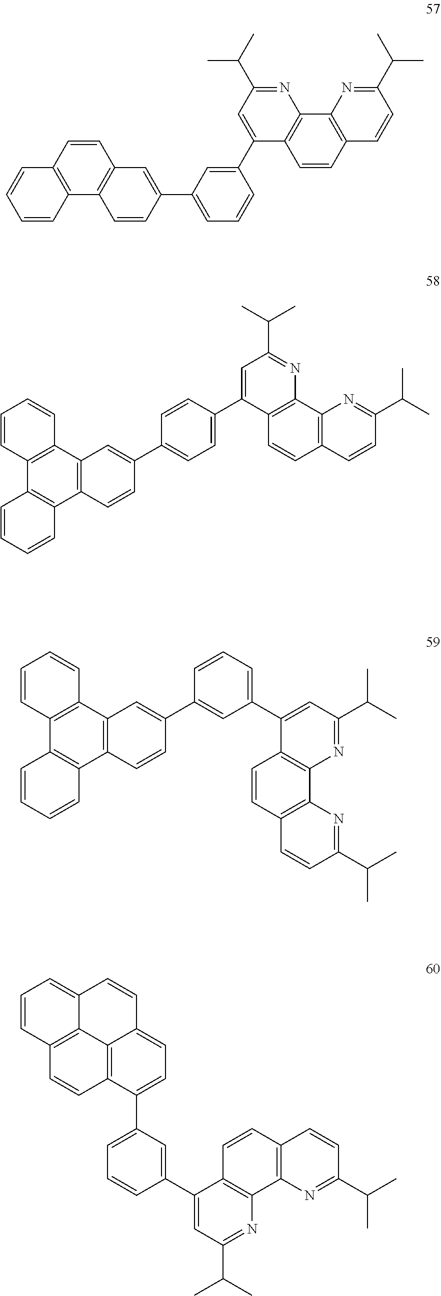 [SMILES: CC(C)c1ccc2ccc3c(-c4cccc(-c5ccc6c(ccc7ccccc76)c5)c4)cc(C(C)C)nc3c2n1 ; CC(C)c1ccc2ccc3c(-c4ccc(-c5ccc6c7ccccc7c7ccccc7c6c5)cc4)cc(C(C)C)nc3c2n1 ; CC(C)c1ccc2ccc3c(-c4cccc(-c5ccc6c7ccccc7c7ccccc7c6c5)c4)cc(C(C)C)nc3c2n1 ; CC(C)c1ccc2ccc3c(-c4cccc(-c5ccc6ccc7cccc8ccc5c6c78)c4)cc(C(C)C)nc3c2n1] 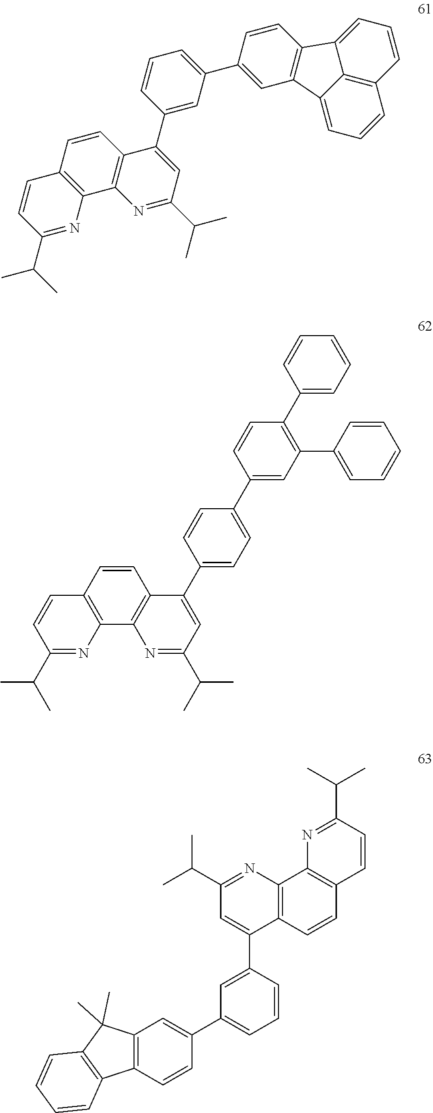 [SMILES: CC(C)c1ccc2ccc3c(-c4cccc(-c5ccc6c(c5)-c5cccc7cccc-6c57)c4)cc(C(C)C)nc3c2n1 ; CC(C)c1ccc2ccc3c(-c4ccc(-c5ccc(-c6ccccc6)c(-c6ccccc6)c5)cc4)cc(C(C)C)nc3c2n1 ; CC(C)c1ccc2ccc3c(-c4cccc(-c5ccc6c(c5)C(C)(C)c5ccccc5-6)c4)cc(C(C)C)nc3c2n1] 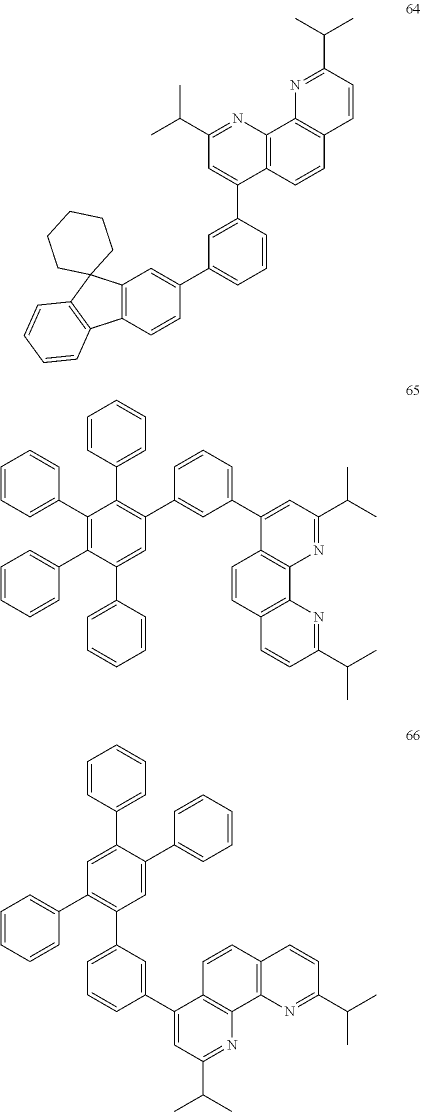 [SMILES: CC(C)c1ccc2ccc3c(-c4cccc(-c5ccc6c(c5)C5(CCCCC5)c5ccccc5-6)c4)cc(C(C)C)nc3c2n1 ; CC(C)c1ccc2ccc3c(-c4cccc(-c5cc(-c6ccccc6)c(-c6ccccc6)c(-c6ccccc6)c5-c5ccccc5)c4)cc(C(C)C)nc3c2n1 ; CC(C)c1ccc2ccc3c(-c4cccc(-c5cc(-c6ccccc6)c(-c6ccccc6)cc5-c5ccccc5)c4)cc(C(C)C)nc3c2n1] 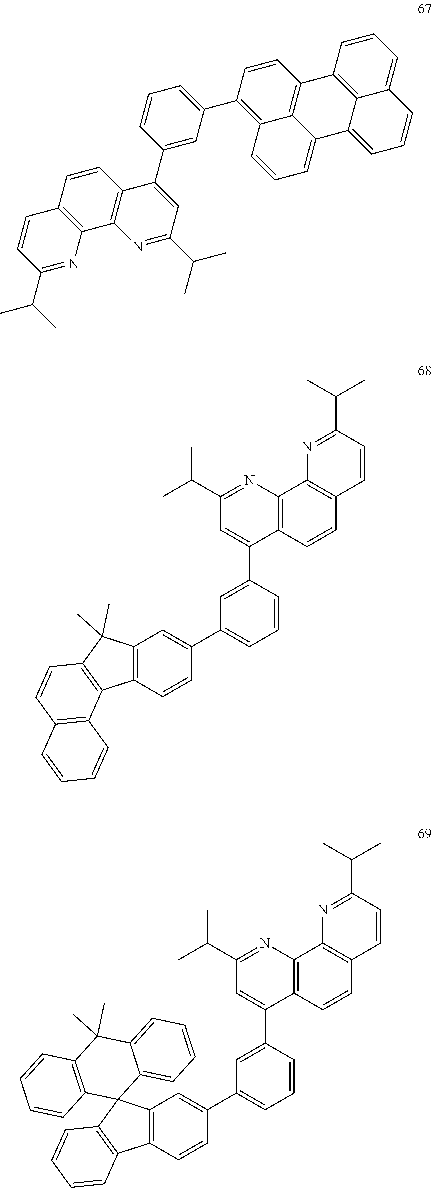 [SMILES: CC(C)c1ccc2ccc3c(-c4cccc(-c5ccc6c7cccc8cccc(c9cccc5c96)c87)c4)cc(C(C)C)nc3c2n1 ; CC(C)c1ccc2ccc3c(-c4cccc(-c5ccc6c(c5)C(C)(C)c5ccc7ccccc7c5-6)c4)cc(C(C)C)nc3c2n1 ; CC(C)c1ccc2ccc3c(-c4cccc(-c5ccc6c(c5)C5(c7ccccc7-6)c6ccccc6C(C)(C)c6ccccc65)c4)cc(C(C)C)nc3c2n1] 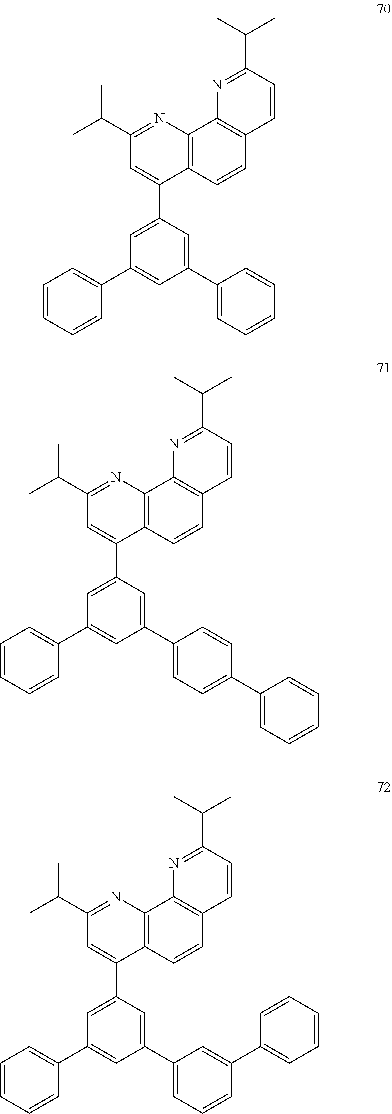 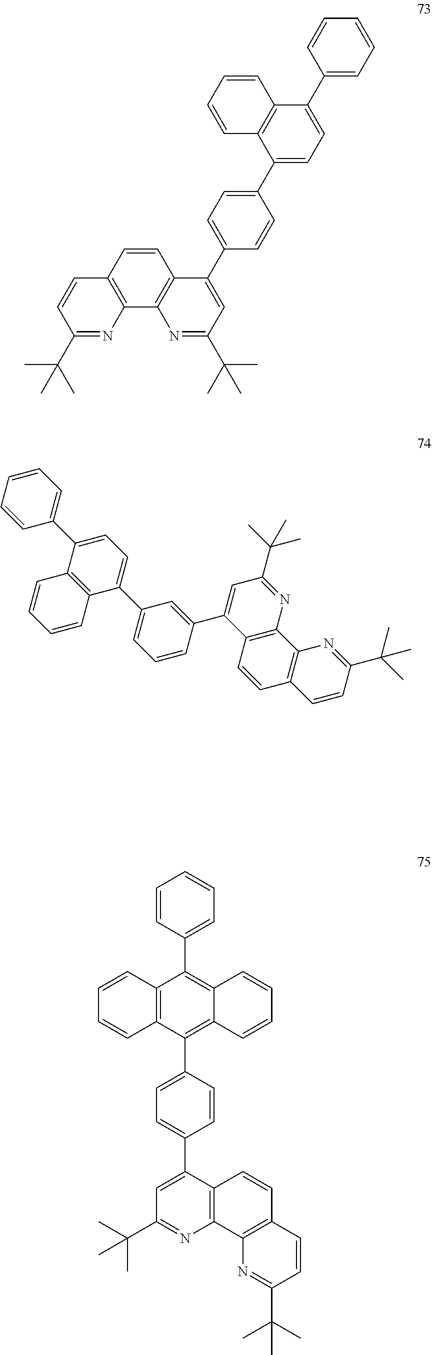 [SMILES: CC(C)(C)c1ccc2ccc3c(-c4ccc(-c5ccc(-c6ccccc6)c6ccccc56)cc4)cc(C(C)(C)C)nc3c2n1 ; CC(C)(C)c1ccc2ccc3c(-c4cccc(-c5ccc(-c6ccccc6)c6ccccc56)c4)cc(C(C)(C)C)nc3c2n1 ; CC(C)(C)c1ccc2ccc3c(-c4ccc(-c5c6ccccc6c(-c6ccccc6)c6ccccc56)cc4)cc(C(C)(C)C)nc3c2n1] 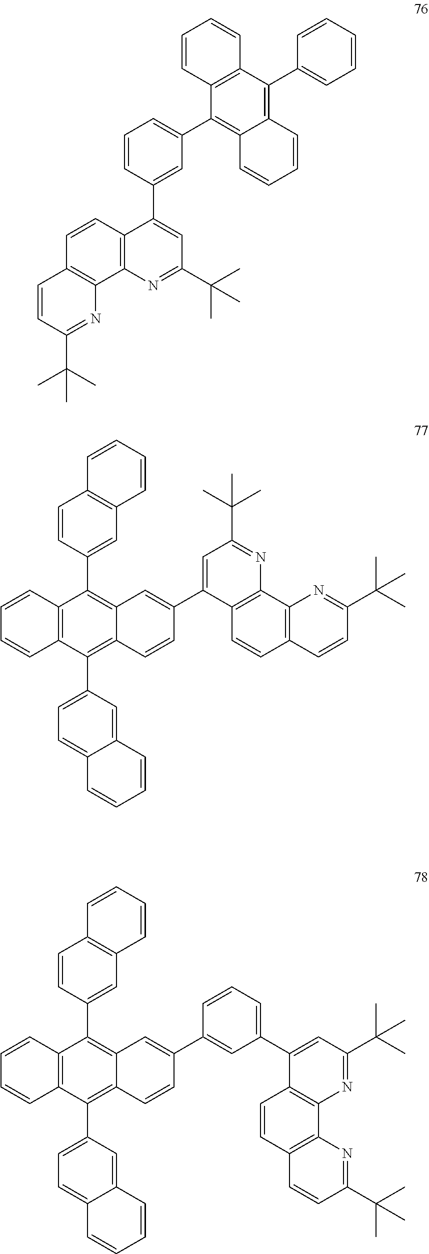 [SMILES: CC(C)(C)c1ccc2ccc3c(-c4cccc(-c5c6ccccc6c(-c6ccccc6)c6ccccc56)c4)cc(C(C)(C)C)nc3c2n1 ; CC(C)(C)c1ccc2ccc3c(-c4ccc5c(-c6ccc7ccccc7c6)c6ccccc6c(-c6ccc7ccccc7c6)c5c4)cc(C(C)(C)C)nc3c2n1 ; CC(C)(C)c1ccc2ccc3c(-c4cccc(-c5ccc6c(-c7ccc8ccccc8c7)c7ccccc7c(-c7ccc8ccccc8c7)c6c5)c4)cc(C(C)(C)C)nc3c2n1] 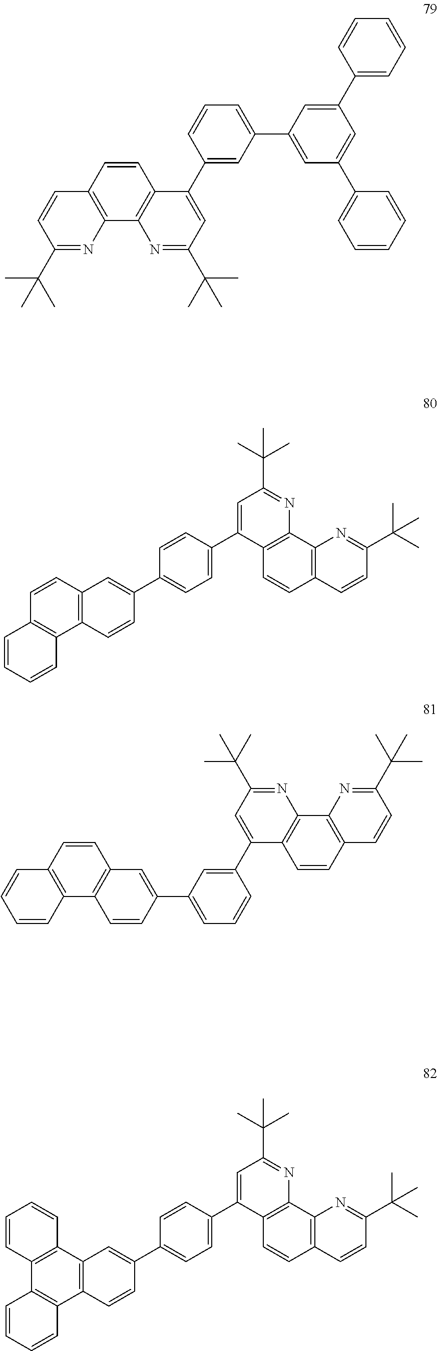 [SMILES: CC(C)(C)c1ccc2ccc3c(-c4cccc(-c5cc(-c6ccccc6)cc(-c6ccccc6)c5)c4)cc(C(C)(C)C)nc3c2n1 ; CC(C)(C)c1ccc2ccc3c(-c4ccc(-c5ccc6c(ccc7ccccc76)c5)cc4)cc(C(C)(C)C)nc3c2n1 ; CC(C)(C)c1ccc2ccc3c(-c4cccc(-c5ccc6c(ccc7ccccc76)c5)c4)cc(C(C)(C)C)nc3c2n1 ; CC(C)(C)c1ccc2ccc3c(-c4ccc(-c5ccc6c7ccccc7c7ccccc7c6c5)cc4)cc(C(C)(C)C)nc3c2n1] 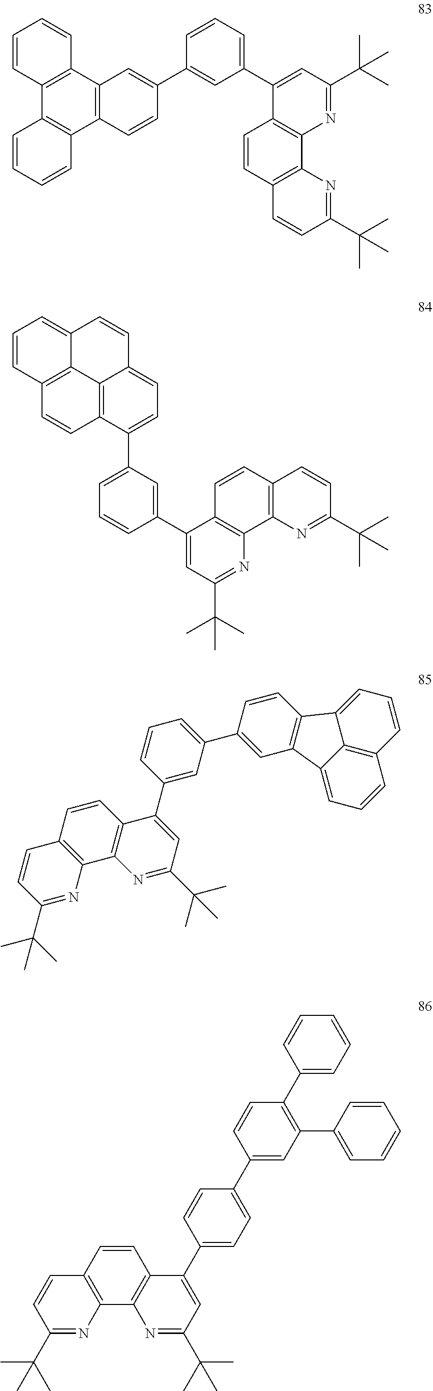 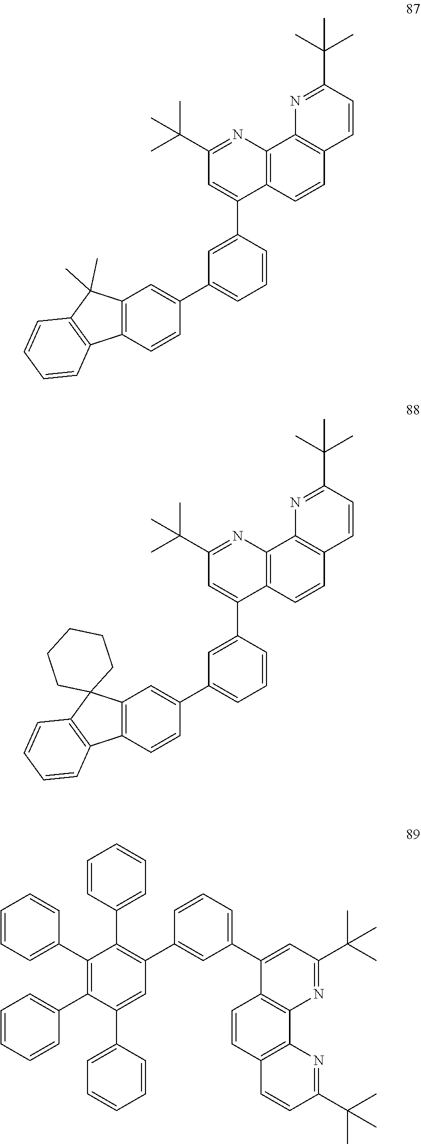 [SMILES: CC(C)(C)c1ccc2ccc3c(-c4cccc(-c5ccc6c(c5)C(C)(C)c5ccccc5-6)c4)cc(C(C)(C)C)nc3c2n1 ; CC(C)(C)c1ccc2ccc3c(-c4cccc(-c5ccc6c(c5)C5(CCCCC5)c5ccccc5-6)c4)cc(C(C)(C)C)nc3c2n1 ; CC(C)(C)c1ccc2ccc3c(-c4cccc(-c5cc(-c6ccccc6)c(-c6ccccc6)c(-c6ccccc6)c5-c5ccccc5)c4)cc(C(C)(C)C)nc3c2n1] 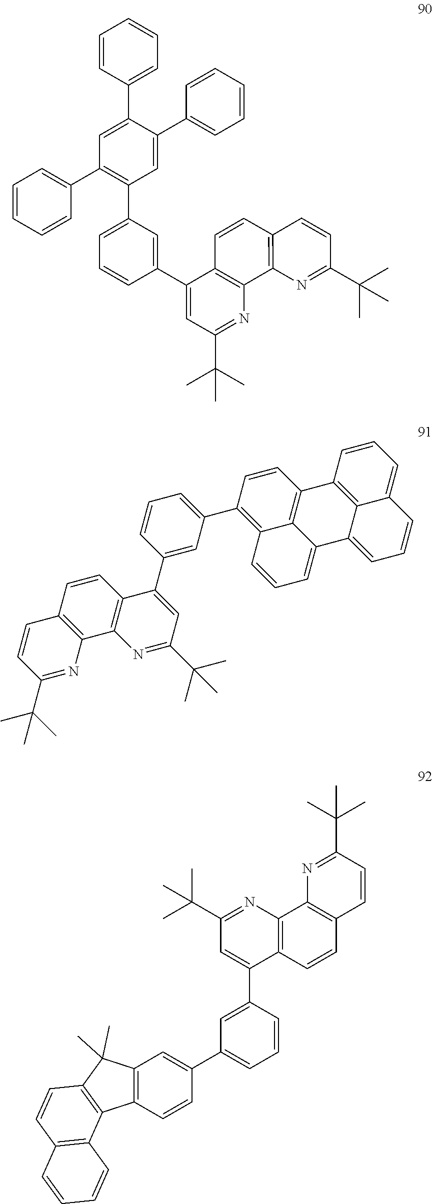 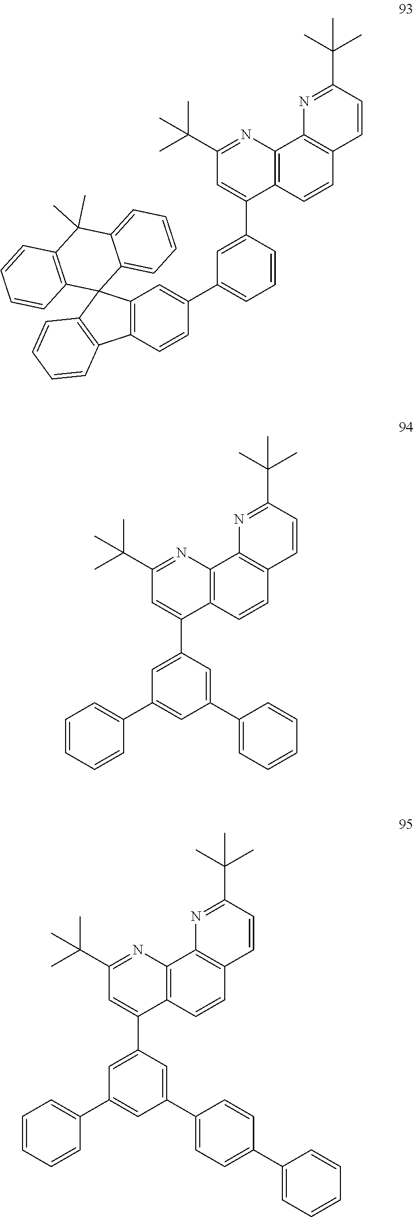 [SMILES: CC(C)(C)c1ccc2ccc3c(-c4cccc(-c5ccc6c(c5)C5(c7ccccc7-6)c6ccccc6C(C)(C)c6ccccc65)c4)cc(C(C)(C)C)nc3c2n1 ; CC(C)(C)c1ccc2ccc3c(-c4cc(-c5ccccc5)cc(-c5ccccc5)c4)cc(C(C)(C)C)nc3c2n1 ; CC(C)(C)c1ccc2ccc3c(-c4cc(-c5ccccc5)cc(-c5ccc(-c6ccccc6)cc5)c4)cc(C(C)(C)C)nc3c2n1] 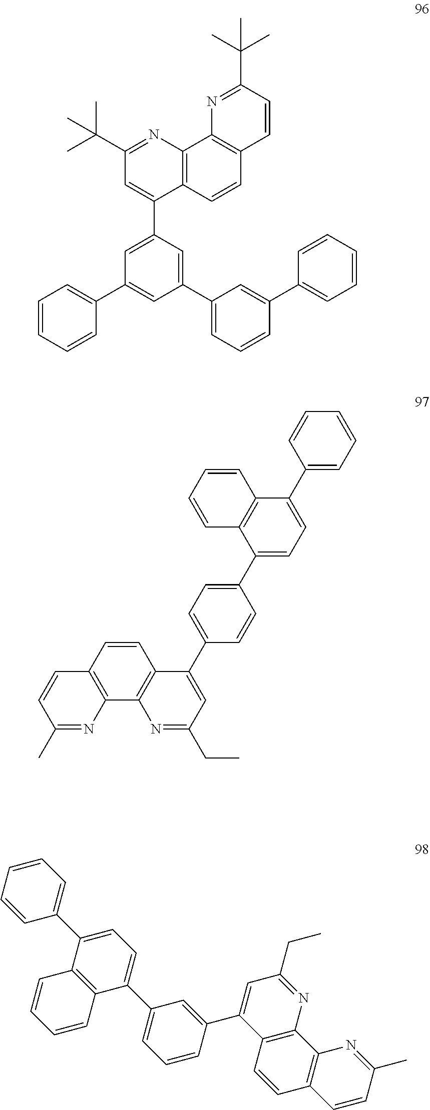 [SMILES: CC(C)(C)c1ccc2ccc3c(-c4cc(-c5ccccc5)cc(-c5cccc(-c6ccccc6)c5)c4)cc(C(C)(C)C)nc3c2n1 ; CCc1cc(-c2ccc(-c3ccc(-c4ccccc4)c4ccccc34)cc2)c2ccc3ccc(C)nc3c2n1 ; CCc1cc(-c2cccc(-c3ccc(-c4ccccc4)c4ccccc34)c2)c2ccc3ccc(C)nc3c2n1] 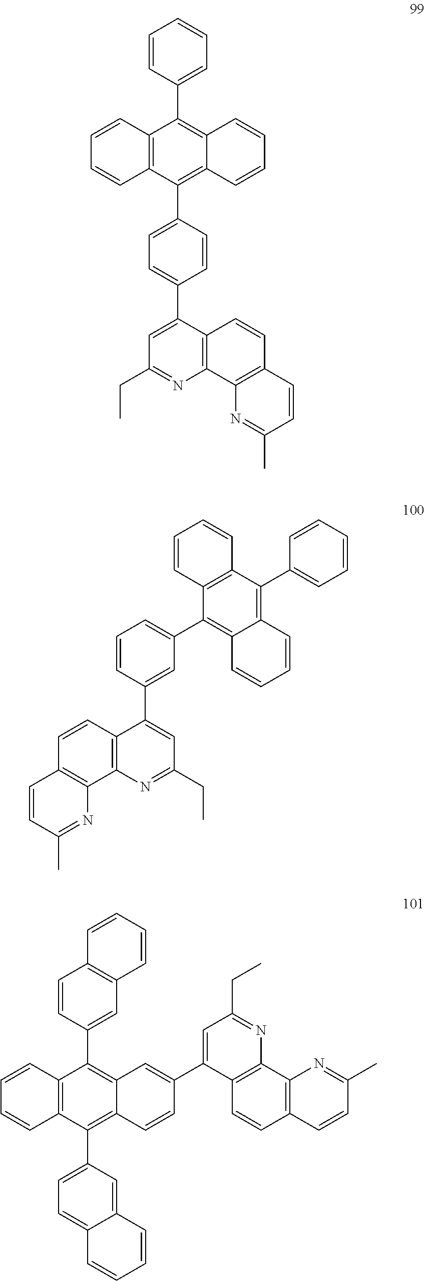 [SMILES: CCc1cc(-c2ccc(-c3c4ccccc4c(-c4ccccc4)c4ccccc34)cc2)c2ccc3ccc(C)nc3c2n1 ; CCc1cc(-c2cccc(-c3c4ccccc4c(-c4ccccc4)c4ccccc34)c2)c2ccc3ccc(C)nc3c2n1 ; CCc1cc(-c2ccc3c(-c4ccc5ccccc5c4)c4ccccc4c(-c4ccc5ccccc5c4)c3c2)c2ccc3ccc(C)nc3c2n1] 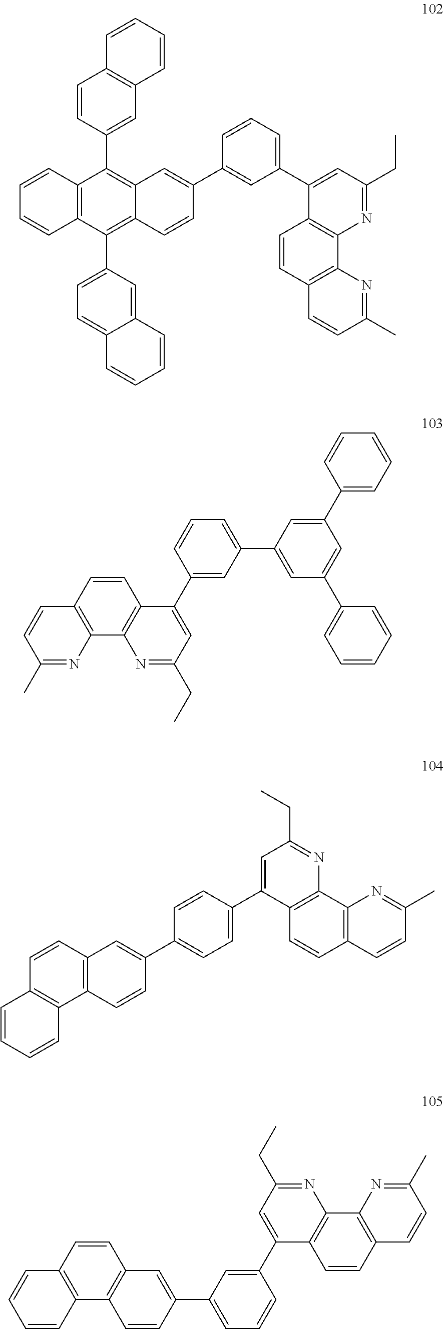 [SMILES: CCc1cc(-c2cccc(-c3ccc4c(-c5ccc6ccccc6c5)c5ccccc5c(-c5ccc6ccccc6c5)c4c3)c2)c2ccc3ccc(C)nc3c2n1 ; CCc1cc(-c2cccc(-c3cc(-c4ccccc4)cc(-c4ccccc4)c3)c2)c2ccc3ccc(C)nc3c2n1 ; CCc1cc(-c2ccc(-c3ccc4c(ccc5ccccc54)c3)cc2)c2ccc3ccc(C)nc3c2n1 ; CCc1cc(-c2cccc(-c3ccc4c(ccc5ccccc54)c3)c2)c2ccc3ccc(C)nc3c2n1] 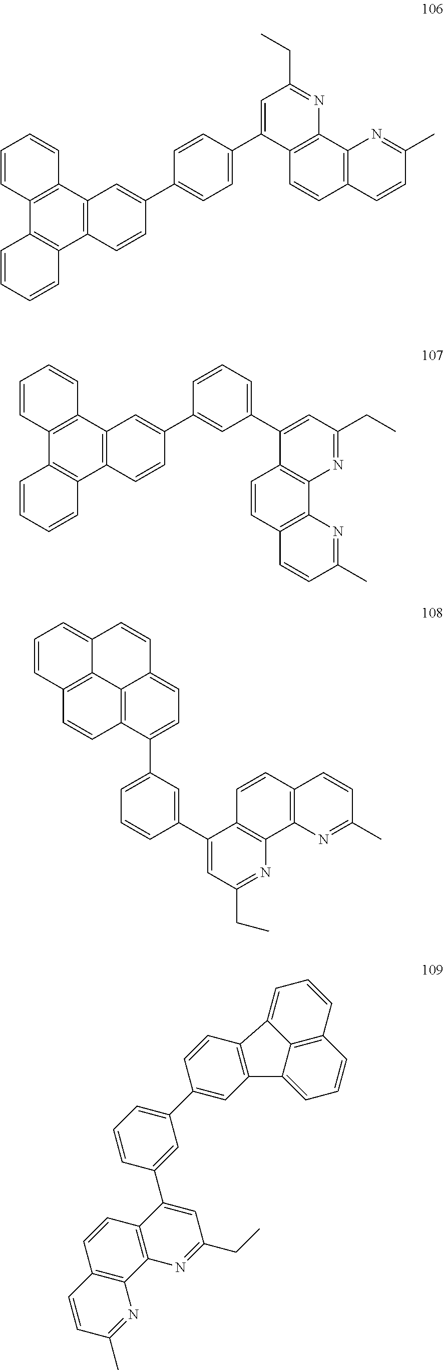 [SMILES: CCc1cc(-c2ccc(-c3ccc4c5ccccc5c5ccccc5c4c3)cc2)c2ccc3ccc(C)nc3c2n1 ; CCc1cc(-c2cccc(-c3ccc4c5ccccc5c5ccccc5c4c3)c2)c2ccc3ccc(C)nc3c2n1 ; CCc1cc(-c2cccc(-c3ccc4ccc5cccc6ccc3c4c56)c2)c2ccc3ccc(C)nc3c2n1 ; CCc1cc(-c2cccc(-c3ccc4c(c3)-c3cccc5cccc-4c35)c2)c2ccc3ccc(C)nc3c2n1] 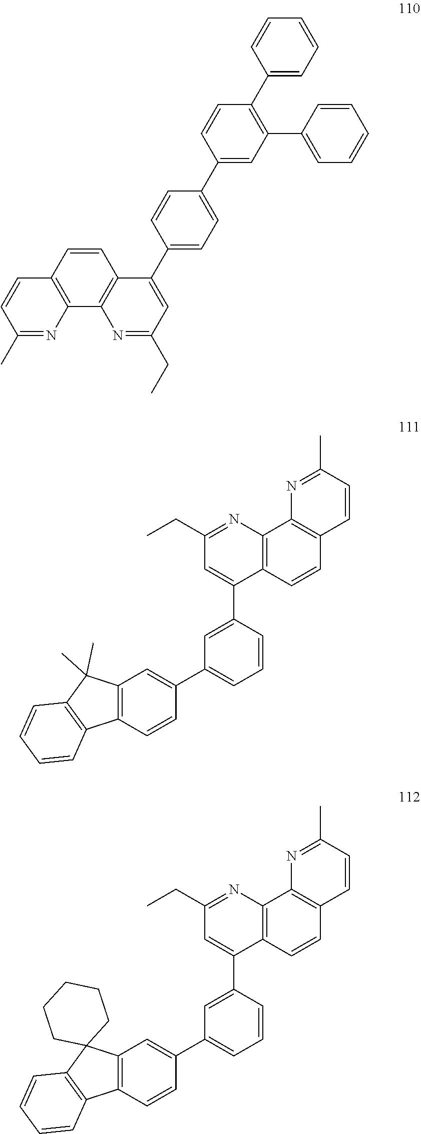 [SMILES: CCc1cc(-c2ccc(-c3ccc(-c4ccccc4)c(-c4ccccc4)c3)cc2)c2ccc3ccc(C)nc3c2n1 ; CCc1cc(-c2cccc(-c3ccc4c(c3)C(C)(C)c3ccccc3-4)c2)c2ccc3ccc(C)nc3c2n1 ; CCc1cc(-c2cccc(-c3ccc4c(c3)C3(CCCCC3)c3ccccc3-4)c2)c2ccc3ccc(C)nc3c2n1] 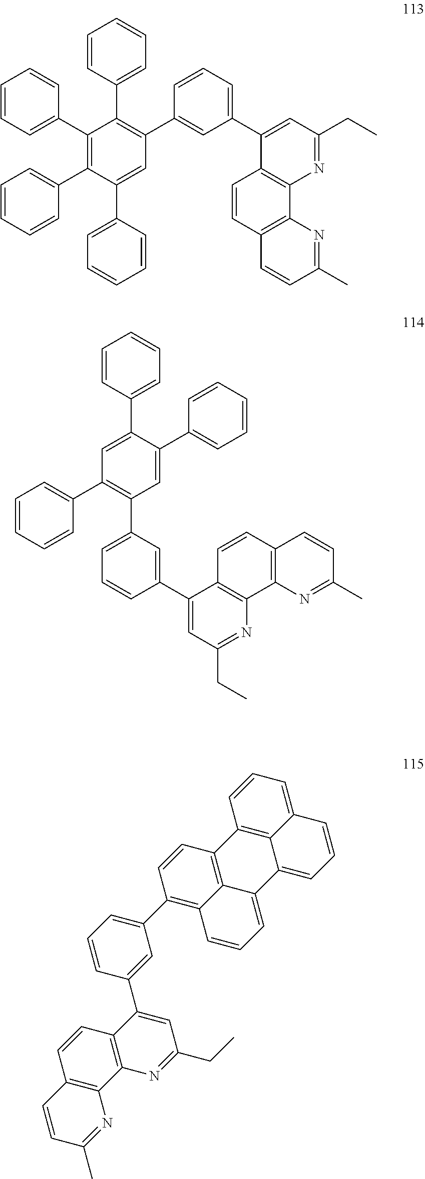 [SMILES: CCc1cc(-c2cccc(-c3cc(-c4ccccc4)c(-c4ccccc4)c(-c4ccccc4)c3-c3ccccc3)c2)c2ccc3ccc(C)nc3c2n1 ; CCc1cc(-c2cccc(-c3cc(-c4ccccc4)c(-c4ccccc4)cc3-c3ccccc3)c2)c2ccc3ccc(C)nc3c2n1 ; CCc1cc(-c2cccc(-c3ccc4c5cccc6cccc(c7cccc3c74)c65)c2)c2ccc3ccc(C)nc3c2n1] 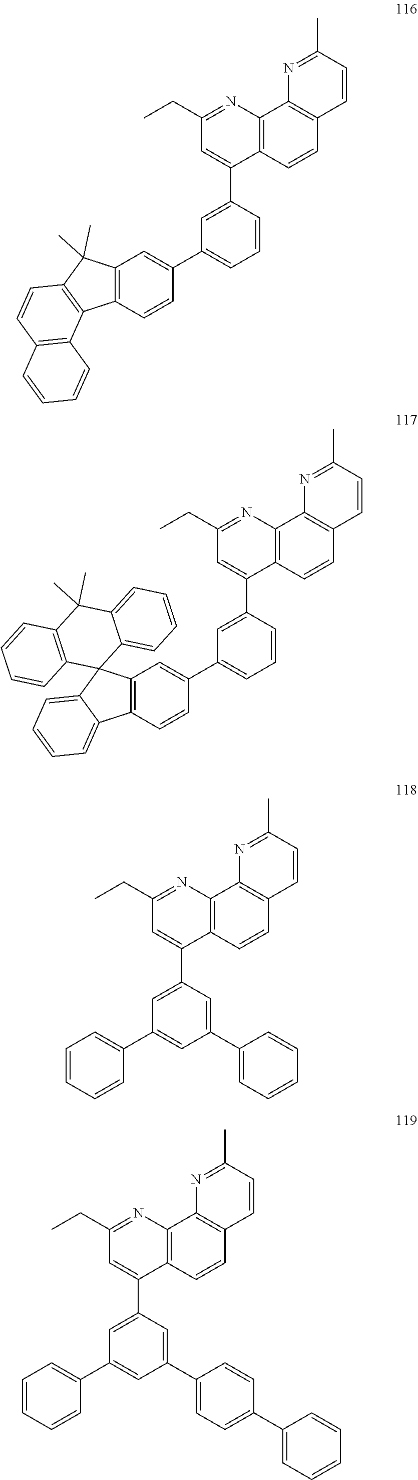 [SMILES: CCc1cc(-c2cccc(-c3ccc4c(c3)C(C)(C)c3ccc5ccccc5c3-4)c2)c2ccc3ccc(C)nc3c2n1 ; CCc1cc(-c2cccc(-c3ccc4c(c3)C3(c5ccccc5-4)c4ccccc4C(C)(C)c4ccccc43)c2)c2ccc3ccc(C)nc3c2n1 ; CCc1cc(-c2cc(-c3ccccc3)cc(-c3ccccc3)c2)c2ccc3ccc(C)nc3c2n1 ; CCc1cc(-c2cc(-c3ccccc3)cc(-c3ccc(-c4ccccc4)cc3)c2)c2ccc3ccc(C)nc3c2n1] 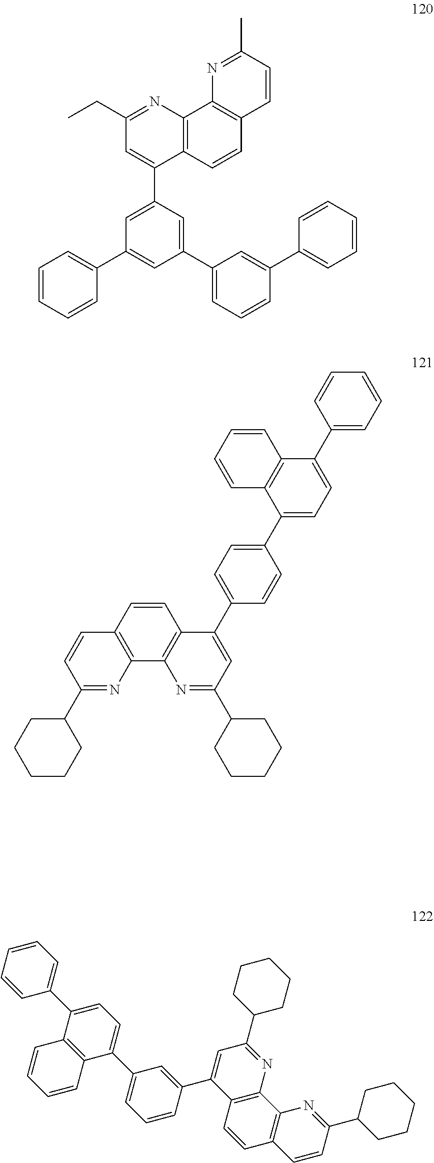 [SMILES: CCc1cc(-c2cc(-c3ccccc3)cc(-c3cccc(-c4ccccc4)c3)c2)c2ccc3ccc(C)nc3c2n1 ; c1ccc(-c2ccc(-c3ccc(-c4cc(C5CCCCC5)nc5c4ccc4ccc(C6CCCCC6)nc45)cc3)c3ccccc23)cc1 ; c1ccc(-c2ccc(-c3cccc(-c4cc(C5CCCCC5)nc5c4ccc4ccc(C6CCCCC6)nc45)c3)c3ccccc23)cc1] 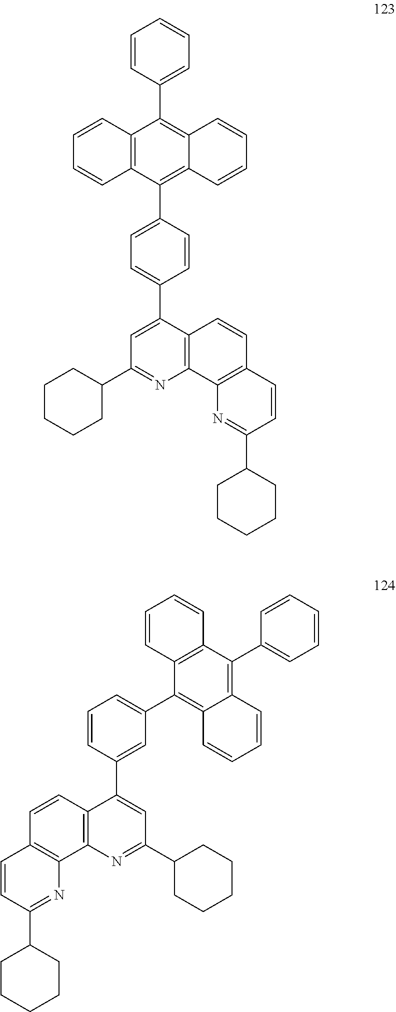 [SMILES: c1ccc(-c2c3ccccc3c(-c3ccc(-c4cc(C5CCCCC5)nc5c4ccc4ccc(C6CCCCC6)nc45)cc3)c3ccccc23)cc1 ; c1ccc(-c2c3ccccc3c(-c3cccc(-c4cc(C5CCCCC5)nc5c4ccc4ccc(C6CCCCC6)nc45)c3)c3ccccc23)cc1] 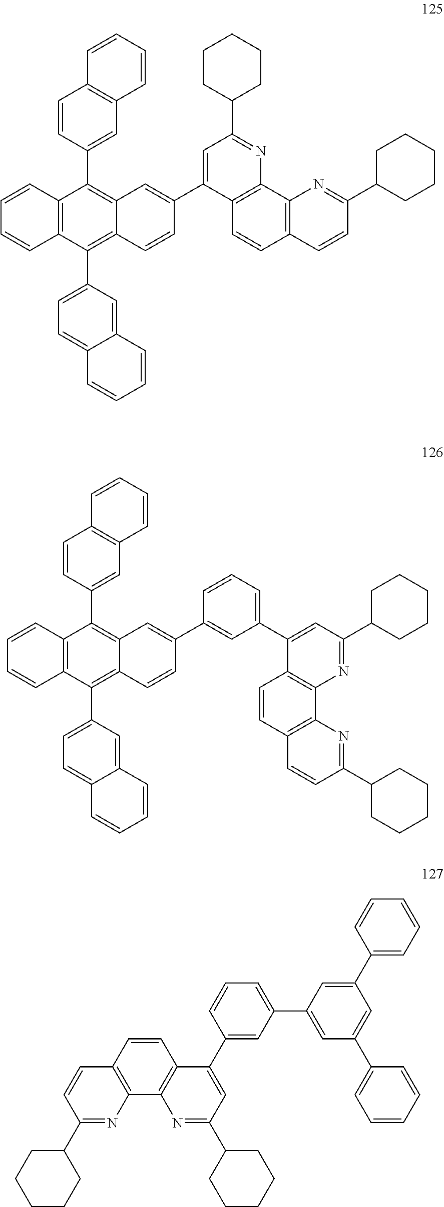 [SMILES: c1ccc2cc(-c3c4ccccc4c(-c4ccc5ccccc5c4)c4cc(-c5cc(C6CCCCC6)nc6c5ccc5ccc(C7CCCCC7)nc56)ccc34)ccc2c1 ; c1cc(-c2ccc3c(-c4ccc5ccccc5c4)c4ccccc4c(-c4ccc5ccccc5c4)c3c2)cc(-c2cc(C3CCCCC3)nc3c2ccc2ccc(C4CCCCC4)nc23)c1 ; c1ccc(-c2cc(-c3ccccc3)cc(-c3cccc(-c4cc(C5CCCCC5)nc5c4ccc4ccc(C6CCCCC6)nc45)c3)c2)cc1] 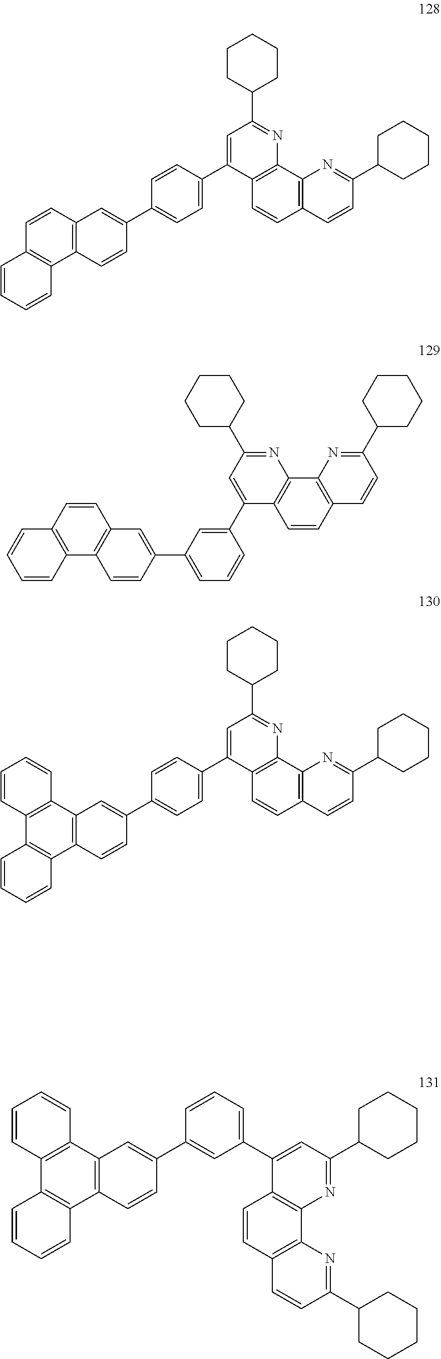 [SMILES: c1ccc2c(c1)ccc1cc(-c3ccc(-c4cc(C5CCCCC5)nc5c4ccc4ccc(C6CCCCC6)nc45)cc3)ccc12 ; c1cc(-c2ccc3c(ccc4ccccc43)c2)cc(-c2cc(C3CCCCC3)nc3c2ccc2ccc(C4CCCCC4)nc23)c1 ; c1ccc2c(c1)c1ccccc1c1cc(-c3ccc(-c4cc(C5CCCCC5)nc5c4ccc4ccc(C6CCCCC6)nc45)cc3)ccc21 ; c1cc(-c2ccc3c4ccccc4c4ccccc4c3c2)cc(-c2cc(C3CCCCC3)nc3c2ccc2ccc(C4CCCCC4)nc23)c1] 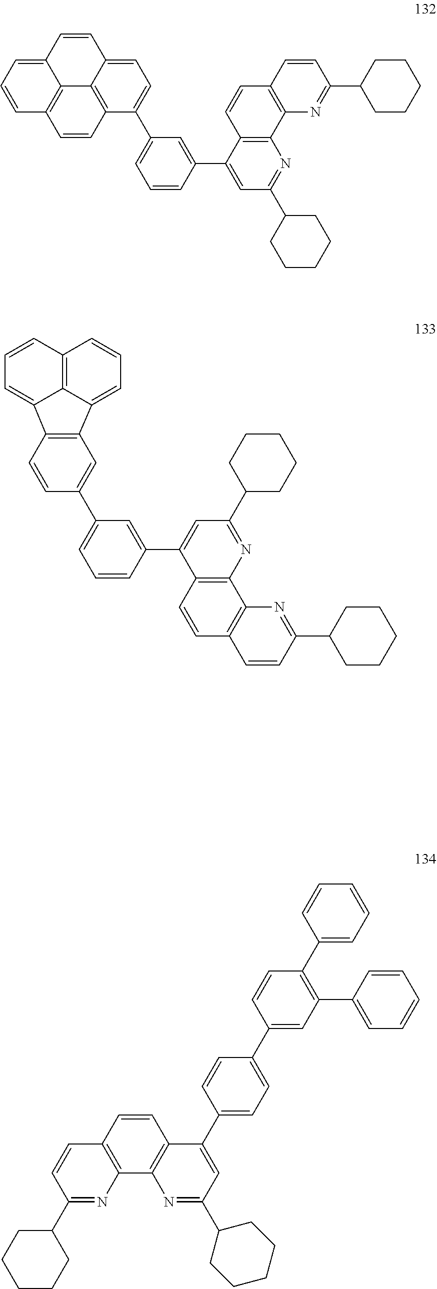 [SMILES: c1cc(-c2cc(C3CCCCC3)nc3c2ccc2ccc(C4CCCCC4)nc23)cc(-c2ccc3ccc4cccc5ccc2c3c45)c1 ; c1cc(-c2ccc3c(c2)-c2cccc4cccc-3c24)cc(-c2cc(C3CCCCC3)nc3c2ccc2ccc(C4CCCCC4)nc23)c1 ; c1ccc(-c2ccc(-c3ccc(-c4cc(C5CCCCC5)nc5c4ccc4ccc(C6CCCCC6)nc45)cc3)cc2-c2ccccc2)cc1] 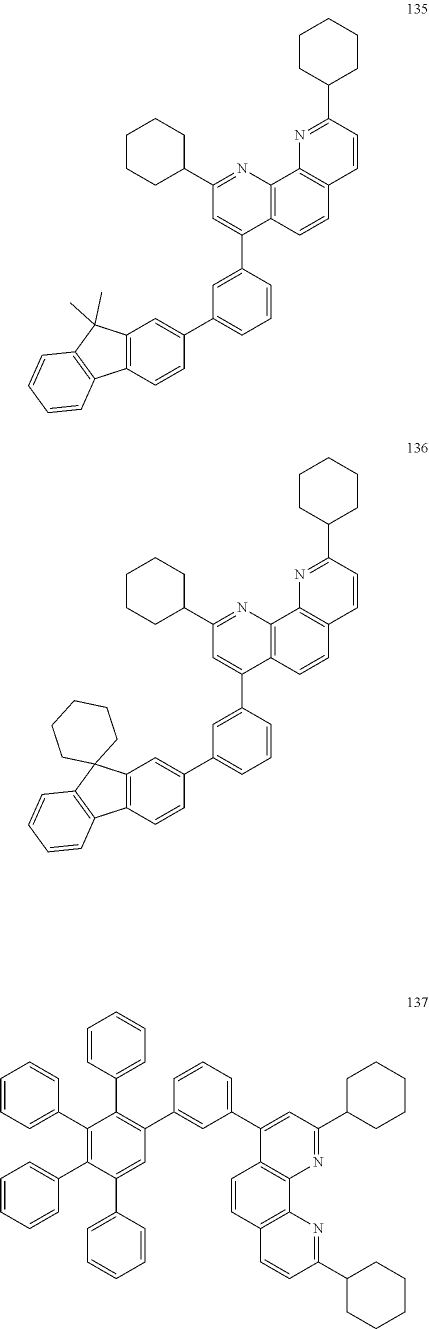 [SMILES: CC1(C)c2ccccc2-c2ccc(-c3cccc(-c4cc(C5CCCCC5)nc5c4ccc4ccc(C6CCCCC6)nc45)c3)cc21 ; c1cc(-c2ccc3c(c2)C2(CCCCC2)c2ccccc2-3)cc(-c2cc(C3CCCCC3)nc3c2ccc2ccc(C4CCCCC4)nc23)c1 ; c1ccc(-c2cc(-c3cccc(-c4cc(C5CCCCC5)nc5c4ccc4ccc(C6CCCCC6)nc45)c3)c(-c3ccccc3)c(-c3ccccc3)c2-c2ccccc2)cc1] 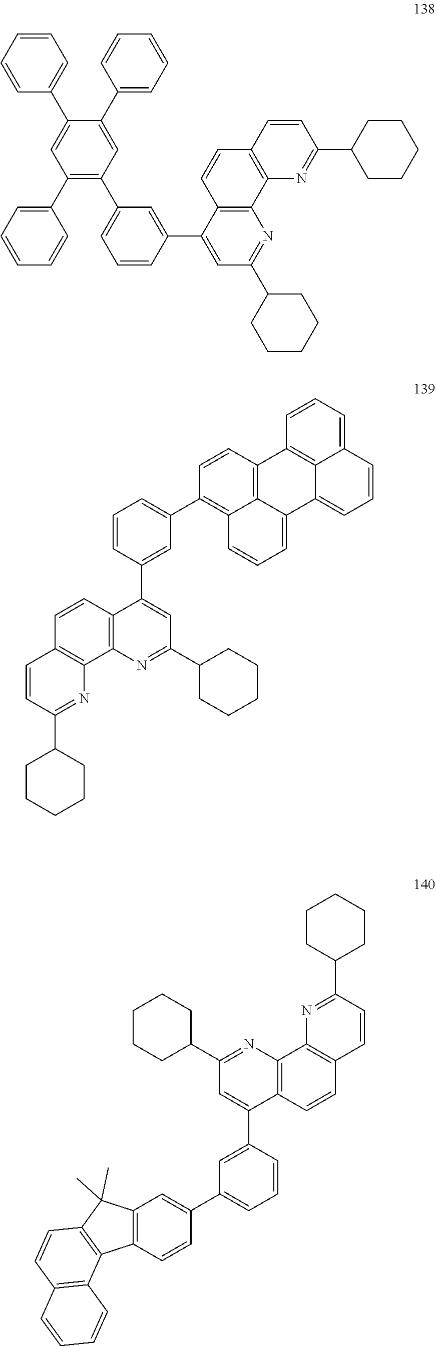 [SMILES: c1ccc(-c2cc(-c3ccccc3)c(-c3cccc(-c4cc(C5CCCCC5)nc5c4ccc4ccc(C6CCCCC6)nc45)c3)cc2-c2ccccc2)cc1 ; c1cc(-c2cc(C3CCCCC3)nc3c2ccc2ccc(C4CCCCC4)nc23)cc(-c2ccc3c4cccc5cccc(c6cccc2c63)c54)c1 ; CC1(C)c2cc(-c3cccc(-c4cc(C5CCCCC5)nc5c4ccc4ccc(C6CCCCC6)nc45)c3)ccc2-c2c1ccc1ccccc21] 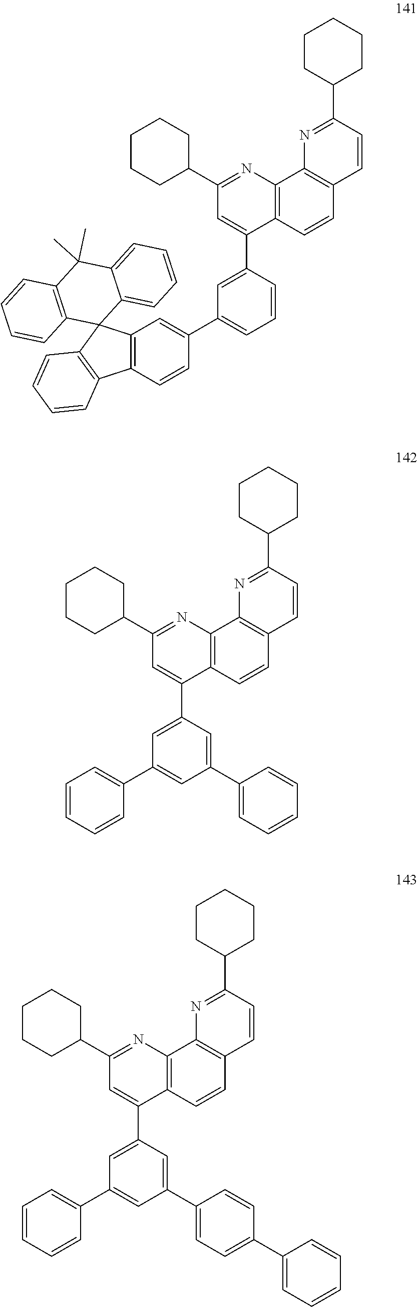 [SMILES: CC1(C)c2ccccc2C2(c3ccccc3-c3ccc(-c4cccc(-c5cc(C6CCCCC6)nc6c5ccc5ccc(C7CCCCC7)nc56)c4)cc32)c2ccccc21 ; c1ccc(-c2cc(-c3ccccc3)cc(-c3cc(C4CCCCC4)nc4c3ccc3ccc(C5CCCCC5)nc34)c2)cc1 ; c1ccc(-c2ccc(-c3cc(-c4ccccc4)cc(-c4cc(C5CCCCC5)nc5c4ccc4ccc(C6CCCCC6)nc45)c3)cc2)cc1] 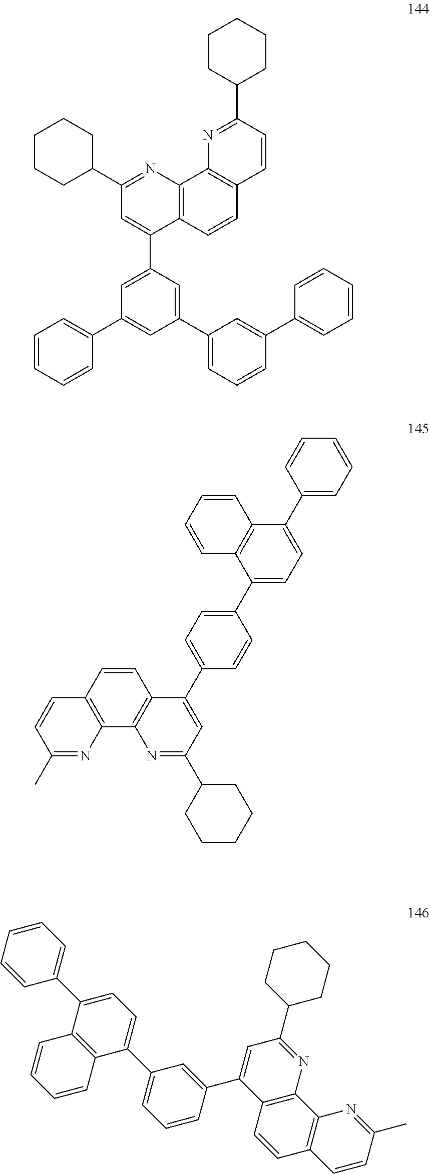 [SMILES: c1ccc(-c2cccc(-c3cc(-c4ccccc4)cc(-c4cc(C5CCCCC5)nc5c4ccc4ccc(C6CCCCC6)nc45)c3)c2)cc1 ; Cc1ccc2ccc3c(-c4ccc(-c5ccc(-c6ccccc6)c6ccccc56)cc4)cc(C4CCCCC4)nc3c2n1 ; Cc1ccc2ccc3c(-c4cccc(-c5ccc(-c6ccccc6)c6ccccc56)c4)cc(C4CCCCC4)nc3c2n1] 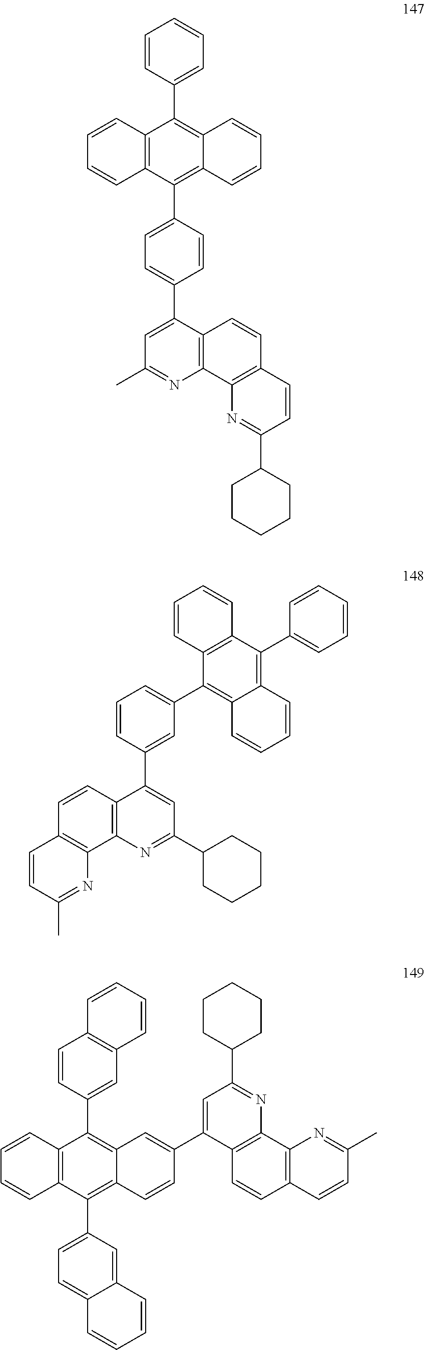 [SMILES: Cc1cc(-c2ccc(-c3c4ccccc4c(-c4ccccc4)c4ccccc34)cc2)c2ccc3ccc(C4CCCCC4)nc3c2n1 ; Cc1ccc2ccc3c(-c4cccc(-c5c6ccccc6c(-c6ccccc6)c6ccccc56)c4)cc(C4CCCCC4)nc3c2n1 ; Cc1ccc2ccc3c(-c4ccc5c(-c6ccc7ccccc7c6)c6ccccc6c(-c6ccc7ccccc7c6)c5c4)cc(C4CCCCC4)nc3c2n1] 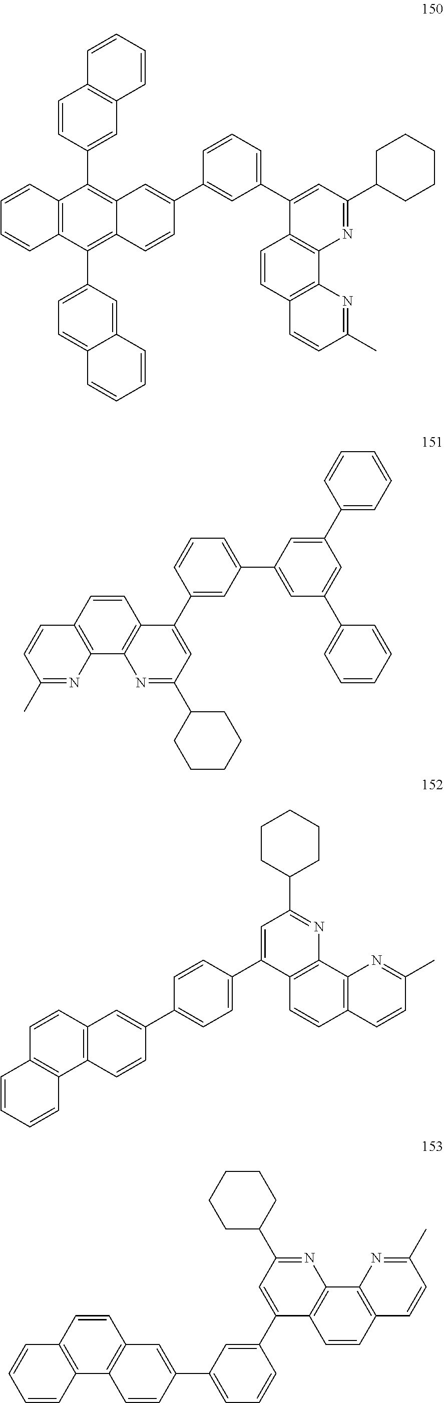 [SMILES: Cc1ccc2ccc3c(-c4cccc(-c5ccc6c(-c7ccc8ccccc8c7)c7ccccc7c(-c7ccc8ccccc8c7)c6c5)c4)cc(C4CCCCC4)nc3c2n1 ; Cc1ccc2ccc3c(-c4cccc(-c5cc(-c6ccccc6)cc(-c6ccccc6)c5)c4)cc(C4CCCCC4)nc3c2n1 ; Cc1ccc2ccc3c(-c4ccc(-c5ccc6c(ccc7ccccc76)c5)cc4)cc(C4CCCCC4)nc3c2n1 ; Cc1ccc2ccc3c(-c4cccc(-c5ccc6c(ccc7ccccc76)c5)c4)cc(C4CCCCC4)nc3c2n1] 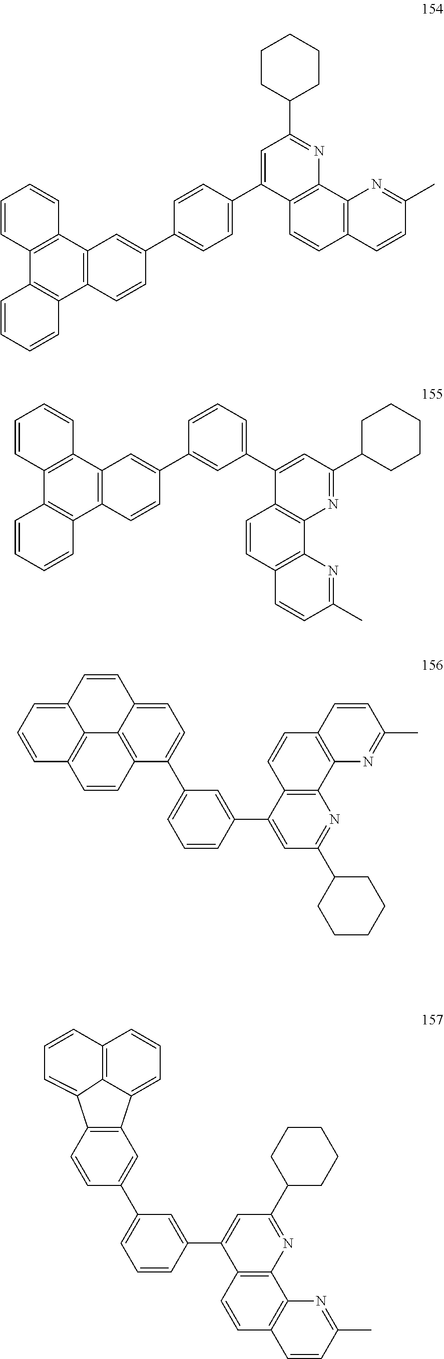 [SMILES: Cc1ccc2ccc3c(-c4ccc(-c5ccc6c7ccccc7c7ccccc7c6c5)cc4)cc(C4CCCCC4)nc3c2n1 ; Cc1ccc2ccc3c(-c4cccc(-c5ccc6c7ccccc7c7ccccc7c6c5)c4)cc(C4CCCCC4)nc3c2n1 ; Cc1ccc2ccc3c(-c4cccc(-c5ccc6ccc7cccc8ccc5c6c78)c4)cc(C4CCCCC4)nc3c2n1 ; Cc1ccc2ccc3c(-c4cccc(-c5ccc6c(c5)-c5cccc7cccc-6c57)c4)cc(C4CCCCC4)nc3c2n1] 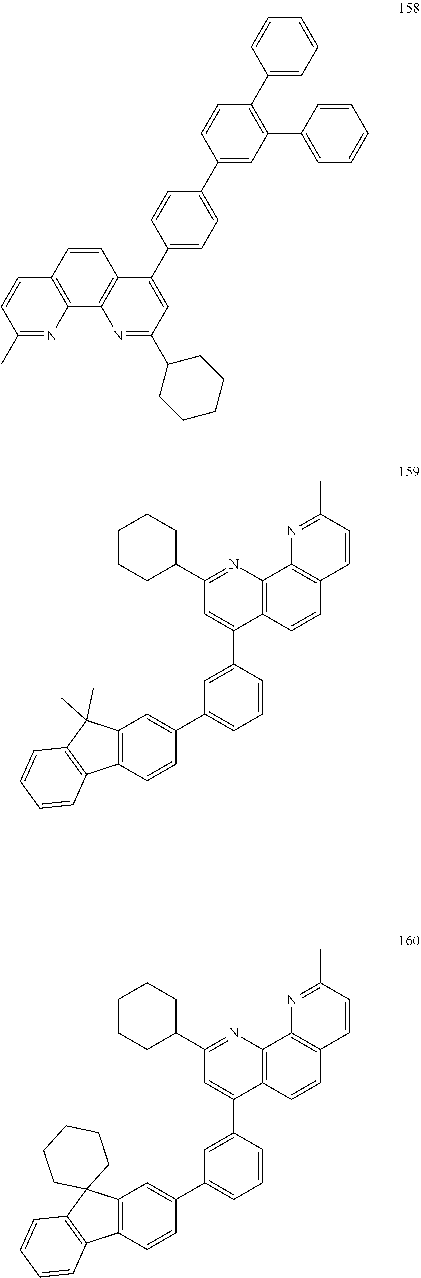 [SMILES: Cc1ccc2ccc3c(-c4ccc(-c5ccc(-c6ccccc6)c(-c6ccccc6)c5)cc4)cc(C4CCCCC4)nc3c2n1 ; Cc1ccc2ccc3c(-c4cccc(-c5ccc6c(c5)C(C)(C)c5ccccc5-6)c4)cc(C4CCCCC4)nc3c2n1 ; Cc1ccc2ccc3c(-c4cccc(-c5ccc6c(c5)C5(CCCCC5)c5ccccc5-6)c4)cc(C4CCCCC4)nc3c2n1] 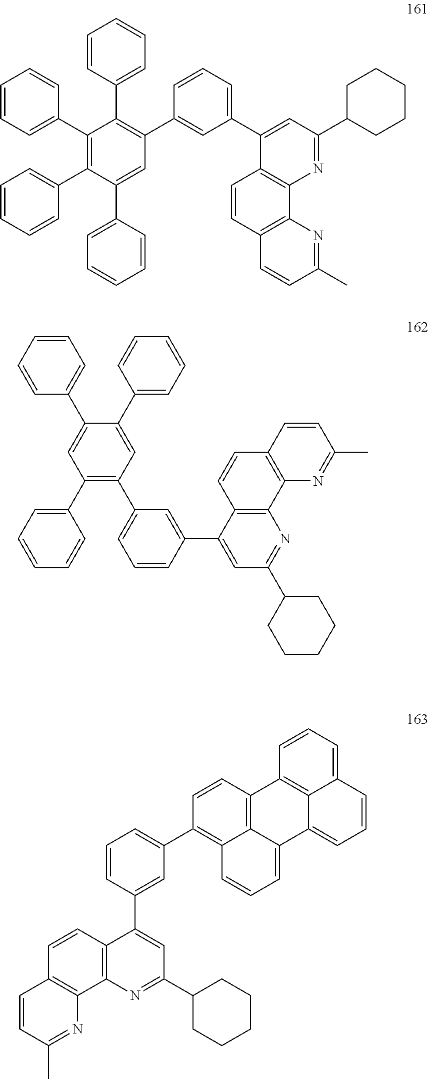 [SMILES: Cc1ccc2ccc3c(-c4cccc(-c5cc(-c6ccccc6)c(-c6ccccc6)c(-c6ccccc6)c5-c5ccccc5)c4)cc(C4CCCCC4)nc3c2n1 ; Cc1ccc2ccc3c(-c4cccc(-c5cc(-c6ccccc6)c(-c6ccccc6)cc5-c5ccccc5)c4)cc(C4CCCCC4)nc3c2n1 ; Cc1ccc2ccc3c(-c4cccc(-c5ccc6c7cccc8cccc(c9cccc5c96)c87)c4)cc(C4CCCCC4)nc3c2n1] 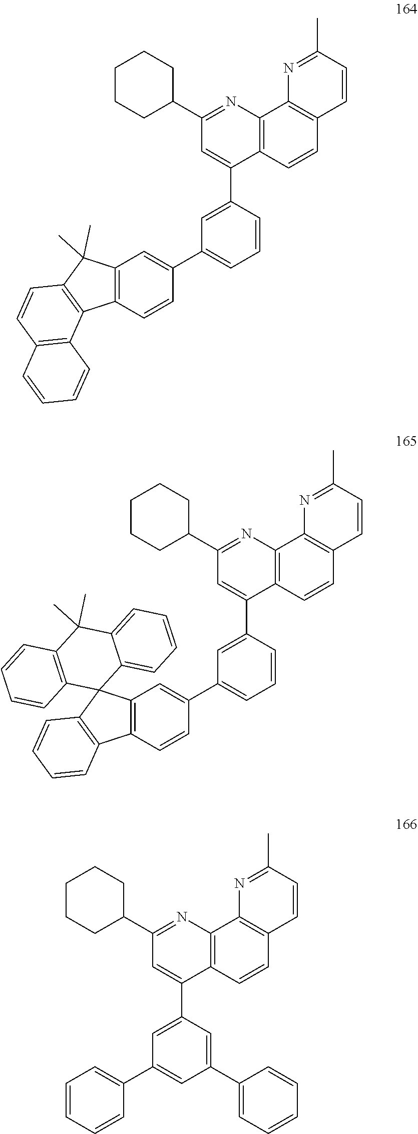 [SMILES: Cc1ccc2ccc3c(-c4cccc(-c5ccc6c(c5)C(C)(C)c5ccc7ccccc7c5-6)c4)cc(C4CCCCC4)nc3c2n1 ; Cc1ccc2ccc3c(-c4cccc(-c5ccc6c(c5)C5(c7ccccc7-6)c6ccccc6C(C)(C)c6ccccc65)c4)cc(C4CCCCC4)nc3c2n1 ; Cc1ccc2ccc3c(-c4cc(-c5ccccc5)cc(-c5ccccc5)c4)cc(C4CCCCC4)nc3c2n1] 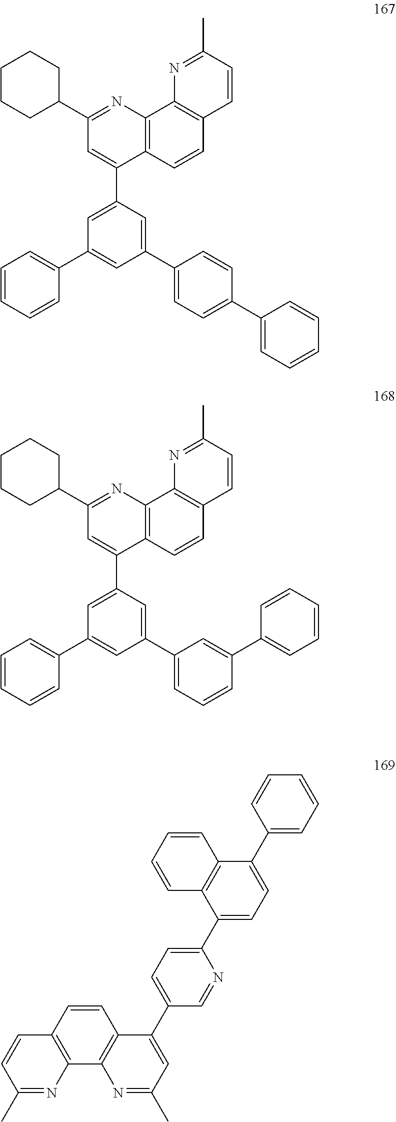 [SMILES: Cc1ccc2ccc3c(-c4cc(-c5ccccc5)cc(-c5ccc(-c6ccccc6)cc5)c4)cc(C4CCCCC4)nc3c2n1 ; Cc1ccc2ccc3c(-c4cc(-c5ccccc5)cc(-c5cccc(-c6ccccc6)c5)c4)cc(C4CCCCC4)nc3c2n1 ; Cc1ccc2ccc3c(-c4ccc(-c5ccc(-c6ccccc6)c6ccccc56)nc4)cc(C)nc3c2n1] 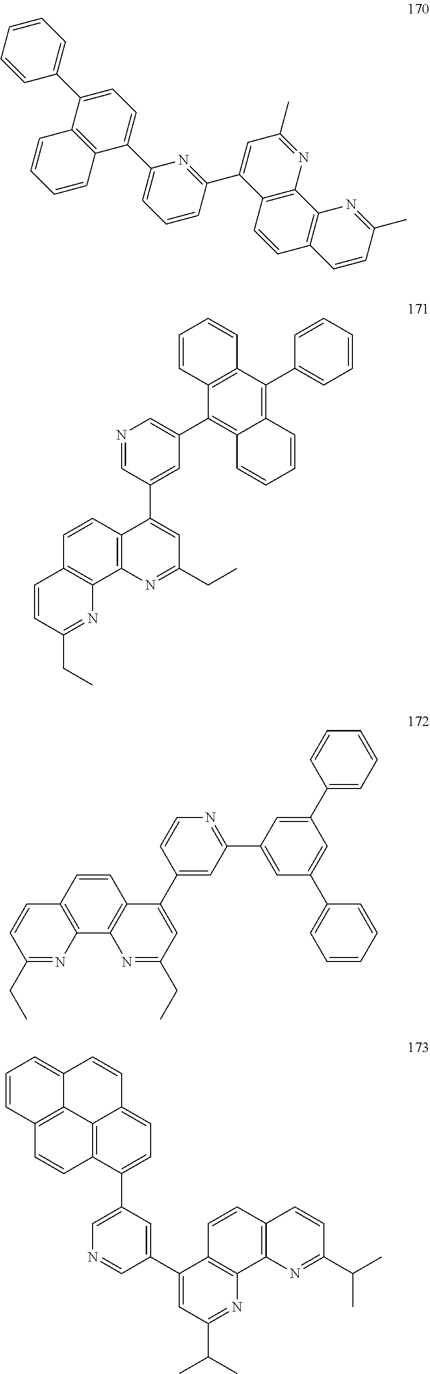 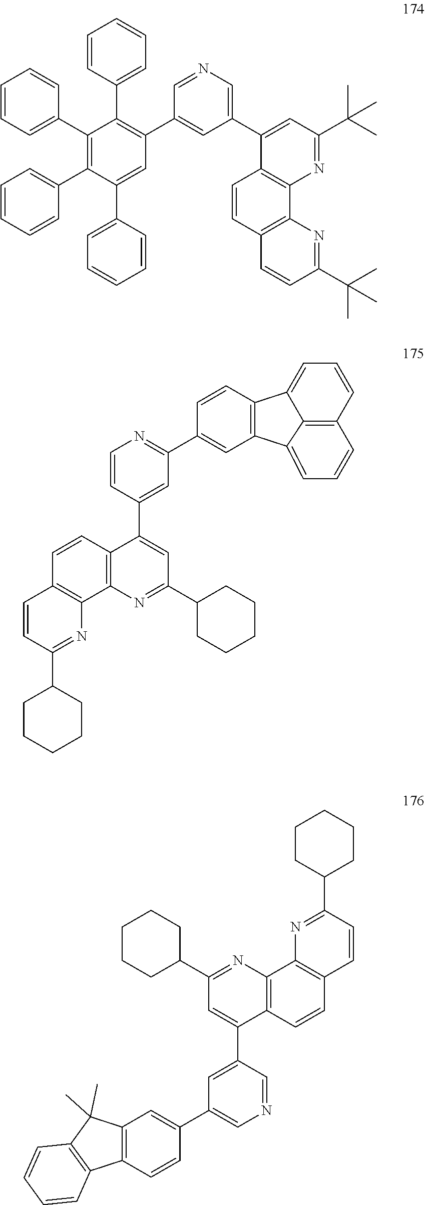 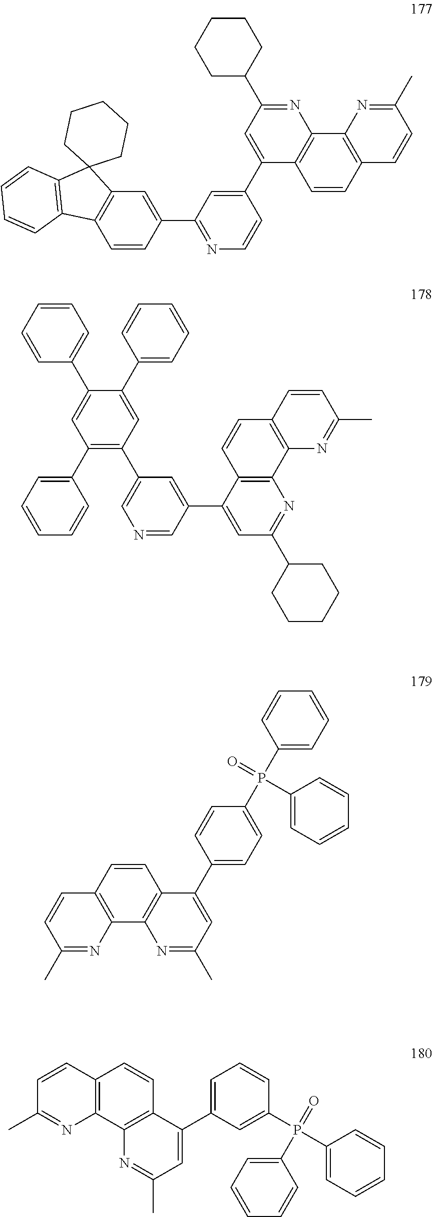 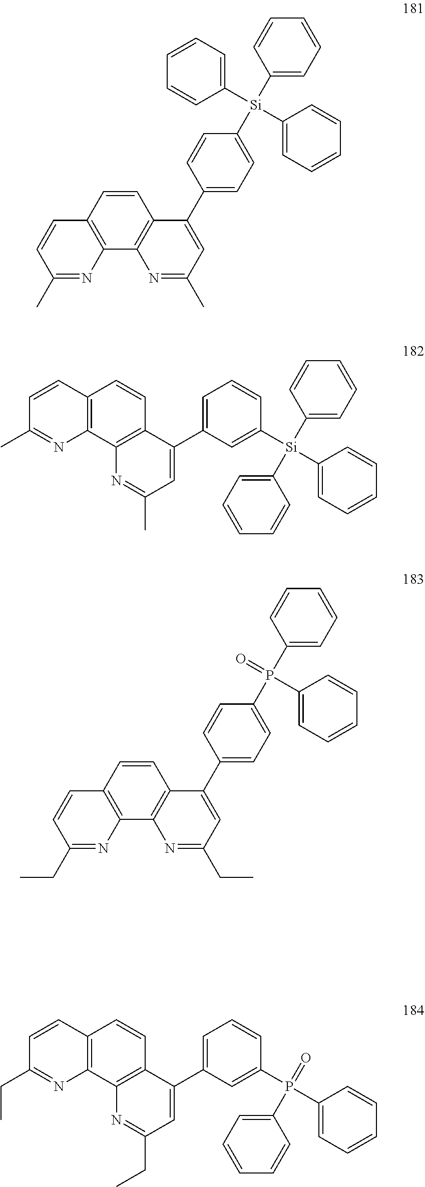 [SMILES: Cc1ccc2ccc3c(-c4ccc([Si](c5ccccc5)(c5ccccc5)c5ccccc5)cc4)cc(C)nc3c2n1 ; Cc1ccc2ccc3c(-c4cccc([Si](c5ccccc5)(c5ccccc5)c5ccccc5)c4)cc(C)nc3c2n1 ; CCc1ccc2ccc3c(-c4ccc(P(=O)(c5ccccc5)c5ccccc5)cc4)cc(CC)nc3c2n1 ; CCc1ccc2ccc3c(-c4cccc(P(=O)(c5ccccc5)c5ccccc5)c4)cc(CC)nc3c2n1] 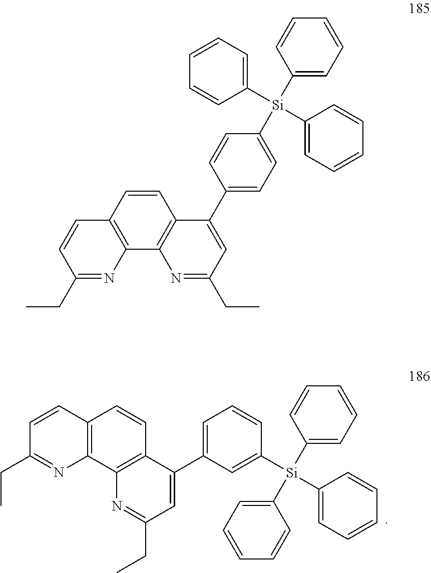 [SMILES: CCc1ccc2ccc3c(-c4ccc([Si](c5ccccc5)(c5ccccc5)c5ccccc5)cc4)cc(CC)nc3c2n1 ; CCc1ccc2ccc3c(-c4cccc([Si](c5ccccc5)(c5ccccc5)c5ccccc5)c4)cc(CC)nc3c2n1]
